(19) United States
(12) Patent Application Publication
(10) Pub. No.: US 2007/0219347 A1
(43) Pub. Date: Sep. 20, 2007

(54) Neisseria genomic sequences and methods of their use

(76) Inventors: Claire Marie Fraser, Potomac, MD (US); Erin Kathleen Hickey, Palatine, IL (US); Jeremy D. Peterson, Arlington, VA (US); Herve Tettelin, Gaithersburg, MD (US); J. Craig Venter, Potomac, MD (US); Vega Masignani, Siena (IT); Cesira Galeotti, Siena (IT); Marirosa Mora, Siena (IT); Giulio Ratti, Siena (IT); Maria Scarselli, Siena (IT); Vincenzo Scarlato, Colle di Val d'Elsa (IT); Rino Rappuoli, Siena (IT); Mariagrazia Pizza, Siena (IT); Guido Grandi, Milan (IT)

Correspondence Address: NOVARTIS VACCINES AND DIAGNOSTICS INC., CORPORATE INTELLECTUAL PROPERTY R338, P.O. BOX 8097, Emeryville, CA, 94662-8097, US

(21) Appl. No.: 11/711,740

(22) Filed: Feb. 26, 2007

Related U.S. Application Data
(62) Division of application No. 10/018,470, filed on Nov. 21, 2002.
(60) Provisional application No. 60/132,068, filed on Apr. 30, 1999.

(30) Foreign Application Priority Data
Feb. 28, 2000 (GB) 0004695.3
Oct. 8, 1999 (US) PCT/US99/23573

(51) Int. Cl. C07K 14/22; C07H 21/04; C07K 16/12; C07K 4/04; C07K 7/00

(52) U.S. Cl. 530/324; 530/325; 530/326; 530/327; 530/328; 530/329; 530/350; 530/387.1; 536/23.1

(57) ABSTRACT

The invention provides methods of obtaining immunogenic proteins from genomic sequences including Neisseria, including the amino acid sequences and the corresponding nucleotide sequences, as well as the genomic sequence of Neisseria meningitidis B. The proteins so obtained are useful antigens for vaccines, immunogenic compositions, and/or diagnostics

### RELATED APPLICATIONS

This application is a divisional of application Ser. No. 10/018,470 ('470), which is the U.S. National Phase of PCT/US00/05928, filed 8 Mar. 2000, which claims benefit to provisional U.S. application Ser. No. 60/132,068, filed 30 Apr. 1999; and priority to PCT/US99/23573, filed 8 Oct. 1999; and Great Britain application serial no. GB-0004695.3, filed 28 Feb. 2000. The above applications are herein incorporated in their entirety by reference.

### INCORPORATION BY REFERENCE OF MATERIAL ON COMPACT DISC

This application hereby incorporates by reference a sequence listing on a compact disc (CD-R) accompanied by two (2) copies (labeled Copy 1 and Copy 2) formatted from an IBM-PC Compatible computer, compatible with MS-Windows. The compact disc contains the following file: SEQLIST_PP000365.352.doc, containing 5 MB, created on Jan. 11, 2007.

This application hereby incorporates by reference Appendix A and Appendix B on a compact disc (CD-R) in duplicate formatted from an IBM-PC Compatible computer, compatible with MS-Windows. The compact disc contains the following files: AppendixA_PP000365.352.doc, containing 3 MB, created on Feb. 8, 2007; and AppendixB_PP000365.352.doc, containing 252 KB, created on Feb. 8, 2007.

### FIELD OF INVENTION

This invention relates to methods of obtaining antigens and immunogens, the antigens and immunogens so obtained, and nucleic acids from the bacterial species: Neisseria meningitidis. In particular, it relates to genomic sequences from the bacterium; more particularly its “B” serogroup.

### BACKGROUND

Neisseria meningitidis is a non-motile, gram negative diplococcus human pathogen. It colonizes the pharynx, causing meningitis and, occasionally, septicaemia in the absence of meningitis. It is closely related to N. gonorrhoea, although one feature that clearly differentiates meningococcus from gonococcus is the presence of a polysaccharide capsule that is present in all pathogenic meningococci.

N. meningitidis causes both endemic and epidemic disease. In the United States the attack rate is 0.6-1 per 100,000 persons per year, and it can be much greater during outbreaks. (see Lieberman et al. (1996) Safety and Immunogenicity of a Serogroups A/C Neisseria meningitidis Oligosaccharide-Protein Conjugate Vaccine in Young Children. JAMA 275(19):1499-1503; Schuchat et al (1997) Bacterial Meningitis in the United States in 1995. N Engl J Med 337(14):970-976). In developing countries, endemic disease rates are much higher and during epidemics incidence rates can reach 500 cases per 100,000 persons per year. Mortality is extremely high, at 10-20% in the United States, and much higher in developing countries. Following the introduction of the conjugate vaccine against Haemophilus influenzae, N. meningitidis is the major cause of bacterial meningitis at all ages in the United States (Schuchat et al (1997) supra).

Based on the organism's capsular polysaccharide, 12 serogroups of N. meningitidis have been identified. Group A is the pathogen most often implicated in epidemic disease in sub-Saharan Africa. Serogroups B and C are responsible for the vast majority of cases in the United States and in most developed countries. Serogroups W135 and Y are responsible for the rest of the cases in the United States and developed countries. The meningococcal vaccine currently in use is a tetravalent polysaccharide vaccine composed of serogroups A, C, Y and W135. Although efficacious in adolescents and adults, it induces a poor immune response and short duration of protection, and cannot be used in infants (e.g., Morbidity and Mortality weekly report, Vol. 46, No. RR-5 (1997)). This is because polysaccharides are T-cell independent antigens that induce a weak immune response that cannot be boosted by repeated immunization. Following the success of the vaccination against H. influenzae, conjugate vaccines against serogroups A and C have been developed and are at the final stage of clinical testing (Zollinger W D “New and Improved Vaccines Against Meningococcal Disease”. In: New Generation Vaccines, supra, pp. 469-488; Lieberman et al (1996) supra; Costantino et al (1992) Development and phase I clinical testing of a conjugate vaccine against meningococcus A (menA) and C (menC) (Vaccine 10:691-698)).

Meningococcus B (MenB) remains a problem, however. This serotype currently is responsible for approximately 50% of total meningitis in the United States, Europe, and South America. The polysaccharide approach cannot be used because the MenB capsular polysaccharide is a polymer of α(2-8)-linked N-acetyl neuraminic acid that is also present in mammalian tissue. This results in tolerance to the antigen; indeed, if an immune response were elicited, it would be anti-self, and therefore undesirable. In order to avoid induction of autoimmunity and to induce a protective immune response, the capsular polysaccharide has, for instance, been chemically modified substituting the N-acetyl groups with N-propionyl groups, leaving the specific antigenicity unaltered (Romero & Outschoorn (1994) Current status of Meningococcal group B vaccine candidates: capsular or non-capsular? Clin Microbiol Rev 7(4):559-575).

Alternative approaches to MenB vaccines have used complex mixtures of outer membrane proteins (OMPs), containing either the OMPs alone, or OMPs enriched in porins, or deleted of the class 4 OMPs that are believed to induce antibodies that block bactericidal activity. This approach produces vaccines that are not well characterized. They are able to protect against the homologous strain, but are not effective at large where there are many antigenic variants of the outer membrane proteins. To overcome the antigenic variability, multivalent vaccines containing up to nine different porins have been constructed (e.g., Poolman J T (1992) Development of a meningococcal vaccine. Infect. Agents Dis. 4:13-28). Additional proteins to be used in outer membrane vaccines have been the opa and opc proteins, but none of these approaches have been able to overcome the antigenic variability (e.g., Ala'Aldeen & Borriello (1996) The meningococcal transferrin-binding proteins 1 and 2 are both surface exposed and generate bactericidal antibodies capable of killing homologous and heterologous strains. Vaccine 14(1):49-53).

A certain amount of sequence data is available for meningococcal and gonococcal genes and proteins (e.g., EP-A-0467714, WO96/29412), but this is by no means complete. The provision of further sequences could provide an opportunity to identify secreted or surface-exposed proteins that are presumed targets for the immune system and which are not antigenically variable or at least are more antigenically conserved than other and more variable regions. Thus, those antigenic sequences that are more highly conserved are preferred sequences. Those sequences specific to Neisseria meningitidis or Neisseria gonorrhoeae that are more highly conserved are further preferred sequences. For instance, some of the identified proteins could be components of efficacious vaccines against meningococcus B, some could be components of vaccines against all meningococcal serotypes, and others could be components of vaccines against all pathogenic Neisseriae. The identification of sequences from the bacterium will also facilitate the production of biological probes, particularly organism-specific probes.

It is thus an object of the invention is to provide Neisserial DNA sequences which (1) encode proteins predicted and/or shown to be antigenic or immunogenic, (2) can be used as probes or amplification primers, and (3) can be analyzed by bioinforrnatics.

### BRIEF DESCRIPTION OF THE DRAWINGS

FIGS. 1A-1E illustrate the products of protein expression and purification of the predicted ORF 919 as cloned and expressed in E. coli.

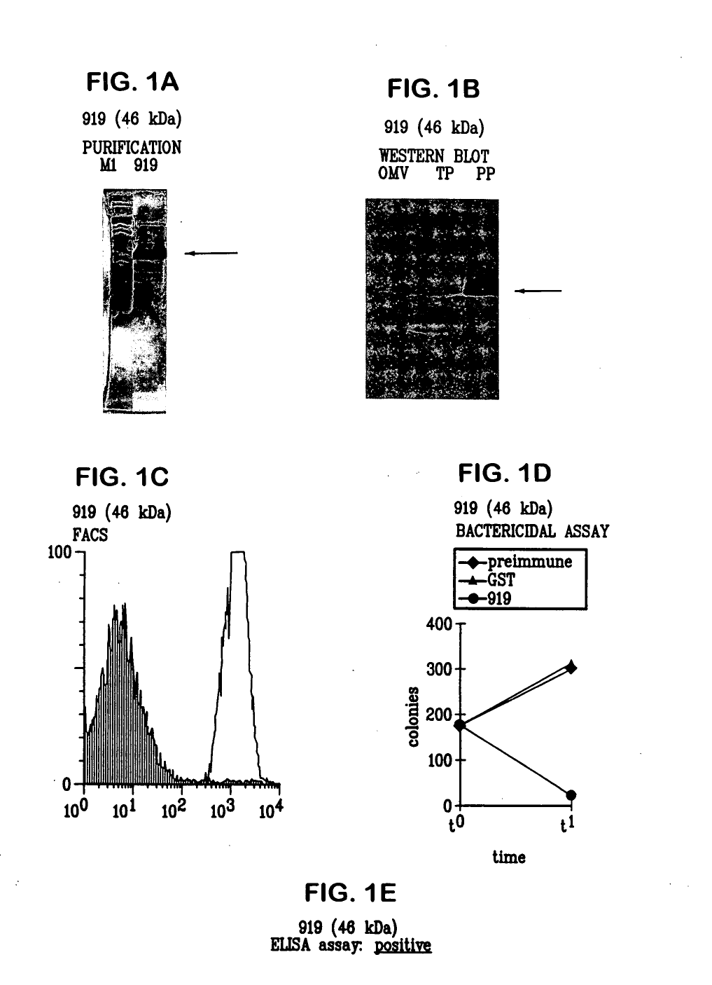

FIGS. 2A-2E illustrate the products of protein expression and purification of the predicted ORF 279 as cloned and expressed in E. coli.

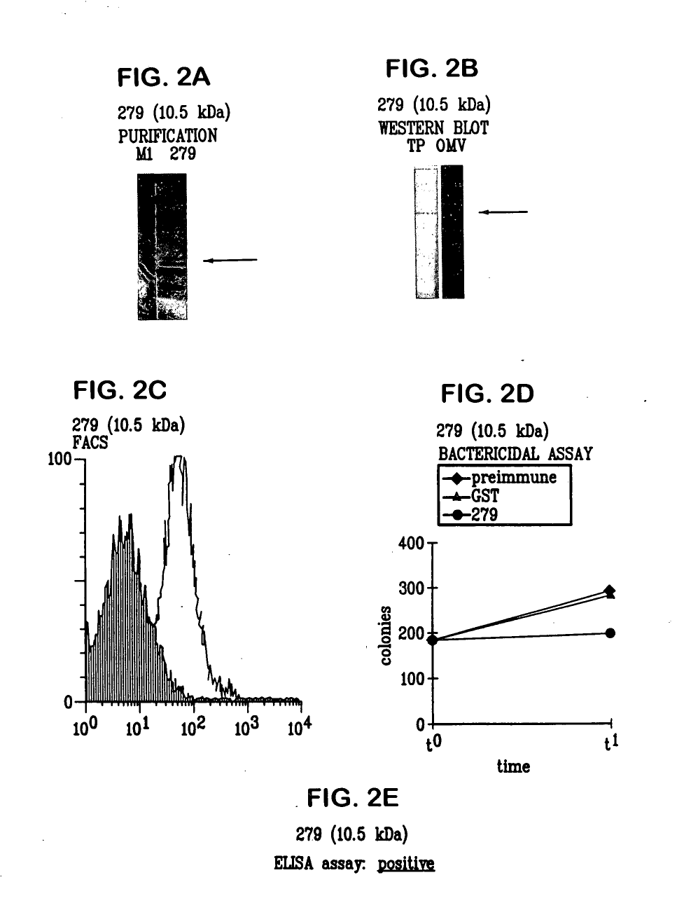

FIGS. 3A-3E illustrate the products of protein expression and purification of the predicted ORF 576-1 as cloned and expressed in E. coli.

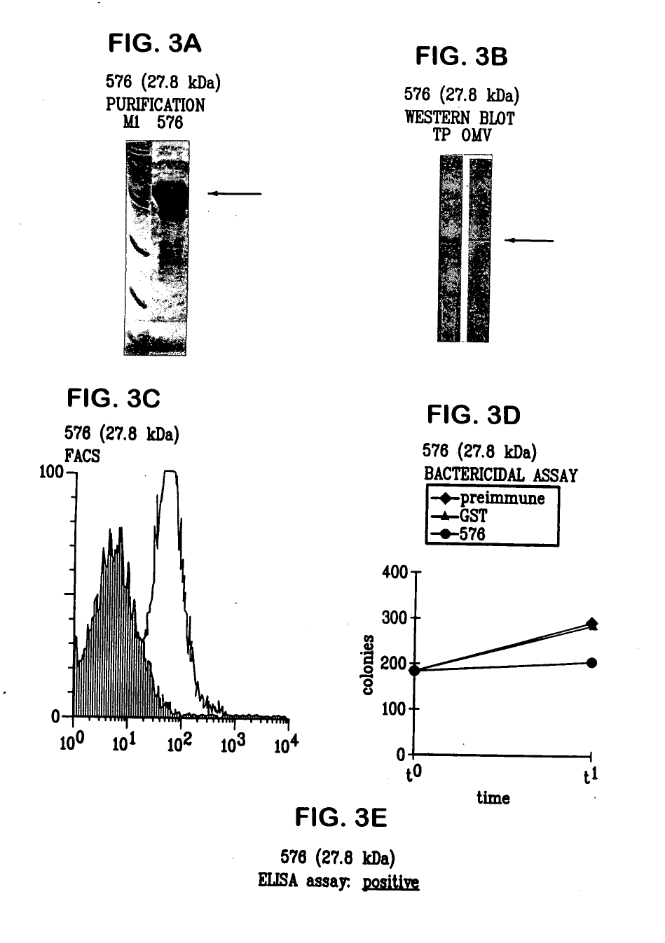

FIGS. 4A-4E illustrate the products of protein expression and purification of the predicted ORF 519-1 as cloned and expressed in E. coli.

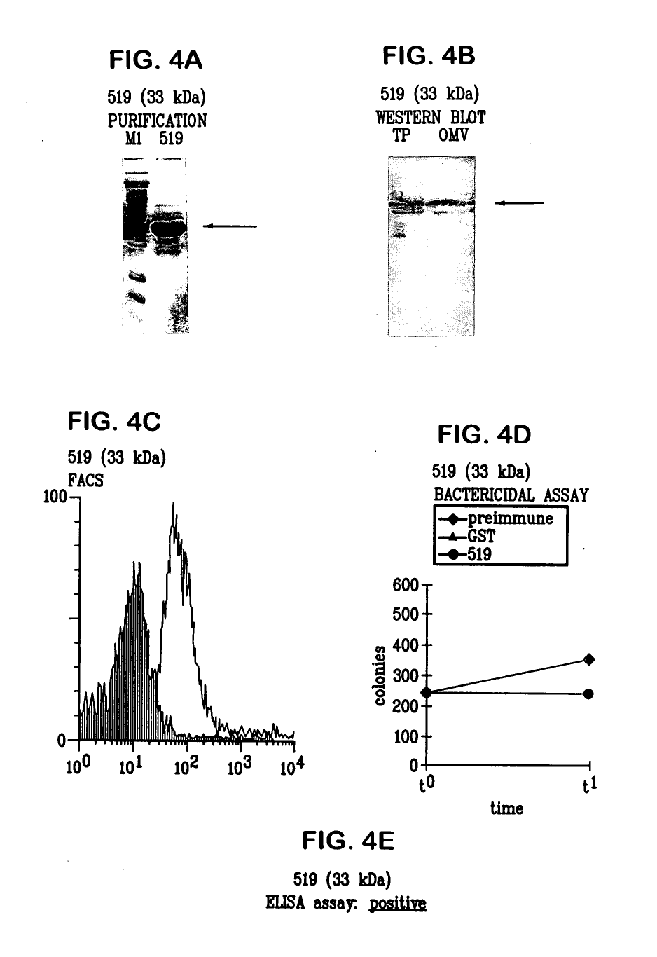

FIGS. 5A-5E illustrate the products of protein expression and purification of the predicted ORF 121-1 as cloned and expressed in E. coli.

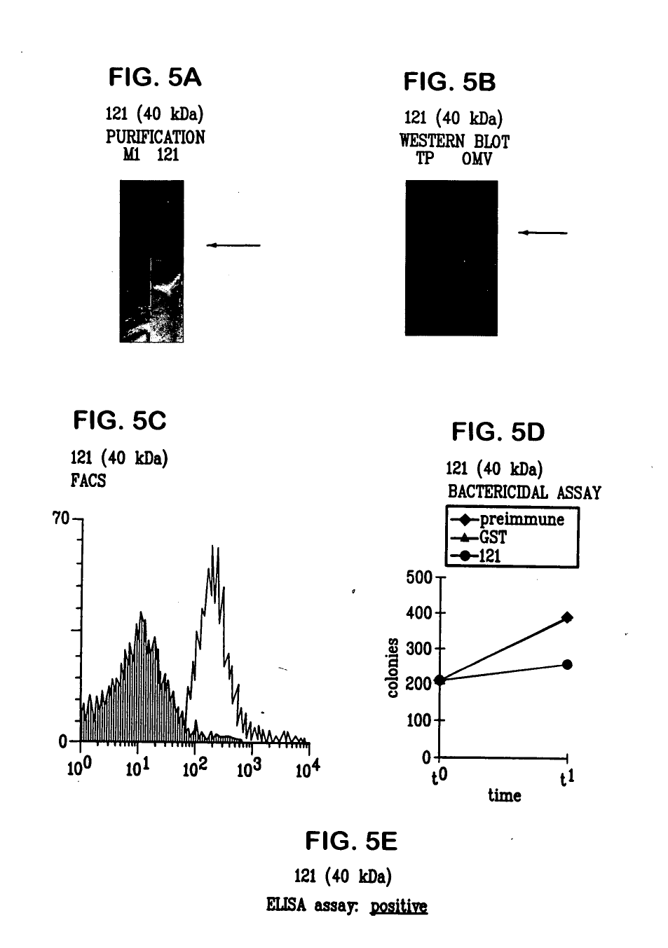

FIGS. 6A-6E illustrate the products of protein expression and purification of the predicted ORF 128-1 as cloned and expressed in E. coli.

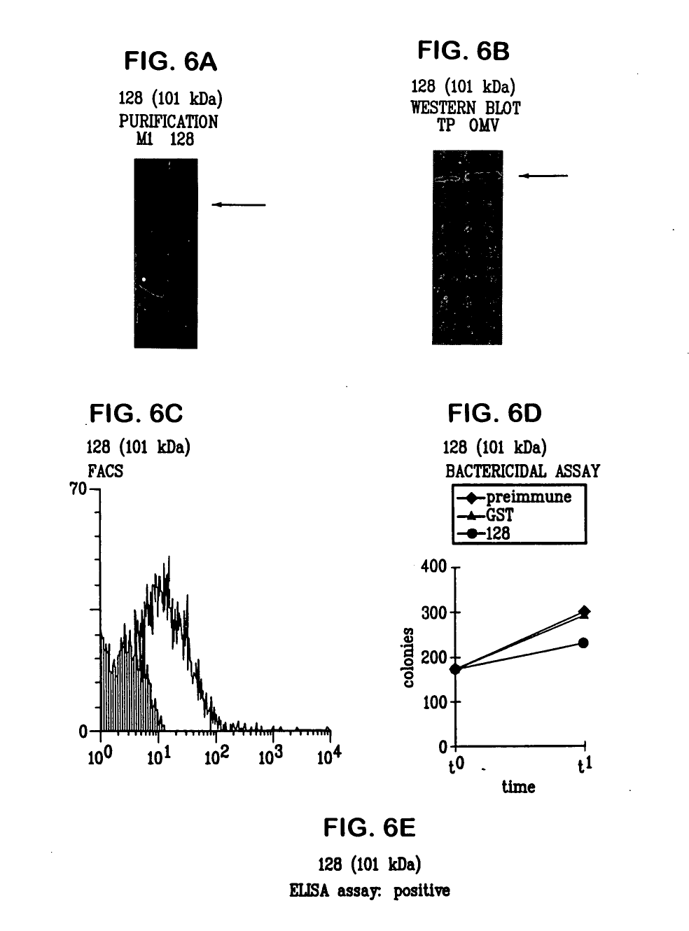

FIGS. 7A-7E illustrate illustrates the products of protein expression and purification of the predicted ORF 206 as cloned and expressed in E. coli.

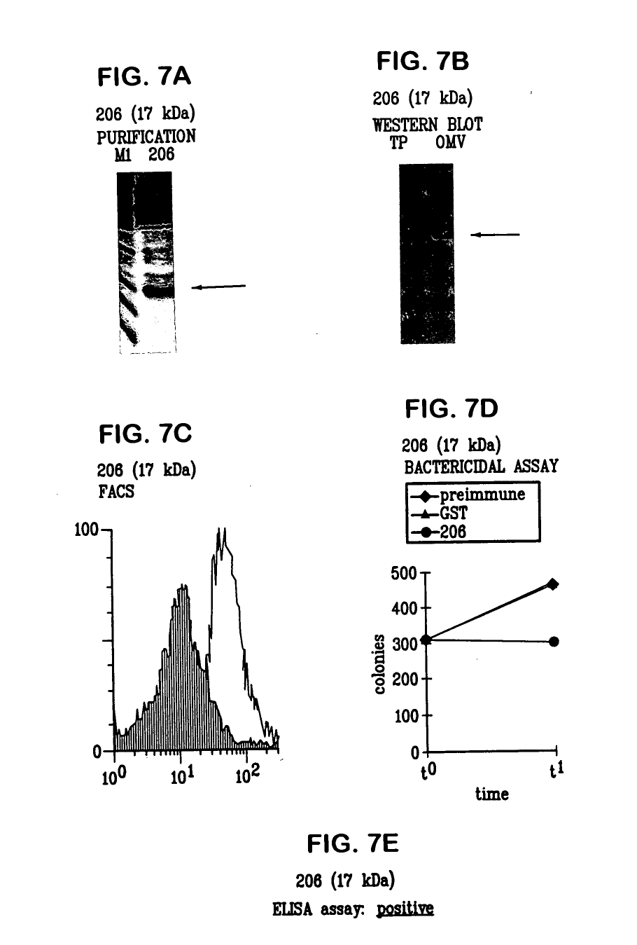

FIGS. 8A-8D illustrate the products of protein expression and purification of the predicted ORF 287 as cloned and expressed in E. coli.

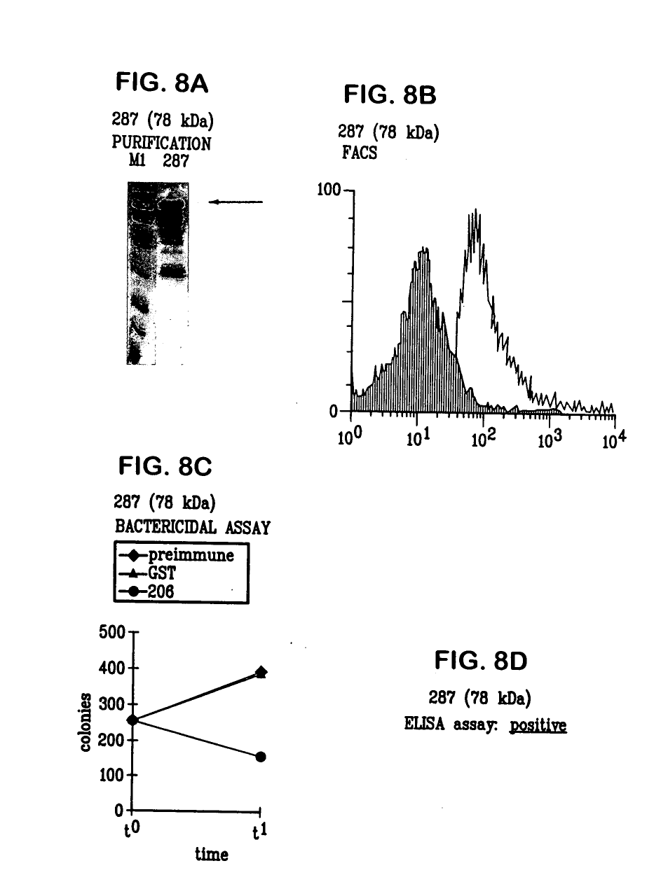

FIGS. 9A-9E illustrate the products of protein expression and purification of the predicted ORF 406 as cloned and expressed in E. coli.

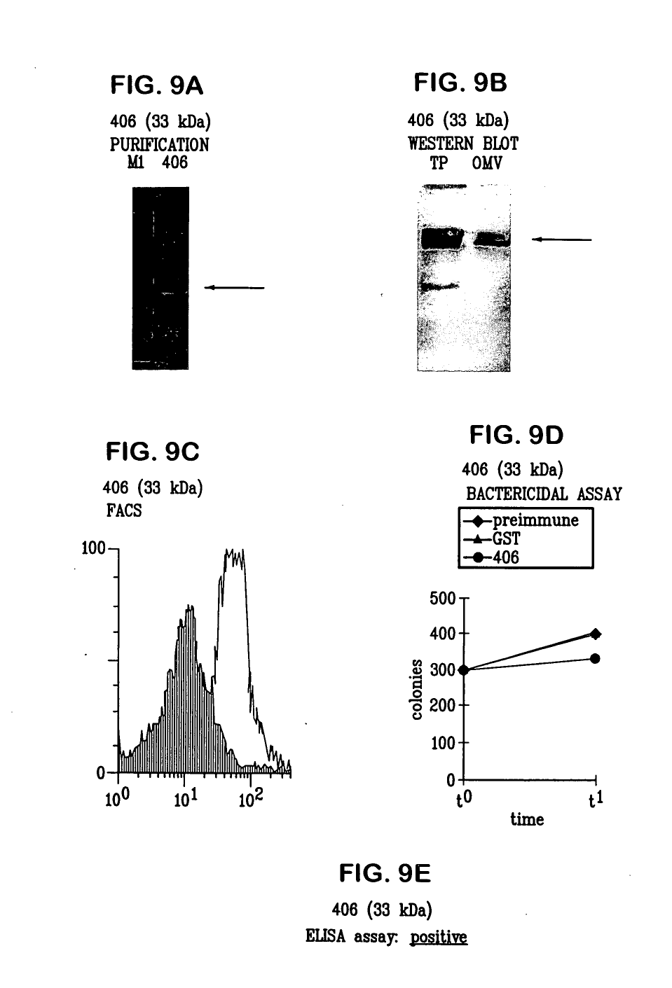

FIG. 10 illustrates the hydrophilicity plot, antigenic index and AMPHI regions of the products of protein expression the predicted ORF 919 as cloned and expressed in E. coli.

FIG. 11 illustrates the hydrophilicity plot, antigenic index and AMPHI regions of the products of protein expression the predicted ORF 279 as cloned and expressed in E. coli.

FIG. 12 illustrates the hydrophilicity plot, antigenic index and AMPHI regions of the products of protein expression the predicted ORF 576-1 as cloned and expressed in E. coli.

FIG. 13 illustrates the hydrophilicity plot, antigenic index and AMPHI regions of the products of protein expression the predicted ORF 519-1 as cloned and expressed in E. coli.

FIG. 14 illustrates the hydrophilicity plot, antigenic index and AMPHI regions of the products of protein expression the predicted ORF 121-1 as cloned and expressed in E. coli.

FIG. 15 illustrates the hydrophilicity plot, antigenic index and AMPHI regions of the products of protein expression the predicted ORF 128-1 as cloned and expressed in E. coli.

FIG. 16 illustrates the hydrophilicity plot, antigenic index and AMPHI regions of the products of protein expression the predicted ORF 206 as cloned and expressed in E. coli.

FIG. 17 illustrates the hydrophilicity plot, antigenic index and AMPHI regions of the products of protein expression the predicted ORF 287 as cloned and expressed in E. coli.

FIG. 18 illustrates the hydrophilicity plot, antigenic index and AMPHI regions of the products of protein expression the predicted ORF 406 as cloned and expressed in E. coli.

### THE INVENTION

The first complete sequence of the genome of N. meningitidis was disclosed as 961 partial contiguous nucleotide sequences, shown as SEQ ID NOs:1-961 of co-owned PCT/US99/23573 (the '573 application), filed 8 Oct. 1999 (to be published April 2000). A single sequence full length genome of N. meningitidis was also disclosed as SEQ ID NO. 1068 of the '573 application. The invention is based on a full length genome of N. meningitidis which appears as SEQ ID NO.1 in the present application as Appendix A hereto. The 961 sequences of the '573 application represent substantially the whole genome of serotype B of N. meningitidis (>99.98%). There is partial overlap between some of the 961 contiguous sequences (“contigs”) shown in the 961 sequences, which overlap was used to construct the single full length sequence shown in SEQ ID NO. 1 in Appendix A hereto, using the TIGR Assembler [G. S. Sutton et al., TIGR Assembler: A New Tool for Assembling Large Shotgun Sequencing Projects, Genome Science and Technology, 1:9-19 (1995)]. Some of the nucleotides in the contigs had been previously released. (available at 11ftp.tigr.org/pub/data/n_meningitidis on the world-wide web or “WWW”). The coordinates of the 2508 released sequences in the present contigs are presented in Appendix A of the '573 application. These data include the contig number (or i.d.) as presented in the first column; the name of the sequence as found on WWW is in the second column; with the coordinates of the contigs in the third and fourth columns, respectively. The sequences of certain MenB ORFs presented in Appendix B of the '573 application feature in International Patent Application filed by Chiron SpA on Oct. 9, 1998 (PCT/IB98/01665) and Jan. 14, 1999 (PCT/IB99/00103) respectively. Appendix B hereto provides a listing of 2158 open reading frames contained within the full length sequence found in SEQ ID NO. 1 in Appendix A hereto. The information set forth in Appendix B hereto includes the “NMB” name of the sequence, the putative translation product, and the beginning and ending nucleotide positions within SEQ ID NO. 1 which comprise the open reading frames. These open reading frames are referred to herein as the “NMB open reading frames”.

In a first aspect, the invention provides nucleic acid including the N. meningitidis nucleotide sequence shown in SEQ ID NO. 1 in Appendix A hereto. It also provides nucleic acid comprising sequences having sequence identity to the nucleotide sequence disclosed herein. Depending on the particular sequence, the degree of sequence identity is preferably greater than 50% (e.g., 60%, 70%, 80%, 90%, 95%, 99% or more). These sequences include, for instance, mutants and allelic variants. The degree of sequence identity cited herein is determined across the length of the sequence determined by the Smith-Waterman homology search algorithm as implemented in MPSRCH program (Oxford Molecular) using an affine gap search with the following parameters: gap open penalty 12, gap extension penalty 1.

The invention also provides nucleic acid including a fragment of one or more of the nucleotide sequences set out herein, including the NMB open reading frames shown in Appendix B hereto. The fragment should comprise at least n consecutive nucleotides from the sequences and, depending on the particular sequence, n is 10 or more (e.g., 11, 12, 13, 14, 15, 16, 17, 18, 19, 20, 21, 22, 23, 24, 25, 30, 35, 40, 45, 50, 60, 75, 100 or more). Preferably, the fragment is unique to the genome of N. meningitidis, that is to say it is not present in the genome of another organism. More preferably, the fragment is unique to the genome of strain B of N. meningitidis. The invention also provides nucleic acid that hybridizes to those provided herein. Conditions for hybridizing are disclosed herein.

The invention also provides nucleic acid including sequences complementary to those described above (e.g., for antisense, for probes, or for amplification primers).

Nucleic acid according to the invention can, of course, be prepared in many ways (e.g., by chemical synthesis, from DNA libraries, from the organism itself, etc.) and can take various forms (e.g., single-stranded, double-stranded, vectors, probes, primers, etc.). The term “nucleic acid” includes DNA and RNA, and also their analogs, such as those containing modified backbones, and also peptide nucleic acid (PNA) etc.

It will be appreciated that, as SEQ ID NOs:1-961 of the '573 application represent the substantially complete genome of the organism, with partial overlap, references to SEQ ID NOs:1-961 of the '573 application include within their scope references to the complete genomic sequence, that is, SEQ ID NO. 1 hereof. For example, where two SEQ ID NOs overlap, the invention encompasses the single sequence which is formed by assembling the two overlapping sequences, which full sequence will be found in SEQ ID NO. 1 hereof. Thus, for instance, a nucleotide sequence which bridges two SEQ ID NOs but is not present in its entirety in either SEQ ID NO is still within the scope of the invention. Such a sequence will be present in its entirety in the single full length sequence of SEQ ID NO. 1 of the present application.

The invention also provides vectors including nucleotide sequences of the invention (e.g., expression vectors, sequencing vectors, cloning vectors, etc.) and host cells transformed with such vectors.

According to a further aspect, the invention provides a protein including an amino acid sequence encoded within a N. meningitidis nucleotide sequence set out herein. It also provides proteins comprising sequences having sequence identity to those proteins. Depending on the particular sequence, the degree of sequence identity is preferably greater than 50% (e.g., 60%, 70%, 80%, 90%, 95%, 99% or more). Sequence identity is determined as above disclosed. These homologous proteins include mutants and allelic variants, encoded within the N. meningitidis nucleotide sequence set out herein.

The invention further provides proteins including fragments of an amino acid sequence encoded within a N. meningitidis nucleotide sequence set out in the sequence listing. The fragments should comprise at least n consecutive amino acids from the sequences and, depending on the particular sequence, n is 7 or more (e.g., 8, 10, 12, 14, 16, 18, 20 or more). Preferably the fragments comprise an epitope from the sequence.

The proteins of the invention can, of course, be prepared by various means (e.g., recombinant expression, purification from cell culture, chemical synthesis, etc.) and in various forms (e.g. native, fusions etc.). They are preferably prepared in substantially isolated form (i.e., substantially free from other N. meningitidis host cell proteins).

Various tests can be used to assess the in vivo immunogenicity of the proteins of the invention. For example, the proteins can be expressed recombinantly or chemically synthesized and used to screen patient sera by immunoblot. A positive reaction between the protein and patient serum indicates that the patient has previously mounted an immune response to the protein in question; i.e., the protein is an immunogen. This method can also be used to identify immunodominant proteins.

The invention also provides nucleic acid encoding a protein of the invention.

In a further aspect, the invention provides a computer, a computer memory, a computer storage medium (e.g., floppy disk, fixed disk, CD-ROM, etc.), and/or a computer database containing the nucleotide sequence of nucleic acid according to the invention. Preferably, it contains one or more of the N. meningitidis nucleotide sequences set out herein.

This may be used in the analysis of the N. meningitidis nucleotide sequences set out herein. For instance, it may be used in a search to identify open reading frames (ORFs) or coding sequences within the sequences.

In a further aspect, the invention provides a method for identifying an amino acid sequence, comprising the step of searching for putative open reading frames or protein-coding sequences within a N. meningitidis nucleotide sequence set out herein. Similarly, the invention provides the use of a N. meningitidis nucleotide sequence set out herein in a search for putative open reading frames or protein-coding sequences.

Open-reading frame or protein-coding sequence analysis is generally performed on a computer using standard bioinformatic techniques. Typical algorithms or program used in the analysis include ORFFINDER (NCBI), GENMARK [Borodovsky & McIninch (1993) Computers Chem 17:122-133], and GLIMMER [Salzberg et al. (1998) Nucl Acids Res 26:544-548].

A search for an open reading frame or protein-coding sequence may comprise the steps of searching a N. meningitidis nucleotide sequence set out herein for an initiation codon and searching the upstream sequence for an in-frame termination codon. The intervening codons represent a putative protein-coding sequence. Typically, all six possible reading frames of a sequence will be searched.

An amino acid sequence identified in this way can be expressed using any suitable system to give a protein. This protein can be used to raise antibodies which recognize epitopes within the identified amino acid sequence. These antibodies can be used to screen N. meningitidis to detect the presence of a protein comprising the identified amino acid sequence.

Furthermore, once an ORF or protein-coding sequence is identified, the sequence can be compared with sequence databases. Sequence analysis tools can be found at NCBI (available at www.ncbi.nlm.nih.gov) e.g., the algorithms BLAST, BLAST2, BLASTn, BLASTp, tBLASTn, BLASTx, & tBLASTx [see also Altschul et al. (1997) Gapped BLAST and PSI-BLAST: new generation of protein database search programs. Nucleic Acids Research 25:2289-3402]. Suitable databases for comparison include the nonredundant GenBank, EMBL, DDBJ and PDB sequences, and the nonredundant GenBank CDS translations, PDB, SwissProt, Spupdate and PIR sequences. This comparison may give an indication of the function of a protein.

Hydrophobic domains in an amino acid sequence can be predicted using algorithms such as those based on the statistical studies of Esposti et al. [Critical evaluation of the hydropathy of membrane proteins (1990) Eur J Biochem 190:207-219]. Hydrophobic domains represent potential transmembrane regions or hydrophobic leader sequences, which suggest that the proteins may be secreted or be surface-located. These properties are typically representative of good immunogens.

Similarly, transmembrane domains or leader sequences can be predicted using the PSORT algorithm (available at www.psort.nibb.ac.jp), and functional domains can be predicted using the MOTIFS program (GCG Wisconsin & PROSITE).

The invention also provides nucleic acid including an open reading frame or protein-coding sequence present in a N. meningitidis nucleotide sequence set out herein. Furthermore, the invention provides a protein including the amino acid sequence encoded by this open reading frame or protein-coding sequence.

According to a further aspect, the invention provides antibodies which bind to these proteins. These may be polyclonal or monoclonal and may be produced by any suitable means known to those skilled in the art.

The antibodies of the invention can be used in a variety of ways, e.g., for confirmation that a protein is expressed, or to confirm where a protein is expressed. Labeled antibody (e.g., fluorescent labeling for FACS) can be incubated with intact bacteria and the presence of label on the bacterial surface confirms the location of the protein, for instance.

According to a further aspect, the invention provides compositions including protein, antibody, and/or nucleic acid according to the invention. These compositions may be suitable as vaccines, as immunogenic compositions, or as diagnostic reagents.

The invention also provides nucleic acid, protein, or antibody according to the invention for use as medicaments (e.g., as vaccines) or as diagnostic reagents. It also provides the use of nucleic acid, protein, or antibody according to the invention in the manufacture of (I) a medicament for treating or preventing infection due to Neisserial bacteria (ii) a diagnostic reagent for detecting the presence of Neisserial bacteria or of antibodies raised against Neisserial bacteria. Said Neisserial bacteria may be any species or strain (such as N. gonorrhoeae) but are preferably N. meningitidis, especially strain A, strain B or strain C.

In still yet another aspect, the present invention provides for compositions including proteins, nucleic acid molecules, or antibodies. More preferable aspects of the present invention are drawn to immunogenic compositions of proteins. Further preferable aspects of the present invention contemplate pharmaceutical immunogenic compositions of proteins or vaccines and the use thereof in the manufacture of a medicament for the treatment or prevention of infection due to Neisserial bacteria, preferably infection of MenB.

The invention also provides a method of treating a patient, comprising administering to the patient a therapeutically effective amount of nucleic acid, protein, and/or antibody according to the invention.

According to further aspects, the invention provides various processes.

A process for producing proteins of the invention is provided, comprising the step of culturing a host cell according to the invention under conditions which induce protein expression. A process which may further include chemical synthesis of proteins and/or chemical synthesis (at least in part) of nucleotides.

A process for detecting polynucleotides of the invention is provided, comprising the steps of: (a) contacting a nucleic probe according to the invention with a biological sample under hybridizing conditions to form duplexes; and (b) detecting said duplexes.

A process for detecting proteins of the invention is provided, comprising the steps of: (a) contacting an antibody according to the invention with a biological sample under conditions suitable for the formation of an antibody-antigen complexes; and (b) detecting said complexes.

Another aspect of the present invention provides for a process for detecting antibodies that selectably bind to antigens or polypeptides or proteins specific to any species or strain of Neisserial bacteria and preferably to strains of N. gonorrhoeae but more preferably to strains of N. meningitidis, especially strain A, strain B or strain C, more preferably MenB, where the process comprises the steps of: (a) contacting antigen or polypeptide or protein according to the invention with a biological sample under conditions suitable for the formation of an antibody-antigen complexes; and (b) detecting said complexes.

Having now generally described the invention, the same will be more readily understood through reference to the following examples which are provided by way of illustration, and are not intended to be limiting of the present invention, unless specified.

Methodology—Summary of Standard Procedures and Techniques.

General

This invention provides Neisseria meningitidis MenB nucleotide sequences, amino acid sequences encoded therein. With these disclosed sequences, nucleic acid probe assays and expression cassettes and vectors can be produced. The proteins can also be chemically synthesized. The expression vectors can be transformed into host cells to produce proteins. The purified or isolated polypeptides can be used to produce antibodies to detect MenB proteins. Also, the host cells or extracts can be utilized for biological assays to isolate agonists or antagonists. In addition, with these sequences one can search to identify open reading frames and identify amino acid sequences. The proteins may also be used in immunogenic compositions and as vaccine components.

The practice of the present invention will employ, unless otherwise indicated, conventional techniques of molecular biology, microbiology, recombinant DNA, and immunology, which are within the skill of the art. Such techniques are explained fully in the literature e.g., Sambrook Molecular Cloning; A Laboratory Manual, Second Edition (1989); DNA Cloning, Volumes I and ii (D. N. Glover ed. 1985); Oligonucleotide Synthesis (M. J. Gait ed, 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Mayer and Walker, eds. (1987), Immunochemical Methods in Cell and Molecular Biology (Academic Press, London); Scopes, (1987) Protein Purification: Principles and Practice, Second Edition (Springer-Verlag, N.Y.), and Handbook of Experimental Immunology, Volumes I-IV (D. M. Weir and C. C. Blackwell eds 1986).

Standard abbreviations for nucleotides and amino acids are used in this specification.

All publications, patents, and patent applications cited herein are incorporated in full by reference.

Expression systems

The Neisseria MenB nucleotide sequences can be expressed in a variety of different expression systems; for example those used with mammalian cells, plant cells, baculoviruses, bacteria, and yeast.

i. Mammalian Systems

Mammalian expression systems are known in the art. A mammalian promoter is any DNA sequence capable of binding mammalian RNA polymerase and initiating the downstream (3′) transcription of a coding sequence (e.g., structural gene) into mRNA. A promoter will have a transcription initiating region, which is usually placed proximal to the 5′ end of the coding sequence, and a TATA box, usually located 25-30 base pairs (bp) upstream of the transcription initiation site. The TATA box is thought to direct RNA polymerase II to begin RNA synthesis at the correct site. A mammalian promoter will also contain an upstream promoter element, usually located within 100 to 200 bp upstream of the TATA box. An upstream promoter element determines the rate at which transcription is initiated and can act in either orientation (Sambrook et al. (1989) “Expression of Cloned Genes in Mammalian Cells.” In Molecular Cloning: A Laboratory Manual, 2nd ed.).

Mammalian viral genes are often highly expressed and have a broad host range; therefore sequences encoding mammalian viral genes provide particularly useful promoter sequences. Examples include the SV40 early promoter, mouse mammary tumor virus LTR promoter, adenovirus major late promoter (Ad MLP), and herpes simplex virus promoter. In addition, sequences derived from non-viral genes, such as the murine metallothionein gene, also provide useful promoter sequences. Expression may be either constitutive or regulated (inducible). Depending on the promoter selected, many promotes may be inducible using known substrates, such as the use of the mouse mammary tumor virus (MMTV) promoter with the glucocorticoid responsive element (GRE) that is induced by glucocorticoid in hormone-responsive transformed cells (see for example, U.S. Pat. No. 5,783,681).

The presence of an enhancer element (enhancer), combined with the promoter elements described above, will usually increase expression levels. An enhancer is a regulatory DNA sequence that can stimulate transcription up to 1000-fold when linked to homologous or heterologous promoters, with synthesis beginning at the normal RNA start site. Enhancers are also active when they are placed upstream or downstream from the transcription initiation site, in either normal or flipped orientation, or at a distance of more than 1000 nucleotides from the promoter (Maniatis et al. (1987) Science 236:1237; Alberts et al. (1989) Molecular Biology of the Cell, 2nd ed.). Enhancer elements derived from viruses may be particularly useful, because they usually have a broader host range. Examples include the SV40 early gene enhancer (Dijkema et al (1985) EMBO J. 4:761) and the enhancer/promoters derived from the long terminal repeat (LTR) of the Rous Sarcoma Virus (Gorman et al. (1982b) Proc. Natl. Acad. Sci. 79:6777) and from human cytomegalovirus (Boshart et al. (1985) Cell 41:521). Additionally, some enhancers are regulatable and become active only in the presence of an inducer, such as a hormone or metal ion (Sassone-Corsi and Borelli (1986) Trends Genet. 2:215; Maniatis et al. (1987) Science 236:1237).

A DNA molecule may be expressed intracellularly in mammalian cells. A promoter sequence may be directly linked with the DNA molecule, in which case the first amino acid at the N-terminus of the recombinant protein will always be a methionine, which is encoded by the ATG start codon. If desired, the N-terminus may be cleaved from the protein by in vitro incubation with cyanogen bromide.

Alternatively, foreign proteins can also be secreted from the cell into the growth media by creating chimeric DNA molecules that encode a fusion protein comprised of a leader sequence fragment that provides for secretion of the foreign protein in mammalian cells. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell. The adenovirus tripartite leader is an example of a leader sequence that provides for secretion of a foreign protein in mammalian cells.

Usually, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-transcriptional cleavage and polyadenylation (Birnstiel et al. (1985) Cell 41:349; Proudfoot and Whitelaw (1988) “Termination and 3′ end processing of eukaryotic RNA.” In Transcription and splicing (ed. B. D. Hames and D. M. Glover); Proudfoot (1989) Trends Biochem. Sci. 14:105). These sequences direct the transcription of an mRNA which can be translated into the polypeptide encoded by the DNA. Examples of transcription terminator/polyadenylation signals include those derived from SV40 (Sambrook et al (1989) “Expression of cloned genes in cultured mammalian cells.” In Molecular Cloning: A Laboratory Manual).

Usually, the above-described components, comprising a promoter, polyadenylation signal, and transcription termination sequence are put together into expression constructs. Enhancers, introns with functional splice donor and acceptor sites, and leader sequences may also be included in an expression construct, if desired. Expression constructs are often maintained in a replicon, such as an extrachromosomal element (e.g., plasmids) capable of stable maintenance in a host, such as mammalian cells or bacteria. Mammalian replication systems include those derived from animal viruses, which require trans-acting factors to replicate. For example, plasmids containing the replication systems of papovaviruses, such as SV40 (Gluzman (1981) Cell 23:175) or polyomavirus, replicate to extremely high copy number in the presence of the appropriate viral T antigen. Additional examples of mammalian replicons include those derived from bovine papillomavirus and Epstein-Barr virus. Additionally, the replicon may have two replication systems, thus allowing it to be maintained, for example, in mammalian cells for expression and in a prokaryotic host for cloning and amplification. Examples of such mammalian-bacteria shuttle vectors include pMT2 (Kaufman et al. (1989) Mol. Cell. Biol. 9:946) and pHEBO (Shimizu et al. (1986) Mol. Cell. Biol. 6:1074).

The transformation procedure used depends upon the host to be transformed. Methods for introduction of heterologous polynucleotides into mammalian cells are known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are known in the art and include many immortalized cell lines available from the American Type Culture Collection (ATCC), including but not limited to, Chinese hamster ovary (CHO) cells, HeLa cells, baby hamster kidney (BHK) cells, monkey kidney cells (COS), human hepatocellular carcinoma cells (e.g., Hep G2), and a number of other cell lines.

ii. Plant Cellular Expression Systems

There are many plant cell culture and whole plant genetic expression systems known in the art. Exemplary plant cellular genetic expression systems include those described in patents, such as: U.S. Pat. No. 5,693,506; U.S. Pat. No. 5,659,122; and U.S. Pat. No. 5,608,143. Additional examples of genetic expression in plant cell culture has been described by Zenk, Phytochemistry 30:3861-3863 (1991). Descriptions of plant protein signal peptides may be found in addition to the references described above in Vaulcombe et al., Mol. Gen. Genet. 209:33-40 (1987); Chandler et al., Plant Molecular Biology 3:407-418 (1984); Rogers, J. Biol. Chem. 260:3731-3738 (1985); Rothstein et al., Gene 55:353-356 (1987); Whittier et al., Nucleic Acids Research 15:2515-2535 (1987); Wirsel et al., Molecular Microbiology 3:3-14 (1989); Yu et al., Gene 122:247-253 (1992). A description of the regulation of plant gene expression by the phytohormone, gibberellic acid and secreted enzymes induced by gibberellic acid can be found in R. L. Jones and J. MacMillin, Gibberellins: in: Advanced Plant Physiology, Malcolm B. Wilkins, ed., 1984 Pitman Publishing Limited, London, pp. 21-52. References that describe other metabolically-regulated genes: Sheen, Plant Cell, 2:1027-1038(1990); Maas et al., EMBO J. 9:3447-3452 (1990); Benkel and Hickey, Proc. Natl. Acad. Sci. 84:1337-1339 (1987)

Typically, using techniques known in the art, a desired polynucleotide sequence is inserted into an expression cassette comprising genetic regulatory elements designed for operation in plants. The expression cassette is inserted into a desired expression vector with companion sequences upstream and downstream from the expression cassette suitable for expression in a plant host. The companion sequences will be of plasmid or viral origin and provide necessary characteristics to the vector to permit the vectors to move DNA from an original cloning host, such as bacteria, to the desired plant host. The basic bacterial/plant vector construct will preferably provide a broad host range prokaryote replication origin; a prokaryote selectable marker; and, for Agrobacterium transformations, T DNA sequences for Agrobacterium-mediated transfer to plant chromosomes. Where the heterologous gene is not readily amenable to detection, the construct will preferably also have a selectable marker gene suitable for determining if a plant cell has been transformed. A general review of suitable markers, for example for the members of the grass family, is found in Wilmink and Dons, 1993, Plant Mol. Biol. Reptr, 11 (2):165-185.

Sequences suitable for permitting integration of the heterologous sequence into the plant genome are also recommended. These might include transposon sequences and the like for homologous recombination as well as Ti sequences which permit random insertion of a heterologous expression cassette into a plant genome. Suitable prokaryote selectable markers include resistance toward antibiotics such as ampicillin or tetracycline. Other DNA sequences encoding additional functions may also be present in the vector, as is known in the art.

The nucleic acid molecules of the subject invention may be included into an expression cassette for expression of the protein(s) of interest. Usually, there will be only one expression cassette, although two or more are feasible. The recombinant expression cassette will contain in addition to the heterologous protein encoding sequence the following elements, a promoter region, plant 5′ untranslated sequences, initiation codon depending upon whether or not the structural gene comes equipped with one, and a transcription and translation termination sequence. Unique restriction enzyme sites at the 5′ and 3′ ends of the cassette allow for easy insertion into a pre-existing vector.

A heterologous coding sequence may be for any protein relating to the present invention. The sequence encoding the protein of interest will encode a signal peptide which allows processing and translocation of the protein, as appropriate, and will usually lack any sequence which might result in the binding of the desired protein of the invention to a membrane. Since, for the most part, the transcriptional initiation region will be for a gene which is expressed and translocated during germination, by employing the signal peptide which provides for translocation, one may also provide for translocation of the protein of interest. In this way, the protein(s) of interest will be translocated from the cells in which they are expressed and may be efficiently harvested. Typically secretion in seeds are across the aleurone or scutellar epithelium layer into the endosperm of the seed. While it is not required that the protein be secreted from the cells in which the protein is produced, this facilitates the isolation and purification of the recombinant protein.

Since the ultimate expression of the desired gene product will be in a eucaryotic cell it is desirable to determine whether any portion of the cloned gene contains sequences which will be processed out as introns by the host's splicosome machinery. If so, site-directed mutagenesis of the “intron” region may be conducted to prevent losing a portion of the genetic message as a false intron code, Reed and Maniatis, Cell 41:95-105, 1985.

The vector can be microinjected directly into plant cells by use of micropipettes to mechanically transfer the recombinant DNA. Crossway, Mol. Gen. Genet, 202:179-185, 1985. The genetic material may also be transferred into the plant cell by using polyethylene glycol, Krens, et al., Nature, 296, 72-74, 1982. Another method of introduction of nucleic acid segments is high velocity ballistic penetration by small particles with the nucleic acid either within the matrix of small beads or particles, or on the surface, Klein, et al., Nature, 327, 70-73, 1987 and Knudsen and Muller, 1991, Planta, 185:330-336 teaching particle bombardment of barley endosperm to create transgenic barley. Yet another method of introduction would be fusion of protoplasts with other entities, either minicells, cells, lysosomes or other fusible lipid-surfaced bodies, Fraley, et al., Proc. Natl. Acad. Sci. USA, 79, 1859-1863, 1982.

The vector may also be introduced into the plant cells by electroporation. (Fromm et al., Proc. Natl. Acad. Sci. USA 82:5824, 1985). In this technique, plant protoplasts are electroporated in the presence of plasmids containing the gene construct. Electrical impulses of high field strength reversibly permeabilize biomembranes allowing the introduction of the plasmids. Electroporated plant protoplasts reform the cell wall, divide, and form plant callus.

All plants from which protoplasts can be isolated and cultured to give whole regenerated plants can be transformed by the present invention so that whole plants are recovered which contain the transferred gene. It is known that practically all plants can be regenerated from cultured cells or tissues, including but not limited to all major species of sugarcane, sugar beet, cotton, fruit and other trees, legumes and vegetables. Some suitable plants include, for example, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersion, Nicotiana, Solanum, Petunia, Digitalis, Majorana, Cichorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura.

Means for regeneration vary from species to species of plants, but generally a suspension of transformed protoplasts containing copies of the heterologous gene is first provided. Callus tissue is formed and shoots may be induced from callus and subsequently rooted. Alternatively, embryo formation can be induced from the protoplast suspension. These embryos germinate as natural embryos to form plants. The culture media will generally contain various amino acids and hormones, such as auxin and cytokinins. It is also advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

In some plant cell culture systems, the desired protein of the invention may be excreted or alternatively, the protein may be extracted from the whole plant. Where the desired protein of the invention is secreted into the medium, it may be collected. Alternatively, the embryos and embryoless-half seeds or other plant tissue may be mechanically disrupted to release any secreted protein between cells and tissues. The mixture may be suspended in a buffer solution to retrieve soluble proteins. Conventional protein isolation and purification methods will be then used to purify the recombinant protein. Parameters of time, temperature pH, oxygen, and volumes will be adjusted through routine methods to optimize expression and recovery of heterologous protein.

iii. Baculovirus Systems

The polynucleotide encoding the protein can also be inserted into a suitable insect expression vector, and is operably linked to the control elements within that vector. Vector construction employs techniques which are known in the art. Generally, the components of the expression system include a transfer vector, usually a bacterial plasmid, which contains both a fragment of the baculovirus genome, and a convenient restriction site for insertion of the heterologous gene or genes to be expressed; a wild type baculovirus with a sequence homologous to the baculovirus-specific fragment in the transfer vector (this allows for the homologous recombination of the heterologous gene in to the baculovirus genome); and appropriate insect host cells and growth media.

After inserting the DNA sequence encoding the protein into the transfer vector, the vector and the wild type viral genome are transfected into an insect host cell where the vector and viral genome are allowed to recombine. The packaged recombinant virus is expressed and recombinant plaques are identified and purified. Materials and methods for baculovirus/insect cell expression systems are commercially available in kit form from, inter alia, Invitrogen, San Diego Calif. (“MaxBac” kit). These techniques are generally known to those skilled in the art and fully described in Summers and Smith, Texas Agricultural Experiment Station Bulletin No. 1555 (1987) (hereinafter “Summers and Smith”).

Prior to inserting the DNA sequence encoding the protein into the baculovirus genome, the above described components, comprising a promoter, leader (if desired), coding sequence of interest, and transcription termination sequence, are usually assembled into an intermediate transplacement construct (transfer vector). This construct may contain a single gene and operably linked regulatory elements; multiple genes, each with its owned set of operably linked regulatory elements; or multiple genes, regulated by the same set of regulatory elements. Intermediate transplacement constructs are often maintained in a replicon, such as an extrachromosomal element (e.g., plasmids) capable of stable maintenance in a host, such as a bacterium. The replicon will have a replication system, thus allowing it to be maintained in a suitable host for cloning and amplification.

Currently, the most commonly used transfer vector for introducing foreign genes into AcNPV is pAc373. Many other vectors, known to those of skill in the art, have also been designed. These include, for example, pVL985 (which alters the polyhedrin start codon from ATG to ATT, and which introduces a BamHI cloning site 32 basepairs downstream from the ATT; see Luckow and Summers, Virology (1989) 17:31.

The plasmid usually also contains the polyhedrin polyadenylation signal (Miller et al. (1988) Ann. Rev. Microbiol., 42:177) and a prokaryotic ampicillin-resistance (amp) gene and origin of replication for selection and propagation in E. coli.

Baculovirus transfer vectors usually contain a baculovirus promoter. A baculovirus promoter is any DNA sequence capable of binding a baculovirus RNA polymerase and initiating the downstream (5′ to 3′) transcription of a coding sequence (e.g., structural gene) into mRNA. A promoter will have a transcription initiation region which is usually placed proximal to the 5′ end of the coding sequence. This transcription initiation region usually includes an RNA polymerase binding site and a transcription initiation site. A baculovirus transfer vector may also have a second domain called an enhancer, which, if present, is usually distal to the structural gene. Expression may be either regulated or constitutive.

Structural genes, abundantly transcribed at late times in a viral infection cycle, provide particularly useful promoter sequences. Examples include sequences derived from the gene encoding the viral polyhedron protein, Friesen et al., (1986) “The Regulation of Baculovirus Gene Expression,” in: The Molecular Biology of Baculoviruses (ed. Walter Doerfler); EPO Publ. Nos. 127 839 and 155 476; and the gene encoding the p10 protein, Vlak et al., (1988), J. Gen. Virol. 69:765.

DNA encoding suitable signal sequences can be derived from genes for secreted insect or baculovirus proteins, such as the baculovirus polyhedrin gene (Carbonell et al. (1988) Gene, 73:409). Alternatively, since the signals for mammalian cell posttranslational modifications (such as signal peptide cleavage, proteolytic cleavage, and phosphorylation) appear to be recognized by insect cells, and the signals required for secretion and nuclear accumulation also appear to be conserved between the invertebrate cells and vertebrate cells, leaders of non-insect origin, such as those derived from genes encoding human (alpha) α-interferon, Maeda et al., (1985), Nature 315:592; human gastrin-releasing peptide, Lebacq-Verheyden et al., (1988), Molec. Cell. Biol. 8:3129; human IL-2, Smith et al., (1985) Proc. Nat'l Acad. Sci. USA, 82:8404; mouse IL-3, (Miyajima et al., (1987) Gene 58:273; and human glucocerebrosidase, Martin et al. (1988) DNA, 7:99, can also be used to provide for secretion in insects.

A recombinant polypeptide or polyprotein may be expressed intracellularly or, if it is expressed with the proper regulatory sequences, it can be secreted. Good intracellular expression of nonfused foreign proteins usually requires heterologous genes that ideally have a short leader sequence containing suitable translation initiation signals preceding an ATG start signal. If desired, methionine at the N-terminus may be cleaved from the mature protein by in vitro incubation with cyanogen bromide.

Alternatively, recombinant polyproteins or proteins which are not naturally secreted can be secreted from the insect cell by creating chimeric DNA molecules that encode a fusion protein comprised of a leader sequence fragment that provides for secretion of the foreign protein in insects. The leader sequence fragment usually encodes a signal peptide comprised of hydrophobic amino acids which direct the translocation of the protein into the endoplasmic reticulum.

After insertion of the DNA sequence and/or the gene encoding the expression product precursor of the protein, an insect cell host is co-transformed with the heterologous DNA of the transfer vector and the genomic DNA of wild type baculovirus—usually by co-transfection. The promoter and transcription termination sequence of the construct will usually comprise a 2-5 kb section of the baculovirus genome. Methods for introducing heterologous DNA into the desired site in the baculovirus virus are known in the art. (See Summers and Smith supra; Ju et al. (1987); Smith et al., Mol. Cell. Biol. (1983) 3:2156; and Luckow and Summers (1989)). For example, the insertion can be into a gene such as the polyhedrin gene, by homologous double crossover recombination; insertion can also be into a restriction enzyme site engineered into the desired baculovirus gene. Miller et al., (1989), Bioessays 4:91. The DNA sequence, when cloned in place of the polyhedrin gene in the expression vector, is flanked both 5′ and 3′ by polyhedrin-specific sequences and is positioned downstream of the polyhedrin promoter.

The newly formed baculovirus expression vector is subsequently packaged into an infectious recombinant baculovirus. Homologous recombination occurs at low frequency (between about 1% and about 5%); thus, the majority of the virus produced after cotransfection is still wild-type virus. Therefore, a method is necessary to identify recombinant viruses. An advantage of the expression system is a visual screen allowing recombinant viruses to be distinguished. The polyhedrin protein, which is produced by the native virus, is produced at very high levels in the nuclei of infected cells at late times after viral infection. Accumulated polyhedrin protein forms occlusion bodies that also contain embedded particles. These occlusion bodies, up to 15 μm in size, are highly refractile, giving them a bright shiny appearance that is readily visualized under the light microscope. Cells infected with recombinant viruses lack occlusion bodies. To distinguish recombinant virus from wild-type virus, the transfection supernatant is plaqued onto a monolayer of insect cells by techniques known to those skilled in the art. Namely, the plaques are screened under the light microscope for the presence (indicative of wild-type virus) or absence (indicative of recombinant virus) of occlusion bodies. Current Protocols in Microbiology Vol. 2 (Ausubel et al. eds) at 16.8 (Supp. 10, 1990); Summers and Smith, supra; Miller et al. (1989).

Recombinant baculovirus expression vectors have been developed for infection into several insect cells. For example, recombinant baculoviruses have been developed for, inter alia: Aedes aegypti , Autographa californica, Bombyx mori, Drosophila melanogaster, Spodoptera frugiperda, and Trichoplusia ni (PCT Pub. No. WO 89/046699; Carbonell et al., (1985) J. Virol. 56:153; Wright (1986) Nature 321:718; Smith et al., (1983) Mol. Cell. Biol. 3:2156; and see generally, Fraser, et al. (1989) In Vitro Cell. Dev. Biol. 25:225).

Cells and cell culture media are commercially available for both direct and fusion expression of heterologous polypeptides in a baculovirus/expression system; cell culture technology is generally known to those skilled in the art. See, e.g., Summers and Smith supra.

The modified insect cells may then be grown in an appropriate nutrient medium, which allows for stable maintenance of the plasmid(s) present in the modified insect host. Where the expression product gene is under inducible control, the host may be grown to high density, and expression induced. Alternatively, where expression is constitutive, the product will be continuously expressed into the medium and the nutrient medium must be continuously circulated, while removing the product of interest and augmenting depleted nutrients. The product may be purified by such techniques as chromatography, e.g., HPLC, affinity chromatography, ion exchange chromatography, etc.; electrophoresis; density gradient centrifugation; solvent extraction, or the like. As appropriate, the product may be further purified, as required, so as to remove substantially any insect proteins which are also secreted in the medium or result from lysis of insect cells, so as to provide a product which is at least substantially free of host debris, e.g., proteins, lipids and polysaccharides.

In order to obtain protein expression, recombinant host cells derived from the transformants are incubated under conditions which allow expression of the recombinant protein encoding sequence. These conditions will vary, dependent upon the host cell selected. However, the conditions are readily ascertainable to those of ordinary skill in the art, based upon what is known in the art.

iv. Bacterial Systems

Bacterial expression techniques are known in the art. A bacterial promoter is any DNA sequence capable of binding bacterial RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site and a transcription initiation site. A bacterial promoter may also have a second domain called an operator, that may overlap an adjacent RNA polymerase binding site at which RNA synthesis begins. The operator permits negative regulated (inducible) transcription, as a gene repressor protein may bind the operator and thereby inhibit transcription of a specific gene. Constitutive expression may occur in the absence of negative regulatory elements, such as the operator. In addition, positive regulation may be achieved by a gene activator protein binding sequence, which, if present is usually proximal (5′) to the RNA polymerase binding sequence. An example of a gene activator protein is the catabolite activator protein (CAP), which helps initiate transcription of the lac operon in Escherichia coli (E. coli) (Raibaud et al. (1984) Annu. Rev. Genet. 18:173). Regulated expression may therefore be either positive or negative, thereby either enhancing or reducing transcription.

Sequences encoding metabolic pathway enzymes provide particularly useful promoter sequences. Examples include promoter sequences derived from sugar metabolizing enzymes, such as galactose, lactose (lac) (Chang et al. (1977) Nature 198:1056), and maltose. Additional examples include promoter sequences derived from biosynthetic enzymes such as tryptophan (trp) (Goeddel et al. (1980) Nuc. Acids Res. 8:4057; Yelverton et al. (1981) Nucl. Acids Res. 9:731; U.S. Pat. No. 4,738,921; EPO Publ. Nos. 036 776 and 121 775). The beta-lactamase (bla) promoter system (Weissmann (1981) “The cloning of interferon and other mistakes.” In Interferon 3 (ed. I. Gresser)), bacteriophage lambda PL (Shimatake et al. (1981) Nature 292:128) and T5 (U.S. Pat. No. 4,689,406) promoter systems also provide useful promoter sequences.

In addition, synthetic promoters which do not occur in nature also function as bacterial promoters. For example, transcription activation sequences of one bacterial or bacteriophage promoter may be joined with the operon sequences of another bacterial or bacteriophage promoter, creating a synthetic hybrid promoter (U.S. Pat. No. 4,551,433). For example, the tac promoter is a hybrid tip-lac promoter comprised of both trp promoter and lac operon sequences that is regulated by the lac repressor (Amann et al. (1983) Gene 25:167; de Boer et al. (1983) Proc. Natl. Acad. Sci. 80:21). Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The bacteriophage T7 RNA polymerase/promoter system is an example of a coupled promoter system (Studier et al. (1986) J. Mol. Biol. 189:113; Tabor et al. (1985) Proc Natl. Acad. Sci. 82:1074). In addition, a hybrid promoter can also be comprised of a bacteriophage promoter and an E. coli operator region (EPO Publ. No. 267 851).

In addition to a functioning promoter sequence, an efficient ribosome binding site is also useful for the expression of foreign genes in prokaryotes. In E. coli, the ribosome binding site is called the Shine-Dalgamo (SD) sequence and includes an initiation codon (ATG) and a sequence 3-9 nucleotides in length located 3-11 nucleotides upstream of the initiation codon (Shine et al. (1975) Nature 254:34). The SD sequence is thought to promote binding of mRNA to the ribosome by the pairing of bases between the SD sequence and the 3′ end of E. coli 16S rRNA (Steitz et al. (1979) “Genetic signals and nucleotide sequences in messenger RNA.” In Biological Regulation and Development: Gene Expression (ed. R. F. Goldberger)). To express eukaryotic genes and prokaryotic genes with weak ribosome-binding site, it is often necessary to optimize the distance between the SD sequence and the ATG of the eukaryotic gene (Sambrook et al. (1989) “Expression of cloned genes in Escherichia coli.” In Molecular Cloning: A Laboratory Manual).

A DNA molecule may be expressed intracellularly. A promoter sequence may be directly linked with the DNA molecule, in which case the first amino acid at the N-terminus will always be a methionine, which is encoded by the ATG start codon. If desired, methionine at the N-terminus may be cleaved from the protein by in vitro incubation with cyanogen bromide or by either in vivo or in vitro incubation with a bacterial methionine N-terminal peptidase (EPO Publ. No. 219 237).

Fusion proteins provide an alternative to direct expression. Usually, a DNA sequence encoding the N-terminal portion of an endogenous bacterial protein, or other stable protein, is fused to the 5′ end of heterologous coding sequences. Upon expression, this construct will provide a fusion of the two amino acid sequences. For example, the bacteriophage lambda cell gene can be linked at the 5′ terminus of a foreign gene and expressed in bacteria. The resulting fusion protein preferably retains a site for a processing enzyme (factor Xa) to cleave the bacteriophage protein from the foreign gene (Nagai et al. (1984) Nature 309:810). Fusion proteins can also be made with sequences from the lacZ (Jia et al. (1987) Gene 60:197), trpE (Allen et al. (1987) J. Biotechnol. 5:93; Makoff et al. (1989) J. Gen. Microbiol. 135:11), and Chey (EPO Publ. No. 324 647) genes. The DNA sequence at the junction of the two amino acid sequences may or may not encode a cleavable site. Another example is a ubiquitin fusion protein. Such a fusion protein is made with the ubiquitin region that preferably retains a site for a processing enzyme (e.g. ubiquitin specific processing-protease) to cleave the ubiquitin from the foreign protein. Through this method, native foreign protein can be isolated (Miller et al. (1989) Bio/Technology 7:698).

Alternatively, foreign proteins can also be secreted from the cell by creating chimeric DNA molecules that encode a fusion protein comprised of a signal peptide sequence fragment that provides for secretion of the foreign protein in bacteria (U.S. Pat. No. 4,336,336). The signal sequence fragment usually encodes a signal peptide comprised of hydrophobic amino acids which direct the secretion of the protein from the cell. The protein is either secreted into the growth media (gram-positive bacteria) or into the periplasmic space, located between the inner and outer membrane of the cell (gram-negative bacteria). Preferably there are processing sites, which can be cleaved either in vivo or in vitro encoded between the signal peptide fragment and the foreign gene.

DNA encoding suitable signal sequences can be derived from genes for secreted bacterial proteins, such as the E. coli outer membrane protein gene (ompA) (Masui et al. (1983), in: Experimental Manipulation of Gene Expression; Ghrayeb et al. (1984) EMBO J. 3:2437) and the E. coli alkaline phosphatase signal sequence (phoA) (Oka et al. (1985) Proc. Natl. Acad. Sci. 82:7212). As an additional example, the signal sequence of the alpha-amylase gene from various Bacillus strains can be used to secrete heterologous proteins from B. subtilis (Palva et al. (1982) Proc. Natl. Acad. Sci. USA 79:5582; EPO Publ. No. 244 042).

Usually, transcription termination sequences recognized by bacteria are regulatory regions located 3′ to the translation stop codon, and thus together with the promoter flank the coding sequence. These sequences direct the transcription of an mRNA which can be translated into the polypeptide encoded by the DNA. Transcription termination sequences frequently include DNA sequences of about 50 nucleotides capable of forming stem loop structures that aid in terminating transcription. Examples include transcription termination sequences derived from genes with strong promoters, such as the trp gene in E. coli as well as other biosynthetic genes.

Usually, the above described components, comprising a promoter, signal sequence (if desired), coding sequence of interest, and transcription termination sequence, are put together into expression constructs. Expression constructs are often maintained in a replicon, such as an extrachromosomal element (e.g., plasmids) capable of stable maintenance in a host, such as bacteria. The replicon will have a replication system, thus allowing it to be maintained in a prokaryotic host either for expression or for cloning and amplification. In addition, a replicon may be either a high or low copy number plasmid. A high copy number plasmid will generally have a copy number ranging from about 5 to about 200, and usually about 10 to about 150. A host containing a high copy number plasmid will preferably contain at least about 10, and more preferably at least about 20 plasmids. Either a high or low copy number vector may be selected, depending upon the effect of the vector and the foreign protein on the host.

Alternatively, the expression constructs can be integrated into the bacterial genome with an integrating vector. Integrating vectors usually contain at least one sequence homologous to the bacterial chromosome that allows the vector to integrate. Integrations appear to result from recombinations between homologous DNA in the vector and the bacterial chromosome. For example, integrating vectors constructed with DNA from various Bacillus strains integrate into the Bacillus chromosome (EPO Publ. No. 127 328). Integrating vectors may also be comprised of bacteriophage or transposon sequences.

Usually, extrachromosomal and integrating expression constructs may contain selectable markers to allow for the selection of bacterial strains that have been transformed. Selectable markers can be expressed in the bacterial host and may include genes which render bacteria resistant to drugs such as ampicillin, chloramphenicol, erythromycin, kanamycin (neomycin), and tetracycline (Davies et al. (1978) Annu. Rev. Microbiol. 32:469). Selectable markers may also include biosynthetic genes, such as those in the histidine, tryptophan, and leucine biosynthetic pathways.

Alternatively, some of the above described components can be put together in transformation vectors. Transformation vectors are usually comprised of a selectable market that is either maintained in a replicon or developed into an integrating vector, as described above.

Expression and transformation vectors, either extra-chromosomal replicons or integrating vectors, have been developed for transformation into many bacteria. For example, expression vectors have been developed for, inter alia, the following bacteria: Bacillus subtilis (Palva et al. (1982) Proc. Natl. Acad. Sci. USA 79:5582; EPO Publ. Nos. 036 259 and 063 953; PCT Publ. No. WO 84/04541), Escherichia coli (Shimatake et al. (1981) Nature 292:128; Amann et al. (1985) Gene 40:183; Studier et al. (1986) J. Mol. Biol. 189:113; EPO Publ. Nos. 036 776, 136 829 and 136 907), Streptococcus cremoris (Powell et al. (1988) Appl. Environ. Microbiol. 54:655); Streptococcus lividans (Powell et al. (1988) Appl. Environ. Microbiol. 54:655), Streptomyces lividans (U.S. Pat. No. 4,745,056).

Methods of introducing exogenous DNA into bacterial hosts are well-known in the art, and usually include either the transformation of bacteria treated with CaCl₂ or other agents, such as divalent cations and DMSO. DNA can also be introduced into bacterial cells by electroporation. Transformation procedures usually vary with the bacterial species to be transformed. (See e.g., use of Bacillus: Masson et al. (1989) FEMS Microbiol. Lett. 60:273; Palva et al. (1982) Proc. Natl. Acad. Sci. USA 79:5582; EPO Publ. Nos. 036 259 and 063 953; PCT Publ. No. WO 84/04541; use of Campylobacter: Miller et al. (1988) Proc. Natl. Acad. Sci. 85:856; and Wang et al. (1990) J. Bacteriol. 172:949; use of Escherichia coli: Cohen et al. (1973) Proc. Natl. Acad. Sci. 69:2110; Dower et al. (1988) Nucleic Acids Res. 16:6127; Kushner (1978) “An improved method for transformation of Escherichia coli with ColE1-derived plasmids.” In Genetic Engineering: Proceedings of the International Symposium on Genetic Engineering (eds. H. W. Boyer and S. Nicosia); Mandel et al. (1970) J. Mol. Biol. 53:159; Taketo (1988) Biochim. Biophys. Acta 949:318; use of Lactobacillus: Chassy et al. (1987) FEMS Microbiol. Lett. 44:173; use of Pseudomonas: Fiedler et al. (1988) Anal. Biochem 170:38; use of Staphylococcus: Augustin et al. (1990) FEMS Microbiol. Lett. 66:203; use of Streptococcus: Barany et al. (1980) J. Bacteriol. 144:698; Harlander (1987) “Transformation of Streptococcus lactis by electroporation,” in: Streptococcal Genetics (ed. J. Ferretti and R. Curtiss III); Perry et al. (1981) Infect. Immun. 32:1295; Powell et al. (1988) Appl. Environ. Microbiol. 54:655; Somkuti et al. (1987) Proc. 4th Evr. Cong. Biotechnology 1:412.

v. Yeast Expression

Yeast expression systems are also known to one of ordinary skill in the art. A yeast promoter is any DNA sequence capable of binding yeast RNA polymerase and initiating the downstream (3′) transcription of a coding sequence (e.g. structural gene) into mRNA. A promoter will have a transcription initiation region which is usually placed proximal to the 5′ end of the coding sequence. This transcription initiation region usually includes an RNA polymerase binding site (the “TATA Box”) and a transcription initiation site. A yeast promoter may also have a second domain called an upstream activator sequence (UAS), which, if present, is usually distal to the structural gene. The UAS permits regulated (inducible) expression. Constitutive expression occurs in the absence of a UAS. Regulated expression may be either positive or negative, thereby either enhancing or reducing transcription.

Yeast is a fermenting organism with an active metabolic pathway, therefore sequences encoding enzymes in the metabolic pathway provide particularly useful promoter sequences. Examples include alcohol dehydrogenase (ADH) (EPO Publ. No. 284 044), enolase, glucokinase, glucose-6-phosphate isomerase, glyceraldehyde-3-phosphate-dehydrogenase (GAP or GAPDH), hexokinase, phosphofructokinase, 3-phosphoglycerate mutase, and pyruvate kinase (PyK) (EPO Publ. No. 329 203). The yeast PHO5 gene, encoding acid phosphatase, also provides useful promoter sequences (Myanohara et al. (1983) Proc. Natl. Acad. Sci. USA 80:1).

In addition, synthetic promoters which do not occur in nature also function as yeast promoters. For example, UAS sequences of one yeast promoter may be joined with the transcription activation region of another yeast promoter, creating a synthetic hybrid promoter. Examples of such hybrid promoters include the ADH regulatory sequence linked to the GAP transcription activation region (U.S. Pat. Nos. 4,876,197 and 4,880,734). Other examples of hybrid promoters include promoters which consist of the regulatory sequences of either the ADH2, GAL4, GAL10, OR PHO5 genes, combined with the transcriptional activation region of a glycolytic enzyme gene such as GAP or PyK (EPO Publ. No. 164 556). Furthermore, a yeast promoter can include naturally occurring promoters of non-yeast origin that have the ability to bind yeast RNA polymerase and initiate transcription. Examples of such promoters include, inter alia, (Cohen et al. (1980) Proc. Natl. Acad. Sci. USA 77:1078; Henikoff et al. (1981) Nature 283:835; Hollenberg et al. (1981) Curr. Topics Microbiol. Immunol. 96:119; Hollenberg et al. (1979) “The Expression of Bacterial Antibiotic Resistance Genes in the Yeast Saccharomyces cerevisiae,” in: Plasmids of Medical, Environmental and Commercial Importance (eds. K. N. Timmis and A. Puhler); Mercerau-Puigalon et al. (1980) Gene 11: 163; Panthier et al. (1980) Curr. Genet. 2:109).

A DNA molecule may be expressed intracellularly in yeast. A promoter sequence may be directly linked with the DNA molecule, in which case the first amino acid at the N-terminus of the recombinant protein will always be a methionine, which is encoded by the ATG start codon. If desired, methionine at the N-terminus may be cleaved from the protein by in vitro incubation with cyanogen bromide.

Fusion proteins provide an alternative for yeast expression systems, as well as in mammalian, plant, baculovirus, and bacterial expression systems. Usually, a DNA sequence encoding the N-terminal portion of an endogenous yeast protein, or other stable protein, is fused to the 5′ end of heterologous coding sequences. Upon expression, this construct will provide a fusion of the two amino acid sequences. For example, the yeast or human superoxide dismutase (SOD) gene, can be linked at the 5′ terminus of a foreign gene and expressed in yeast. The DNA sequence at the junction of the two amino acid sequences may or may not encode a cleavable site. See e.g., EPO Publ. No. 196056. Another example is a ubiquitin fusion protein. Such a fusion protein is made with the ubiquitin region that preferably retains a site for a processing enzyme (e.g. ubiquitin-specific processing protease) to cleave the ubiquitin from the foreign protein. Through this method, therefore, native foreign protein can be isolated (e.g., WO88/024066).

Alternatively, foreign proteins can also be secreted from the cell into the growth media by creating chimeric DNA molecules that encode a fusion protein comprised of a leader sequence fragment that provide for secretion in yeast of the foreign protein. Preferably, there are processing sites encoded between the leader fragment and the foreign gene that can be cleaved either in vivo or in vitro. The leader sequence fragment usually encodes a signal peptide comprised of hydrophobic amino acids which direct the secretion of the protein from the cell.

DNA encoding suitable signal sequences can be derived from genes for secreted yeast proteins, such as the yeast invertase gene (EPO Publ. No. 012 873; JPO Publ. No. 62:096,086) and the A-factor gene (U.S. Pat. No. 4,588,684). Alternatively, leaders of non-yeast origin, such as an interferon leader, exist that also provide for secretion in yeast (EPO Publ. No. 060 057).

A preferred class of secretion leaders are those that employ a fragment of the yeast alpha-factor gene, which contains both a “pre” signal sequence, and a “pro” region. The types of alpha-factor fragments that can be employed include the full-length pre-pro alpha factor leader (about 83 amino acid residues) as well as truncated alpha-factor leaders (usually about 25 to about 50 amino acid residues) (U.S. Pat. Nos. 4,546,083 and 4,870,008; EPO Publ. No. 324 274). Additional leaders employing an alpha-factor leader fragment that provides for secretion include hybrid alpha-factor leaders made with a presequence of a first yeast, but a pro-region from a second yeast alpha factor. (See e.g., PCT Publ. No. WO 89/02463.)

Usually, transcription termination sequences recognized by yeast are regulatory regions located 3′ to the translation stop codon, and thus together with the promoter flank the coding sequence. These sequences direct the transcription of an mRNA which can be translated into the polypeptide encoded by the DNA. Examples of transcription terminator sequence and other yeast-recognized termination sequences, such as those coding for glycolytic enzymes.

Usually, the above described components, comprising a promoter, leader (if desired), coding sequence of interest, and transcription termination sequence, are put together into expression constructs. Expression constructs are often maintained in a replicon, such as an extrachromosomal element (e.g., plasmids) capable of stable maintenance in a host, such as yeast or bacteria. The replicon may have two replication systems, thus allowing it to be maintained, for example, in yeast for expression and in a prokaryotic host for cloning and amplification. Examples of such yeast-bacteria shuttle vectors include YEp24 (Botstein et al. (1979) Gene 8:17-24), pCl/1 (Brake et al. (1984) Proc. Natl. Acad. Sci USA 81:4642-4646), and YRp17 (Stinchcomb et al. (1982) J. Mol. Biol. 158:157). In addition, a replicon may be either a high or low copy number plasmid. A high copy number plasmid will generally have a copy number ranging from about 5 to about 200, and usually about 10 to about 150. A host containing a high copy number plasmid will preferably have at least about 10, and more preferably at least about 20. Enter a high or low copy number vector may be selected, depending upon the effect of the vector and the foreign protein on the host. See e.g., Brake et al., supra.

Alternatively, the expression constructs can be integrated into the yeast genome with an integrating vector. Integrating vectors usually contain at least one sequence homologous to a yeast chromosome that allows the vector to integrate, and preferably contain two homologous sequences flanking the expression construct. Integrations appear to result from recombinations between homologous DNA in the vector and the yeast chromosome (Orr-Weaver et al. (1983) Methods in Enzymol. 101:228-245). An integrating vector may be directed to a specific locus in yeast by selecting the appropriate homologous sequence for inclusion in the vector. See Orr-Weaver et al., supra. One or more expression construct may integrate, possibly affecting levels of recombinant protein produced (Rine et al. (1983) Proc. Natl. Acad. Sci. USA 80:6750). The chromosomal sequences included in the vector can occur either as a single segment in the vector, which results in the integration of the entire vector, or two segments homologous to adjacent segments in the chromosome and flanking the expression construct in the vector, which can result in the stable integration of only the expression construct.

Usually, extrachromosomal and integrating expression constructs may contain selectable markers to allow for the selection of yeast strains that have been transformed. Selectable markers may include biosynthetic genes that can be expressed in the yeast host, such as ADE2, HIS4, LEU2, TRP1, and ALG7, and the G418 resistance gene, which confer resistance in yeast cells to tunicamycin and G418, respectively. In addition, a suitable selectable marker may also provide yeast with the ability to grow in the presence of toxic compounds, such as metal. For example, the presence of CUP1 allows yeast to grow in the presence of copper ions (Butt et al. (1987) Microbiol, Rev. 51:351).

Alternatively, some of the above described components can be put together into transformation vectors. Transformation vectors are usually comprised of a selectable marker that is either maintained in a replicon or developed into an integrating vector, as described above.

Expression and transformation vectors, either extrachromosomal replicons or integrating vectors, have been developed for transformation into many yeasts. For example, expression vectors and methods of introducing exogenous DNA into yeast hosts have been developed for, inter alia, the following yeasts: Candida albicans (Kurtz, et al. (1986) Mol. Cell. Biol. 6:142); Candida maltosa (Kunze, et al. (1985) J. Basic Microbiol. 25:141); Hansenula polymorpha (Gleeson, et al. (1986) J. Gen. Microbiol. 132:3459; Roggenkamp et al. (1986) Mol. Gen. Genet. 202:302); Kluyveromyces fragilis (Das, et al. (1984) J. Bacteriol. 158:1165); Kluyveromyces lactis (De Louvencourt et al. (1983) J. Bacteriol. 154:737; Van den Berg et al. (1990) Bio/Technology 8:135); Pichia guillerimondii (Kunze et al. (1985) J. Basic Microbiol. 25:141); Pichia pastoris (Cregg, et al. (1985) Mol. Cell. Biol. 5:3376; U.S. Pat. Nos. 4,837,148 and 4,929,555); Saccharomyces cerevisiae (Hinnen et al. (1978) Proc. Natl. Acad. Sci. USA 75:1929; Ito et al. (1983) J. Bacteriol. 153:163); Schizosaccharomyces pombe (Beach and Nurse (1981) Nature 300:706); and Yarrowia lipolytica (Davidow, et al. (1985) Curr. Genet. 10:380471 Gaillardin, et al. (1985) Curr. Genet. 10:49).

Methods of introducing exogenous DNA into yeast hosts are well-known in the art, and usually include either the transformation of spheroplasts or of intact yeast cells treated with alkali cations. Transformation procedures usually vary with the yeast species to be transformed. See e.g., [Kurtz et al. (1986) Mol. Cell. Biol. 6:142; Kunze et al. (1985) J. Basic Microbiol. 25:141; Candida]; [Gleeson et al. (1986) J. Gen. Microbiol. 132:3459; Roggenkamp et al. (1986) Mol. Gen. Genet. 202:302; Hansenula]; [Das et al. (1984) J. Bacteriol. 158:1165; De Louvencourt et al. (1983) J. Bacteriol. 154:1165; Van den Berg et al. (1990) Bio/Technology 8:135; Kluyveromyces]; [Cregg et al. (1985) Mol. Cell. Biol. 5:3376; Kunze et al. (1985) J. Basic Microbiol. 25:141; U.S. Pat. Nos. 4,837,148 and 4,929,555; Pichia]; [Hinnen et al. (1978) Proc. Natl. Acad. Sci. USA 75;1929; Ito et al. (1983) J. Bacteriol. 153:163 Saccharomyces]; [Beach and Nurse (1981) Nature 300:706; Schizosaccharomyces]; [Davidow et al. (1985) Curr. Genet. 10:39; Gaillardin et al. (1985) Curr. Genet. 10:49; Yarrowia].

Definitions

A composition containing X is “substantially free of” Y when at least 85% by weight of the total X+Y in the composition is X. Preferably, X comprises at least about 90% by weight of the total of X+Y in the composition, more preferably at least about 95% or even 99% by weight.

The term “heterologous” refers to two biological components that are not found together in nature. The components may be host cells, genes, or regulatory regions, such as promoters. Although the heterologous components are not found together in nature, they can function together, as when a promoter heterologous to a gene is operably linked to the gene. Another example is where a Neisserial sequence is heterologous to a mouse host cell.

An “origin of replication” is a polynucleotide sequence that initiates and regulates replication of polynucleotides, such as an expression vector. The origin of replication behaves as an autonomous unit of polynucleotide replication within a cell, capable of replication under its own control. An origin of replication may be needed for a vector to replicate in a particular host cell. With certain origins of replication, an expression vector can be reproduced at a high copy number in the presence of the appropriate proteins within the cell. Examples of origins are the autonomously replicating sequences, which are effective in yeast; and the viral T-antigen, effective in COS-7 cells.

A “mutant” sequence is defined as a DNA, RNA or amino acid sequence differing from but having homology with the native or disclosed sequence. Depending on the particular sequence, the degree of homology between the native or disclosed sequence and the mutant sequence is preferably greater than 50% (e.g., 60%, 70%, 80%, 90%, 95%, 99% or more) which is calculated as described above. As used herein, an “allelic variant” of a nucleic acid molecule, or region, for which nucleic acid sequence is provided herein is a nucleic acid molecule, or region, that occurs at essentially the same locus in the genome of another or second isolate, and that, due to natural variation caused by, for example, mutation or recombination, has a similar but not identical nucleic acid sequence. A coding region allelic variant typically encodes a protein having similar activity to that of the protein encoded by the gene to which it is being compared. An allelic variant can also comprise an alteration in the 5′ or 3′ untranslated regions of the gene, such as in regulatory control regions. (see, for example, U.S. Pat. No. 5,753,235).

Antibodies

As used herein, the term “antibody” refers to a polypeptide or group of polypeptides composed of at least one antibody combining site. An “antibody combining site” is the three-dimensional binding space with an internal surface shape and charge distribution complementary to the features of an epitope of an antigen, which allows a binding of the antibody with the antigen. “Antibody” includes, for example, vertebrate antibodies, hybrid antibodies, chimeric antibodies, humanized antibodies, altered antibodies, univalent antibodies, Fab proteins, and single domain antibodies.

Antibodies against the proteins of the invention are useful for affinity chromatography, immunoassays, and distinguishing/identifying Neisseria MenB proteins. Antibodies elicited against the proteins of the present invention bind to antigenic polypeptides or proteins or protein fragments that are present and specifically associated with strains of Neisseria meningitidis MenB. In some instances, these antigens may be associated with specific strains, such as those antigens specific for the MenB strains. The antibodies of the invention may be immobilized to a matrix and utilized in an immunoassay or on an affinity chromatography column, to enable the detection and/or separation of polypeptides, proteins or protein fragments or cells comprising such polypeptides, proteins or protein fragments. Alternatively, such polypeptides, proteins or protein fragments may be immobilized so as to detect antibodies bindably specific thereto.

Antibodies to the proteins of the invention, both polyclonal and monoclonal, may be prepared by conventional methods. In general, the protein is first used to immunize a suitable animal, preferably a mouse, rat, rabbit or goat. Rabbits and goats are preferred for the preparation of polyclonal sera due to the volume of serum obtainable, and the availability of labeled anti-rabbit and anti-goat antibodies. Immunization is generally performed by mixing or emulsifying the protein in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization. Polyclonal antisera is obtained by bleeding the immunized animal into a glass or plastic container, incubating the blood at 25° C. for one hour, followed by incubating at 4° C. for 2-18 hours. The serum is recovered by centrifugation (e.g., 1,000 g for 10 minutes). About 20-50 ml per bleed may be obtained from rabbits.

Monoclonal antibodies are prepared using the standard method of Kohler & Milstein (Nature (1975) 256:495-96), or a modification thereof. Typically, a mouse or rat is immunized as described above. However, rather than bleeding the animal to extract serum, the spleen (and optionally several large lymph nodes) is removed and dissociated into single cells. If desired, the spleen cells may be screened (after removal of nonspecifically adherent cells) by applying a cell suspension to a plate or well coated with the protein antigen. B-cells that express membrane-bound immunoglobulin specific for the antigen bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution, and are assayed for the production of antibodies which bind specifically to the immunizing antigen (and which do not bind to unrelated antigens). The selected MAb-secreting hybridomas are then cultured either in vitro (e.g., in tissue culture bottles or hollow fiber reactors), or in vivo (as ascites in mice).

If desired, the antibodies (whether polyclonal or monoclonal) may be labeled using conventional techniques. Suitable labels include fluorophores, chromophores, radioactive atoms (particularly ³²P and ¹²⁵I), electron-dense reagents, enzymes, and ligands having specific binding partners. Enzymes are typically detected by their activity. For example, horseradish peroxidase is usually detected by its ability to convert 3,3′,5,5′-tetramethylbenzidine (TMB) to a blue pigment, quantifiable with a spectrophotometer. “Specific binding partner” refers to a protein capable of binding a ligand molecule with high specificity, as for example in the case of an antigen and a monoclonal antibody specific therefor. Other specific binding partners include biotin and avidin or streptavidin, IgG and protein A, and the numerous receptor-ligand couples known in the art. It should be understood that the above description is not meant to categorize the various labels into distinct classes, as the same label may serve in several different modes. For example, ¹²⁵I, may serve as a radioactive label or as an electron-dense reagent. HRP may serve as enzyme or as antigen for a MAb. Further, one may combine various labels for desired effect. For example, MAbs and avidin also require labels in the practice of this invention: thus, one might label a MAb with biotin, and detect its presence with avidin labeled with ¹²⁵I, or with an anti-biotin MAb labeled with HRP. Other permutations and possibilities will be readily apparent to those of ordinary skill in the art, and are considered as equivalents within the scope of the instant invention.

Antigens, immunogens, polypeptides, proteins or protein fragments of the present invention elicit formation of specific binding partner antibodies. These antigens, immunogens, polypeptides, proteins or protein fragments of the present invention comprise immunogenic compositions of the present invention. Such immunogenic compositions may further comprise or include adjuvants, carriers, or other compositions that promote or enhance or stabilize the antigens, polypeptides, proteins or protein fragments of the present invention. Such adjuvants and carriers will be readily apparent to those of ordinary skill in the art.

Pharmaceutical Compositions

Pharmaceutical compositions can include either polypeptides, antibodies, or nucleic acid of the invention. The pharmaceutical compositions will comprise a therapeutically effective amount of either polypeptides, antibodies, or polynucleotides of the claimed invention.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature, when given to a patient that is febrile. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgment of the clinician.

For purposes of the present invention, an effective dose will be from about 0.01 mg/kg to 50 mg/kg or 0.05 mg/kg to about 10 mg/kg of the DNA constructs in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

Delivery Methods

Once formulated, the compositions of the invention can be administered directly to the subject. The subjects to be treated can be animals; in particular, human subjects can be treated.

Direct delivery of the compositions will generally be accomplished by injection, either subcutaneously, intraperitoneally, intravenously or intramuscularly or delivered to the interstitial space of a tissue. The compositions can also be administered into a lesion. Other modes of administration include oral and pulmonary administration, suppositories, and transdermal and transcutaneous applications, needles, and gene guns or hyposprays. Dosage treatment may be a single dose schedule or a multiple dose schedule.

Vaccines

Vaccines according to the invention may either be prophylactic (i.e., to prevent infection) or therapeutic (i.e., to treat disease after infection).

Such vaccines comprise immunizing antigen(s) or immunogen(s), immunogenic polypeptide, protein(s) or protein fragments, or nucleic acids (e.g., ribonucleic acid or deoxyribonucleic acid), usually in combination with “pharmaceutically acceptable carriers,” which include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Additionally, these carriers may function as immunostimulating agents (“adjuvants”). Furthermore, the immunogen or antigen may be conjugated to a bacterial toxoid, such as a toxoid from diphtheria, tetanus, cholera, H. pylori, etc. pathogens.

Preferred adjuvants to enhance effectiveness of the composition include, but are not limited to: (1) aluminum salts (alum), such as aluminum hydroxide, aluminum phosphate, aluminum sulfate, etc.; (2) oil-in-water emulsion formulations (with or without other specific immunostimulating agents such as muramyl peptides (see below) or bacterial cell wall components), such as for example (a) MF59 (PCT Publ. No. WO 90/14837), containing 5% Squalene, 0.5% Tween 80, and 0.5% Span 85 (optionally containing various amounts of MTP-PE (see below), although not required) formulated into submicron particles using a microfluidizer such as Model 110Y microfluidizer (Microfluidics, Newton, Mass.), (b) SAF, containing 10% Squalane, 0.4% Tween 80, 5% pluronic-blocked polymer L121, and thr-MDP (see below) either microfluidized into a submicron emulsion or vortexed to generate a larger particle size emulsion, and (c) Ribi™ adjuvant system (RAS), (Ribi Immunochem, Hamilton, Mont.) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); (3) saponin adjuvants, such as Stimulon™ (Cambridge Bioscience, Worcester, Mass.) may be used or particles generated therefrom such as ISCOMs (immunostimulating complexes); (4) Complete Freund's Adjuvant (CFA) and Incomplete Freund's Adjuvant (IFA); (5) cytokines, such as interleukins (e.g., IL-1, IL-2, IL-4, IL-5, IL-6, IL-7, IL-12, etc.), interferons (e.g., gamma interferon), macrophage colony stimulating factor (M-CSF), tumor necrosis factor (TNF), etc; (6) detoxified mutants of a bacterial ADP-ribosylating toxin such as a cholera toxin (CT), a pertussis toxin (PT), or an E. coli heat-labile toxin (LT), particularly LT-K63, LT-R72, CT-S109, PT-K9/G129; see, e.g., WO 93/13302 and WO 92/19265; and (7) other substances that act as immunostimulating agents to enhance the effectiveness of the composition. Alum and MF59 are preferred.

As mentioned above, muramyl peptides include, but are not limited to, N-acetyl-muramyl-L-threonyl-D-isoglutamine (thr-MDP), N-acetyl-normuramyl-1-alanyl-d-isoglutamine (nor-MDP), N-acetylmuramyl-1-alanyl-d-isoglutaminyl-1-alanine-2-(1′-2′-dipalmitoyl-sn-glycero-3-huydroxyphosphoryloxy)-ethylamine (MTP-PE), etc.

The vaccine compositions comprising immunogenic compositions (e.g., which may include the antigen, pharmaceutically acceptable carrier, and adjuvant) typically will contain diluents, such as water, saline, glycerol, ethanol, etc. Additionally, auxiliary substances, such as wetting or emulsifying agents, pH buffering substances, and the like, may be present in such vehicles. Alternatively, vaccine compositions comprising immunogenic compositions may comprise an antigen, polypeptide, protein, protein fragment or nucleic acid in a pharmaceutically acceptable carrier.

More specifically, vaccines comprising immunogenic compositions comprise an immunologically effective amount of the immunogenic polypeptides, as well as any other of the above-mentioned components, as needed. By “immunologically effective amount”, it is meant that the administration of that amount to an individual, either in a single dose or as part of a series, is effective for treatment or prevention. This amount varies depending upon the health and physical condition of the individual to be treated, the taxonomic group of individual to be treated (e.g., nonhuman primate, primate, etc.), the capacity of the individual's immune system to synthesize antibodies, the degree of protection desired, the formulation of the vaccine, the treating doctor's assessment of the medical situation, and other relevant factors. It is expected that the amount will fall in a relatively broad range that can be determined through routine trials.

Typically, the vaccine compositions or immunogenic compositions are prepared as injectables, either as liquid solutions or suspensions; solid forms suitable for solution in, or suspension in, liquid vehicles prior to injection may also be prepared. The preparation also may be emulsified or encapsulated in liposomes for enhanced adjuvant effect, as discussed above under pharmaceutically acceptable carriers.

The immunogenic compositions are conventionally administered parenterally, e.g., by injection, either subcutaneously or intramuscularly. Additional formulations suitable for other modes of administration include oral and pulmonary formulations, suppositories, and transdermal and transcutaneous applications. Dosage treatment may be a single dose schedule or a multiple dose schedule. The vaccine may be administered in conjunction with other immunoregulatory agents.

As an alternative to protein-based vaccines, DNA vaccination may be employed (e.g., Robinson & Torres (1997) Seminars in Immunology 9:271-283; Donnelly et al. (1997) Annu Rev Immunol 15:617-648).

Gene Delivery Vehicles

Gene therapy vehicles for delivery of constructs, including a coding sequence of a therapeutic of the invention, to be delivered to the mammal for expression in the mammal, can be administered either locally or systemically. These constructs can utilize viral or non-viral vector approaches in in vivo or ex vivo modality. Expression of such coding sequence can be induced using endogenous mammalian or heterologous promoters. Expression of the coding sequence in vivo can be either constitutive or regulated.

The invention includes gene delivery vehicles capable of expressing the contemplated nucleic acid sequences. The gene delivery vehicle is preferably a viral vector and, more preferably, a retroviral, adenoviral, adeno-associated viral (AAV), herpes viral, or alphavirus vector. The viral vector can also be an astrovirus, coronavirus, orthomyxovirus, papovavirus, paramyxovirus, parvovirus, picornavirus, poxvirus, or togavirus viral vector. See generally, Jolly (1994) Cancer Gene Therapy 1:51-64; Kimura (1994) Human Gene Therapy 5:845-852; Connelly (1995) Human Gene Therapy 6:185-193; and Kaplitt (1994) Nature Genetics 6:148-153.

Retroviral vectors are well known in the art, including B, C and D type retroviruses, xenotropic retroviruses (for example, NZB-X1, NZB-X2 and NZB9-1 (see O'Neill (1985) J. Virol. 53:160) polytropic retroviruses e.g., MCF and MCF-MLV (see Kelly (1983) J. Virol. 45:291), spumaviruses and lentiviruses. See RNA Tumor Viruses, Second Edition, Cold Spring Harbor Laboratory, 1985.

Portions of the retroviral gene therapy vector may be derived from different retroviruses. For example, retrovector LTRs may be derived from a Murine Sarcoma Virus, a tRNA binding site from a Rous Sarcoma Virus, a packaging signal from a Murine Leukemia Virus, and an origin of second strand synthesis from an Avian Leukosis Virus.

These recombinant retroviral vectors may be used to generate transduction competent retroviral vector particles by introducing them into appropriate packaging cell lines (see U.S. Pat. No. 5,591,624). Retrovirus vectors can be constructed for site-specific integration into host cell DNA by incorporation of a chimeric integrase enzyme into the retroviral particle (see WO96/37626). It is preferable that the recombinant viral vector is a replication defective recombinant virus.

Packaging cell lines suitable for use with the above-described retrovirus vectors are well known in the art, are readily prepared (see WO95/30763 and WO92/05266), and can be used to create producer cell lines (also termed vector cell lines or “VCLs”) for the production of recombinant vector particles. Preferably, the packaging cell lines are made from human parent cells (e.g., HT1080 cells) or mink parent cell lines, which eliminates inactivation in human serum.

Preferred retroviruses for the construction of retroviral gene therapy vectors include Avian Leukosis Virus, Bovine Leukemia, Virus, Murine Leukemia Virus, Mink-Cell Focus-Inducing Virus, Murine Sarcoma Virus, Reticuloendotheliosis Virus and Rous Sarcoma Virus. Particularly preferred Murine Leukemia Viruses include 4070A and 1504A (Hartley and Rowe (1976) J Virol 19:19-25), Abelson (ATCC No. VR-999), Friend (ATCC No. VR-245), Graffi, Gross (ATCC No. VR-590), Kirsten, Harvey Sarcoma Virus and Rauscher (ATCC No. VR-998) and Moloney Murine Leukemia Virus (ATCC No. VR-190). Such retroviruses may be obtained from depositories or collections such as the American Type Culture Collection (“ATCC”) in Rockville, Md. or isolated from known sources using commonly available techniques.

Exemplary known retroviral gene therapy vectors employable in this invention include those described in patent applications GB2200651, EP0415731, EP0345242, EP0334301, WO89/02468; WO89/05349, WO89/09271, WO90/02806, WO90/07936, WO94/03622, WO93/25698, WO93/25234, WO93/11230, WO93/10218, WO91/02805, WO91/02825, WO95/07994, U.S. Pat. No. 5,219,740, U.S. Pat. No. 4,405,712, U.S. Pat. No. 4,861,719, U.S. Pat. No. 4,980,289, U.S. Pat. No. 4,777,127, U.S. Pat. No. 5,591,624. See also Vile (1993) Cancer Res 53:3860-3864; Vile (1993) Cancer Res 53:962-967; Ram (1993) Cancer Res 53 (1993) 83-88; Takamiya (1992) J Neurosci Res 33:493-503; Baba (1993) J Neurosurg 79:729-735; Mann (1983) Cell 33:153; Cane (1984) Proc Natl Acad Sci 81:6349; and Miller (1990) Human Gene Therapy 1.

Human adenoviral gene therapy vectors are also known in the art and employable in this invention. See, for example, Berkner (1988) Biotechniques 6:616 and Rosenfeld (1991) Science 252:431, and WO93/07283, WO93/06223, and WO93/07282. Exemplary known adenoviral gene therapy vectors employable in this invention include those described in the above referenced documents and in WO94/12649, WO93/03769, WO93/19191, WO94/28938, WO95/1 1984, WO95/00655, WO95/27071, WO95/29993, WO95/34671, WO96/05320, WO94/08026, WO94/1 1506, WO93/06223, WO94/24299, WO95/14102, WO95/24297, WO95/02697, WO94/28152, WO94/24299, WO95/09241, WO95/25807, WO95/05835, WO94/18922 and WO95/09654. Alternatively, administration of DNA linked to killed adenovirus as described in Curiel (1992) Hum. Gene Ther. 3:147-154 may be employed. The gene delivery vehicles of the invention also include adenovirus associated virus (AAV) vectors. Leading and preferred examples of such vectors for use in this invention are the AAV-2 based vectors disclosed in Srivastava, WO93/09239. Most preferred AAV vectors comprise the two AAV inverted terminal repeats in which the native D-sequences are modified by substitution of nucleotides, such that at least 5 native nucleotides and up to 18 native nucleotides, preferably at least 10 native nucleotides up to 18 native nucleotides, most preferably 10 native nucleotides are retained and the remaining nucleotides of the D-sequence are deleted or replaced with non-native nucleotides. The native D-sequences of the AAV inverted terminal repeats are sequences of 20 consecutive nucleotides in each AAV inverted terminal repeat (i.e., there is one sequence at each end) which are not involved in HP formation. The non-native replacement nucleotide may be any nucleotide other than the nucleotide found in the native D-sequence in the same position. Other employable exemplary AAV vectors are pWP-19, pWN-1, both of which are disclosed in Nahreini (1993) Gene 124:257-262. Another example of such an AAV vector is psub201 (see Samulski (1987) J. Virol. 61:3096). Another exemplary AAV vector is the Double-D ITR vector. Construction of the Double-D ITR vector is disclosed in U.S. Pat. No. 5,478,745. Still other vectors are those disclosed in Carter U.S. Pat. No. 4,797,368 and Muzyczka U.S. Pat. No. 5,139,941, Chartejee U.S. Pat. No. 5,474,935, and Kotin WO94/288157. Yet a further example of an AAV vector employable in this invention is SSV9AFABTKneo, which contains the AFP enhancer and albumin promoter and directs expression predominantly in the liver. Its structure and construction are disclosed in Su (1996) Human Gene Therapy 7:463-470. Additional AAV gene therapy vectors are described in U.S. Pat. No. 5,354,678, U.S. Pat. No. 5,173,414, U.S. Pat. No. 5,139,941, and U.S. Pat. No. 5,252,479.

The gene therapy vectors comprising sequences of the invention also include herpes vectors. Leading and preferred examples are herpes simplex virus vectors containing a sequence encoding a thymidine kinase polypeptide such as those disclosed in U.S. Pat. No. 5,288,641 and EP0176170 (Roizman). Additional exemplary herpes simplex virus vectors include HFEM/ICP6-LacZ disclosed in WO95/04139 (Wistar Institute), pHSVlac described in Geller (1988) Science 241:1667-1669 and in WO90/09441 and WO92/07945, HSV Us3::pgC-lacZ described in Fink (1992) Human Gene Therapy 3:11-19 and HSV 7134, 2 RH 105 and GAL4 described in EP 0453242 (Breakefield), and those deposited with the ATCC as accession numbers ATCC VR-977 and ATCC VR-260.

Also contemplated are alpha virus gene therapy vectors that can be employed in this invention. Preferred alpha virus vectors are Sindbis viruses vectors. Togaviruses, Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309, 5,217,879, and WO92/10578. More particularly, those alpha virus vectors described in U.S. Ser. No. 08/405,627, filed Mar. 15, 1995,WO94/21792, WO92/10578, WO95/07994, U.S. Pat. No. 5,091,309 and U.S. Pat. No. 5,217,879 are employable. Such alpha viruses may be obtained from depositories or collections such as the ATCC in Rockville, Md. or isolated from known sources using commonly available techniques. Preferably, alphavirus vectors with reduced cytotoxicity are used (see U.S. Ser. No. 08/679640).

DNA vector systems such as eukarytic layered expression systems are also useful for expressing the nucleic acids of the invention. See WO95/07994 for a detailed description of eukaryotic layered expression systems. Preferably, the eukaryotic layered expression systems of the invention are derived from alphavirus vectors and most preferably from Sindbis viral vectors.

Other viral vectors suitable for use in the present invention include those derived from poliovirus, for example ATCC VR-58 and those described in Evans, Nature 339 (1989) 385 and Sabin (1973) J. Biol. Standardization 1:115; rhinovirus, for example ATCC VR-1110 and those described in Arnold (1990) J Cell Biochem L401; pox viruses such as canary pox virus or vaccinia virus, for example ATCC VR-111 and ATCC VR-2010 and those described in Fisher-Hoch (1989) Proc Natl Acad Sci 86:317; Flexner (1989) Ann NY Acad Sci 569:86, Flexner (1990) Vaccine 8:17; in U.S. Pat. No. 4,603,112 and U.S. Pat. No. 4,769,330 and WO89/01973; SV40 virus, for example ATCC VR-305 and those described in Mulligan (1979) Nature 277:108 and Madzak (1992) J Gen Virol 73:1533; influenza virus, for example ATCC VR-797 and recombinant influenza viruses made employing reverse genetics techniques as described in U.S. Pat. No. 5,166,057 and in Enami (1990) Proc Natl Acad Sci 87:3802-3805; Enami & Palese (1991) J Virol 65:2711-2713 and Luytjes (1989) Cell 59:110, (see also McMichael (1983) NEJ Med 309:13, and Yap (1978) Nature 273:238 and Nature (1979) 277:108); human immunodeficiency virus as described in EP-0386882 and in Buchschacher (1992) J. Virol. 66:2731; measles virus, for example ATCC VR-67 and VR-1247 and those described in EP-0440219; Aura virus, for example ATCC VR-368; Bebaru virus, for example ATCC VR-600 and ATCC VR-1240; Cabassou virus, for example ATCC VR-922; Chikungunya virus, for example ATCC VR-64 and ATCC VR-1241; Fort Morgan Virus, for example ATCC VR-924; Getah virus, for example ATCC VR-369 and ATCC VR-1243; Kyzylagach virus, for example ATCC VR-927; Mayaro virus, for example ATCC VR-66; Mucambo virus, for example ATCC VR-580 and ATCC VR-1244; Ndumu virus, for example ATCC VR-371; Pixuna virus, for example ATCC VR-372 and ATCC VR-1245; Tonate virus, for example ATCC VR-925; Triniti virus, for example ATCC VR-469; Una virus, for example ATCC VR-374; Whataroa virus, for example ATCC VR-926; Y-62-33 virus, for example ATCC VR-375; O'Nyong virus, Eastern encephalitis virus, for example ATCC VR-65 and ATCC VR-1242; Western encephalitis virus, for example ATCC VR-70, ATCC VR-1251, ATCC VR-622 and ATCC VR-1252; and coronavirus, for example ATCC VR-740 and those described in Hamre (1966) Proc Soc Exp Biol Med 121:190.

Delivery of the compositions of this invention into cells is not limited to the above mentioned viral vectors. Other delivery methods and media may be employed such as, for example, nucleic acid expression vectors, polycationic condensed DNA linked or unlinked to killed adenovirus alone, for example see U.S. Ser. No. 08/366,787, filed Dec. 30, 1994 and Curiel (1992) Hum Gene Ther 3:147-154 ligand linked DNA, for example see Wu (1989) J Biol Chem 264:16985-16987, eucaryotic cell delivery vehicles cells, for example see U.S. Ser. No.08/240,030, filed May 9, 1994, and U.S. Ser. No. 08/404,796, deposition of photopolymerized hydrogel materials, hand-held gene transfer particle gun, as described in U.S. Pat. No. 5,149,655, ionizing radiation as described in U.S. Pat. No. 5,206,152 and in WO92/11033, nucleic charge neutralization or fusion with cell membranes. Additional approaches are described in Philip (1994) Mol Cell Biol 14:2411-2418 and in Woffendin (1994) Proc Natl Acad Sci 91:1581-1585.

Particle mediated gene transfer may be employed, for example see U.S. Ser. No. 60/023,867. Briefly, the sequence can be inserted into conventional vectors that contain conventional control sequences for high level expression, and then incubated with synthetic gene transfer molecules such as polymeric DNA-binding cations like polylysine, protamine, and albumin, linked to cell targeting ligands such as asialoorosomucoid, as described in Wu & Wu (1987) J. Biol. Chem. 262:4429-4432, insulin as described in Hucked (1990) Biochem Pharmacol 40:253-263, galactose as described in Plank (1992) Bioconjugate Chem 3:533-539, lactose or transferrin.

Naked DNA may also be employed to transform a host cell. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm.

Liposomes that can act as gene delivery vehicles are described in U.S. Pat. No. 5,422,120, WO95/13796, WO94/23697, WO91/14445 and EP-524,968. As described in U.S. Ser. No. 60/023,867, on non-viral delivery, the nucleic acid sequences encoding a polypeptide can be inserted into conventional vectors that contain conventional control sequences for high level expression, and then be incubated with synthetic gene transfer molecules such as polymeric DNA-binding cations like polylysine, protamine, and albumin, linked to cell targeting ligands such as asialoorosomucoid, insulin, galactose, lactose, or transferrin. Other delivery systems include the use of liposomes to encapsulate DNA comprising the gene under the control of a variety of tissue-specific or ubiquitously-active promoters. Further non-viral delivery suitable for use includes mechanical delivery systems such as the approach described in Woffendin et al (1994) Proc. Natl. Acad. Sci. USA 91(24):11581-11585. Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and WO92/11033

Exemplary liposome and polycationic gene delivery vehicles are those described in U.S. Pat. Nos. 5,422,120 and 4,762,915; in WO 95/13796; WO94/23697; and WO91/14445; in EP-0524968; and in Stryer, Biochemistry, pages 236-240 (1975) W. H. Freeman, San Francisco; Szoka (1980) Biochem Biophys Acta 600: 1; Bayer (1979) Biochem Biophys Acta 550:464; Rivnay (1987) Meth Enzymol 149:119; Wang (1987) Proc Natl Acad Sci 84:7851; Plant (1989) Anal Biochem 176:420.

A polynucleotide composition can comprise a therapeutically effective amount of a gene therapy vehicle, as the term is defined above. For purposes of the present invention, an effective dose will be from about 0.01 mg/kg to 50 mg/kg or 0.05 mg/kg to about 10 mg/kg of the DNA constructs in the individual to which it is administered.

Delivery Methods

Once formulated, the polynucleotide compositions of the invention can be administered (1) directly to the subject; (2) delivered ex vivo, to cells derived from the subject; or (3) in vitro for expression of recombinant proteins. The subjects to be treated can be mammals or birds. Also, human subjects can be treated.

Direct delivery of the compositions will generally be accomplished by injection, either subcutaneously, intraperitoneally, transdermally or transcutaneously, intravenously or intramuscularly or delivered to the interstitial space of a tissue. The compositions can also be administered into a tumor or lesion. Other modes of administration include oral and pulmonary administration, suppositories, and transdermal applications, needles, and gene guns or hyposprays. Dosage treatment may be a single dose schedule or a multiple dose schedule. See WO98/20734.

Methods for the ex vivo delivery and reimplantation of transformed cells into a subject are known in the art and described in e.g., WO93/14778. Examples of cells useful in ex vivo applications include, for example, stem cells, particularly hematopoetic, lymph cells, macrophages, dendritic cells, or tumor cells.

Generally, delivery of nucleic acids for both ex vivo and in vitro applications can be accomplished by the following procedures, for example, dextran-mediated transfection, calcium phosphate precipitation, polybrene mediated transfection, protoplast fusion, electroporation, encapsulation of the polynucleotide(s) in liposomes, and direct microinjection of the DNA into nuclei, all well known in the art.

Polynucleotide and Polypeptide Pharmaceutical Compositions

In addition to the pharmaceutically acceptable carriers and salts described above, the following additional agents can be used with polynucleotide and/or polypeptide compositions.

A. Polypeptides

One example are polypeptides which include, without limitation: asialoorosomucoid (ASOR); transferrin; asialoglycoproteins; antibodies; antibody fragments; ferritin; interleukins; interferons, granulocyte, macrophage colony stimulating factor (GM-CSF), granulocyte colony stimulating factor (G-CSF), macrophage colony stimulating factor (M-CSF), stem cell factor and erythropoietin. Viral antigens, such as envelope proteins, can also be used. Also, proteins from other invasive organisms, such as the 17 amino acid peptide from the circumsporozoite protein of plasmodium falciparum known as RII.

B. Hormones, Vitamins, Etc.

Other groups that can be included in a pharmaceutical composition include, for example: hormones, steroids, androgens, estrogens, thyroid hormone, or vitamins, folic acid.

C. Polyalkylenes, Polysaccharides, Etc.

Also, polyalkylene glycol can be included in a pharmaceutical compositions with the desired polynucleotides and/or polypeptides. In a preferred embodiment, the polyalkylene glycol is polyethlylene glycol. In addition, mono-, di-, or polysaccarides can be included. In a preferred embodiment of this aspect, the polysaccharide is dextran or DEAE-dextran. Also, chitosan and poly(lactide-co-glycolide) may be included in a pharmaceutical composition.

D. Lipids, and Liposomes

The desired polynucleotide or polypeptide can also be encapsulated in lipids or packaged in liposomes prior to delivery to the subject or to cells derived therefrom.

Lipid encapsulation is generally accomplished using liposomes which are able to stably bind or entrap and retain nucleic acid or polypeptide. The ratio of condensed polynucleotide to lipid preparation can vary but will generally be around 1:1 (mg DNA:micromoles lipid), or more of lipid. For a review of the use of liposomes as carriers for delivery of nucleic acids, see, Hug and Sleight (1991) Biochim. Biophys. Acta. 1097:1-17; Straubinger (1983) Meth. Enzymol. 101:512-527.

Liposomal preparations for use in the present invention include cationic (positively charged), anionic (negatively charged) and neutral preparations. Cationic liposomes have been shown to mediate intracellular delivery of plasmid DNA (Felgner (1987) Proc. Natl. Acad. Sci. USA 84:7413-7416); mRNA (Malone (1989) Proc. Natl. Acad. Sci. USA 86:6077-6081); and purified transcription factors (Debs (1990) J. Biol. Chem. 265:10189-10192), in functional form.

Cationic liposomes are readily available. For example, N(1-2,3-dioleyloxy)propyl)-N,N,N-triethylammonium (DOTMA) liposomes are available under the trademark Lipofectin, from GIBCO BRL, Grand Island, N.Y. (See, also, Felgner supra). Other commercially available liposomes include transfectace (DDAB/DOPE) and DOTAP/DOPE (Boerhinger). Other cationic liposomes can be prepared from readily available materials using techniques well known in the art. See, e.g., Szoka (1978) Proc. Natl. Acad. Sci. USA 75:4194-4198; WO90/11092 for a description of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See e.g., Straubinger (1983) Meth. Immunol. 101:512-527; Szoka (1978) Proc. Natl. Acad. Sci. USA 75:4194-4198; Papahadjopoulos (1975) Biochim. Biophys. Acta 394:483; Wilson (1979) Cell 17:77); Deamer & Bangham (1976) Biochim. Biophys. Acta 443:629; Ostro (1977) Biochem. Biophys. Res. Commun. 76:836; Fraley (1979) Proc. Natl. Acad. Sci. USA 76:3348); Enoch & Strittmatter (1979) Proc. Natl. Acad. Sci. USA 76:145; Fraley (1980) J. Biol. Chem. (1980) 255:10431; Szoka & Papahadjopoulos (1978) Proc. Natl. Acad. Sci. USA 75:145; and Schaefer-Ridder (1982) Science 215:166.

E. Lipoproteins

In addition, lipoproteins can be included with the polynucleotide or polypeptide to be delivered. Examples of lipoproteins to be utilized include: chylomicrons, HDL, IDL, LDL, and VLDL. Mutants, fragments, or fusions of these proteins can also be used. Also, modifications of naturally occurring lipoproteins can be used, such as acetylated LDL. These lipoproteins can target the delivery of polynucleotides to cells expressing lipoprotein receptors. Preferably, if lipoproteins are including with the polynucleotide to be delivered, no other targeting ligand is included in the composition.

Naturally occurring lipoproteins comprise a lipid and a protein portion. The protein portion are known as apoproteins. At the present, apoproteins A, B, C, D, and E have been isolated and identified. At least two of these contain several proteins, designated by Roman numerals, AI, AII, AIV; CI, CII, CIII.

A lipoprotein can comprise more than one apoprotein. For example, naturally occurring chylomicrons comprises of A, B, C, and E; over time these lipoproteins lose A and acquire C and E apoproteins. VLDL comprises A, B, C, and E apoproteins, LDL comprises apoprotein B; and HDL comprises apoproteins A, C, and E.

The amino acid sequences of these apoproteins are known and are described in, for example, Breslow (1985) Annu Rev. Biochem 54:699; Law (1986) Adv. Exp Med. Biol. 151:162; Chen (1986) J Biol Chem 261:12918; Kane (1980) Proc Natl Acad Sci USA 77:2465; and Utermann (1984) Hum Genet 65:232.

Lipoproteins contain a variety of lipids including, triglycerides, cholesterol (free and esters), and phopholipids. The composition of the lipids varies in naturally occurring lipoproteins. For example, chylomicrons comprise mainly triglycerides. A more detailed description of the lipid content of naturally occurring lipoproteins can be found, for example, in Meth. Enzymol. 128 (1986). The composition of the lipids are chosen to aid in conformation of the apoprotein for receptor binding activity. The composition of lipids can also be chosen to facilitate hydrophobic interaction and association with the polynucleotide binding molecule.

Naturally occurring lipoproteins can be isolated from serum by ultracentrifugation, for instance. Such methods are described in Meth. Enzymol. (supra); Pitas (1980) J. Biochem. 255:5454-5460 and Mahey (1979) J Clin. Invest 64:743-750.

Lipoproteins can also be produced by in vitro or recombinant methods by expression of the apoprotein genes in a desired host cell. See, for example, Atkinson (1986) Annu Rev Biophys Chem 15:403 and Radding (1958) Biochim Biophys Acta 30: 443.

Lipoproteins can also be purchased from commercial suppliers, such as Biomedical Technologies, Inc., Stoughton, Mass., USA.

Further description of lipoproteins can be found in Zuckermann et al., PCT. Appln. No. US97/14465.

F. Polycationic Agents

Polycationic agents can be included, with or without lipoprotein, in a composition with the desired polynucleotide and/or polypeptide to be delivered.

Polycationic agents, typically, exhibit a net positive charge at physiological relevant pH and are capable of neutralizing the electrical charge of nucleic acids to facilitate delivery to a desired location. These agents have both in vitro, ex vivo, and in vivo applications. Polycationic agents can be used to deliver nucleic acids to a living subject either intramuscularly, subcutaneously, etc.

The following are examples of useful polypeptides as polycationic agents: polylysine, polyarginine, polyornithine, and protamine. Other examples of useful polypeptides include histones, protamines, human serum albumin, DNA binding proteins, non-histone chromosomal proteins, coat proteins from DNA viruses, such as ΦX174, transcriptional factors also contain domains that bind DNA and therefore may be useful as nucleic aid condensing agents. Briefly, transcriptional factors such as C/CEBP, c-jun, c-fos, AP-1, AP-2, AP-3, CPF, Prot-1, Sp-1, Oct-1, Oct-2, CREP, and TFIID contain basic domains that bind DNA sequences.

Organic polycationic agents include: spermine, spermidine, and purtrescine.

The dimensions and of the physical properties of a polycationic agent can be extrapolated from the list above, to construct other polypeptide polycationic agents or to produce synthetic polycationic agents.

G. Synthetic Polycationic Agents

Synthetic polycationic agents which are useful in pharmaceutical compositions include, for example, DEAE-dextran, polybrene. Lipofectin™, and lipofectAMINE™ are monomers that form polycationic complexes when combined with polynucleotides or polypeptides.

Immunodiagnostic Assays

Neisseria MenB antigens, or antigenic fragments thereof, of the invention can be used in immunoassays to detect antibody levels (or, conversely, anti-Neisseria MenB antibodies can be used to detect antigen levels). Immunoassays based on well defined, recombinant antigens can be developed to replace invasive diagnostics methods. Antibodies to Neisseria MenB proteins or fragments thereof within biological samples, including for example, blood or serum samples, can be detected. Design of the immunoassays is subject to a great deal of variation, and a variety of these are known in the art. Protocols for the immunoassay may be based, for example, upon competition, or direct reaction, or sandwich type assays. Protocols may also, for example, use solid supports, or may be by immunoprecipitation. Most assays involve the use of labeled antibody or polypeptide; the labels may be, for example, fluorescent, chemiluminescent, radioactive, or dye molecules. Assays which amplify the signals from the probe are also known; examples of which are assays which utilize biotin and avidin, and enzyme-labeled and mediated immunoassays, such as ELISA assays.

Kits suitable for immunodiagnosis and containing the appropriate labeled reagents are constructed by packaging the appropriate materials, including the compositions of the invention, in suitable containers, along with the remaining reagents and materials (for example, suitable buffers, salt solutions, etc.) required for the conduct of the assay, as well as suitable set of assay instructions.

Nucleic Acid Hybridization

“Hybridization” refers to the association of two nucleic acid sequences to one another by hydrogen bonding. Typically, one sequence will be fixed to a solid support and the other will be free in solution. Then, the two sequences will be placed in contact with one another under conditions that favor hydrogen bonding. Factors that affect this bonding include: the type and volume of solvent; reaction temperature; time of hybridization; agitation; agents to block the non-specific attachment of the liquid phase sequence to the solid support (Denhardt's reagent or BLOTTO); concentration of the sequences; use of compounds to increase the rate of association of sequences (dextran sulfate or polyethylene glycol); and the stringency of the washing conditions following hybridization. See Sambrook et al. (supra) Volume 2, chapter 9, pages 9.47 to 9.57.

“Stringency” refers to conditions in a hybridization reaction that favor association of very similar sequences over sequences that differ. For example, the combination of temperature and salt concentration should be chosen that is approximately 120 to 200° C. below the calculated Tm of the hybrid under study. The temperature and salt conditions can often be determined empirically in preliminary experiments in which samples of genomic DNA immobilized on filters are hybridized to the sequence of interest and then washed under conditions of different stringencies. See Sambrook et al. at page 9.50.

Variables to consider when performing, for example, a Southern blot are (1) the complexity of the DNA being blotted and (2) the homology between the probe and the sequences being detected. The total amount of the fragment(s) to be studied can vary a magnitude of 10, from 0.1 to 1 μg for a plasmid or phage digest to 10⁻⁹ to 10⁻⁸ g for a single copy gene in a highly complex eukaryotic genome. For lower complexity polynucleotides, substantially shorter blotting, hybridization, and exposure times, a smaller amount of starting polynucleotides, and lower specific activity of probes can be used. For example, a single-copy yeast gene can be detected with an exposure time of only 1 hour starting with 1 μg of yeast DNA, blotting for two hours, and hybridizing for 4-8 hours with a probe of 10⁸ cpm/μg. For a single-copy mammalian gene a conservative approach would start with 10 μg of DNA, blot overnight, and hybridize overnight in the presence of 10% dextran sulfate using a probe of greater than 10⁸ cpm/μg, resulting in an exposure time of ˜24 hours.

Several factors can affect the melting temperature (Tm) of a DNA-DNA hybrid between the probe and the fragment of interest, and consequently, the appropriate conditions for hybridization and washing. In many cases the probe is not 100% homologous to the fragment. Other commonly encountered variables include the length and total G+C content of the hybridizing sequences and the ionic strength and formamide content of the hybridization buffer. The effects of all of these factors can be approximated by a single equation:
Tm=81+16.6(log₁₀Ci)+0.4(% (G+C))−0.6(% formamide)−600/n−1.5(% mismatch)
where Ci is the salt concentration (monovalent ions) and n is the length of the hybrid in base pairs (slightly modified from Meinkoth & Wahl (1984) Anal. Biochem. 138:267-284).

In designing a hybridization experiment, some factors affecting nucleic acid hybridization can be conveniently altered. The temperature of the hybridization and washes and the salt concentration during the washes are the simplest to adjust. As the temperature of the hybridization increases (i.e., stringency), it becomes less likely for hybridization to occur between strands that are nonhomologous, and as a result, background decreases. If the radiolabeled probe is not completely homologous with the immobilized fragment (as is frequently the case in gene family and interspecies hybridization experiments), the hybridization temperature must be reduced, and background will increase. The temperature of the washes affects the intensity of the hybridizing band and the degree of background in a similar manner. The stringency of the washes is also increased with decreasing salt concentrations.

In general, convenient hybridization temperatures in the presence of 50% formamide are 42° C. for a probe with is 95% to 100% homologous to the target fragment, 37° C. for 90% to 95% homology, and 32° C. for 85% to 90% homology. For lower homologies, formamide content should be lowered and temperature adjusted accordingly, using the equation above. If the homology between the probe and the target fragment are not known, the simplest approach is to start with both hybridization and wash conditions which are nonstringent. If non-specific bands or high background are observed after autoradiography, the filter can be washed at high stringency and reexposed. If the time required for exposure makes this approach impractical, several hybridization and/or washing stringencies should be tested in parallel.

Nucleic Acid Probe Assays

Methods such as PCR, branched DNA probe assays, or blotting techniques utilizing nucleic acid probes according to the invention can determine the presence of cDNA or mRNA. A probe is said to “hybridize” with a sequence of the invention if it can form a duplex or double stranded complex, which is stable enough to be detected.

The nucleic acid probes will hybridize to the Neisserial nucleotide sequences of the invention (including both sense and antisense strands). Though many different nucleotide sequences will encode the amino acid sequence, the native Neisserial sequence is preferred because it is the actual sequence present in cells. mRNA represents a coding sequence and so a probe should be complementary to the coding sequence; single-stranded cDNA is complementary to mRNA, and so a cDNA probe should be complementary to the non-coding sequence.

The probe sequence need not be identical to the Neisserial sequence (or its complement)—some variation in the sequence and length can lead to increased assay sensitivity if the nucleic acid probe can form a duplex with target nucleotides, which can be detected. Also, the nucleic acid probe can include additional nucleotides to stabilize the formed duplex. Additional Neisserial sequence may also be helpful as a label to detect the formed duplex. For example, a non-complementary nucleotide sequence may be attached to the 5′ end of the probe, with the remainder of the probe sequence being complementary to a Neisserial sequence. Alternatively, non-complementary bases or longer sequences can be interspersed into the probe, provided that the probe sequence has sufficient complementarity with the a Neisserial sequence in order to hybridize therewith and thereby form a duplex which can be detected.

The exact length and sequence of the probe will depend on the hybridization conditions, such as temperature, salt condition and the like. For example, for diagnostic applications, depending on the complexity of the analyte sequence, the nucleic acid probe typically contains at least 10-20 nucleotides, preferably 15-25, and more preferably at least 30 nucleotides, although it may be shorter than this. Short primers generally require cooler temperatures to form sufficiently stable hybrid complexes with the template.

Probes may be produced by synthetic procedures, such as the triester method of Matteucci et al. (J. Am. Chem. Soc. (1981) 103:3185), or according to Urdea et al. (Proc. Natl. Acad. Sci. USA (1983) 80: 7461), or using commercially available automated oligonucleotide synthesizers.

The chemical nature of the probe can be selected according to preference. For certain applications, DNA or RNA are appropriate. For other applications, modifications may be incorporated e.g., backbone modifications, such as phosphorothioates or methylphosphonates, can be used to increase in vivo half-life, alter RNA affinity, increase nuclease resistance etc. (e.g., see Agrawal & Iyer (1995) Curr Opin Biotechnol 6:12-19; Agrawal (1996) TIBTECH 14:376-387); analogues such as peptide nucleic acids may also be used (e.g., see Corey (1997) TIBTECH 15:224-229; Buchardt et al. (1993) TIBTECH 11:384-386).

One example of a nucleotide hybridization assay is described by Urdea et al. in international patent application WO92/02526 (see also U.S. Pat. No. 5,124,246).

Alternatively, the polymerase chain reaction (PCR) is another well-known means for detecting small amounts of target nucleic acids. The assay is described in: Mullis et al. (Meth. Enzyrnol. (1987) 155: 335-350); U.S. Pat. No. 4,683,195; and U.S. Pat. No. 4,683,202. Two “primer” nucleotides hybridize with the target nucleic acids and are used to prime the reaction. The primers can comprise sequence that does not hybridize to the sequence of the amplification target (or its complement) to aid with duplex stability or, for example, to incorporate a convenient restriction site. Typically, such sequence will flank the desired Neisserial sequence.

A thermostable polymerase creates copies of target nucleic acids from the primers using the original target nucleic acids as a template. After a threshold amount of target nucleic acids are generated by the polymerase, they can be detected by more traditional methods, such as Southern blots. When using the Southern blot method, the labeled probe will hybridize to the Neisserial sequence (or its complement).

Also, mRNA or cDNA can be detected by traditional blotting techniques described in Sambrook et al (supra). mRNA, or cDNA generated from mRNA using a polymerase enzyme, can be purified and separated using gel electrophoresis. The nucleic acids on the gel are then blotted onto a solid support, such as nitrocellulose. The solid support is exposed to a labeled probe and then washed to remove any unhybridized probe. Next, the duplexes containing the labeled probe are detected. Typically, the probe is labeled with a radioactive moiety.

### EXAMPLES

The invention is based on the 961 nucleotide sequences from the genome of N. meningitidis set out in Appendix C, SEQ ID NOs:1-961 of the '573 application, which together represent substantially the complete genome of serotype B of N. meningitidis, as well as the full length genome sequence shown in Appendix D, SEQ ID NO 1068 of the '573 application, and the full length genome sequence shown in Appendix A hereto, SEQ ID NO. 1.

It will be self-evident to the skilled person how this sequence information can be utilized according to the invention, as above described.

The standard techniques and procedures which may be employed in order to perform the invention (e.g. to utilize the disclosed sequences to predict polypeptides useful for vaccination or diagnostic purposes) were summarized above. This summary is not a limitation on the invention but, rather, gives examples that may be used, but are not required.

These sequences are derived from contigs shown in Appendix C (SEQ ID NOs 1-961) and from the full length genome sequence shown in Appendix D (SEQ ID NO 1068), which were prepared during the sequencing of the genome of N. meningitidis (strain B). The full length sequence was assembled using the TIGR Assembler as described by G. S. Sutton et al., TIGR Assembler: A New Tool for Assembling Large Shotgun Sequencing Projects, Genome Science and Technology, 1:9-19 (1995) [see also R. D. Fleischmann, et al., Science 269, 496-512 (1995); C. M. Fraser, et al., Science 270, 397-403 (1995); C. J. Bult, et al., Science 273, 1058-73 (1996); C. M. Fraser, et al, Nature 390, 580-586 (1997); J.-F. Tomb, et al., Nature 388, 539-547 (1997); H. P. Klenk, et al., Nature 390, 364-70 (1997); C. M. Fraser, et al., Science 281, 375-88 (1998); M. J. Gardner, et al., Science 282, 1126-1132 (1998); K. E. Nelson, et al., Nature 399, 323-9 (1999)]. Then, using the above-described methods, putative translation products of the sequences were determined. Computer analysis of the translation products were determined based on database comparisons. Corresponding gene and protein sequences, if any, were identified in Neisseria meningitidis (Strain A) and Neisseria gonorrhoeae. Then the proteins were expressed, purified, and characterized to assess their antigenicity and immunogenicity.

In particular, the following methods were used to express, purify, and biochemically characterize the proteins of the invention.

Chromosomal DNA Preparation

N. meningitidis strain 2996 was grown to exponential phase in 100 ml of GC medium, harvested by centrifugation, and resuspended in 5 ml buffer (20% Sucrose, 50 mM Tris-HCl, 50 mM EDTA, adjusted to pH 8.0). After 10 minutes incubation on ice, the bacteria were lysed by adding 10 ml lysis solution (50 mM NaCl, 1% Na-Sarkosyl, 50 μg/ml Proteinase K), and the suspension was incubated at 37° C. for 2 hours. Two phenol extractions (equilibrated to pH 8) and one ChCl₃/isoamylalcohol (24:1) extraction were performed. DNA was precipitated by addition of 0.3M sodium acetate and 2 volumes ethanol, and was collected by centrifugation. The pellet was washed once with 70% ethanol and redissolved in 4 ml buffer (10 mM Tris-HCl, 1 mM EDTA, pH 8). The DNA concentration was measured by reading the OD at 260 nm.

Oligonucleotide Design

Synthetic oligonucleotide primers were designed on the basis of the coding sequence of each ORF, using (a) the meningococcus B sequence when available, or (b) the gonococcus/meningococcus A sequence, adapted to the codon preference usage of meningococcus. Any predicted signal peptides were omitted, by deducing the 5′-end amplification primer sequence immediately downstream from the predicted leader sequence.

For most ORFs, the 5′ primers included two restriction enzyme recognition sites (BamHI-NdeI, BamHI-NheI, or EcoRI-NheI, depending on the gene's restriction pattern); the 3′ primers included a XhoI restriction site. This procedure was established in order to direct the cloning of each amplification product (corresponding to each ORF) into two different expression systems: pGEX-KG (using either BamHI-XhoI or EcoRI-XhoI), and pET21b+ (using either NdeI-XhoI or NheI-XhoI).

| | | |
|---|---|---|
| 5′-end primer tail: | | |
| CGCGGATCCCATATG | (BamHI-NdeI) | SEQ ID NO: 108 |
| CGCGGATCCGCTAGC | (BamHI-NheI) | SEQ ID NO: 109 |
| CCGGAATTCTAGCTAGC | (EcoRI-NheI) | SEQ ID NO: 110 |
| 3′-end primer tail: | | |
| CCCGCTCGAG | (XhoI) | SEQ ID NO: 111 |

For some ORFs, two different amplifications were performed to clone each ORF in the two expression systems. Two different 5′ primers were used for each ORF; the same 3′ XhoI primer was used as before:

| | | |
|---|---|---|
| 5′-end primer tail: | | |
| GGAATTCCATATGGCCATGG | (NdeI) | SEQ ID NO: 112 |
| 5′-end primer tail: | | |
| CGGGATCC | (BamHI) | SEQ ID NO: 113 |

Other ORFs were cloned in the pTRC expression vector and expressed as an amino-terminus His-tag fusion. The predicted signal peptide may be included in the final product. NheI-BamHI restriction sites were incorporated using primers:

| | | |
|---|---|---|
| 5′-end primer tail: | | |
| GATCAGCTAGCCATATG | (NheI) | SEQ ID NO: 114 |
| 3′-end primer tail: | | |
| CGGGATCC | (BamHI) | SEQ ID NO: 115 |

As well as containing the restriction enzyme recognition sequences, the primers included nucleotides which hybridized to the sequence to be amplified. The number of hybridizing nucleotides depended on the melting temperature of the whole primer, and was determined for each primer using the formulae:
Tm=4(G+C)+2(A+T) (tail excluded)
Tm=64.9+0.41 (% GC)−600/N (whole primer)

The average melting temperature of the selected oligos were 65-70° C. for the whole oligo and 50-55° C. for the hybridising region alone.

Oligos were synthesized by a Perkin Elmer 394 DNA/RNA Synthesizer, eluted from the columns in 2 ml NH₄—OH, and deprotected by 5 hours incubation at 56° C. The oligos were precipitated by addition of 0.3M Na-Acetate and 2 volumes ethanol. The samples were then centrifuged and the pellets resuspended in either 100 μl or 1 ml of water. OD₂₆₀ was determined using a Perkin Elmer Lambda Bio spectophotometer and the concentration was determined and adjusted to 2-10 pmol/μl.

Table 1 shows the forward and reverse primers used for each amplification. In certain cases, it might be noted that the sequence of the primer does not exactly match the sequence in the ORF. When initial amplifications are performed, the complete 5′ and/or 3′ sequence may not be known for some meningococcal ORFs, although the corresponding sequences may have been identified in gonoccus. For amplification, the gonococcal sequences could thus be used as the basis for primer design, altered to take account of codon preference. In particular, the following codons may be changed: ATA→ATT; TCG→TCT; CAG→CAA; AAG→AAA; GAG→GAA; CGA and CGG→CGC; GGG→GGC.

Amplification

The standard PCR protocol was as follows: 50-200 ng of genomic DNA were used as a template in the presence of 20-40 μM of each oligo, 400-800 μM dNTPs solution, 1×PCR buffer (including 1.5 mM MgCl₂), 2.5 units TaqI DNA polymerase (using Perkin-Elmer AmpliTaQ, GIBCO Platinum, Pwo DNA polymerase, or Tahara Shuzo Taq polymerase).

In some cases, PCR was optimised by the addition of 10 μl DMSO or 50 μl 2M betaine.

After a hot start (adding the polymerase during a preliminary 3 minute incubation of the whole mix at 95° C.), each sample underwent a double-step amplification: the first 5 cycles were performed using as the hybridization temperature the one of the oligos excluding the restriction enzymes tail, followed by 30 cycles performed according to the hybridization temperature of the whole length oligos. The cycles were followed by a final 10 minute extension step at 72° C.

The standard cycles were as follows:

| | Denaturation | Hybridisation | Elongation |
|---|---|---|---|
| First 5 cycles | 30 seconds | 30 seconds | 30-60 seconds |
| | 95° C. | 50-55° C. | 72° C. |
| Last 30 cycles | 30 seconds | 30 seconds | 30-60 seconds |
| | 95° C. | 65-70° C. | 72° C. |

The elongation time varied according to the length of the ORF to be amplified.

The amplifications were performed using either a 9600 or a 2400 Perkin Elmer GeneAmp PCR System. To check the results, 1/10 of the amplification volume was loaded onto a 1-1.5% agarose gel and the size of each amplified fragment compared with a DNA molecular weight marker.

The amplified DNA was either loaded directly on a 1% agarose gel or first precipitated with ethanol and resuspended in a suitable volume to be loaded on a 1% agarose gel. The DNA fragment corresponding to the right size band was then eluted and purified from gel, using the Qiagen Gel Extraction Kit, following the instructions of the manufacturer. The final volume of the DNA fragment was 30 μl or 50 μl of either water or 10 mM Tris, pH 8.5.

Digestion of PCR Fragments

The purified DNA corresponding to the amplified fragment was split into 2 aliquots and double-digested with:

NdeI/AhoI or NheI/XhoI for cloning into pET-21b+ and further expression of the protein as a C-terminus His-tag fusion

BamHI/XhoI or EcoRI/AhoI for cloning into pGEX-KG and further expression of the protein as a GST N-terminus fusion.

For ORF 76, NheI/BamHI for cloning into pTRC-HisA vector and further expression of the protein as N-terminus His-tag fusion.

Each purified DNA fragment was incubated (37° C. for 3 hours to overnight) with 20 units of each restriction enzyme (New England Biolabs) in a either 30 or 40 μl final volume in the presence of the appropriate buffer. The digestion product was then purified using the QIAquick PCR purification kit, following the manufacturer's instructions, and eluted in a final volume of 30 (or 50) μl of either water or 10 mM Tris-HCl, pH 8.5. The final DNA concentration was determined by 1% agarose gel electrophoresis in the presence of titrated molecular weight marker.

Digestion of the Cloning Vectors (pET22B, pGEX-KG and pTRC-His A)

10 μg plasmid was double-digested with 50 units of each restriction enzyme in 200 μl reaction volume in the presence of appropriate buffer by overnight incubation at 37° C. After loading the whole digestion on a 1% agarose gel, the band corresponding to the digested vector was purified from the gel using the Qiagen QIAquick Gel Extraction Kit and the DNA was eluted in 50 μl of 10 mM Tris-HCl, pH 8.5. The DNA concentration was evaluated by measuring OD₂₆₀ of the sample, and adjusted to 50 μg/μl. 1 μl of plasmid was used for each cloning procedure.

Cloning

The fragments corresponding to each ORF, previously digested and purified, were ligated in both pET22b and pGEX-KG. In a final volume of 20 μl, a molar ratio of 3:1 fragment/vector was ligated using 0.5 μl of NEB T4 DNA ligase (400 units/μl), in the presence of the buffer supplied by the manufacturer. The reaction was incubated at room temperature for 3 hours. In some experiments, ligation was performed using the Boheringer “Rapid Ligation Kit”, following the manufacturer's instructions.

In order to introduce the recombinant plasmid in a suitable strain, 100 μl E. coli DH5 competent cells were incubated with the ligase reaction solution for 40 minutes on ice, then at 37° C. for 3 minutes, then, after adding 800 μl LB broth, again at 37° C. for 20 minutes. The cells were then centrifuged at maximum speed in an Eppendorf microfuge and resuspended in approximately 200 μl of the supernatant. The suspension was then plated on LB ampicillin (100 mg/ml).

The screening of the recombinant clones was performed by growing 5 randomly-chosen colonies overnight at 37° C. in either 2 ml (PGEX or pTC clones) or 5 ml (pET clones) LB broth+100 μg/ml ampicillin. The cells were then pelletted and the DNA extracted using the Qiagen QIAprep Spin Miniprep Kit, following the manufacturer's instructions, to a final volume of 30 μl. 5 μl of each individual miniprep (approximately 1 g) were digested with either NdeI/XhoI or BamHI/XhoI and the whole digestion loaded onto a 1-1.5% agarose gel (depending on the expected insert size), in parallel with the molecular weight marker (1 Kb DNA Ladder, GIBCO). The screening of the positive clones was made on the base of the correct insert size.

Cloning

Certain ORFs may be cloned into the pGEX-HIS vector using EcoRI-PstI, EcoRI-SalI, or SalI-PstI cloning sites. After cloning, the recombinant plasmids may be introduced in the E. coli host W3110.

Expression

Each ORF cloned into the expression vector may then be transformed into the strain suitable for expression of the recombinant protein product. 1 μl of each construct was used to transform 30 μl of E. coli BL21 (pGEX vector), E. coli TOP 10 (pTRC vector) or E. coli BL21-DE3 (pET vector), as described above. In the case of the pGEX-His vector, the same E. coli strain (W3110) was used for initial cloning and expression. Single recombinant colonies were inoculated into 2 ml LB+Amp (100 μg/ml), incubated at 37° C. overnight, then diluted 1:30 in 20 ml of LB+Amp (100 μg/ml) in 100 ml flasks, making sure that the OD₆₀₀ ranged between 0.1 and 0.15. The flasks were incubated at 30° C. into gyratory water bath shakers until OD indicated exponential growth suitable for induction of expression (0.4-0.8 OD for pET and pTRC vectors; 0.8-1 OD for pGEX and pGEX-His vectors). For the pET, pTRC and pGEX-His vectors, the protein expression was induced by addiction of 1 mM IPTG, whereas in the case of pGEX system the final concentration of IPTG was 0.2 mM. After 3 hours incubation at 30° C., the final concentration of the sample was checked by OD. In order to check expression, 1 ml of each sample was removed, centrifuged in a microfuge, the pellet resuspended in PBS, and analysed by 12% SDS-PAGE with Coomassie Blue staining. The whole sample was centrifuged at 6000 g and the pellet resuspended in PBS for further use.

GST-Fusion Proteins Large-Scale Purification.

A single colony was grown overnight at 37° C. on LB+Amp agar plate. The bacteria were inoculated into 20 ml of LB+Amp liquid culture in a water bath shaker and grown overnight. Bacteria were diluted 1:30 into 600 ml of fresh medium and allowed to grow at the optimal temperature (20-37° C.) to OD₅₅₀ 0.8-1. Protein expression was induced with 0.2 mM IPTG followed by three hours incubation. The culture was centrifuged at 8000 rpm at 4° C. The supernatant was discarded and the bacterial pellet was resuspended in 7.5 ml cold PBS. The cells were disrupted by sonication on ice for 30 sec at 40 W using a Branson sonifier B-15, frozen and thawed two times and centrifuged again. The supernatant was collected and mixed with 150 μl Glutatione-Sepharose 4B resin (Pharmacia) (previously washed with PBS) and incubated at room temperature for 30 minutes. The sample was centrifuged at 700 g for 5 minutes at 4 C. The resin was washed twice with 10 ml cold PBS for 10 minutes, resuspended in 1 ml cold PBS, and loaded on a disposable column. The resin was washed twice with 2 ml cold PBS until the flow-through reached OD₂₈₀ of 0.02-0.06. The GST-fusion protein was eluted by addition of 700 μl cold Glutathione elution buffer 10 mM reduced glutathione, 50 mM Tris-HCl) and fractions collected until the OD₂₈₀ was 0.1. 21 μl of each fraction were loaded on a 12% SDS gel using either Biorad SDS-PAGE Molecular weight standard broad range (M1) (200, 116.25, 97.4, 66.2, 45, 31, 21.5, 14.4, 6.5 kDa) or Amersham Rainbow Marker (M″) (220, 66, 46, 30, 21.5, 14.3 kDa) as standards. As the MW of GST is 26 kDa, this value must be added to the MW of each GST-fusion protein.

His-Fusion Soluble Proteins Large-Scale Purification.

A single colony was grown overnight at 37° C. on a LB+Amp agar plate. The bacteria were inoculated into 20 ml of LB+Amp liquid culture and incubated overnight in a water bath shaker. Bacteria were diluted 1:30 into 600 ml fresh medium and allowed to grow at the optimal temperature (20-37° C.) to OD₅₅₀ 0.6-0.8. Protein expression was induced by addition of 1 mM IPTG and the culture further incubated for three hours. The culture was centrifuged at 8000 rpm at 4° C., the supernatant was discarded and the bacterial pellet was resuspended in 7.5 ml cold 10 mM imidazole buffer (300 mM NaCl, 50 mM phosphate buffer, 10 mM imidazole, pH 8). The cells were disrupted by sonication on ice for 30 sec at 40 W using a Branson sonifier B-15, frozen and thawed two times and centrifuged again. The supernatant was collected and mixed with 150 μl Ni²⁺-resin (Pharmacia) (previously washed with 10 mM imidazole buffer) and incubated at room temperature with gentle agitation for 30 minutes. The sample was centrifuged at 700 g for 5 minutes at 4° C. The resin was washed twice with 10 ml cold 10 mM imidazole buffer for 10 minutes, resuspended in 1 ml cold 10 mM imidazole buffer and loaded on a disposable column. The resin was washed at 4° C. with 2 ml cold 10 mM imidazole buffer until the flow-through reached the O.D₂₈₀ of 0.02-0.06. The resin was washed with 2 ml cold 20 mM imidazole buffer (300 mM NaCl, 50 mM phosphate buffer, 20 mM imidazole, pH 8) until the flow-through reached the O.D₂₈₀ of 0.02-0.06. The His-fusion protein was eluted by addition of 700 μl cold 250 mM imidazole buffer (300 mM NaCl, 50 mM phosphate buffer, 250 mM imidazole, pH 8) and fractions collected until the O.D₂₈₀ was 0.1. 21 μl of each fraction were loaded on a 12% SDS gel.

His-Fusion Insoluble Proteins Large-Scale Purification.

A single colony was grown overnight at 37° C. on a LB+Amp agar plate. The bacteria were inoculated into 20 ml of LB+Amp liquid culture in a water bath shaker and grown overnight. Bacteria were diluted 1:30 into 600 ml fresh medium and let to grow at the optimal temperature (37° C.) to O.D₅₅₀ 0.6-0.8. Protein expression was induced by addition of 1 mM IPTG and the culture further incubated for three hours. The culture was centrifuged at 8000 rpm at 4° C. The supernatant was discarded and the bacterial pellet was resuspended in 7.5 ml buffer B (urea 8M, 10 mM Tris-HCl, 100 mM phosphate buffer, pH 8.8). The cells were disrupted by sonication on ice for 30 sec at 40 W using a Branson sonifier B-15, frozen and thawed twice and centrifuged again. The supernatant was stored at −20° C., while the pellets were resuspended in 2 ml guanidine buffer (6M guanidine hydrochloride, 100 mM phosphate buffer, 10 mM Tris-HCl, pH 7.5) and treated in a homogenizer for 10 cycles. The product was centrifuged at 13000 rpm for 40 minutes. The supernatant was mixed with 150 μl Ni²⁺-resin (Pharmacia) (previously washed with buffer B) and incubated at room temperature with gentle agitation for 30 minutes. The sample was centrifuged at 700 g for 5 minutes at 4° C. The resin was washed twice with 10 ml buffer B for 10 minutes, resuspended in 1 ml buffer B, and loaded on a disposable column. The resin was washed at room temperature with 2 ml buffer B until the flow-through reached the OD₂₈₀ of 0.02-0.06. The resin was washed with 2 ml buffer C (urea 8M, 10 mM Tris-HCl, 100 mM phosphate buffer, pH 6.3) until the flow-through reached the O.D₂₈₀ of 0.02-0.06. The His-fusion protein was eluted by addition of 700 μl elution buffer (urea 8M, 10 mM Tris-HCl, 100 mM phosphate buffer, pH 4.5) and fractions collected until the OD₂₈₀ was 0.1. 21 μl of each fraction were loaded on a 12% SDS gel.

His-Fusion Proteins Renaturation

10% glycerol was added to the denatured proteins. The proteins were then diluted to 20 μg/ml using dialysis buffer I (10% glycerol, 0.5M arginine, 50 mM phosphate buffer, 5 mM reduced glutathione, 0.5 mM oxidised glutathione, 2M urea, pH 8.8) and dialysed against the same buffer at 4° C. for 12-14 hours. The protein was further dialysed against dialysis buffer II (10% glycerol, 0.5M arginine, 50 mM phosphate buffer, 5 mM reduced glutathione, 0.5 mM oxidised glutathione, pH 8.8) for 12-14 hours at 4° C. Protein concentration was evaluated using the formula:
Protein (mg/ml)=(1.55×OD₂₈₀)−(0.76×OD₂₆₀)
Mice Immunisations

20 μg of each purified protein were used to immunise mice intraperitoneally. In the case of some ORFs, Balb-C mice were immunised with Al(OH)₃ as adjuvant on days 1, 21 and 42, and immune response was monitored in samples taken on day 56. For other ORFs, CD1 mice could be immunised using the same protocol. For other ORFs, CD1 mice could be immunised using Freund's adjuvant, and the same immunisation protocol was used, except that the immune response was measured on day 42, rather than 56. Similarly, for still other ORFs, CD1 mice could be immunised with Freund's adjuvant, but the immune response was measured on day 49.

ELISA Assay (Sera Analysis)

The capsulated MenB M7 strain was plated on chocolate agar plates and incubated overnight at 37° C. Bacterial colonies were collected from the agar plates using a sterile dracon swab and inoculated into 7 ml of Mueller-Hinton Broth (Difco) containing 0.25% Glucose. Bacterial growth was monitored every 30 minutes by following OD₆₂₀. The bacteria were let to grow until the OD reached the value of 0.3-0.4. The culture was centrifuged for 10 minutes at 10000 rpm. The supernatant was discarded and bacteria were washed once with PBS, resuspended in PBS containing 0.025% formaldehyde, and incubated for 2 hours at room temperature and then overnight at 4° C. with stirring. 100 μl bacterial cells were added to each well of a 96 well Greiner plate and incubated overnight at 4° C. The wells were then washed three times with PBT washing buffer (0.1% Tween-20 in PBS). 200 μl of saturation buffer (2.7% Polyvinylpyrrolidone 10 in water) was added to each well and the plates incubated for 2 hours at 37° C. Wells were washed three times with PBT. 200 μl of diluted sera (Dilution buffer: 1% BSA, 0.1% Tween-20, 0.1% NaN₃ in PBS) were added to each well and the plates incubated for 90 minutes at 37° C. Wells were washed three times with PBT. 100 μl of HRP-conjugated rabbit anti-mouse (Dako) serum diluted 1:2000 in dilution buffer were added to each well and the plates were incubated for 90 minutes at 37° C. Wells were washed three times with PBT buffer. 100 μl of substrate buffer for HRP (25 ml of citrate buffer pH5, 10 mg of O-phenildiamine and 10 μl of H₂O) were added to each well and the plates were left at room temperature for 20 minutes. 100 μl H₂SO₄ was added to each well and OD₄₉₀ was followed. The ELISA was considered positive when OD490 was 2.5 times the respective pre-immune sera.

FACScan Bacteria Binding Assay Procedure.

The acapsulated MenB M7 strain was plated on chocolate agar plates and incubated overnight at 37° C. Bacterial colonies were collected from the agar plates using a sterile dracon swab and inoculated into 4 tubes containing 8 ml each Mueller-Hinton Broth (Difco) containing 0.25% glucose. Bacterial growth was monitored every 30 minutes by following OD₆₂₀. The bacteria were let to grow until the OD reached the value of 0.35-0.5. The culture was centrifuged for 10 minutes at 4000 rpm. The supernatant was discarded and the pellet was resuspended in blocking buffer (1% BSA, 0.4% NaN₃) and centrifuged for 5 minutes at 4000 rpm. Cells were resuspended in blocking buffer to reach OD₆₂₀ of 0.07. 100μl bacterial cells were added to each well of a Costar 96 well plate. 100 μl of diluted (1:200) sera (in blocking buffer) were added to each well and plates incubated for 2 hours at 4° C. Cells were centrifuged for 5 minutes at 4000 rpm, the supernatant aspirated and cells washed by addition of 200 μl/well of blocking buffer in each well. 100 μl of R-Phicoerytrin conjugated F(ab)₂ goat anti-mouse, diluted 1:100, was added to each well and plates incubated for 1 hour at 4° C. Cells were spun down by centrifugation at 400 rpm for 5 minutes and washed by addition of 200 μl/well of blocking buffer. The supernatant was aspirated and cells resuspended in 200 μl/well of PBS, 0.25% formaldehyde. Samples were transferred to FACScan tubes and read. The condition for FACScan setting were: FL1 on, FL2 and FL3 off; FSC-H Threshold: 92; FSC PMT Voltage: E 02; SSC PMT: 474; Amp. Gains 7.1; FL-2 PMT: 539. Compensation values: 0.

OMV Preparations

Bacteria were grown overnight on 5 GC plates, harvested with a loop and resuspended in 10 ml 20 mM Tris-HCl. Heat inactivation was performed at 56° C. for 30 minutes and the bacteria disrupted by sonication for 10′ on ice (50% duty cycle, 50% output). Unbroken cells were removed by centrifugation at 5000 g for 10 minutes and the total cell envelope fraction recovered by centrifugation at 50000 g at 4° C. for 75 minutes. To extract cytoplasmic membrane proteins from the crude outer membranes, the whole fraction was resuspended in 2% sarkosyl (Sigma) and incubated at room temperature for 20 minutes. The suspension was centrifuged at 10000 g for 10 minutes to remove aggregates, and the supernatant further ultracentrifuged at 50000 g for 75 minutes to pellet the outer membranes. The outer membranes were resuspended in 10 mM Tris-HCl, pH8 and the protein concentration measured by the Bio-Rad Protein assay, using BSA as a standard.

Whole Extracts Preparation

Bacteria were grown overnight on a GC plate, harvested with a loop and resuspended in 1 ml of 20 mM Tris-HCl. Heat inactivation was performed at 56° C. for 30′ minutes.

Western Blotting

Purified proteins (500 ng/lane), outer membrane vesicles (5 μg) and total cell extracts (25 μg) derived from MenB strain 2996 were loaded on 15% SDS-PAGE and transferred to a nitrocellulose membrane. The transfer was performed for 2 hours at 150 mA at 4° C., in transferring buffer (0.3% Tris base, 1.44% glycine, 20% methanol). The membrane was saturated by overnight incubation at 4° C. in saturation buffer (10% skimmed milk, 0.1% Triton X100 in PBS). The membrane was washed twice with washing buffer (3% skimmed milk, 0.1% Triton X100 in PBS) and incubated for 2 hours at 37° C. with 1:200 mice sera diluted in washing buffer. The membrane was washed twice and incubated for 90 minutes with a 1:2000 dilution of horseradish peroxidase labeled anti-mouse Ig. The membrane was washed twice with 0.1% Triton X100 in PBS and developed with the Opti-4CN Substrate Kit (Bio-Rad). The reaction was stopped by adding water.

Bactericidal Assay

MC58 strain was grown overnight at 37° C. on chocolate agar plates. 5-7 colonies were collected and used to inoculate 7 ml Mueller-Hinton broth. The suspension was incubated at 37° C. on a nutator and let to grow until OD₆₂₀ was in between 0.5-0.8. The culture was aliquoted into sterile 1.5 ml Eppendorf tubes and centrifuged for 20 minutes at maximum speed in a microfuge. The pellet was washed once in Gey's buffer (Gibco) and resuspended in the same buffer to an OD₆₂₀ of 0.5, diluted 1:20000 in Gey's buffer and stored at 25° C.

50 μl of Gey's buffer/1% BSA was added to each well of a 96-well tissue culture plate. 25 μl of diluted (1:100) mice sera (dilution buffer: Gey's buffer/0.2% BSA) were added to each well and the plate incubated at 4° C. 25 μl of the previously described bacterial suspension were added to each well. 25 μl of either heat-inactivated (56° C. waterbath for 30 minutes) or normal baby rabbit complement were added to each well. Immediately after the addition of the baby rabbit complement, 22 μl of each sample/well were plated on Mueller-Hinton agar plates (time 0). The 96-well plate was incubated for 1 hour at 37° C. with rotation and then 22 μl of each sample/well were plated on Mueller-Hinton agar plates (time 1). After overnight incubation the colonies corresponding to time 0 and time 1 h were counted.

The following DNA and amino acid sequences are identified by titles of the following form: [g, m, or a][#].[seq or pep], where “g” means a sequence from N. gonorrhoeae, “m” means a sequence from N. meningitidis B, and “a” means a sequence from N. meningitidis A; “#” means the number of the sequence; “seq” means a DNA sequence, and “pep” means an amino acid sequence. For example, “g001.seq” refers to an N. gonorrohoeae DNA sequence, number 1. The presence of the suffix “-1” or “-2” to these sequences indicates an additional sequence found for the same ORF. Further, open reading frames are identified as ORF #, where “#” means the number of the ORF, corresponding to the number of the sequence which encodes the ORF, and the ORF designations may be suffixed with “.ng” or “.a”, indicating that the ORF corresponds to a N. gonorrhoeae sequence or a N. meningitidis A sequence, respectively. Computer analysis was performed for the comparisons that follow between “g”, “m”, and “a” peptide sequences; and therein the “pep” suffix is implied where not expressly stated.

### EXAMPLE 1

The following ORFs were predicted from the contig sequences and/or the full length sequences using the methods herein described.

Localization of the ORFs

ORF: contig:
279 gnm4.seq

The following partial DNA sequence was identified in N. meningitidis <SEQ ID 2>:

| m279.seq | |
|---|---|
| 1 | ATAACGCGGA TTTGCGGCTG CTTGATTTCA ACGGTTTTCA GGGCTTCGGC |
| 51 | AAGTTTGTCG GCGGCGGGTT TCATCAGGCT GCAATGGGAA GGTACGGACA |
| 101 | CGGGCAGCGG CAGGGCGCGT TTGGCACCGG CTTCTTTGGC GGCAGCCATG |
| 151 | GCGCGTCCGA CGGCGGCGGC GTTGCCTGCA ATCACGATTT GTCCGGGTGA |
| 201 | GTTGAAGTTG ACGGCTTCGA CCACTTCGCT TTGGGCGGCT TCGGCACAAA |
| 251 | TGGCTTTAAC CTGCTCATCT TCCAAGCCGA GAATCGCCGC CATTGCGCCC |
| 301 | ACGCCTTGCG GTACGGCGGA CTGCATCAGT TCGGCGCGCA GGCGCACGAG |
| 351 | TTTGACCGCG TCGGCAAAAT TCAATGCGCC GGCGGCAACG AGTGCGGTGT |
| 401 | ATTCGCCGAG GCTGTGTCCG GCAACGGCGG CAGGCGTTTT GCCGCCCGCT |
| 451 | TCTAAATAG |

This corresponds to the amino acid sequence <SEQ ID 963; ORF 3>:

| m279.pep | |
|---|---|
| 1 | ITRICGCLIS TVFRASASLS AAGFIRLQWE GTDTGSGRAR LAPASLAAAM |
| 51 | ARPTAAALPA ITICPGELKL TASTTSLWAA SAQMALTCSS SKPRIAAIAP |
| 101 | TPCGTADCIS SARRRTSLTA SAKFNAPAAT SAVYSPRLCP ATAAGVLPPA |
| 151 | SK* |

The following partial DNA sequence was identified in N. gonorrhoeae <SEQ ID 4>:

| g279.seq | |
|---|---|
| 1 | atgacgcgga tttgcggctg cttgatttca acggttttga gtgtttcggc |
| 51 | aagtttgtcg gcggcgggtt tcatcaggct gcaatgggaa ggaacggata |
| 101 | ccggcagcgg cagggcgcgt ttggctccgg cttctttggc ggcagccatg |
| 151 | gtgcgtccga cggcggcggc gttgcctgca atcacgactt gtccgggcga |
| 201 | gttgaagttg acggcttcga ccacttcgcc ctgtgcggat tcggcacaaa |
| 251 | tctgcctgac ctgttcatct tccaaaccca aaatggccgc cattgcgcct |
| 301 | acgccttgcg gtacggcgga ctgcatcagt tcggcgcgca ggcggacgag |
| 351 | tttgacggca tcggcaaaat ccaatgcttc ggcggcgaca agcgcggtgt |
| 401 | attcgccgag gctgtgtccg gcaacggcgg caggcgtttt gccgcccact |
| 451 | tccaaatag |

This corresponds to the amino acid sequence <SEQ ID 5; ORF 279.ng>:

| g279.pep | |
|---|---|
| 1 | MTRICGCLIS TVLSVSASLS AAGFIRLQWE GTDTGSGRAR LAPASLAAAM |
| 51 | VRPTAAALPA ITTCPGELKL TASTTSPCAD SAQICLTCSS SKPKMAAIAP |
| 101 | TPCGTADCIS SARRRTSLTA SAKSNASAAT SAVYSPRLCP ATAAGVLPPT |
| 151 | SK* |

ORF 279 shows 89.5% identity over a 152 aa overlap with a predicted ORF (ORF 279.ng) from N. gonorrhoeae:

```
                  10 20 30 40 50 60
m279.pep
ITRICGCLISTVFRASASLSAAGFIRLQWEGTDTGSGRARLAPASLAAAMARPTAAALPA
:|||||||||||::||||||||||||||||||||||||||||||||||||:|||||||||
g279
MTRICGCLISTVLSVSASLSAAGFIRLQWEGTDTGSGRARLAPASLAAAMVRPTAAALPA
                        10 20 30 40 50 60
                        70 80 90 100 110 120
m279.pep
ITICPGELKLTASTTSLWAASAQMALTCSSSKPRIAAIAPTPCGTADCISSARRRTSLTA
            || ||||||||||||| | |||: ||||||||::|||||||||||||||||||||||||
g279
ITTCPGELKLTASTTSPCADSAQICLTCSSSKPKMAAIAPTPCGTADCISSARRRTSLTA
                        70 80 90 100 110 120
                  130 140 150
m279.pep SAKFNAPAATSAVYSPRLCPATAAGVLPPASKX
          ||| || ||||||||||||||||||||||:|||
g279 SAKSNASAATSAVYSPRLCPATAAGVLPPTSKX
                  130 140 150
```

The following partial DNA sequence was identified in N. meningitidis <SEQ ID 6>:

| a279.seq | |
|---|---|
| 1 | ATGACNCNGA TTTGCGGCTG CTTGATTTCA ACGGTTTNNA GGGCTTCGGC |
| 51 | GAGTTTGTCG GCGGCGGGTT TCATGAGGCT GCAATGGGAA GGTACNGACA |
| 101 | CNGGCAGCGG CAGGGCGCGT TTGGCGCCGG CTTCTTTGGC GGCAAGCATA |
| 151 | GCGCGCTCGA CGGCGGCGGC ATTGCCTGCA ATCACGACTT GTCCGGGCGA |
| 201 | GTTGAAGTTG ACGGCTTCAA CCACTTCATC CTGTGCGGAT TCGGCGCAAA |
| 251 | TTTGTTTTAC CTGTTCATCT TCCAAGCCGA GAATCGCCGC CATTGCGCCC |
| 301 | ACGCCTTGCG GTACGGCGGA CTGCATCAGT TCGGCGCGCA NGCGCACGAG |
| 351 | TTTGACCGCG TCGGCAAAAT CCAATGCGCC GGCGGCAACN AGTGCGGTGT |
| 401 | ATTCGCCGAN GCTGTGTCCG GCAACGGCGG CAGGCGTTTT GCCGCCCGCT |
| 451 | TCCGAATAG |

This corresponds to the amino acid sequence <SEQ ID 7; ORF 279.a>:

a279.pep

| | |
|---|---|
| 1 | MTXICGCLIS TVXRASASLS AAGFMRLQWE GTDTGSGRAR LAPASLAASI |
| 51 | ARSTAAALPA ITTCPGELKL TASTTSSCAD SAQICFTCSS SKPRIAAIAP |
| 101 | TPCGTADCIS SARXRTSLTA SAKSNAPAAT SAVYSPXLGP ATAAGVLPPA |
| 151 | SE* |

| m279/a279 | ORFs 279 and 279.a showed a 88.2% identity in 152 an overlap |
|---|---|
| | 10 20 30 40 50 60 |
| m279.pep | ITRICGCLISTVFRASASLSAAGFIRLQWEGTDTGSGRARLAPASLAAAMARPTAAALPA |
| :\| \|\|\|\|\|\|\|\|\| \|\|\|\|\|\|\|\|\|\|\|:\|\|\|\|\|\|\|\|\|\|\|\|\|\|\|\|\|\|\|\|\|\|\|::\|\| \|\|\|\|\|\|\| | |
| a279 | MTXICGCLISTVXRASASLSAAGFMRLQWEGTDTGSGRARLAPASLAASIARSTAAALPA |
| | 10 20 30 40 50 60 |
| | 70 80 90 100 110 120 |
| m279.pep | ITICPGELKLTASTTSLWAASAQMALTCSSSKPRIAAIAPTPCGTADCISSARRRTSLTA |

|| ||||||||||||| | |||: :||||||||||||||||||||||||||| ||||||

| | |
|---|---|
| a279 | ITTCPGELKLTASTTSSCADSAQICFTCSSSKPRIAAIAPTGCGTADCISSARXRTSLTA |
| | 70 80 90 100 110 120 |
| | 130 140 150 |
| m279.pep | SAKFNAPAATSAVYSPRLCPATAAGVLPPASKX |

||| |||||||||||| ||||||||||||||:|

| | |
|---|---|
| a279 | SAKSNAPAATSAVYSPXLCPATAAGVLPPASEX |
| | 130 140 150 |

519 and 519-1 gnm7.seq

The following partial DNA sequence was identified in N. meningitidis <SEQ ID 8>:

m519.seq (partial)

| | |
|---|---|
| 1 | TCCGTTATCG GGCGTATGGA GTTGGACAAA ACGTTTGAAG |
| | AACGCGACGA |
| 51 | AATCAACAGT ACTGTTGTTG CGGCTTTGGA CGAGGCGGCC |
| | GGGgCTTgGG |
| 101 | GTGTGAAGGT TTTGCGTTAT GAGATTAAAG ACTTGGTTCC |
| | GCCGCAAGAA |
| 151 | ATCCTTCGCT CAATGCAGGC GCAAATTACT GCCGAACGCG |
| | AAAAACGCGC |
| 201 | CCGTATCGCC GAATCCGAAG GTCGTAAAAT CGAACAAATC |
| | AACCTTGCCA |
| 251 | GTGGTCAGCG CGAAGCCGAA ATCCAACAAT CCGAAGGCGA |
| | GGCTCAGGCT |
| 301 | GCGGTCAATG CGTCAAATGC CGAGAAAATC GCCCGCATCA |
| | ACCGCGCCAA |
| 351 | AGGTGAAGCG GAATCCTTGC GCCTTGTTGC CGAAGCCAAT |
| | GCCGAAGCCA |
| 401 | TCCGTCAAAT TGCCGCCGCC CTTCAAACCC AAGGCGGTGC |
| | GGATGCGGTC |
| 451 | AATCTGAAGA TTGCGGAACA ATACGTCGCT GCGTTCAACA |
| | ATCTTGCCAA |
| 501 | AGAAAGCAAT ACGCTGATTA TGCCCGCCAA TGTTGCCGAC |
| | ATCGGCAGCC |
| 551 | TGATTTCTGC CGGTATGAAA ATTATCGACA GCAGCAAAAC |
| | CGCCAAaTAA |

This corresponds to the amino acid sequence <SEQ ID 9; ORF 519>:

m519.pep (partial)

| | |
|---|---|
| 1 | SVIGRMELDK TFEERDEINS TVVAALDEAA GAWGVKVLRY |
| | EIKDLVPPQE |
| 51 | ILRSMQAQIT AEREKRARIA ESEGRKIEQI NLASGQREAE |
| | IQQSEGEAQA |
| 101 | AVNASNAEKI ARINRAKGEA ESLRLVAEAN AEAIRQIAAA |
| | LQTQGGADAV |
| 151 | NLKIAEQYVA AFNNLAKESN TLIMPANVAD IGSLISAGMK |
| | IIDSSKTAK* |

The following partial DNA sequence was identified in N. gonorrhoeae <SEQ ID 10>:

g519.seq

| | |
|---|---|
| 1 | atggaatttt tcattatctt gttggcagcc gtcgccgttt |
| | tcggcttcaa |
| 51 | atcctttgtc gtcatccccc agcaggaagt ccacgttgtc |
| | gaaaggctcg |
| 101 | ggcgtttcca tcgcgccctg acggccggtt tgaatatttt |
| | gattcccttt |
| 151 | atcgaccgcg tcgcctaccg ccattcgctg aaagaaatcc |
| | ctttagacgt |
| 201 | acccagccag gtctgcatca cgcgcgataa tacgcaattg |
| | actgttgacg |
| 251 | gcatcatcta tttccaagta accgatccca aactcgcctc |
| | atacggttcg |
| 301 | agcaactaca ttatggcaat tacccagctt gcccaaacga |
| | cgctgcgttc |
| 351 | cgttatcggg cgtatggagt tggacaaaac gtttgaagaa |
| | cgcgacgaaa |
| 401 | tcaacagtac cgtcgtctcc gccctcgatg aagccgccgg |
| | ggcttggggt |
| 451 | gtgaaagtcc tccgttacga aatcaaggat ttggttccgc |
| | cgcaagaaat |
| 501 | ccttcgcgca atgcaggcac aaattaccgc cgaacgcgaa |
| | aaacgcgccc |
| 551 | gtattgccga atccgaaggc cgtaaaatcg aacaaatcaa |
| | ccttgccagt |
| 601 | ggtcagcgtg aagccgaaat ccaacaatcc gaaggcgagg |
| | ctcaggctgc |
| 651 | ggtcaatgcg tccaatgccg agaaaatcgc ccgcatcaac |
| | cgcgccaaag |
| 701 | gcgaagcgga atccctgcgc cttgttgccg aagccaatgc |
| | cgaagccaac |
| 751 | cgtcaaattg ccgccgccct tcaaacccaa agcggggcgg |
| | atgcggtcaa |
| 801 | tctgaagatt gcgggacaat acgttaccgc gttcaaaaat |
| | cttgccaaag |
| 851 | aagacaatac gcggattaag cccgccaagg ttgccgaaat |
| | cgggaaccct |
| 901 | aattttcggc ggcatgaaaa attttcgcca gaagcaaaaa |
| | cggccaaata |
| 951 | a |

This corresponds to the amino acid sequence <SEQ ID 11; ORF 519.ng>:

g519.pep

| | |
|---|---|
| 1 | MEFFIILLAA VAVFGFKSFV VIPQQEVHVV ERLGRFHRAL |
| | TAGLNILIPF |
| 51 | IDRVAYRHSL KEIPLDVPSQ VCITRDNTQL TVDGIIYFQV |
| | TDPKLASYGS |
| 101 | SNYIMAITQL AQTTLRSVIG RMELDKTFEE RDEINSTVVS |
| | ALDEAAGAWG |
| 151 | VKVLRYEIKD LVPPQEILRA MQAQITAERE KRARIAESEG |
| | RKIEQINLAS |
| 201 | GQREAEIQQS EGEAQAAVNA SNAEKIARIN RAKGEAESLR |
| | LVAEANAEAN |
| 251 | RQIAAALQTQ SGADAVNLKI AGQYVTAFKN LAKEDNTRIK |
| | PAKVAEIGNP |
| 301 | NFRRHEKFSP EAKTAK* |

ORF 519 shows 87.5% identity over a 200 aa overlap with a predicted ORF (ORF 519.ng) from N. gonorrhoeae:

| m519/g519 | |
|---|---|
| | 10 20 30 |
| m519.pep | SVIGRMELDKTFEERDEINSTVVAALDEAA |
| | \|\|\|\|\|\|\|\|\|\|\|\|\|\|\|\|\|\|\|\|\|\|\|\|:\|\|\|\|\|\| |
| g519 | YFQVTDPKLASYGSSNYIMAITQLAQTTLRSVIGRMELDKTFEERDEINSTVVSALDEAA |
| | 90 100 110 120 130 140 |
| | 40 50 60 70 80 90 |
| m519.pep | GAWGVKVLRYEIKDLVPPQEILRSMQAQITAEREKRARIAESEGRKIEQINLASGQREAE |
| | \|\|\|\|\|\|\|\|\|\|\|\|\|\|\|\|\|\|\|\|\|\|\|:\|\|\|\|\|\|\|\|\|\|\|\|\|\|\|\|\|\|\|\|\|\|\|\|\|\|\|\|\|\|\|\|\|\|\|\| |
| g519 | GAWGVKVLRYEIKDLVPPQEILRAMQAQITAEREKRARIAESEGRKIEQINLASGQREAE |
| | 150 160 170 180 190 200 |
| | 100 110 120 130 140 150 |
| m519.pep | IQQSEGEAQAAVNASNAEKIARINRAKGEAESLRLVAEANAEAIRQIAAALQTQGGADAV |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|\|\|:\|\|\|\|\| |
| g519 | IQQSEGEAQAAVNASNAEKIARINRAKGEAESLRLVAEANAEANRQIAAALQTQSGADAV |
| | 210 220 230 240 250 260 |
| | 160 170 180 190 200 |
| m519.pep | NLKIAEQYVAAFNNLAKESNTLIMPANVADIGSL-ISAGMKIIDSSKTAK |
| | \|\|\|\|\| \|\|\|:\|\|:\|\|\|\|\|:\|\| \| \|\|:\|\|:\|\|: : \|: :\|\|\|\| |
| g519 | NLKIAGQYVTAFKNLAKEDNTRIKPAKVAEIGNPNFRRHEKFSPEAKTAK |
| | 270 280 290 300 310 |

The following partial DNA sequence was identified in N. meningitidis <SEQ ID 12>:

a519.seq

| | |
|---|---|
| 1 | ATGGAATTTT TCATTATCTT GCTGGCAGCC GTCGTTGTTT |
| | TCGGCTTCAA |
| 51 | ATCCTTTGTT GTCATCCCAC AGCAGGAAGT CCACGTTGTC |
| | GAAAGGCTCG |
| 101 | GGCGTTTCCA TCGCGCCCTG ACGGCCGGTT TGAATATTTT |
| | GATTCCCTTT |
| 151 | ATCGACCGCG TCGCCTACCG CCATTCGCTG AAAGAAATCC |
| | CTTTAGACGT |
| 201 | ACCCAGCCAG GTCTGCATCA CGCGCGACAA TACGCAGCTG |
| | ACTGTTGACG |
| 251 | GTATCATCTA TTTCCAAGTA ACCGACCCCA AACTCGCCTC |
| | ATACGGTTCG |
| 301 | AGCAACTACA TTATGGCGAT TACCCAGCTT GCCCAAACGA |
| | CGCTGCGTTC |
| 351 | CGTTATCGGG CGTATGGAAT TGGACAAAAC GTTTGAAGAA |
| | CGCGACGAAA |
| 401 | TCAACAGCAC CGTCGTCTCC GCCCTCGATG AAGCCGCCGG |
| | AGCTTGGGGT |
| 451 | GTGAAGGTTT TGCGTTATGA GATTAAAGAC TTGGTTCCGC |
| | CGCAAGAAAT |
| 501 | CCTTCGCTCA ATGCAGGCGC AAATTACTGC TGAACGCGAA |
| | AAACGCGCCC |
| 551 | GTATCGCCGA ATCCGAAGGT CGTAAAATCG AACAAATCAA |
| | CCTTGCCAGT |
| 601 | GGTCAGCGCG AAGCCGAAAT CCAACAATCC GAAGGCGAGG |
| | CTCAGGCTGC |
| 651 | GGTCAATGCG TCAAATGCCG AGAAAATCGC CCGCATCAAC |
| | CGCGCCAAAG |
| 701 | GTGAAGCGGA ATCCTTGCGC CTTGTTGCCG AAGCCAATGC |
| | CGAAGCCATC |
| 751 | CGTCAAATTG CCGCCGCCCT TCAAACCCAA GGCGGTGCGG |
| | ATGCGGTCAA |
| 801 | TCTGAAGATT GCGGAACAAT ACGTCGCCGC GTTCAACAAT |
| | CTTGCCAAAG |
| 851 | AAAGCAATAC GCTGATTATG CCCGCCAATG TTGCCGACAT |
| | CGGCAGCCTG |
| 901 | ATTTCTGCCG GTATGAAAAT TATCGACAGC AGCAAAACCG |
| | CCAAATAA |

This corresponds to the amino acid sequence <SEQ ID 13; ORF 519.a>:

| a519.pep | |
|---|---|
| 1 | MEFFIILLAA VVVFGFKSFV VIPQQEVHVV ERLGRFHRAL TAGLNILIPF |
| 51 | IDRVAYRHSL KEIPLDVPSQ VCITRDNTQL TVDGIIYFQV TDPKLASYGS |
| 101 | SNYIMAITQL AQTTLRSVIG RMELDKTFEE RDEINSTVVS ALDEAAGAWG |
| 151 | VKVLRYEIKD LVPPQEILRS MQAQITAERE KRARIAESEG RKIEQINLAS |
| 201 | GQREAEIQQS EGEAQAAVNA SNAEKIARIN RAKGEAESLR LVAEANAEAI |
| 251 | RQIAAALQTQ GGADAVNLKI AEQYVAAFNN LAKESNTLIM PANVADIGSL |
| 301 | ISAGMKIIDS SKTAK* |

| | |
|---|---|
| m519/a519 | ORFs 519 and 519.a showed a 99.5% identity in 199 an overlap |
| | 10 20 30 |
| m519.pep | SVIGRMELDKTFEERDEINSTVVSALDEAA |
| | \|\|\|\|\|\|\|\|\|\|\|\|\|\|\|\|\|\|\|\|\|\|\|\|:\|\|\|\|\|\| |
| a519 | YFQVTDPKLASYGSSNYIMAITQLAQTTLRSVIGRMELDKTFEERDEINSTVVSALDEAA |
| | 90 100 110 120 130 140 |
| | 40 50 60 70 80 90 |
| m519.pep | GAWGVKVLRYEIKDLVPPQEILRSMQAQITAEREKRARIAESEGRKIEQINLASGQREAE |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| a519 | GAWGVKVLRYEIKDLVPPQEILRSMQAQITAEREKRARIAESEGRKIEQINLASGQREAE |
| | 150 160 170 180 190 200 |
| | 100 110 120 130 140 150 |
| m519.pep | IQQSEGEAQAAVNASNAEKIARINRAKGEAESLRLVAEANAEAIRQIAAALQTQGGADAV |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| a519 | IQQSEGEAQAAVNASNAEKIARINRAKGEAESLRLVAEANAEAIRQIAAALQTQGGADAV |
| | 210 220 230 240 250 260 |
| | 160 170 180 190 200 |
| m519.pep | NLKIAEQYVAAFNNLAKESNTLIMPANVADIGSLISAGMKIIDSSKTAKX |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| a519 | NLKIAEQYVAAFNNLAKESNTLIMPANVADIQSLISAGMKIIDSSKTAKX |
| | 270 280 290 300 310 |

Further work revealed the following DNA sequence identified in N. meningitidis <SEQ ID 14>:

m519-1.seq

| | |
|---|---|
| 1 | ATGGAATTTT TCATTATCTT GTTGGTAGCC GTCGCCGTTT |
| | TCGGTTTCAA |
| 51 | ATCCTTTGTT GTCATCCCAC AACAGGAAGT CCACGTTGTC |
| | GAAAGGCTGG |
| 101 | GGCGTTTCCA TCGCGCCCTG ACGGcCGGTT TGAATATTTT |
| | GATTCCCTTT |
| 151 | ATCGACCGCG TCGCCTACCG CCATTCGCTG AAAGAAATCC |
| | CTTTAGACGT |
| 201 | ACCCAGCCAG GTCTGCATCA CGCGCGACAA TACGCAGCTG |
| | ACTGTTGACG |
| 251 | GCATCATCTA TTTCCAAGTA ACCGACCCCA AACTCGCCTC |
| | ATACGGTTCG |
| 301 | AGCAACTACA TTATGGCGAT TACCCAGCTT GCCCAAACGA |
| | CGCTGCGTTC |
| 351 | CGTTATCGGG CGTATGGAGT TGGACAAAAC GTTTGAAGAA |
| | CGCGACGAAA |
| 401 | TCAACAGTAC TGTTGTTGCG GCTTTGGACG AGGCGGCCGG |
| | GGCTTGGGGT |
| 451 | GTGAAGGTTT TGCGTTATGA GATTAAAGAC TTGGTTCCGC |
| | CGCAAGAAAT |
| 501 | CCTTCGCTCA ATGCAGGCGC AAATTACTGC CGAACGCGAA |
| | AAACGCGCCC |
| 551 | GTATCGCCGA ATCCGAAGGT CGTAAAATCG AACAAATCAA |
| | CCTTGCCAGT |
| 601 | GGTCAGCGCG AAGCCGAAAT CCAACAATCC GAAGGCGAGG |
| | CTCAGGCTGC |
| 651 | GGTCAATGCG TCAAATGCCG AGAAAATCGC CCGCATCAAC |
| | CGCGCCAAAG |
| 701 | GTGAAGCGGA ATCCTTGCGC CTTGTTGCCG AAGCCAATGC |
| | CGAAGCCATC |
| 751 | CGTCAAATTG CCGCCGCCCT TCAAACCCAA GGCGGTGCGG |
| | ATGCGGTCAA |
| 801 | TCTGAAGATT GCGGAACAAT ACGTCGCTGC GTTCAACAAT |
| | CTTGCCAAAG |
| 851 | AAAGCAATAC GCTGATTATG CCCGCCAATG TTGCCGACAT |
| | CGGCAGCCTG |
| 901 | ATTTCTGCCG GTATGAAAAT TATCGACAGC AGCAAAACCG |
| | CCAAATAA |

This corresponds to the amino acid sequence <SEQ ID 15; ORF 519-1>:

m519-1

| | |
|---|---|
| 1 | MEFFIILLVA VAVFGFKSFV VIPQQEVHVV ERLGRFHRAL |
| | TAGLNILIPF |
| 51 | IDRVAYRHSL KEIPLDVPSQ VCITRDNTQL TVDGIIYFQV |
| | TDPKLASYGS |
| 101 | SNYIMAITQL AQTTLRSVIG RMELDKTFEE RDEINSTVVA |
| | ALDEAAGAWG |
| 151 | VKVLRYEIKD LVPPQEILRS MQAQITAERE KRARIAESEG |
| | RKIEQINLAS |
| 201 | GQREAEIQQS EGEAQAAVNA SNAEKIARIN RAKGEAESLR |
| | LVAEANAEAI |
| 251 | RQIAAALQTQ GGADAVNLKI AEQYVAAFNN LAKESNTLIM |
| | PANVADIGSL |
| 301 | ISAGMKIIDS SKTAK* |

The following DNA sequence was identified in N. gonorrhoeae <SEQ ID 16>:

g519-1.seq

| | |
|---|---|
| 1 | ATGGAATTTT TCATTATCTT GTTGGCAGCC GTCGCCGTTT |
| | TCGGCTTCAA |
| 51 | ATCCTTTGTC GTCATCCCCC AGCAGGAAGT CCACGTTGTC |
| | GAAAGGCTCG |
| 101 | GGCGTTTCCA TCGCGCCCTG ACGGCCGGTT TGAATATTTT |
| | GATTCCCTTT |
| 151 | ATCGACCGCG TCGCCTACCG CCATTCGCTG AAAGAAATCC |
| | CTTTAGACGT |
| 201 | ACCCAGCCAG GTCTGCATCA CGCGCGATAA TACGCAATTG |
| | ACTGTTGACG |
| 251 | GCATCATCTA TTTCCAAGTA ACCGATCCCA AACTCGCCTC |
| | ATACGGTTCG |
| 301 | AGCAACTACA TTATGGCAAT TACCCAGCTT GCCCAAACGA |
| | CGCTGCGTTC |
| 351 | CGTTATCGGG CGTATGGAGT TGGACAAAAC GTTTGAAGAA |
| | CGCGACGAAA |
| 401 | TCAACAGTAC CGTCGTCTCC GCCCTCGATG AAGCCGCCGG |
| | GGCTTGGGGT |
| 451 | GTGAAAGTCC TCCGTTACGA AATCAAGGAT TTGGTTCCGC |
| | CGCAAGAAAT |
| 501 | CCTTCGCGCA ATGCAGGCAC AAATTACCGC CGAACGCGAA |
| | AAACGCGCCC |
| 551 | GTATTGCCGA ATCCGAAGGC CGTAAAATCG AACAAATCAA |
| | CCTTGCCAGT |
| 601 | GGTCAGCGTG AAGCCGAAAT CCAACAATCC GAAGGCGAGG |
| | CTCAGGCTGC |
| 651 | GGTCAATGCG TCCAATGCCG AGAAAATCGC CCGCATCAAC |
| | CGCGCCAAAG |
| 701 | GCGAAGCGGA ATCCCTGCGC CTTGTTGCCG AAGCCAATGC |
| | CGAAGCCATC |
| 751 | CGTCAAATTG CCGCCGCCCT TCAAACCCAA GGCGGGGCGG |
| | ATGCGGTCAA |
| 801 | TCTGAAGATT GCGGAACAAT ACGTAGCCGC GTTCAACAAT |
| | CTTGCCAAAG |
| 851 | AAAGCAATAC GCTGATTATG CCCGCCAATG TTGCCGACAT |
| | CGGCAGCCTG |
| 901 | ATTTCTGCCG GCATGAAAAT TATCGACAGC AGCAAAACCG |
| | CCAAATAA |

This corresponds to the amino acid sequence <SEQ ID 17; ORF 519-1.ng>:

| g519-1.pep | |
|---|---|
| 1 | MEFFIILLAA VAVFGFKSFV VIPQQEVHVV ERLGRFHRAL TAGLNILIPF |
| 51 | IDRVAYRHSL KEIPLDVPSQ VCITRDNTQL TVDGIIYFQV TDPKLASYGS |
| 101 | SNYIMAITQL AQTTLRSVIG RMELDKTFEE RDEINSTVVS ALDEAAGAWG |
| 151 | VKVLRYEIKD LVPPQEILRA MQAQITAERE KRARIAESEG RKIEQINLAS |
| 201 | GQREAEIQQS EGEAQAAVNA SNAEKIARIN RAKGEAESLR LVAEANAEAI |
| 251 | RQIAAALQTQ GGADAVNLKI AEQYVAAFNN LAKESNTLIM PANVADIGSL |
| 301 | ISAGMKIIDS SKTAK* |

| | |
|---|---|
| m519-1/g519-1 | ORFs 519-1 and 519-1.ng showed a 99.0% identity in 315 aa overlap |
| | 10 20 30 40 50 60 |
| g519-1.pep | MEFFIILLAAVAVFGFKSFVVIPQQEVHVVERLGRFHRALTAGLNILIPFIDRVAYRHSL |
| | \|\|\|\|\|\|\|\|:\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m519-1 | MEFFIILLVAVAVFGFKSFVVIPQQEVHVVERLGRFHRALTAGLNILIPFIDRVAYRHSL |
| | 10 20 30 40 50 60 |
| | 70 80 90 100 110 120 |
| g519-1.pep | KEIPLDVPSQVCITRDNTQLTVDGIIYFQVTDPKLASYGSSNYIMAITQLAQTTLRSVIG |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m519-1 | KEIPLDVPSQVCITRDNTQLTVDGIIYFQVTDPKLASYGSSNYIMAITQLAQTTLRSVIG |
| | 70 80 90 100 110 120 |
| | 130 140 150 160 170 180 |
| g519-1.pep | RMELDKTFEERDEINSTVVSALDEAAGAWGVKVLRYEIKDLVPPQEILRAMQAQITERE |
| | \|\|\|\|\|\|\|\|\|\|\|\|\|\|\|\|\|\|\|:\|\|\|\|\|\|\|\|\|\|\|\|\|\|\|\|\|\|\|\|\|\|\|\|\|\|\|\|\|:\|\|\|\|\|\|\|\|\|\| |
| m519-1 | RMELDKTFEERDEINSTVVALDEAAGAWGVKVLRYEIKDLVPPQEILRSMQAQITAERE |
| | 130 140 150 160 170 180 |
| | 190 200 210 220 230 240 |
| g519-1.pep | KRARIAESEGRKIEQINLASGQREAEIQQSEGEAQAAVNASNAEKIARINRAKGEAESLR |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m519-1 | KRARIAESEGRKIEQINLASGQREAEIQQSEGEAQAAVNASNAEKIARINRAKGEAESLR |
| | 190 200 210 220 230 240 |
| | 250 260 270 280 290 300 |
| g519-1.pep | LVAEANAEAIRQIAAALQTQGGADAVNLKIAEQYVAAFNNLAKESNTLIMPANVADIGSL |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m519-1 | LVAEANAEAIRQIAAALQTQGGADAVNLKIAEQYVAAFNNLAKESNTLIMPANVADIGSL |
| | 250 260 270 280 290 300 |
| | 310 |
| g519-1.pep | ISAGMKIIDSSKTAKX |
| | \|\|\|\|\|\|\|\|\|\|\|\|\|\|\|\| |
| m519-1 | ISAGMKIIDSSKTAKX |
| | 310 |

The following DNA sequence was identified in N. meningitidis <SEQ ID 18>:

a519-1.seq

| | |
|---|---|
| 1 | ATGGAATTTT TCATTATCTT GCTGGCAGCC GTCGTTGTTT |
| | TCGGCTTCAA |
| 51 | ATCCTTTGTT GTCATCCCAC AGCAGGAAGT CCACGTTGTC |
| | GAAAGGCTCG |
| 101 | GGCGTTTCCA TCGCGCCCTG ACGGCCGGTT TGAATATTTT |
| | GATTCCCTTT |
| 151 | ATCGACCGCG TCGCCTACCG CCATTCGCTG AAAGAAATCC |
| | CTTTAGACGT |
| 201 | ACCCAGCCAG GTCTGCATCA CGCGCGACAA TACGCAGCTG |
| | ACTGTTGACG |
| 251 | GTATCATCTA TTTCCAAGTA ACCGACCCCA AACTCGCCTC |
| | ATACGGTTCG |
| 301 | AGCAACTACA TTATGGCGAT TACCCAGCTT GCCCAAACGA |
| | CGCTGCGTTC |
| 351 | CGTTATCGGG CGTATGGAAT TGGACAAAAC GTTTGAAGAA |
| | CGCGACGAAA |
| 401 | TCAACAGCAC CGTCGTCTCC GCCCTCGATG AAGCCGCCGG |
| | AGCTTGGGGT |
| 451 | GTGAAGGTTT TGCGTTATGA GATTAAAGAC TTGGTTCCGC |
| | CGCAAGAAAT |
| 501 | CCTTCGCTCA ATGCAGGCGC AAATTACTGC TGAACGCGAA |
| | AAACGCGCCC |
| 551 | GTATCGCCGA ATCCGAAGGT CGTAAAATCG AACAAATCAA |
| | CCTTGCCAGT |
| 601 | GGTCAGCGCG AAGCCGAAAT CCAACAATCC GAAGGCGAGG |
| | CTCAGGCTGC |
| 651 | GGTCAATGCG TCAAATGCCG AGAAAATCGC CCGCATCAAC |
| | CGCGCCAAAG |
| 701 | GTGAAGCGGA ATCCTTGCGC CTTGTTGCCG AAGCCAATGC |
| | CGAAGCCATC |
| 751 | CGTCAAATTG CCGCCGCCCT TCAAACCCAA GGCGGTGCGG |
| | ATGCGGTCAA |
| 801 | TCTGAAGATT GCGGAACAAT ACGTCGCCGC GTTCAACAAT |
| | CTTGCCAAAG |
| 851 | AAAGCAATAC GCTGATTATG CCCGCCAATG TTGCCGACAT |
| | CGGCAGCCTG |
| 901 | ATTTCTGCCG GTATGAAAAT TATCGACAGC AGCAAAACCG |
| | CCAAATAA |

This corresponds to the amino acid sequence <SEQ ID 19; ORF 519-1.a>:

| a519-1.pep | |
|---|---|
| 1 | MEFFIILLAA VVVFGFKSFV VIPQQEVHVV ERLGRFHRAL TAGLNILIPF |
| 51 | IDRVAYRHSL KEIPLDVPSQ VCITRDNTQL TVDGIIYFQV TDPKLASYGS |
| 101 | SNYIMAITQL AQTTLRSVIG RMELDKTFEE RDEINSTVVS ALDEAAGAWG |
| 151 | VKVLRYEIKD LVPPQEILRS MQAQITAERE KRARIAESEG RKIEQINLAS |
| 201 | GQREAEIQQS EGEAQAAVNA SNAEKIARIN RAKGEAESLR LVAEANAEAI |
| 251 | RQIAAALQTQ GGADAVNLKI AEQYVAAFNN LKAESNTLIM PANVADIGSL |
| 301 | ISAGMKIIDS SKTAK* |
| m519-1/a519-1 | ORFs 519-1 and 519-1.a showed a 99.0% identity in 315 an overlap |
| | 10 20 30 40 50 60 |
| a519-1.pep | MEFFIILLAAVVVFGFKSFVVIPQQEVHVVERIGREHRALTAGLNILIPFIDRVAYRHSL |
| | \|\|\|\|\|\|\|\|:\|\|:\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m519-1 | MEFFIILLVAVAVFGFKSFVVIPQQEVHVVERIGRFHRALTAGLNILIPFIDRVAYRHSL |
| | 10 20 30 40 50 60 |
| | 70 80 90 100 110 120 |
| a519-1.pep | KEIPLDVPSQVCITRDNTQLTVDGIIYFQVTDPKLASYGSSNYIMMTQLAQTTLRSVIG |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m519-1 | KEIPLDVPSQVCITRDNTQLTVDGIIYFQVTDPKLASYGSSNYIMMTQLAQTTLRSVIG |
| | 70 80 90 100 110 120 |
| | 130 140 150 160 170 180 |
| a519-1.pep | RMELDKTFEERDEINSTVVSALDEAAGAWGVKVLRYEIKDLVPPQEILRSMQAQITAERE |
| | \|\|\|\|\|\|\|\|\|\|\|\|\|\|\|\|\|\|\|:\|\|\|\|\|\|\|\|\|\|\|\|\|\|\|\|\|\|\|\|\|\|\|\|\|\|\|\|\|\|\|\|\|\|\|\|\|\|\|\| |
| m519-1 | RMELDKTFEERDEINSTVVAALDEAAGAWGVKVLRYEIKDLVPPQEILRSMQAQITAERE |
| | 130 140 150 160 170 180 |
| | 190 200 210 220 230 240 |
| a519-1.pep | KRARIAESEGRKIEQINLASGQREAEIQQSEGEAQAAVNASNAEKIARINRAKGEAESLR |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m519-1 | KRARIAESEGRKIEQINLASGQREAEIQQSEGEAQAAVNASNAEKIARINRAKGEAESLR |
| | 190 200 210 220 230 240 |
| | 250 260 270 280 290 300 |
| a519-1.pep | LVAEANAEAIRQIAAALQTQGGADAVNLKIAEQYVAAFNNLAKESNTLIMPANVADIGSL |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m519-1 | LVAEANAEAIRQIAAALQTQGGADAVNLKIAEQYVAAFNNLAKESNTLIMPANVADIGSL |
| | 250 260 270 280 290 300 |
| | 310 |
| a519-1.pep | ISAGMKIIDSSKTAKX |
| | \|\|\|\|\|\|\|\|\|\|\|\|\|\|\|\| |
| m519-1 | ISAGMKIIDSSKTAKX |
| | 310 |

576 and 576-1 gnm22.seq

The following partial DNA sequence was identified in N. meningitidis <SEQ ID 20>:

m576.seq (partial)

| | |
|---|---|
| 1 | ATGCAGCAGG CAAGCTATGC GATGGGCGTG GACATCGGAC |
| | GCTCCCTGAA |
| 51 | GCAAATGAAG GAACAGGGCG CGGAAATCGA TTTGAAAGTC |
| | TTTACCGAAG |
| 101 | CCATGCAGGC AGTGTATGAC GGCAAAGAAA TCAAAATGAC |
| | CGAAGAGCAG |
| 151 | GCTCAGGAAG TGATGATGAA ATTCCTTCAG GAACAACAGG |
| | CTAAAGCCGT |
| 201 | AGAAAAACAC AAGGCGGACG CGAAGGCCAA TAAAGAAAAA |
| | GGCGAAGCCT |
| 251 | TTCTGAAAGA AAATGCCGCC AAAGACGGCG TGAAGACCAC |
| | TGCTTCCGGC |
| 301 | CTGCAATACA AAATCACCAA ACAGGGCGAA GGCAAACAGC |
| | CGACCAAAGA |
| 351 | CGACATCGTT ACCGTGGAAT ACGAAGGCCG CCTGATTGAC |
| | GGTACGGTAT |
| 401 | TCGACAGCAG CAAAGCCAAC GGCGGCCCGG TCACCTTCCC |
| | TTTGAGCCAA |
| 451 | GTGATTCCGG GTTGGACCGA AGgCGTACAG CTTCTGAAAG |
| | AAGGCGGCGA |
| 501 | AGCCACGTTC TACATCCCGT CCAACCTTGC CTACCGCGAA |
| | CAGGGTGCGG |
| 551 | GCGACAAAAT CGGTCCGAAC GCCACTTTGG TATTTGATGT |
| | GAAACTGGTC |
| 601 | AAAATCGGCG CACCCGAAAA CGCGCCCGCC AAGCAGCCGG |
| | CTCAAGTCGA |
| 651 | CATCAAAAAA GTAAATTAA |

This corresponds to the amino acid sequence <SEQ ID 21; ORF 576>:

m576.pep (partial)

| | |
|---|---|
| 1 | MQQASYAMGV DIGRSLKQMK EQGAEIDLKV FTEAMQAVYD |
| | GKEIKMTEEQ |
| 51 | AQEVMMKFLQ EQQAKAVEKH KADAKANKEK GEAFLKENAA |
| | KDGVKTTASG |
| 101 | LQYKITKQGE GKQPTKDDIV TVEYEGRLID GTVFDSSKAN |
| | GGPVTFPLSQ |
| 151 | VIPGWTEGVQ LLKEGGEATF YIPSNLAYRE QGAGDKIGPN |
| | ATLVFDVKLV |
| 201 | KIGAPENAPA KQPAQVDIKK VN* |

The following partial DNA sequence was identified in N. gonorrhoeae <SEQ ID 22>:

g576.seq (partial)

| | |
|---|---|
| 1 | atgggcgtgg acatcggacg ctccctgaaa caaatgaagg |
| | aacagggcgc |
| 51 | ggaaatcgat ttgaaagtct ttaccgatgc catgcaggca |
| | gtgtatgacg |
| 101 | gcaaagaaat caaaatgacc gaagagcagg cccaggaagt |
| | gatgatgaaa |
| 151 | ttcctgcagg agcagcaggc taaagccgta gaaaaacaca |
| | aggcggatgc |
| 201 | gaaggccaac aaagaaaaag gcgaagcctt cctgaaggaa |
| | aatgccgccg |
| 251 | aagacggcgt gaagaccact gcttccggtc tgcagtacaa |
| | aatcaccaaa |
| 301 | cagggtgaag gcaaacagcc gacaaaagac gacatcgtta |
| | ccgtggaata |
| 351 | cgaaggccgc ctgattgacg gtaccgtatt cgacagcagc |
| | aaagccaacg |
| 401 | gcggcccggc caccttccct ttgagccaag tgattccggg |
| | ttggaccgaa |
| 451 | ggcgtacggc ttctgaaaga aggcggcgaa gccacgttct |
| | acatcccgtc |
| 501 | caaccttgcc taccgcgaac agggtgcggg cgaaaaaatc |
| | ggtccgaacg |
| 551 | ccactttggt atttgacgtg aaactggtca aaatcggcgc |
| | acccgaaaac |
| 601 | gcgcccgcca agcagccgga tcaagtcgac atcaaaaaag |
| | taaattaa |

This corresponds to the amino acid sequence <SEQ ID 23; ORF 576.ng>:

g576.pep (partial)

| | |
|---|---|
| 1 | MGVDIGRSLK QMKEQGAEID LKVFTDAMQA VYDGKEIKMT |
| | EEQAQEVMMK |
| 51 | FLQEQQAKAV EKHKADAKAN KEKGEAFLKE NAAEDGVKTT |
| | ASGLQYKITK |
| 101 | QGEGKQPTKD DIVTVEYEGR LIDGTVFDSS KANGGPATFP |
| | LSQVIPGWTE |
| 151 | GVRLLKEGGE ATFYIPSNLA YREQGAGEKI GPNATLVFDV |
| | KLVKIGAPEN |
| 201 | APAKQPDQVD IKKVN* |

Computer analysis of this amino acid sequence gave the following results:

Homology with a Predicted ORF from N. gonorrhoeae

| m576/g576 | 97.2% identity in 215 aa overlap |
|---|---|
| | 10 20 30 40 50 60 |
| m576.pep | MQQASYAMGVDIGRSLKQMKEQGAEIDLKVFTEAMQAVYDGKEIKMTEEQAQEVMMKFLQ |
| | \|\|\|\|\|\|\|\|\|\|\|\|\|\|\|\|\|\|\|\|\|\|\|\|\|:\|\|\|\|\|\|\|\|\|\|\|\|\|\|\|\|\|\|\|\|\|\|\|\|\|\|\|\|\|\|\|\|\|\| |
| g576 | MGVDIGRSLKQMKEQGAEIDLKVFTDAMQAVYDGKEIKMTEEQAQEVMMKFLQ |
| | 10 20 30 40 50 |
| | 70 80 90 100 110 120 |
| m576.pep | EQQAKAVEKHKADAKANKEKGEAFLKENAAKDGVKTTASGLQYKITKQGEGKQPTKDDIV |
| | \|\|\|\|\|\|\|\|\|\|\|\|\|\|\|\|\|\|\|\|\|\|\|\|\|\|\|\|\|\|:\|\|\|\|\|\|\|\|\|\|\|\|\|\|\|\|\|\|\|\|\|\|\|\|\|\|\|\|\| |
| g576 | EQQAKAVEKHKADAKANKEKGEAFLKENAAEDGVKTTASGLQYKITKQGEGKQPTKDDIV |
| | 60 70 80 90 100 110 |
| | 130 140 150 160 170 180 |
| m576.pep | TVEYEGRLIDGTVFDSSKANGGPVTFPLSQVIPGWTEGVQLLKEGGEATFYIPSNLAYRE |
| | \|\|\|\|\|\|\|\|\|\|\|\|\|\|\|\|\|\|\|\|\|\|\|:\|\|\|\|\|\|\|\|\|\|\|\|\|\|\|:\|\|\|\|\|\|\|\|\|\|\|\|\|\|\|\|\|\|\|\| |
| g576 | TVEYEGRLIDGTVFDSSKANGGPATFPLSQVIPGWTEGVRLLKEGGEATFYIPSNLAYRE |
| | 120 130 140 150 160 170 |
| | 190 200 210 220 |
| m576.pep | QGAGDKIGPNATLVFDVKLVKIGAPENAPAKQPAQVDIKKVNIX |
| | \|\|\|:\|\|\|\|\|\|\|\|\|\|\|\|\|\|\|\|\|\|\|\| \|\|\|\|\|\|\|\|\|\|\|\|\|\|\|\|\|\|\| |
| g576 | QGAGEKIGPNATLVFDVKLVKIGAPENAPAKQPDQVDIKKVNIX |
| | 180 190 200 210 |

The following partial DNA sequence was identified in N. meningitidis <SEQ ID 24>:

a576.seq

| | |
|---|---|
| 1 | ATGAACACCA TTTTCAAAAT CAGCGCACTG ACCCTTTCCG |
| | CCGCTTTGGC |
| 51 | ACTTTCCGCC TGCGGCAAAA AAGAAGCCGC CCCCGCATCT |
| | GCATCCGAAC |
| 101 | CTGCCGCCGC TTCTTCCGCG CAGGGCGACA CCTCTTCGAT |
| | CGGCAGCACG |
| 151 | ATGCAGCAGG CAAGCTATGC GATGGGCGTG GACATCGGAC |
| | GCTCCCTGAA |
| 201 | GCAAATGAAG GAACAGGGCG CGGAAATCGA TTTGAAAGTC |
| | TTTACCGAAG |
| 251 | CCATGCAGGC AGTGTATGAC GGCAAAGAAA TCAAAATGAC |
| | CGAAGAGCAG |
| 301 | GCTCAGGAAG TCATGATGAA ATTCCTTCAG GAACAACAGG |
| | CTAAAGGCGT |
| 351 | AGAAAAACAC AAGGCGGACG CGAAGGCCAA TAAAGAAAAA |
| | GGCGAAGCCT |
| 401 | TTCTGAAAGA AAATGCCGCC AAAGACGGCG TGAAGACCAC |
| | TGCTTCCGGC |
| 451 | CTGCAATACA AAATCACCAA ACAGGGCGAA GGCAAACAGC |
| | CGACCAAAGA |
| 501 | CGACATCGTT ACCGTGGAAT ACGAAGGCCG CCTGATTGAC |
| | GGTACGGTAT |
| 551 | TCGACAGCAG CAAAGCCAAC GGCGGCCCGG TCACCTTGCC |
| | TTTGAGCCAA |
| 601 | GTGATTCTGG GTTGGACCGA AGGCGTACAG CTTCTGAAAG |
| | AAGGCGGCGA |
| 651 | AGCCACGTTC TACATCCCGT CCAACCTTGC CTACCGCGAA |
| | CAGGGTGCGG |
| 701 | GCGACAAAAT CGGCCCGAAC GCCACTTTGG TATTTGATGT |
| | GAAACTGGTC |
| 751 | AAAATCGGCG CACCCGAAAA CGCGCCCGCC AAGCAGCCGG |
| | CTCAAGTCGA |
| 801 | CATCAAAAAA GTAAATTAA |

This corresponds to the amino acid sequence <SEQ ID 25; ORF 576.a>:

| a576.pep | |
|---|---|
| 1 | MNTIFKISAL TLSAALALSA CGKKEAAPAS ASEPAAASSA QGDTSSIGST |
| 51 | MQQASYAMGV DIGRSLKQMK EQGAEIDLKV FTEAMQAVYD GKEIKMTEEQ |
| 101 | AQEVMMKFLQ EQQAKAVEKH KADAKANKEK GEAFLKENAA KDGVKTTASG |
| 151 | LQYKITKQGE GKQPTKDDIV TVEYEGRLID GTVFDSSKAN GGPVTFPLSQ |
| 201 | VILGWTEGVQ LLKEGGEATF YIPSNLAYRE QGAGKDIGPN ATLVFDVKLV |
| 251 | KIGAPENAPA KQPAYVDIKK VN* |

| | |
|---|---|
| m576/a576 | ORFs 576 and 576.a showed a 99.5% identity in 222 an overlap |
| | 10 20 30 |
| m576.pep | MQQASYAMGVDIGRSLKQMKEQGAEIDLKV |
| | \|\|\|\|\|\|\|\|\|\|\|\|\|\|\|\|\|\|\|\|\|\|\|\|\|\|\|\|\|\| |
| a576 | CGKKEAAPASASEPAAASSAQGDTSSIGSTMQQASYAMGVDIGRSLKQMKEQGAEIDLKV |
| | 30 40 50 60 70 80 |
| | 40 50 60 70 80 90 |
| m576.pep | FTEAMQAVYDGKEIKMTEEQAQEVMMKFLQEQQAKAVEKHKADAKANKEKGEAFLKENAA |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| a576 | FTEAMQAVYDGKEIKMTEEQAQEVMMKFLQEQQAKAVEKHKADAKANKEKGEAFLKENAA |
| | 90 100 110 120 130 140 |
| | 100 110 120 130 140 150 |
| m576.pep | KDGVKTTASGLQYKITKQGEGKQPTKDDIVTVEYEGRLIDGTVFDSSKANGGPVTFPLSQ |
| a576 | KDGVKTTASGLQYKITKQGEGKQPTKDDIVTVEYEGRLIDGTVFDSSKANGGPVTFPLSQ |
| | 150 160 170 180 190 200 |
| | 160 170 180 190 200 210 |
| m576.pep | VIPGWTEGVQLLKEGGEATFYIPSNLAYREQGAGDKIGPNATLVFDVKLVKIGAPENAPA |
| a576 | VILGWTEGVQLLKEGGEATFYIPSNLAYREQGAGDKIGPNATLVFDVKLVKIGAPENAPA |
| | 210 220 230 240 250 260 |
| | 220 |
| m576.pep | KQPAQVDIKKVNX |
| | \|\|\|\|\|\|\|\|\|\|\|\|\| |
| a576 | KQPAQVDIKKVNX |
| | 270 |

Further work revealed the following DNA sequence identified in N. meningitidis <SEQ ID 26>:

m576-1.seq

| | |
|---|---|
| 1 | ATGAACACCA TTTTCAAAAT CAGCGCACTG ACCCTTTCCG |
| | CCGCTTTGGC |
| 51 | ACTTTCCGCC TGCGGCAAAA AAGAAGCCGC CCCCGCATCT |
| | GCATCCGAAC |
| 101 | CTGCCGCCGC TTCTTCCGCG CAGGGCGACA CCTCTTCGAT |
| | CGGCAGCACG |
| 151 | ATGCAGCAGG CAAGCTATGC GATGGGCGTG GACATCGGAC |
| | GCTCCCTGAA |
| 201 | GCAAATGAAG GAACAGGGCG CGGAAATCGA TTTGAAAGTC |
| | TTTACCGAAG |
| 251 | CCATGCAGGC AGTGTATGAC GGCAAAGAAA TCAAAATGAC |
| | CGAAGAGCAG |
| 301 | GCTCAGGAAG TCATGATGAA ATTCCTTCAG GAACAACAGG |
| | CTAAAGCCGT |
| 351 | AGAAAAACAC AAGGCGGACG CGAAGGCCAA TAAAGAAAAA |
| | GGCGAAGCCT |
| 401 | TTCTGAAAGA AAATGCCGCC AAAGACGGCG TGAAGACCAC |
| | TGCTTCCGGC |
| 451 | CTGCAATACA AAATCACCAA ACAGGGCGAA GGCAAACAGC |
| | CGACCAAAGA |
| 501 | CGACATCGTT ACCGTGGAAT ACGAAGGCCG CCTGATTGAC |
| | GGTACGGTAT |
| 551 | TCGACAGCAG CAAAGCCAAC GGCGGCCCGG TCACCTTCCC |
| | TTTGAGCCAA |
| 601 | GTGATTCCGG GTTGGACCGA AGGCGTACAG CTTCTGAAAG |
| | AAGGCGGCGA |
| 651 | AGCCACGTTC TACATCCCGT CCAACGTTGC CTACCGCGAA |
| | CAGGGTGCGG |
| 701 | GCGACAAAAT CGGTCCGAAC GCCACTTTGG TATTTGATGT |
| | GAAACTGGTC |
| 751 | AAAATCGGCG CACCCGAAAA CGCGCCCGCC AAGCAGCCGG |
| | CTCAAGTCGA |
| 801 | CATCAAAAAA GTAAATTAA |

This corresponds to the amino acid sequence <SEQ ID 27; ORF 576-1>:

m576-1.pep

| | |
|---|---|
| 1 | MNTIFKISAL TLSAALALSA CGKKEAAPAS ASEPAAASSA |
| | QGDTSSIGST |
| 51 | MQQASYAMGV DIGRSLKQMK EQGAEIDLKV FTEAMQAVYD |
| | GKEIKMTEEQ |
| 101 | AQEVMMKFLQ EQQAKAVEKH KADAKANKEK GEAFLKENAA |
| | KDGVKTTASG |
| 151 | LQYKITKQGE GKQPTKDDIV TVEYEGRLID GTVFDSSKAN |
| | GGPVTFPLSQ |
| 201 | VIPGWTEGVQ LLKEGGEATF YIPSNLAYRE QGAGDKIGPN |
| | ATLVFDVKLV |
| 251 | KIGAPENAPA KQPAQVDIKK VN* |

The following DNA sequence was identified in N. gonorrhoeae <SEQ ID 28>:

g576-1.seq

| | |
|---|---|
| 1 | ATGAACACCA TTTTCAAAAT CAGCGCACTG ACCCTTTCCG |
| | CCGCTTTGGC |
| 51 | ACTTTCCGCC TGCGGCAAAA AAGAAGCCGC CCCCGCATCT |
| | GCATCCGAAC |
| 101 | CTGCCGCCGC TTCTGCCGCG CAGGGCGACA CCTCTTCAAT |
| | CGGCAGCACG |
| 151 | ATGCAGCAGG CAAGCTATGC AATGGGCGTG GACATCGGAC |
| | GCTCCCTGAA |
| 201 | ACAAATGAAG GAACAGGGCG CGGAAATCGA TTTGAAAGTC |
| | TTTACCGATG |
| 251 | CCATGCAGGC AGTGTATGAC GGCAAAGAAA TCAAAATGAC |
| | CGAAGAGCAG |
| 301 | GCCCAGGAAG TGATGATGAA ATTCCTGCAG GAGCAGCAGG |
| | CTAAAGCCGT |
| 351 | AGAAAAACAC AAGGCGGATG CGAAGGCCAA CAAAGAAAAA |
| | GGCGAACCT |
| 401 | TCCTGAAGGA AAATGCCGCC AAAGACGGCG TGAAGACCAC |
| | TGCTTCCGGT |
| 451 | CTGCAGTACA AAATCACCAA ACAGGGTGAA GGCAAACAGC |
| | CGACAAAAGA |
| 501 | CGACATCGTT ACCGTGGAAT ACGAAGGCCG CCTGATTGAC |
| | GGTACCGTAT |
| 551 | TCGACAGCAG CAAAGCCAAC GGCGGCCCGG CCACCTTCCC |
| | TTTGAGCCAA |
| 601 | GTGATTCCGG GTTGGACCGA AGGCGTACGG CTTCTGAAAG |
| | AAGGCGGCGA |
| 651 | AGCCACGTTC TACATCCCGT CCAACCTTGC CTACCGCGAA |
| | CAGGGTGCGG |
| 701 | GCGAAAAAAT CGGTCCGAAC GCCACTTTGG TATTTGACGT |
| | GAAACTGGTC |
| 751 | AAAATCGGCG CACCCGAAAA CGCGCCCGCC AAGCAGCCGG |
| | ATCAAGTCGA |
| 801 | CATCAAAAAA GTAAATTAA |

This corresponds to the amino acid sequence <SEQ ID 29; ORF 576-1.ng>:

| g576-1.pep | |
|---|---|
| 1 | MNTIFKISAL TLSAALALSA CGKKEAAPAS ASEPAAASAA QGDTSSIGST |
| 51 | MQQASYAMGV DIGRSLKQMK EQGAEIDLKV FTDAMQAVYD GKEIKMTEEQ |
| 101 | AQEVMMKFLQ EQQAKAVEKH KADAKANKEK GEAFLKENAA KDGVKTTASG |
| 151 | LQYKITKQGE GKQPTKDDIV TVEYEGRLID GTVFDSSKAN GGPATFPLSQ |
| 201 | VIPGWTEGVR LLKEGGEATF YIPSNLAYRE QGAGEKIGPN ALTVFDVKLV |
| 251 | KIGAPENAPA DQPDQVDIKK VN* |

| | |
|---|---|
| g576-1/m576-1 | ORFs 576-1 and 576-1.ng showed a 97.8% identity in 272 an overlap |
| | 10 20 30 40 50 60 |
| g576-1.pep | MNTIFKISALTLSAALALSACGKKEAAPASASEPAAASAAQGDTSSIGSTMQQASYAMGV |
| | \|\|\|\|\|\|\|\|\|\|\|\|\|\|\|\|\|\|\|\|\|\|\|\|\|\|\|\|\|\|\|\|\|\|\|\|\|\|\|\|:\|\|\|\|\|\|\|\|\|\|\|\|\|\|\|\|\|\|\| |
| m576-1 | MNTIFKISALTLSAALALSACGKKEAAPASASEPAAASSAQGDTSSIGSTMQQASYAMGV |
| | 10 20 30 40 50 60 |
| | 70 80 90 100 110 120 |
| g576-1.pep | DIGRSLKQMKEQGAEIDLKVFTDAMQAVYDGKEIKMTEEQAQEVMMKFLQEQQAKAVEKH |
| | \|\|\|\|\|\|\|\|\|\|\|\|\|\|\|\|\|\|\|\|\|\|:\|\|\|\|\|\|\|\|\|\|\|\|\|\|\|\|\|\|\|\|\|\|\|\|\|\|\|\|\|\|\|\|\|\|\|\|\| |
| m576-1 | DIGRSLKQMKEQGAEIDLKVFTEAMQAVYDGKEIKMTEEQAQEVMMKFLQEQQAKAVEKH |
| | 70 80 90 100 110 120 |
| | 130 140 150 160 170 180 |
| g576-1.pep | KADAKANKEKGEAFLKENAAKDGVKTTASGLQYKITKQGEGKQPTKDDIVTVEYEGRLID |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m576-1 | KADAKANKEKGEAFLKENAAKDGVKTTASGLQYKITKQGEGKQPTKDDIVTVEYEGRLID |
| | 130 140 150 160 170 180 |
| | 190 200 210 220 230 240 |
| g576-1.pep | GTVFDSSKANGGPATFPLSQVIPGWTEGVRLLKEGGEATFYIPSNLAYREQGAGEKIGPN |
| | \|\|\|\|\|\|\|\|\|\|\|\|\|:\|\|\|\|\|\|\|\|\|\|\|\|\|\|\|:\|\|\|\|\|\|\|\|\|\|\|\|\|\|\|\|\|\|\|\|\|\|\|\|:\|\|\|\|\| |
| m576-1 | GTVFDSSKANGGPVTFPLSQVIPGWTEGVQLLKEGGEATFYIPSNLAYREQGAGDKIGPN |
| | 190 200 210 220 230 240 |
| | 250 260 270 |
| g576-1.pep | ATLVFDVKLVKIGAPENAPAKQPDQVDIKKVNX |
| | \|\|\|\|\|\|\|\|\|\|\|\|\|\|\|\|\|\|\|\|\|\|\| \|\|\|\|\|\|\|\|\| |
| m576-1 | ATLVFDVKLVKIGAPENAPAKQPAQVDIKKVNX |
| | 250 260 270 |

The following partial DNA sequence was identified in N. meningitidis <SEQ ID 30>:

a576-1.seq

| | |
|---|---|
| 1 | ATGAACACCA TTTTCAAAAT CAGCGCACTG ACCCTTTCCG |
| | CCGCTTTGGC |
| 51 | ACTTTCGCCC TGCGGCAAAA AAGAAGCCGC CCCCGCATCT |
| | GCATCCGAAC |
| 101 | CTGCCGCCGC TTCTTCCGCG CAGGGCGACA CCTCTTCGAT |
| | CGGCAGCACG |
| 151 | ATGCAGCAGG CAAGCTATGC GATGGGCGTG GACATCGGAC |
| | GCTCCCTGAA |
| 201 | GCAAATGAAG GAACAGGGCG CGGAAATCGA TTTGAAAGTC |
| | TTTACCGAAG |
| 251 | CCATGCAGGC AGTGTATGAC GGCAAAGAAA TCAAAATGAC |
| | CGAAGAGCAG |
| 301 | GCTCAGGAAG TCATGATGAA ATTCCTTCAG GAACAACAGG |
| | CTAAAGCCGT |
| 351 | AGAAAAACAC AAGGCGGACG CGAAGGCCAA TAAAGAAAAA |
| | GGCGAAGCCT |
| 401 | TTCTGAAAGA AAATGCCGCC AAAGACGGCG TGAAGACCAC |
| | TGCTTCCGGC |
| 451 | CTGCAATACA AAATCACCAA ACAGGGCGAA GGCAAACAGC |
| | CGACCAAAGA |
| 501 | CGACATCGTT ACCGTGGAAT ACGAAGGCCG CCTGATTGAC |
| | GGTACGGTAT |
| 551 | TCGACAGCAG CAAAGCCAAC GGCGGCCCGG TCACCTTCCC |
| | TTTGAGCCAA |
| 601 | GTGATTCTGG GTTGGAGCGA AGGCGTACAG CTTCTGAAAG |
| | AAGGCGGCGA |
| 651 | AGCCACGTTC TACATCCCGT CCAACCTTGC CTACCGCGAA |
| | CAGGGTGCGG |
| 701 | GCGACAAAAT CGGCCCGAAC GCCACTTTGG TATTTGATGT |
| | GAAACTGGTC |
| 751 | AAAATCGGCG CACCCGAAAA CGCGCCCGCC AAGCAGCCGG |
| | CTCAAGTCGA |
| 801 | CATGAAAAAA GTAAATTTAA |

This corresponds to the amino acid sequence <SEQ ID 31; ORF 576-1.a>:

| a576-1.pep | |
|---|---|
| 1 | MNTIFKISAL TLSAALALSA CGKKEAAPAS ASEPAAASSA QGDTSSIGST |
| 51 | MQQASYAMGV DIGRSLKQMK EQGAEIDLKV FTEAMQAVYD GKEIKMTEEQ |
| 101 | AQEVMMKFLQ EQQAKAVEKH KADAKANKEK GEAFLKENAA KDGVKTTASG |
| 151 | LQYKITKQGE GKQPTKDDIV TVEYEGRLID GTVFDSSKAN GGPVTFPLSQ |
| 201 | VILGWTEGVQ LLKEGGEATF YIPSNLAYRE QGAGDKIGPN ATLVFDVKLV |
| 251 | KIGAPENAPA KQPAQVDIKK VN* |

| | |
|---|---|
| a576-1/m576-1 | ORFs 576-1 and 576-1.a 99.6% identity in 272 aa overlap |
| | 10 20 30 40 50 60 |
| a576-1.pep | MNTIFKISALTLSAALALSACGKKEAAPASASEPAAASSAQGDTSSIGSTMQQASYAMGV |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m576-1 | MNTIFKISALTLSAALALSACGKKEAAPASASEPAAASSAQGDTSSIGSTMQQASYAMGV |
| | 10 20 30 40 50 60 |
| | 70 80 90 100 110 120 |
| a576-1.pep | DIGRSLKQMKEQGAEIDLKVFTEAMQAVYDGKEIKMTEEQAQEVMMKFLQEQQAKAVEKH |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m576-1 | DIGRSLKQMKEQGAEIDLKVFTEAMQAVYDGKEIKMTEEQAQEVMMKFLQEQQAKAVEKH |
| | 70 80 90 100 110 120 |
| | 130 140 150 160 170 180 |
| a576-1.pep | KADAKANKEKGEAFLKENAAKDGVKTTASGLQYKITKQGEGKQPTKDDIVTVEYEGRLID |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m576-1 | KADAKANKEKGEAFLKENAAKDGVKTTASGLQYKITKQGEGKQPTKDDIVTVEYEGRLID |
| | 130 140 150 160 170 180 |
| | 190 200 210 220 230 240 |
| a576-1.pep | GTVFDSSKANGGPVTFPLSQVILGWTEGVQLLKEGGEATFYIPSNLAYREQGAGDKIGPN |
| | \|\|\|\|\|\|\|\|\|\|\|\|\|\|\|\|\|\|\|\|\|\|:\|\|\|\|\|\|\|\|\|\|\|\|\|\|\|\|\|\|\|\|\|\|\|\|\|\|\|\|\|\|\|\|\|\|\|\|\| |
| m576-1 | GTVFDSSKANGGPVTFPLSQVIPGWTEGVQLLKEGGEATFYIPSNLAYREQGAGDKIGPN |
| | 190 200 210 220 230 240 |
| | 250 260 270 |
| a576-1.pep | ATLVFDVKLVKIGAPENAPAKQPAQVDIKKVNX |
| | \|\|\|\|\|\|\|\|\|\|\|\|\|\|\|\|\|\|\|\|\|\|\|\|\|\|\|\|\|\|\|\|\| |
| m576-1 | ATLVFDVKLVKIGAPENAPAKQPAQVDIKKVNX |
| | 250 260 270 |

919 and 919-2 gnm43.seq

The following partial DNA sequence was identified in N. meningitidis <SEQ ID 32>:

m919.seq

| | |
|---|---|
| 1 | ATGAAAAAAT ACCTATTCCG CGCCGCCCTG TACGGCATCG |
| | CCGCCGCCAT |
| 51 | CCTCGCCGCC TGCCAAAGCA AGAGCATCCA AACCTTTCCG |
| | CAACCCGACA |
| 101 | CATCCGTCAT CAACGGCCCG GACCGGCCGG TCGGCATCCC |
| | CGACCCCGCC |
| 151 | GGAACGACGG TCGGCGGCGG CGGGGCCGTC TATACCGTTG |
| | TACCGCACCT |
| 201 | GTCCCTGCCC CACTGGGCGG CGCAGGATTT CGCCAAAAGC |
| | CTGCAATCCT |
| 251 | TCCGCCTCGG CTGCGCCAAT TTGAAAAACC GCCAAGGCTG |
| | GCAGGATGTG |
| 301 | TGCGCCCAAG CCTTTCAAAC CCCCGTCCAT TCCTTTCAGG |
| | CAAAACAGTT |
| 351 | TTTTGAACGC TATTTCACGC CGTGGCAGGT TGCAGGCAAC |
| | GGAAGCCTTG |
| 401 | CCGGTACGGT TACCGGCTAT TACGAACCGG TGCTGAAGGG |
| | CGACGACAGG |
| 451 | CGGACGGCAC AAGCCCGCTT CCCGATTTAC GGTATTCCCG |
| | ACGATTTTAT |
| 501 | CTCCGTCCCC CTGCCTGCCG GTTTGCGGAG CGGAAAAGCC |
| | CTTGTCCGCA |
| 551 | TCAGGCAGAC GGGAAAAAAC AGCGGCACAA TCGACAATAC |
| | CGGCGGCACA |
| 601 | CATACCGCCG ACCTCTCCcG ATTCCCCATC ACCGCGCGCA |
| | CAACAGCAAT |
| 651 | CAAAGGCAGG TTTGAAGGAA GCCGCTTCCT CCCCTACCAC |
| | ACGCGCAACC |
| 701 | AAATCAACGG CGGCGCGCTT GACGGCAAAG CCCCGATACT |
| | CGGTTACGCC |
| 751 | GAAGACCCTG TCGAACTTTT TTTTATGCAC ATCCAAGGCT |
| | CGGGCCGTCT |
| 801 | GAAAACCCCG TCCGGCAAAT ACATCCGCAT CGGCTATGCC |
| | GACAAAAACG |
| 851 | AACATCCyTA CGTTTCCATC GGACGCTATA TGGCGGATAA |
| | GGGCTACCTC |
| 901 | AAACTCGGAC AAACCTCCAT GCAGGGCATT AAGTCTTATA |
| | TGCGGCAAAA |
| 951 | TCCGCAACGC CTCGCCGAAG TTTTGGGTCA AAACCCCAGC |
| | TATATCTTTT |
| 1001 | TCCGCGAGCT TGCCGGAAGC AGCAATGACG GCCCTGTCGG |
| | CGCACTGGGC |
| 1051 | ACGCCGCTGA TGGGGGAATA TGCCGGCGCA GTCGACCGGC |
| | ACTACATTAC |
| 1101 | CTTGGGTGCG CCCTTATTTG TCGCCACCGC CCATCCGGTT |
| | ACCCGCAAAG |
| 1151 | CCCTCAACCG CCTGATTATG GCGCAGGATA CCGGCAGCGC |
| | GATTAAAGGC |
| 1201 | GCGGTGCGCG TGGATTATTT TTGGGGATAC GGCGACGAAG |
| | CCGGCGAACT |
| 1251 | TGCCGGCAAA CAGAAAACCA CGGGATATGT CTGGCAGCTC |
| | CTACCCAACG |
| 1301 | GTATGAAGCC CGAATACCGc CCGTAA |

This corresponds to the amino acid sequence <SEQ ID 33; ORF 919>:

m919.pep

| | |
|---|---|
| 1 | MKKYLFRAAL YGIAAAILAA CQSKSIQTFP QPDTSVINGP |
| | DRPVGIPDPA |
| 51 | GTTVGGGGAV YTVVPHLSLP HWAAQDFAKS LQSFRLGCAN |
| | LKNRQGWQDV |
| 101 | CAQAFQTPVH SFQAKQFFER YFTPWQVAGN GSLAGTVTGY |
| | YEPVLKGDDR |
| 151 | RTAQARFPIY GIPDDFISVP LPAGLRSGKA LVRIRQTGKN |
| | SGTIDNTGGT |
| 201 | HTADLSRFPI TARTTAIKGR FEGSRFLPYH TRNQINGGAL |
| | DGKAPILGYA |
| 251 | EDPVELFFMH IQGSGRLKTP SGKYIRIGYA DKNEHPYVSI |
| | GRYMADKGYL |
| 301 | KLGQTSMQGI KSYMRQNPQR LAEVLGQNPS YIFFRELAGS |
| | SNDGPVGALG |
| 351 | TPLMGEYAGA VDRHYITLGA PLFVATAHPV TRKALNRLIM |
| | AQDTGSAIKG |
| 401 | AVRYDYFWGY GDEAGELAGK QKTTGYVWQL LPNGMKPEYR |
| | P* |

The following partial DNA sequence was identified in N. meningitidis <SEQ ID 34>:

m919-2.seq

| | |
|---|---|
| 1 | ATGAAAAAAT ACCTATTCCG CGCCGCCCTG TACGGCATCG |
| | CCGCCGCCAT |
| 51 | CCTCGCCGCC TGCCAAAGCA AGAGCATCCA AACCTTTCCG |
| | CAACCCGACA |
| 101 | CATCCGTCAT CAACGGCCCG GACCGGCCGG TCGGCATCCC |
| | CGACCCCGCC |
| 151 | GGAACGACGG TCGGCGGCGG CGGGGCCGTC TATACCGTTG |
| | TACCGCACCT |
| 201 | GTCCCTGCCC CACTGGGCGG CGCAGGATTT CGCCAAAAGC |
| | CTGCAATCCT |
| 251 | TCCGCCTCGG CTGCGCCAAT TTGAAAAACC GCCAAGGCTG |
| | GCAGGATGTG |
| 301 | TGCGCCCAAG CCTTTCAAAC CCCCGTCCAT TCCTTTCAGG |
| | CAAAACAGTT |
| 351 | TTTTGAACGC TATTTCACGC CGTGGCAGGT TGCAGGCAAC |
| | GGAAGCCTTG |
| 401 | CCGGTACGGT TACCGGCTAT TACGAACCGG TGCTGAAGGG |
| | CGACGACAGG |
| 451 | CGGACGGCAC AAGCCCGCTT CCCGATTTAC GGTATTCCCG |
| | ACGATTTTAT |
| 501 | CTCCGTCCCC CTGCCTGCCG GTTTGCGGAG CGGAAAAGCC |
| | CTTGTCCGCA |
| 551 | TCAGGCAGAC GGGAAAAAAC AGCGGCACAA TCGACAATAC |
| | CGGCGGCACA |
| 601 | CATACCGCCG ACCTCTCCCG ATTCCCCATC ACCGCGCGCA |
| | CAACAGCAAT |
| 651 | CAAAGGCAGG TTTGAAGGAA GCCGCTTCCT CCCCTACCAC |
| | ACGCGCAACC |
| 701 | AAATCAACGG CGGCGCGCTT GACGGCAAAG CCCCGATACT |
| | CGGTTACGCC |
| 751 | GAAGACCCTG TCGAACTTTT TTTTATGCAC ATCCAAGGCT |
| | CGGGCCGTCT |
| 801 | GAAAACCCCG TCCGGCAAAT ACATCCGCAT CGGCTATGCC |
| | GACAAAAACG |
| 851 | AACATCCCTA CGTTTCCATC GGACGCTATA TGGCGGATAA |
| | GGGCTACCTC |
| 901 | AAACTCGGAC AAACCTCCAT GCAGGGCATT AAGTCTTATA |
| | TGCGGCAAAA |
| 951 | TCCGCAACGC CTCGCCGAAG TTTTGGGTCA AAACCCCAGC |
| | TATATCTTTT |
| 1001 | TCCGCGAGCT TGCCGGAAGC AGCAATGACG GCCCTGTCGG |
| | CGCACTGGGC |
| 1051 | ACGCCGCTGA TGGGGGAATA TGCCGGCGCA GTCGACCGGC |
| | ACTACATTAC |
| 1101 | CTTGGGTGCG CCCTTATTTG TCGCCACCGC CCATCCGGTT |
| | ACCCGCAAAG |
| 1151 | CCCTCAACCG CCTGATTATG GCGCAGGATA CCGGCAGCGC |
| | GATTAAAGGC |
| 1201 | GCGGTGCGCG TGGATTATTT TTGGGGATAC GGCGACGAAG |
| | CCGGCGAACT |
| 1251 | TGCCGGCAAA CAGAAAACCA CGGGATATGT CTGGCAGCTC |
| | CTACCCAACG |
| 1301 | GTATGAAGCC CGAATACCGC CCGTAA |

This corresponds to the amino acid sequence <SEQ ID 35; ORF 919-2>:

m919-2.pep

| | |
|---|---|
| 1 | MKKYLFRAAL YGIAAAILAA CQSKSIQTFP QPDTSVINGP |
| | DRPVGIPDPA |
| 51 | GTTVGGGGAV YTVVPHLSLP HWAAQDFAKS LQSFRLGCAN |
| | LKNRQGWQDV |
| 101 | CAQAFQTPVH SFQAKQFFER YFTPWQVAGN GSLAGTVTGY |
| | YEPVLKGDDR |
| 151 | RTAQARFPIY GIPDDFISVP LPAGLRSGKA LVRIRQTGKN |
| | SGTIDNTGGT |
| 201 | HTADLSRFPI TARTTAIKGR FEGSRFLPYH TRNQINGGAL |
| | DGKAPILGYA |
| 251 | EDPVELFFMH IQGSGRLKTP SGKYIRIFYA DKNEHPYVSI |
| | GRYMADKGYL |
| 301 | KLGQTSMQGI KSYMRQNPQR LAEVLGQNPS YIFFRELAGS |
| | SNDGPVGALG |
| 351 | TPLMGEYAGA VDRHYITLGA PLFVATAHPV TRKALNRLIM |
| | AQDTGSAIKG |
| 401 | AVRVDYFWGY GDEAGELAGK QKTTGYVWQL LPNGMKPEYR |
| | P* |

The following partial DNA sequence was identified in N. gonorrhoeae <SEQ ID 36>:

```
g919.seq
      1 ATGAAAAAAC ACCTGCTCCG CTCCGCCCTG TACGGcatCG CCGCCgccAT
    51 CctcgCCGCC TGCCAAAgca gGAGCATCCA AACCTTTCCG CAACCCGACA
  101 CATCCGTCAT CAACGGCCCG GACCGGCCGG CCGGCATCCC CGACCCCGCC
  151 GGAACGACGG TTGCCGGCGG CGGGGCCGTC TATACCGTTG TGCCGCACCT
  201 GTCCATGCCC CACTGGGCGG CGCaggATTT TGCCAAAAGC CTGCAATCCT
  251 TCCGCCTCGG CTGCGCCAAT TTGAAAAACC GCCAAGGCTG GCAGGATGTG
  301 TGCGCCCAAG CCTTTCAAAC CCCCGTGCAT TCCTTTCAGG CAAAGcGgTT
  351 TTTTGAACGC TATTTCACGC cgtGGCaggt tgcaggcaAC GGAAGcCTTG
  401 Caggtacggt TACCGGCTAT TACGAACCGG TGCTGAAGGG CGACGGCAGG
  451 CGGACGGAAC GGGCCCGCTT CCCGATTTAC GGTATTCCCG ACGATTTTAT
  501 CTCCGTCCCG CTGCCTGCCG GTTTGCGGGG CGGAAAAAAC CTTGTCCGCA
  551 TCAGGCAGac ggGGAAAAAC AGCGGCACGA TCGACAATGC CGGCGGCACG
  601 CATACCGCCG ACCTCTCCCG ATTCCCCATC ACCGCGCGCA CAACGGcaat
  651 caaaGGCAGG TTTGAaggAA GCCGCTTCCT CCCTTACCAC ACGCGCAACC
  701 AAAtcaacGG CGGCgcgcTT GACGGCAAag cccCCATCCT CggttacgcC
  751 GAagaccCcG tcgaacttTT TTTCATGCAC AtccaaggCT CGGGCCGCCT
  801 GAAAACCCcg tccggcaaat acatCCGCAt cggaTacgcc gacAAAAACG
  851 AACAtccgTa tgtttccatc ggACGctaTA TGGCGGACAA AGGCTACCTC
  901 AAGctcgggc agACCTCGAT GCAGGgcatc aaagcCTATA TGCGGCAAAA
  951 TCCGCAACGC CTCGCCGAAG TTTTGGGTCA AAACCCCAGC TATATCTTTT
1001 TCCGCGAGCT TGCCGGAAGC GGCAATGAGG GCCCCGTCGG CGCACTGGGC
1051 ACGCCACTGA TGGGGGAATA CGCCGGCGCA ATCGACCGGC ACTACATTAC
1101 CTTGGGCGCG CCCTTATTTG TCGCCACCGC CCATCCGGTT ACCCGGAAAG
1151 CCCTCAACCG CCTGATTATG GCGCAGGATA CAGGCAGCGC GATCAAAGGC
1201 GGGGTGCGCG TGGATTATTT TTGGGGTTAC GGCGACGAAG CCGGCGAACT
1251 TGCCGGCAAA CAGAAAACCA CGGGATACGT CTGGCAGCTC CTGCCCAACG
1301 GCATGAAGCC CGAATACCGC CGGTGA
```

This corresponds to the amino acid sequence <SEQ ID 37; ORF 919.ng>:

```
g919.pep
    1 MKKHLLRSAL YGIAAAILAA CQSRSIQTFP QPDTSVINGP DRPAGIPDPA
  51 GTTVAGGGAV YTVVPHLSMP HWAAQDFAKS LQSFRLGCAN LKNRQGWQDV
101 CAQAFQTPVH SFQAKRFFER YFTPWQVAGN GSLAGTVTGY YEPVLKGDGR
151 RTERARFPIY GIPDDFISVP LPAGLRGGKN LVRIRQTGKN SGTIDNAGGT
201 HTADLSRFPI TARTTAIKGR FEGSRFLPYH TRNQINGGAL DGKAPILGYA
251 EDPVELFFMH IQGSGRLKTP SGKYIRIGYA DKNEHPYVSI GRYMADKGYL
301 KLGQTSMQGI KAYMRQNPQR LAEVLGQNPS YIFFRELAGS GNEGPVGALG
351 TPLMGEYAGA IDRHYITLGA PLFVATAHPV TRKALNRLIM AQDTGSAIKG
401 AVRVDYFWGY GDEAGELAGK QKTTGYVWQL LPNGMKPEYR P*
```

ORF 919 shows 95.9% identity over a 441 aa overlap with a predicted ORF (ORF 919.ng) from N. gonorrhoeae:

```
m919/g919
                  10 20 30 40 50 60
m919.pep MKKYLFRAALYGIAAAILAACQSKSIQTFPQPDTSVINGPDRPVGIPDPAGTTVGGGGAV
            |||:|:|:|||||||||||||||:|||||||||||||||||||:||||||||||:|||||
g919 MKKHLLRSALYGIAAAILAACQSRSIQTFPQPDTSVINGPDRPAGIPDPAGTTVAGGGAV
                  10 20 30 40 50 60
                  70 80 90 100 110 120
m919.pep YTVVPHLSLPHWAAQDFAKSLQSFRLGCANLKNRQGWQDVCAQAFQTPVHSFQAKQFFER
            ||||||||:||||||||||||||||||||||||||||||||||||||||||||||:||||
g919 YTVVPHLSMPHWAAQDFAKSLQSFRLGCANLKNRQGWQDVCAQAFQTPVHSFQAKRFFER
                  70 80 90 100 110 120
                  130 140 150 160 170 180
m919.pep YFTPWQVAGNGSLAGTVTGYYEPVLKGDDRRTAQARFPIYGIPDDFISVPLPAGLRSGKA
            |||||||||||||||||||||||||||| ||| :|||||||||||||||||||||||:||
g919 YFTPWQVAGNGSLAGTVTGYYEPVLKGDGRRTERARFPIYGIPDDFISVPLPAGLRGGKN
                  130 140 150 160 170 180
                  190 200 210 220 230 240
m919.pep LVRIRQTGKNSGTIDNTGGTHTADLSRFPITARTTAIKGRFEGSRFLPYHTRNQINGGAL
            ||||||||||||||||:|||||||||||||||||||||||||||||||||||||||||||
g919 LVRIRQTGKNSGTIDNAGGTHTADLSRFPITARTTAIKGRFEGSRFLPYHTRNQINGGAL
                  190 200 210 220 230 240
                  250 260 270 280 290 300
m919.pep DGKAPILGYAEDPVELFFMHIQGSGRLKTPSGKYIRIGYADKNEHPYVSIGRYMADKGYL
            ||||||||||||||||||||||||||||||||||||||||||||||||||||||||||||
g919 DGKAPILGYAEDPVELFFMHIQGSGRLKTPSGKYIRIGYADKNEHPYVSIGRYMADKGYL
                  250 260 270 280 290 300
                  310 320 330 340 350 360
m919.pep KLGQTSMQGIKSYMRQNPQRLAEVLGQNPSYIFFRELAGSSNDGPVGALGTPLMGEYAGA
            |||||||||||:||||||||||||||||||||||||||||:|:|||||||||||||||||
g919 KLGQTSMQGIKAYMRQNPQRLAEVLGQNPSYIFFRELAGSGNEGPVGALGTPLMGEYAGA
                  310 320 330 340 350 360
                  370 380 390 400 410 420
m919.pep VDRHYITLGAPLFVATAHPVTRKALNRLIMAQDTGSAIKGAVRVDYFWGYGDEAGELAGK
            :|||||||||||||||||||||||||||||||||||||||||||||||||||||||||||
g919 IDRHYITLGAPLFVATAHPVTRKALNRLIMAQDTGSAIKGAVRVDYFWGYGDEAGELAGK
                  370 380 390 400 410 420
                  430 440
m919.pep QKTTGYVWQLLPNGMKPEYRPX
            ||||||||||||||||||||||
g919 QKTTGYVWQLLPNGMKPEYRPX
                  430 440
```

The following partial DNA sequence was identified in N. meningitidis <SEQ ID 38>:

```
a919.seq
      1 ATGAAAAAAT ACCTATTCCG CGCCGCCCTG TGCGGCATCG CCGCCGCCAT
    51 CCTCGCCGCC TGCCAAAGCA AGAGCATCCA AACCTTTCCG CAACCCGACA
  101 CATCCGTCAT CAACGGCCCG GACCGGCCGG TCGGCATCCC CGACCCCGCC
  151 GGAACGACGG TCGGCGGCGG CGGGGCCGTT TATACCGTTG TGCCGCACCT
  201 GTCCCTGCCC CACTGGGCGG CGCAGGATTT CGCCAAAAGC CTGCAATCCT
  251 TCCGCCTCGG CTGCGCCAAT TTGAAAAACC GCCAAGGCTG GCAGGATGTG
  301 TGCGCCCAAG CCTTTCAAAC CCCCGTCCAT TCCGTTCAGG CAAAACAGTT
  351 TTTTGAACGC TATTTCACGC CGTGGCAGGT TGCAGGCAAC GGAAGCCTTG
  401 CCGGTACGGT TACCGGCTAT TACGAGCCGG TGCTGAAGGG CGACGACAGG
  451 CGGACGGCAC AAGCCCGCTT CCCGATTTAC GGTATTCCCG ACGATTTTAT
  501 CTCCGTCCCC CTGCCTGCCG GTTTGCGGAG CGGAAAAGCC CTTGTCGGCA
  551 TCAGGCAGAC GGGAAAAAAC AGCGGCACAA TCGACAATAC CGGCGGCACA
  601 CATACCGCCG ACCTCTCCGA ATTCCCCATC ACTGCGCGCA CAACGGCAAT
  651 CAAAGGCAGG TTTGAAGGAA GCCGCTTCCT CCCCTACCAC ACGCGCAACC
  701 AAATCAACGG CGGCGCGCTT GACGGCAAAG CCCCGATACT CGGTTACGCC
  751 GAAGACCCCG TCGAACTTTT TTTTATGCAC ATCCAAGGCT CGGGCCGTCT
  801 GAAAACCCCG TCCGGCAAAT ACATCCGCAT CGGCTATGCC GACAAAAACG
  851 AACATCCCTA CGTTTCCATC GGACGCTATA TGGCGGACAA AGGCTACCTC
  901 AAGCTCGGGC AGACCTCGAT GCAGGGCATC AAAGCCTATA TGCAGCAAAA
  951 CCCGCAACGC CTCGCCGAAG TTTTGGGGCA AAACCCCAGC TATATCTTTT
1001 TCCGAGAGCT TACCGGAAGC AGCAATGACG GCCCTGTCGG CGCACTGGGC
1051 ACGCCGCTGA TGGGCGAGTA CGCCGGCGCA GTCGACCGGC ACTACATTAC
1101 CTTGGGCGCG CCCTTATTTG TCGCCACCGC CCATCCGGTT ACCCGCAAAG
1151 CCCTCAACCG CCTGATTATG GCGCAGGATA CCGGCAGCGC GATTAAAGGC
1201 GCGGTGCGCG TGGATTATTT TTGGGGATAC GGCGACGAAG CCGGCGAACT
1251 TGCCGGCAAA CAGAAAACCA CGGGATATGT CTGGCAGCTT CTGCCCAACG
1301 GTATGAAGCC CGAATACCGC CCGTAA
```

This corresponds to the amino acid sequence <SEQ ID 39; ORF 919.a>:

```
a919.pep
    1 MKKYLFRAAL CGIAAAILAA CQSKSIQTFP QPDTSVINGP DRPVGIPDPA
  51 GTTVGGGGAV YTVVPHLSLP HWAAQDFAKS LQSFRLGCAN LKNRQGWQDV
101 CAQAFQTPVH SVQAKQFFER YFTPWQVAGN GSLAGTVTGY YEPVLKGDDR
151 RTAQARFPIY GIPDDFISVP LPAGLRSGKA LVRIRQTGKN SGTIDNTGGT
201 HTADLSQFPI TARTTAIKGR FEGSRFLPYH TRNQINGGAL DGKAPILGYA
251 EDPVELFFMH IQGSGRLKTP SGKYIRIGYA DKNEHPYVSI GRYMADKGYL
301 KLGQTSMQGI KAYMQQNPQR LAEVLGQNPS YIFFRELTGS SNDGPVGALG
351 TPLMGEYAGA VDRHYITLGA PLFVATAHPV TRKALNRLIM AQDTGSAIKG
401 AVRVDYFWGY GDEAGELAGK QKTTGYVWQL LPNGMKPEYR P*
m919/a919 ORFs 919 and 919.a showed a 98.6% identity in 441 aa
overlap
                  10 20 30 40 50 60
m919.pep MKKYLFRAALYGIAAAILAACQSKSIQTFPQPDTSVINGPDRPVGIPDPAGTTVGGGGAV
            |||||||||| |||||||||||||||||||||||||||||||||||||||||||||||||
a919 MKKYLFRAALCGIAAAILAAGQSKSIQTFPQPDTSVINGPDRPVGIPDPAGTTVGGGGAV
                  10 20 30 40 50 60
                  70 80 90 100 110 120
m919.pep YTVVPHLSLPHWAAQDFAKSLQSFRLGCANLKNRQGWQDVCAQAFQTPVHSFQAKQFFER
            ||||||||||||||||||||||||||||||||||||||||||||||||||| ||||||||
a919 YTVVPHLSLPHWAAQDFAKSLQSFRLGCANLKNRQGWQDVCAQAFQTPVHSVQAKQFFER
                  70 80 90 100 110 120
                  130 140 150 160 170 180
m919.pep YFTPWQVAGNGSLAGTVTGYYEPVLKGDDRRTAQARFPIYGIPDDFISVPLPAGLRSGKA
            ||||||||||||||||||||||||||||||||||||||||||||||||||||||||||||
a919 YFTPWQVAGNGSLAGTVTGYYEPVLKGDDRRTAQARFPIYGIPDDFISVPLPAGLRSGKA
                  130 140 150 160 170 180
                  190 200 210 220 230 240
m919.pep LVRIRQTGKNSGTIDNTGGTHTADLSRFPITARTTAIKGRFEGSRFLPYHTRNQINGGAL
            ||||||||||||||||||||||||||:|||||||||||||||||||||||||||||||||
a919 LVRIRQTGKNSGTIDNTGGThTADLSQFPITARTTAIKGRFEGSRFLPYHTRNQINGGAL
                  190 200 210 220 230 240
                  250 260 270 280 290 300
m919.pep DGKAPILGYAEDPVELFFMHIQGSGRLKTPSGKYIRIGYADKNEHPYVSIGRYMADKGYL
            ||||||||||||||||||||||||||||||||||||||||||||||||||||||||||||
a919 DGKAPILGYAEDPVELFFMHIQGSGRLKTPSGKYIRIGYADKNEHPYVSIGRYMADKGYL
                  250 260 270 280 290 300
                  310 320 330 340 350 360
m919.pep KLGQTSMQGIKSYMRQNPQRLAEVLGQNPSYIFFRELAGSSNDGPVGALGTPLMGEYAGA
            |||||||||||:||:||||||||||||||||||||||:||||||||||||||||||||||
a919 KLGQTSMQGIKAYMQQNPQRLAEVLGQNPSYIFFRELTGSSNDGPVGALGTPLMGEYAGA
                  310 320 330 340 350 360
                  370 380 390 400 410 420
m919.pep VDRHYITLGAPLFVATAHPVTRKALNRLIMAQDTGSAIKGAVRVDYFWGYGDEAGELAGK
            ||||||||||||||||||||||||||||||||||||||||||||||||||||||||||||
a919 VDRHYITLGAPLFVATAHPVTRKALNRLIMAQDTGSAIKGAVRVDYFWGYGDEAGELAGK
                  370 380 390 400 410 420
                  430 440
m919.pep QKTTGYVWQLLPNGMKPEYRPX
            ||||||||||||||||||||||
a919 QKTTGYVWQLLPNGMKPEYRPX
                  430 440
121 and 121-1
```

The following partial DNA sequence was identified in N. meningitidis <SEQ ID 40>:

```
m121.seq
      1 ATGGAAACAC AGCTTTACAT CGGCATCATG TCGGGAACCA GCATGGACGG
    51 GGCGGATGCC GTACTGATAC GGATGGACGG CGGCAAATGG CTGGGCGCGG
  101 AAGGGCACGC CTTTACCCCC TACCCCGGCA GGTTACGCCG CCAATTGCTG
  151 GATTTGCAGG ACACAGGCGC AGACGAACTG CACCGCAGCA GGATTTTGTC
  201 GCAAGAACTC AGCCGCCTAT ATGCGCAAAC CGCCGCCGAA CTGCTGTGCA
  251 GTCAAAACCT CGCACCGTCC GACATTACCG CCCTCGGCTG CCACGGGCAA
  301 ACCGTCCGAC ACGCGCCGGA ACACGGTTAC AGCATACAGC TTGCCGATTT
  351 GCCGCTGCTG GCGxxxxxxx xxxxxxxxxx xxxxxxxxxx xxxxxxxxxx
  401 xxxxxxxxxx xxxxxxxxxx xxxxxxxxxx xxxxxxxxxx xxxxxxxxxx
  451 xxxxxxxxxx xxxxxxxxxx xxxxxxxxxx xxxxxxxxxx xxxxxxxxxx
  501 xxxxxxxxxx xxxxxxxxxx xxxxxxxxxx xxxxxxxxxx xxxxxxxxxx
  551 xxxxxxxxxx xxxxxxxxxx xxxxxxxxxx xxxxxxxxxx xxxxxxxxxx
  601 xxxxxxCAGC TTCCTTACGA CAAAAACGGT GCAAAGTCGG CACAAGGCAA
  651 CATATTGCCG CAACTGCTCG ACAGGCTGCT CGCCCACCCG TATTTCGCAC
  701 AACGCCACCC TAAAAGCACG GGGCGCGAAC TGTTTGCCAT AAATTGGCTC
  751 GAAACCTACC TTGACGGCGG CGAAAACCGA TACGACGTAT TGCGGACGCT
  801 TTCCCGTTTT ACCGCGCAAA CCGTTTGCGA CGCCGTCTCA CAGGCAGCGG
  851 CAGATGCCCG TCAAATGTAC ATTTGCGACG GCGGCATCCG CAATCCTGTT
  901 TTAATGGCGG ATTTGGCAGA ATGTTTCGGC ACACGCGTTT CCCTGCACAG
  951 CACCGCCGAC CTGAACCTCG ATCCGCAATG GGTGGAAGCC GCCGnATTTG
1001 CGTGGTTGGC GGCGTGTTGG ATTAATCGCA TTCCCGGTAG TCCGCACAAA
1051 GCAACCGGCG CATCCAAACC GTGTATTCTG AnCGCGGGAT ATTATTATTG
1101 A
```

This corresponds to the amino acid sequence <SEQ ID 41; ORF 121>:

```
m121.pep
    1 METQLYIGIM SGTSMDGADA VLIRMDGGKW LGAEGHAFTP YPGRLRRQLL
  51 DLQDTGADEL HRSRILSQEL SRLYAQTAAE LLCSQNLAPS DITALGCHGQ
101 TVRHAPEHGY SIQLADLPLL Axxxxxxxxx xxxxxxxxxx xxxxxxxxxx
151 xxxxxxxxxx xxxxxxxxxx xxxxxxxxxx xxxxxxxxxx xxxxxxxxxx
201 xxQLPYDKNG AKSAQGNILP QLLDRLLAHP YFAQRHPKST GRELFAINWL
251 ETYLDGGENR YDVLRTLSRF TAQTVCDAVS HAAADARQMY ICDGGIRNPV
301 LMADLAECFG TRVSLHSTAD LNLDPQWVEA AXFAWLAACW INRIPGSPHK
351 ATGASKPCIL XAGYYY*
```

The following partial DNA sequence was identified in N. gonorrhoeae <SEQ ID 42>:

```
g121.seq
      1 ATGGAAACAC AGCTTTACAT CGGCATTATG TCGGGAACCA GTATGGACGG
    51 GGCGGATGCC GTGCTGGTAC GGATGGACGG CGGCAAATGG CTGGGCGCGG
  101 AAGGGCACGC CTTTACCCCC TACCCTGACC GGTTGCGCCG CAAATTGCTG
  151 GATTTGCAGG ACACAGGCAC AGACGAACTG CACCGCAGCA GGATGTTGTC
  201 GCAAGAACTC AGCCGCCTGT ACGCGCAAAC CGCCGCCGAA CTGCTGTGCA
  251 GTCAAAACCT CGCTCCGTGC GACATTACCG CCCTCGGCTG CCACGGGCAA
  301 ACCGTCCGAC ACGCGCCGGA ACACGGTtac AGCATACAGC TTGCCGATTT
  351 GCCGCTGCTG GCGGAACTGa cgcggatttT TACCGTCggc gacttcCGCA
  401 GCCGCGACCT TGCTGCCGGC GGacaAGGTG CGCCGCTCGT CCCCGCCTTT
  451 CACGAAGCCC TGTTCCGCGA TGACAGGGAA ACACGCGTGG TACTGAACAT
  501 CGGCGGGATT GCCAACATCA GCGTACTCCC CCCCGGCGCA CCCGCCTTCG
  551 GCTTCGACAC AGGGCCGGGC AATATGCTGA TGGAcgcgtg gacgcaggca
  601 cacTGGcagc TGCCTTACGA CAAAAacggt gcAAAGgcgg cacAAGGCAA
  651 catatTGCcg cAACTGCTCG gcaggctGCT CGCCcaccCG TATTTCTCAC
  701 AACCCcaccc aaAAAGCACG GGgcGCGaac TgtttgcccT AAattggctc
  751 gaaacctAcc ttgacggcgg cgaaaaccga tacgacgtat tgcggacgct
  801 ttcccgattc accgcgcaaA ccgTttggga cgccgtctca CACGCAGCGG
  851 CAGATGCCCG TCAAATGTAC ATTTGCGGCG GCGGCATCCG CAATCCTGTT
  901 TTAATGGCGG ATTTGGCAGA ATGTTTCGGC ACACGCGTTT CCCTGCACAG
  951 CACCGCCGAA CTGAACCTCG ATCCTCAATG GGTGGAGGCG gccgCATTtg
1001 cgtggttggC GGCGTGTTGG ATTAACCGCA TTCCCGGTAG TCCGCACAAA
1051 GCGACCGGCG CATCCAAACC GTGTATTCTG GGCGCGGGAT ATTATTATTG
1101 A
```

This corresponds to the amino acid sequence <SEQ ID 43; ORF 121.ng>:

```
g121.pep
    1 METQLYIGIM SGTSMDGADA VLVRMDGGKW LGAEGHAFTP YPDRLRRKLL
  51 DLQDTGTDEL HRSRMLSQEL SRLYAQTAAE LLCSQNLAPC DITALGCHGQ
101 TVRHAPEHGY SIQLADLPLL AELTRIFTVG DFRSRDLAAG GQGAPLVPAF
151 HEALFRDDRE TRVVLNIGGI ANISVLPPGA PAFGFDTGPG NMLMDAWTQA
201 HWQLPYDKNG AKAAQGNILP QLLGRLLAHP YFSQPHPKST GRELFALNWL
251 ETYLDGGENR YDVLRTLSRF TAQTVWDAVS HAAADARQMY ICGGGIRNPV
301 LMADLAECFG TRVSLHSTAE LNLDPQWVEA AAFAWLAACW INRIPGSPHK
351 ATGASKPCIL GAGYYY*
```

ORF 121 shows 73.5% identity over a 366 aa overlap with a predicted ORF (ORF121.ng) from N. gonorrhoeae:

```
m121/g121
                  10 20 30 40 50 60
m121.pep METQLYIGIMSGTSMDGADAVLIRMDGGKWLGAEGHAFTPYPGRLRRQLLDLQDTGADEL
            ||||||||||||||||||||||:||||||||||||||||||| ||||:||||||||:|||
g121 METQLYIGIMSGTSMDGADAVLVRMDGGKWLGAEGHAFTPYPDRLRRKLLDLQDTGTDEL
                  10 20 30 40 50 60
                  70 80 90 100 110 120
m121.pep HRSRILSQELSRLYAQTAAELLCSQNLAPSDITALGCHGQTVRHAPEHGYSIQLADLPLL
            ||||:|||||||||||||||||||||||| ||||||||||||||||||||||||||||||
g121 HRSRMLSQELSRLYAQTAAELLCSQNLAPCDITALGCHGQTVRHAPEHGYSIQLADLPLL
                  70 80 90 100 110 120
                    130 140 150 160 170 180
m121.pep AXXXXXXXXXXXXXXXXXXXXXXXXXXXXXXXXXXXXXXXXXXXXXXXXXXXXXXXXXXX
            | : : :
g121 AELTRIFTVGDFRSRDLAAGGQGAPLVPAFHEALFRDDRETRVVLNIGGIANISVLPPGA
                    130 140 150 160 170 180
                    190 200 210 220 230 240
m121.pep XXXXXXXXXXXXXXXXXXXXXXQLPYDKNGAKSAQGNILPQLLDRLLAHPYFAQRHPKST
                : : ||||||||||:|||||||||| ||||||||:| |||||
g121 PAFGFDTGPGNMLMDAWTQAHWQLPYDKNGAKAAQGNILPQLLGRLLAHPYFSQPHPKST
                    190 200 210 220 230 240
                  250 260 270 280 290 300
m121.pep GRELFAINWLETYLDGGENRYDVLRTLSRFTAQTVCDAVSHAAADARQMYICDGGIRNPV
            ||||||:|||||||||||||||||||||||||||| |||||||||||||||| |||||||
g121 GRELFALNWLETYLDGGENRYDVLRTLSRFTAQTVWDAVSHAAADARQMYICGGGIRNPV
                  250 260 270 280 290 300
                  310 320 330 340 350 360
m121.pep LMADLAECFGTRVSLHSTADLNLDPQWVEAAXFAWLAACWINRIPGSPHKATGASKPCIL
            |||||||||||||||||||:||||||||||| ||||||||||||||||||||||||||||
g121 LMADLAECFGTRVSLHSTAELNLDPQWVEAAAFAWLAACWINRIPGSPHKATGASKPCIL
                  310 320 330 340 350 360
m121.pep XAGYYYX
            ||||||
g121 GAGYYYX
```

The following partial DNA sequence was identified in N. meningitidis <SEQ ID 44>:

a121.seq

| | |
|---|---|
| 1 | ATGGAAACAC AGCTTTACAT CGGCATCATG TCGGGAACCA |
| | GCATGGACGG |
| 51 | GGCGGATGCC GTACTGATAC GGATGGACGG CGGCAAATGG |
| | CTGGGCGCGG |
| 101 | AAGGGCACGC CTTTACCCCC TACCCCGGCA GGTTACGCCG |
| | CAAATTGCTG |
| 151 | GATTTGCAGG ACACAGGGGC GGACGAACTG CACCGCAGCA |
| | GGATGTTGTC |
| 201 | GCAAGAACTC AGCCGCCTGT ACGCGCAAAC CGCCGCCGAA |
| | CTGCTGTGCA |
| 251 | GTCAAAACCT CGCGCCGTCC GACATTACCG CCCTCGGCTG |
| | CCACGGGCAA |
| 301 | ACCGTCAGAC ACGCGCCGGA ACACAGTTAG AGCGTACAGC |
| | TTGCCGATTT |
| 351 | GCCGCTGCTG GGGGAACGGA CTCAGATTTT TACCGTCGGC |
| | GACTTGCGCA |
| 401 | GCCGCGACCT TGCGGCCGGC GGACAAGGGG GGCCGCTCGT |
| | CCCCGCGTTT |
| 451 | CAGGAAGCGG TGTTGCGGGA CGACAGGGAA AGACGCGCGG |
| | TACTGAACAT |
| 501 | CGGCGGGATT GCCAACATCA GCGTACTCCC CCCCGACGCA |
| | CCCGCCTTCG |
| 551 | GCTTCGACAC AGGACCGGGC AATATGCTGA TGGACGCGTG |
| | GATGCAGGCA |
| 601 | CACTGGCAGC TTCCTTACGA CAAAAACGGT GCAAAGGCGG |
| | CACAAGGCAA |
| 651 | CATATTGCCG CAAGTGCTCG ACAGGCTGCT CGGCCACCCG |
| | TATTTCGCAC |
| 701 | AACCCCACCC TAAAAGCACG GGGCGGGAAC TGTTTGCCCT |
| | AAATTGGCTC |
| 751 | GAAACCTACC TTGACGGCGG CGAAAACCGA TACGACGTAT |
| | TGCGGACGCT |
| 801 | TTCCCGATTC ACCGCGCAAA CCGTTTTCGA CGGCGTCTCA |
| | CACGCAGCGG |
| 851 | CAGATGCCCG TCAAATGTAC ATTTGCGGCG GCGGCATCCG |
| | CAATCCTGTT |
| 901 | TTAATGGCGG ATTTGGCAGA ATGTTTCGGC ACACGCGTTT |
| | CCCTGCACAG |
| 951 | CACCGCCGAA CTGAACCTCG ATCCGCAATG GGTAGAAGCC |
| | GCCGCGTTCG |
| 1001 | CATGGATGGC GGCGTGTTGG GTCAACCGGA TTCCCGGTAG |
| | TCCGCACAAA |
| 1051 | GCAACCGGGG CATCCAAACC GTGTATTCTG GGCGCGGGAT |
| | ATTATTATTG |
| 1101 | A |

This corresponds to the amino acid sequence <SEQ ID 45; ORF 121.a>:

| a121.pep | | |
|---|---|---|
| | 1 | METQLYIGIM SGTSMDGADA VLIRMDGGKW LGAEGHAFTP YPGRLRRKLL |
| | 51 | DLQDTGADEL HRSRMLSQEL SRLYAQTAAE LLCSQNLAPS DITALGCHGQ |
| | 101 | TVRHAPEHSY SVQLADLPLL AERTQIFTVG DFRSRDLAAG GQGAPLVPAF |
| | 151 | HEALFRDDRE TRAVLNIGGI ANISVLPPDA PAFGFDTGPG NMLMDAWMQA |
| | 201 | HWQLPYDKNG AKAAQGNILP QLLDRLLAHP YFAQPHPKST GRELFALNWL |
| | 251 | ETYLDGGENR YDVLRTLSRF TAQTVFDAVS HAAADARQMY ICGGGIRNPV |
| | 301 | LMADLAECFG TRVSLHSTAE LNLDPQWVEA AAFAWMAACW VNRIPGSPHK |
| | 351 | ATGASKPCIL GAGYYY* |

| | |
|---|---|
| m121/a121 | ORFs 121 and 121.a 74.0% identity in 366 aa overlap |
| | 10 20 30 40 50 60 |
| m121.pep | METQLYIGIMSGTSMDGADAVLIRMDGGKWLGAEGHAFTPYPGRLRRQLLDLQDTGADEL |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|:\|\|\|\|\|\|\|\|\|\|\|\| |
| a121 | METQLYIGIMSGTSMDGADAVLIRMDGGKWLGAEGHAFTPYPGRLRRKLLDLQDTGADEL |
| | 10 20 30 40 50 60 |
| | 70 80 90 100 110 120 |
| m121.pep | HRSRILSQELSRLYAQTAAELLCSQNLAPSDITALGCHGQTVRHAPEHGYSIQLADLPLL |
| | \|\|\|\|:\|\|\|\|\|\|\|\|\|\|\|\|\|\|\|\|\|\|\|\|\|\|\|\|\|\|\|\|\|\|\|\|\|\|\|\|\|\|\|\|\|\|:\|\|:\|\|\|\|\|\|\|\| |
| a121 | HRSRMLSQELSRLYAQTAAELLCSQNLAPSDITALGCHGQTVRHAPEHSYSVQLADLPLL |
| | 70 80 90 100 110 120 |
| | 130 140 150 160 170 180 |
| m121.pep | AXXXXXXXXXXXXXXXXXXXXXXXXXXXXXXXXXXXXXXXXXXXXXXXXXXXXXXXXXXX |
| | \| : : : |
| a121 | AERTQIFTVGDFRSRDLAAGGQGAPLVPAFHEALFRDDRETRAVLNIGGIANISVLPPDA |
| | 130 140 150 160 170 180 |
| | 190 200 210 220 230 240 |
| m121.pep | XXXXXXXXXXXXXXXXXXXXXQLPYDKNGAKSAQGNILPQLLDRLLAHPYFAQRHPKST |
| | : \|\|\|\|\|\|\|\|\|\|:\|\|\|\|\|\|\|\|\|\|\|\|\|\|\|\|\|\|\|\|\| \|\|\|\|\| |
| a121 | PAFGFDTGPGNMLMDAWMQAHWQLPYDKNGAKAAQGNILPQLLDRLLAHPYFAQPHPKST |
| | 190 200 210 220 230 240 |
| | 250 260 270 280 290 300 |
| m121.pep | GRELFAINWLETYLDGGENRYDVLRThSRFTAQTVCDAVSHAAADARQMYICDGGIRNPV |
| | \|\|\|\|\|\|:\|\|\|\|\|\|\|\|\|\|\|\|\|\|\|\|\|\|\|\|\|\|\|\|\|\|\|\| \|\|\|\|\|\|\|\|\|\|\|\|\|\|\|\| \|\|\|\|\|\|\| |
| a121 | GRELFALNWLETYLDGGENRYDVLRTLSRFTAQTVFDAVSHAAADARQMYICGGGIRNPV |
| | 250 260 270 280 290 300 |
| | 310 320 330 340 350 360 |
| m121.pep | LMADLAECFGTRVSLHSTADLNLDPQWVEAAXFAWLAACWINRIPGSPHKATGASKPCIL |
| | \|\|\|\|\|\|\|\|\|\|\|\|\|\|\|\|\|\|\|:\|\|\|\|\|\|\|\|\|\|\| \|\|\|:\|\|\|\|:\|\|\|\|\|\|\|\|\|\|\|\|\|\|\|\|\|\|\| |
| a121 | LMADLAECFGTRVSLHSTAELNLDPQWVEAAAFAWMAACWVNRIPGSPHKATGASKPCIL |
| | 310 320 330 340 350 360 |
| m121.pep | XAGYYYX |
| | \|\|\|\|\|\| |
| a121 | GAGYYYX |

Further work revealed the DNA sequence identified in N. meningitidis <SEQ ID 46>:

m121-1.seq

| | |
|---|---|
| 1 | ATGGAAAGAC AGCTTTACAT CGGCATCATG TCGGGAACCA |
| | GCATGGACGG |
| 51 | GGCGGATGGC GTACTGATAC GGATGGACGG CGGCAAATGG |
| | CTGGGCGCGG |
| 101 | AAGGGCAGGC CTTTACCCCC TACGCGGGCA GGTTACGCCG |
| | CCAATTGCTG |
| 151 | GATTTGGAGG ACACAGGCGC AGACGAACTG CACCGCAGCA |
| | GGATTTTGTC |
| 201 | GCAAGAACTC AGCCGCCTAT ATGCGCAAAC CGCCGCCGAA |
| | CTGCTGTGCA |
| 251 | GTCAAAACCT GGCACCGTCC GACATTACCG CCCTCGGCTG |
| | CCACGGGGAA |
| 301 | ACCGTCCGAC ACGCGCCGGA ACACGGTTAC AGCATACAGC |
| | TTGCCGATTT |
| 351 | GCCGCTGCTG GCGGAACGGA CGCGGATTTT TACCGTCGGC |
| | GACTTCCGCA |
| 401 | GCCGCGACCT TGCGGCCGGC GGACAAGGCG CGCCACTCGT |
| | CCCCGCCTTT |
| 451 | CACGAAGCCC TGTTCCGCGA CAACAGGGAA ACACGGGCGG |
| | TACTGAACAT |
| 501 | CGGCGGGATT GCCAACATCA GCGTACTCCC CCCCGACGCA |
| | CCCGCCTTCG |
| 551 | GCTTCGACAC AGGGCCGGGC AATATGCTGA TGGACGCGTG |
| | GACGCAGGCA |
| 601 | CACTGGCAGC TTCCTTACGA CAAAAACGGT GCAAAGGCGG |
| | CACAAGGCAA |
| 651 | CATATTGCCG CAACTGCTCG ACAGGCTGCT CGCCCACCCG |
| | TATTTCGCAC |
| 701 | AACCCCACCC TAAAAGCACG GGGCGCGAAC TGTTTGCCCT |
| | AAATTGGCTC |
| 751 | GAAACCTACC TTGACGGCGG CGAAAACCGA TACGACGTAT |
| | TGCGGACGCT |
| 801 | TTCCCGTTTT ACCGCGCAAA CCGTTTGCGA CGCCGTCTCA |
| | CACGCAGCGG |
| 851 | CAGATGCCCG TCAAATGTAC ATTTGCGGCG GCGGGATCCG |
| | CAATCCTGTT |
| 901 | TTAATGGCGG ATTTGGCAGA ATGTTTCGGC ACACGCGTTT |
| | CCCTGCACAG |
| 951 | CACCGCCGAC CTGAACCTCG ATCCGCAATG GGTGGAAGCC |
| | GCCGNATTTG |
| 1001 | CGTGGTTGGC GGCGTGTTGG ATTAATCGCA TTCCCGGTAG |
| | TCCGCACAAA |
| 1051 | GCAACCGGCG CATCCAAACC GTGTATTCTG ANCGCGGGAT |
| | ATTATTATTG |
| 1101 | A |

This corresponds to the amino acid sequence <SEQ ID 47; ORF 121-1>:

| m121-1.pep | | |
|---|---|---|
| | 1 | METQLYIGIM SGTSMDGADA VLIRMDGGKW LGAEGHAFTP YPGRLRRQLL |
| | 51 | DLQDTGADEL HRSRILSQEL SRLYAQTAAE LLCSQNLAPS DITALGCHGQ |
| | 101 | TVRHAPEHGY SIQLADLPLL AERTRIFTVG DFRSRDLAAG GQGAPLVPAF |
| | 151 | HEALFRDNRE TRAVLNIGGI ANISVLPPDA PAFGFDTGPG NMLMDAWTQA |
| | 201 | HWQLPYDKNG AKAAQGNILP QLLDRLLAHP YFAQPHPKST GRELFALNWL |
| | 251 | ETYLDGGENR YDVLRTLSRF TAQTVCDAVS HAAADARQMY ICGGGIRNPV |
| | 301 | LMADLAECFG TRVSLHSTAD LNLDPQWVEA AXFAWLAACW INRIPGSPHK |
| | 351 | ATGASKPCIL XAGYYY* |

| | |
|---|---|
| m121-1/g121 | ORFs 121-1 and 121-1.ng showed a 95.6% identity in 366 aa overlap |
| | 10 20 30 40 50 60 |
| m121-1.pep | METQLYIGIMSGTSMDGADAVLIRMDGGKWLGAEGHAFTPYPGRLRRQLLDLQDTGADEL |
| | \|\|\|\|\|\|\|\|\|\|\|\|\|\|\|\|\|\|\|\|\|\|:\|\|\|\|\|\|\|\|\|\|\|\|\|\|\|\|\|\|\| \|\|\|\|:\|\|\|\|\|\|\|\|:\|\|\| |
| g121 | METQLYIGIMSGTSMDGADAVLVRMDGGKWLGAEGHAFTPYPDRLRRKLLDLQDTGTDEL |
| | 10 20 30 40 50 60 |
| | 70 80 90 100 110 120 |
| m121-1.pep | HRSRILSQELSRLYAQTAAELLGSQNLAPSDITALGCHGQTVRHAPEHGYSIQLADLPLL |
| | \|\|\|\|:\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\| |
| g121 | HRSRMLSQELSRLYAQTAAELLCSQNLAPCDITALGCHGQTVRHAPEHGYSIQLADLPLL |
| | 70 80 90 100 110 120 |
| | 130 140 150 160 170 180 |
| m121-1.pep | AERTRIFTVGDFRSRDLAAGGQGAPLVPAFHEALFRDNRETRAVLNIGGIANISVLPPDA |
| | \|\| \|\|\|\|\|\|\|\|\|\|\|\|\|\|\|\|\|\|\|\|\|\|\|\|\|\|\|\|\|\|\|\|\|\|:\|\|\|\|:\|\|\|\|\|\|\|\|\|\|\|\|\|\|\| \| |
| g121 | AELTRIFTVGDFRSRDLAAGGQGAPLVPAFHEALFRDDRETRVVLNIGGIANISVLPPGA |
| | 130 140 150 160 170 180 |
| | 190 200 210 220 230 240 |
| m121-1.pep | PAFGFDTGPGNMLMDAWTQAHWQLPYDKNGAKAAQGNILPQLLDRLLAHPYFAQPHPKST |
| | \|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\|\|\|\|\|:\|\|\|\|\|\|\| |
| g121 | PAFGFDTGPGNMLMDAWTQAHWQLPYDKNGAKAAQGNILPQLLGRLLAHPYFSQPHPKST |
| | 190 200 210 220 230 240 |
| | 250 260 270 280 290 300 |
| m121-1.pep | GRELFALNWLETYLDGGENRYDVLRTLSRFTAQTVCDAVSHAAADARQMYICGGGIRNPV |
| | \|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\| |
| g121 | GRELFALNWLETYLDGGENRYDVLRTLSRFTAQTVWDAVSHAAADARQMYICGGGIRNPV |
| | 250 260 270 280 290 300 |
| | 310 320 330 340 350 360 |
| m121-1.pep | LMADLAECFGTRVSLHSTADLNLDPQWVEAAXFAWLAACWINRIPGSPHKATGASKPCIL |
| | \|\|\|\|\|\|\|\|\|\|\|\|\|\|\|\|\|\|\|:\|\|\|\|\|\|\|\|\|\|\| \|\|\|\|\|\|\|\|\|\|\|\|\|\|\|\|\|\|\|\|\|\|\|\|\|\|\|\| |
| g121 | LMADLAECFGTRVSLHSTAELNLDPQWVEAAAFAWLAACWINRIPGSPHKATGASKPCIL |
| | 310 320 330 340 350 360 |
| m121-1.pep | XAGYYYX |
| | \|\|\|\|\|\| |
| g121 | GAGYYYX |

The following partial DNA sequence was identified in N. meningitidis <SEQ ID 48>:

a121-1.seq

| | |
|---|---|
| 1 | ATGGAAACAC AGCTTTACAT CGGCATCATG TCGGGAACCA |
| | GCATGGACGG |
| 51 | GGGGGATGCC GTACTGATAC GGATGGACGG CGGCAAATGG |
| | CTGGGCGCGG |
| 101 | AAGGGCACGC CTTTACCCCC TACCCCGGCA GGTTACGCCG |
| | CAAATTGCTG |
| 151 | GATTTGCAGG ACACAGGCGC GGACGAACTG CACCGCAGCA |
| | GGATGTTGTC |
| 201 | GCAAGAACTC AGCCGCCTGT ACGCGCAAAC CGCCGCCGAA |
| | CTGCTGTGCA |
| 251 | GTCAAAACCT CGCGCCGTCC GACATTACCG CCCTCGGCTG |
| | CCACGGGCAA |
| 301 | ACCGTCAGAC ACGCGCCGGA ACACAGTTAC AGCGTACAGC |
| | TTGCCGATTT |
| 351 | GCCGCTGCTG GCGGAACGGA CTCAGATTTT TACCGTCGGC |
| | GACTTCCGCA |
| 401 | GCCGCGACCT TGCGGCCGGC GGACAAGGCG CGCCGCTCGT |
| | CCCCGCCTTT |
| 451 | CACGAAGCCC TGTTCCGCGA CGACAGGGAA ACACGCGCGG |
| | TACTGAACAT |
| 501 | CGGCGGGATT GCCAACATCA GCGTACTCCC CCCCGACGCA |
| | CCCGCCTTCG |
| 551 | GCTTCGACAC AGGACCGGGC AATATGCTGA TGGACGCGTG |
| | GATGCAGGCA |
| 601 | CACTGGCAGC TTCCTTAGGA CAAAAACGGT GCAAAGGCGG |
| | CACAAGGCAA |
| 651 | CATATTGCCG CAACTGCTCG ACAGGCTGCT CGCCCACCCG |
| | TATTTCGCAC |
| 701 | AACCCCACCC TAAAAGCACG GGGCGCGAAC TGTTTGCCCT |
| | AAATTGGCTC |
| 751 | GAAACCTACC TTGACGGCGG CGAAAACCGA TACGACGTAT |
| | TGCGGACGCT |
| 801 | TTCCCGATTC ACCGCGCAAA CCGTTTTCGA CGCCGTCTCA |
| | CACGCAGCGG |
| 851 | CAGATGCCCG TCAAATGTAC ATTTGCGGCG GCGGCATCCG |
| | CAATCCTGTT |
| 901 | TTAATGGCGG ATTTGGCAGA ATGTTTCGGC ACACGCGTTT |
| | CCCTGCACAG |
| 951 | GAGGGCGGAA CTGAACGTCG ATCCGCAATG GGTAGAAGCC |
| | GCCGCGTTCG |
| 1001 | CATGGATGGC GGCGTGTTGG GTCAACCGGA TTCCCGGTAG |
| | TCCGCACAAA |
| 1051 | GCAAGCGGCG CATCCAAACC GTGTATTCTG GGCGCGGGAT |
| | ATTATTATTG |
| 1101 | A |

This corresponds to the amino acid sequence <SEQ ID 49; ORF 121-1.a>:

| a121-1.pep | | |
|---|---|---|
| | 1 | METQLYIGIM SGTSMDGADA VLIRMDGGKW LGAEGHAFTP YPGRLRRKLL |
| | 51 | DLQDTGADEL HRSRMLSQEL SRLYAQTAAE LLCSQNLAPS DITALGCHGQ |
| | 101 | TVRHAPEHSY SVQLADLPLL AERTQIFTVG DFRSRDLAAG GQGAPLVPAF |
| | 151 | HEALFRDDRE TRAVLNIGGI ANISVLPPDA PAFGFDTGPG NMLMDAWMQA |
| | 201 | HWQLPYDKNG AKAAQGNILP QLLDRLLAHP YFAQPHPKST GRELFALNWL |
| | 251 | ETYLDGGENR YDVLRTLSRF TAQTVFDAVS HAAADARQMY ICGGGIRNPV |
| | 301 | LMADLAECFG TRVSLHSTAE LNLDPQWVEA AAFAWMAACW VNRIPGSPHK |
| | 351 | ATGASKPCIL GAGYYY* |

| | |
|---|---|
| m121-1/a121-1 | ORFs 121-1 and 121-1.a showed a 96.4% identity in 366 aa overlap |
| | 10 20 30 40 50 60 |
| m121-1.pep | METQLYIGIMSGTSMDGADAVLIRMDGGKWLGAEGHAFTPYPGRLRRQLLDLQDTGADEL |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|:\|\|\|\|\|\|\|\|\|\|\|\| |
| a121-1 | METQLYIGIMSGTSMDGADAVLIRMDGGKWLGAEGHAFTPYPGRLRRKLLDLQDTGADEL |
| | 10 20 30 40 50 60 |
| | 70 80 90 100 110 120 |
| m121-1.pep | HRSRILSQELSRLYAQTAAELLCSQNLAPSDITALGCHGQTVRHAPEHGYSIQLADLPLL |
| | \|\|\|\|:\|\|\|\|\|\|\|\|\|\|\|\|\|\|\|\|\|\|\|\|\|\|\|\|\|\|\|\|\|\|\|\|\|\|\|\|\|\|\|\|\|\|\|:\|\|:\|\|\|\|\|\|\|\| |
| a121-1 | HRSRMLSQELSRLYAQTAAELLCSQNLAPSDITALGCHGQTVRHAPEHSYSVQLADLPLL |
| | 70 80 90 100 110 120 |
| | 130 140 150 160 170 180 |
| m121-1.pep | AERTRIFTVGDFRSRDLAAGGQGAPLVPAFHEALFRDNRETRAVLNIGGIANISVLPPDA |
| | \|\|\|\|:\|\|\|\|\|\|\|\|\|\|\|\|\|\|\|\|\|\|\|\|\|\|\|\|\|\|\|\|\|\|\|\|:\|\|\|\|\|\|\|\|\|\|\|\|\|\|\|\|\|\|\|\|\|\| |
| a121-1 | AERTQIFTVGDFRSRDLAAGGQGAPLVPAFHEALFRDDRETRAVLNIGGIANISVLPPDA |
| | 130 140 150 160 170 180 |
| | 190 200 210 220 230 240 |
| m121-1.pep | PAFGFDTGPGNMLMDAWTQAHWQLPYDKNGAKAAQGNILPQLLDRLLAHPYFAQPHPKST |
| | \|\|\|\|\|\|\|\|\|\|\|\|\|\|\|\|\| \|\|\|\|\|\|\|\|\|\|\|\|\|\|\|\|\|\|\|\|\|\|\|\|\|\|\|\|\|\|\|\|\|\|\|\|\|\|\|\|\|\| |
| a121-1 | PAFGFDTGPGNMLMDAWMQAHWQLPYDKNGAKAAQGNILPQLLDRLLAHPYFAQPHPKST |
| | 190 200 210 220 230 240 |
| | 250 260 270 280 290 300 |
| m121-1.pep | GRELFALNWLETYLDGGENRYDVLRTLSRFTAQTVCDAVSHAAADARQMYICGGGIRNPV |
| | \|\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\|\|\|\|\|\|\|\|\|\|\|\|\| |
| a121-1 | GRELFALNWLETYLDGGENRYDVLRTLSRFTAQTVFDAVSHAAADARQMYICGGGIRNPV |
| | 250 260 270 280 290 300 |
| | 310 320 330 340 350 360 |
| m121-1.pep | LMADLAECFGTRVSLHSTADLNLDPQWVEAAXFAWLAACWINRIPGSPHKATGASKPCIL |
| | \|\|\|\|\|\|\|\|\|\|\|\|\|\|\|\|\|\|\|:\|\|\|\|\|\|\|\|\|\|\| \|\|\|:\|\|\|\|:\|\|\|\|\|\|\|\|\|\|\|\|\|\|\|\|\|\|\| |
| a121 | LMADLAECFGTRVSLHSTAELNLDPQWVEAAAFAWMAACWVNRIPGSPHKATGASKPCIL |
| | 310 320 330 340 350 360 |
| m121-1.pep | XAGYYYX |
| | \|\|\|\|\|\| |
| a121 | GAGYYYX |
| 128 and 128-1 | |

The following partial DNA sequence was identified in N. meningitidis <SEQ ID 50>:

m128.seq (partial)

| | |
|---|---|
| 1 | ATGACTGACA ACGCACTGCT CCATTTGGGC GAAGAACCCC |
| | GTTTTGATCA |
| 51 | AATCAAAACC GAAGACATCA AACCCGCCCT GCAAACCGCC |
| | ATCGCCGAAG |
| 101 | CGCGGGAACA AATCGCCGCC ATCAAAGCCC AAACGCACAC |
| | CGGGTGGGCA |
| 151 | AACACTGTCG AACCCCTGAC CGGCATCAGC GAACGCGTCG |
| | GCAGGATTTG |
| 201 | GGGCGTGGTG TCGCACCTCA ACTGCGTCGC CGACACGCCC |
| | GAACTGCGCG |
| 251 | CCGTCTATAA CGAACTGATG GCCGAAATCA CCGTCTTCTT |
| | CACCGAAATC |
| 301 | GGACAAGACA TCGAGCTGTA CAACCGCTTC AAAACCATCA |
| | AAAATTCCCC |
| 351 | CGAATTCGAC ACCCTCTCCC CCGCACAAAA AACCAAACTC |
| | AACCAC |
| 1 | TACGCCAGCG AAAAACTGCG CGAAGCCAAA TACGCGTTCA |
| | GGGAAACGGA |
| 51 | wGTCAAAAAA TAyTTCCCyG TCGGCAAwGT ATTAAACGGA |
| | CTGTTCGCCC |
| 101 | AAmTCAAAAA AGTmTAGGGG ATCGGATTTA CCGAAAAAAC |
| | yGTCCCCGTC |
| 151 | TGGGACAAAG ACGTGGGCTA TTkTGAATTG CAACAAAACG |
| | GCGAAmCCAT |
| 201 | AGGCGGCGTT TATATGGATT TGTACGCACG CGAAGGGAAA |
| | CGCGGCGGCG |
| 251 | CGTGGATGAA CGACTACAAA GGCCGCCGCC GTTTTTCAGA |
| | CGGCACGCTG |
| 301 | CAAyTGCCCA CCGCCTACCT CGTCTGCAAC TTCGCGCCAC |
| | CCGTCGGCGG |
| 351 | CAGGGAAGCC CGCyTGAGCC ACGACGAAAT CCTCATCCTC |
| | TTCCACGAAA |
| 401 | CCGGACACGG GCTGCACCAC CTGCTTACCC AAGTGGACGA |
| | ACTGGGCGTA |
| 451 | TCCGGCATCA ACGGCGTAkA ATGGGACGCG GTCGAACTGC |
| | CCAGCCAGTT |
| 501 | TATGGAAAAT TTCGTTTGGG AATACAATGT CTTGGGACAA |
| | mTGTCAGCCC |
| 551 | ACGAAGAAAC CGGcgTTCCC yTGCCGAAAG AACTCTTsGA |
| | CAAAwTGCTC |
| 601 | GCCGCCAAAA ACTTCCAAsG CGGCATGTTC yTsGTCCGGC |
| | AAwTGGAGTT |
| 651 | CGCCCTCTTT GATATGATGA TTTACAGCGA AGACGACGAA |
| | GGCCGTCTGA |
| 701 | AAAACTGGCA ACAGGTTTTA GACAGGGTGC GCAAAAAAGT |
| | CGCCGTCATC |
| 751 | CAGCCGCCCG AATACAACCG CTTCGGCTTG AGCTTCGGCC |
| | ACATCTTCGC |
| 801 | AGGCGGCTAT TCCGCAGCTn ATTACAGCTA CGCGTGGGCG |
| | GAAGTATTGA |
| 851 | GCGCGGACGC ATACGCCGCC TTTGAAGAAA GCGACGATGT |
| | CGCCGCCACA |
| 901 | GGCAAACGCT TTTGGCAGGA AATCCTCGCC GTCGGGGnAT |
| | CGCGCAGCGG |
| 951 | nGCAGAATCC TTCAAAGCCT TCCGCGGCCG CGAACCGAGC |
| | ATAGACGCAC |
| 1001 | TCTTGCGCCA CAGCGGTTTC GACAACGCGG TCTGA |

This corresponds to the amino acid sequence <SEQ ID 51; ORF 128>:

m128.pep (partial)

| | |
|---|---|
| 1 | MTDNALLHLG EEPRFDQIKT EDIKPALQTA IAEAREQIAA |
| | IKAQTHTGWA |
| 51 | NTVEPLTGIT ERVGRIWGVV SHLNCVADTP ELRAVYNELM |
| | PEITVFFEI |
| 101 | GQDIELYNRF KTIKNSPEFD TLSPAQKTKL NH |

//

| | |
|---|---|
| 1 | YASEKLREAK YAFSETXVKK YFPVGXVLNG LFAQXKKLYG |
| | IGFTEKTVPV |
| 51 | WHKDVRYXEL QQNGEXIGGV YMDLYAREGK RGGAWMNDYK |
| | GRRRFSDGTL |
| 101 | QLPTAYLVCN FAPPVGGREA RLSHDEILIL FHETGHGLHH |
| | LLTQVDELGV |
| 151 | SGINGVXWDA VELPSQFMEN FVWEYNVLAQ XSAHEETGVP |
| | LPKELXDKXL |
| 201 | AAKNFQXGMF XVRQXEFALF DMMIYSEDDE GRLKNWQQVL |
| | DSVRKKVAVI |
| 251 | QPPEYNRFAL SFGHIFAGGY SAAXYSYAWA EVLSADAYAA |
| | FEESDDVAAT |
| 301 | GKRFWQEILA VGXSRSGAES FKAFRGREPS IDALLRHSGF |
| | DNAV* |

The following partial DNA sequence was identified in N. gonorrhoeae <SEQ ID 52>:

g128.seq

| | |
|---|---|
| 1 | atgattgaca acgCActgct ccacttgggc gaagaaccCC |
| | GTTTTaatca |
| 51 | aatccaaacc gaagACAtca AACCCGCCGT CCAAACCGCC |
| | ATCGCCGAAG |
| 101 | CGCGCGGACA AATCGCCGCC GTCAAAGCGC AAACGCACAC |
| | CGGCTGGGCG |
| 151 | AACACCGTCG AGCGTCTGAC CGGGATCACC GAACGCGTCG |
| | GCAGGATTTG |
| 201 | GGGCGTCGTG TCCCATCTCA ACTCCGTCGT CGACACGCCC |
| | GAACTGCGCG |
| 251 | CCGTCTATAA CGAACTGATG CCTGAAATCA CCGTCTTCTT |
| | CACCGAAATC |
| 301 | GGACAAGACA TCGAACTGTA CAACCGCTTC AAAACCATCA |
| | AAAATTCCCC |
| 351 | CGAATTTGCA ACGCTTTCCC CCGCACAAAA AACCAAGCTC |
| | GATCACGACC |
| 401 | TGGGCGATTT CGTATTGAGG GGCGCGGAAC TGCCGCCCGA |
| | ACGGCAGGCA |
| 451 | GAACTGGCAA AACTGCAAAC CGAAGGCGCG CAACTTTCCG |
| | CCAAATTCTC |
| 501 | CCAAAACGTC CTAGACGCGA CCGACGCGTT CGGCATTTAC |
| | TTTGACGATG |
| 551 | CCGCACCGCT TGCCGGCATT CCCGAAGACG CGCTCGCCAT |
| | GTTTGCCGCC |
| 601 | GCCGCGCAAA GCGAAGGCAA AACAGGTTAC AAAATCGGCT |
| | TGCAGATTCC |
| 651 | GCACTACCTT GGCGTTATCC AATACGCCGG CAACCGCGAA |
| | CTGCGCGAAC |
| 701 | AAATCTACCG CGCCTACGTT ACCCGTGCCA GGGAACTTTC |
| | AAACGACGGC |
| 751 | AAATTCGACA ACACCGCCAA CATCGACCGC ACGCTCGAAA |
| | ACGCATTGAA |
| 801 | AACCGccaaa cTGCTCGGCT TTAAAAATTA CGCCGAATTG |
| | TCGCTGGCAA |
| 851 | CCAAAATGGC GGACACGCCC GAACAGGTTT TAAACTTCCT |
| | GCACGACCTC |
| 901 | GCCCGCCGCG CCAAACCCTA CGCCGAAAAA GACCTCGCCG |
| | AAGTCAAAGC |
| 951 | CTTCGCCCGC GAACACCTCG GTCTCGCCGA CCCGCAGCCG |
| | TGGGACTTGA |
| 1001 | GCTACGCCGG CGAAAAACTG CGCGAAGCCA AATACGCATT |
| | CAGCGAAACC |
| 1051 | GAAGTCAAAA AATACTTCCC CGTCGGCAAA GTTCTGGCAG |
| | GCCTGTTCGC |
| 1101 | CCAAATCAAA AAACTCTACG GCATCGGATT CGCCGAAAAA |
| | ACCGTTCCCG |
| 1151 | TCTGGCACAA AGACGTGCGC TATTTTGAAT TGCAACAAAA |
| | CGGCAAAACC |
| 1201 | ATCGGCGGGG TTTATATGGA TTTGTACGCA CGCGAAGGCA |
| | AACGCGGCGG |
| 1251 | CGCGTGGATG AACGACtaca AAGGCCGCCG CCGCTTTGCC |
| | GACGgcacGC |
| 1301 | TGCAACTGCC CACCGCCTAC CTCGTCTGCA ACTTCGCCCC |
| | GCCCGTCGGC |
| 1351 | GGCAAAGAAG CGCGTTTAAG CCACGACGAA ATGGTCACCC |
| | TCTTCCACGA |
| 1401 | AacCGGCCAC GGACTGCACC ACCTGCTTAG GCAAGTGGAC |
| | GAACTGGGCG |
| 1451 | TGTCCGGCAT CAAcggcgtA GAATGGGACG CGGTCGAACT |
| | GCCCAGCCAG |
| 1501 | TTTATGGAAA ACTTCGTTTG GGAATACAAT GTATTGGCAC |
| | AAATGTCCGC |
| 1551 | CCACGAAGAA AccgGCGAGC CCCTGCCGAA AGAACTCTTC |
| | GAGAAAATGC |
| 1601 | TcgcCGCCAA AAACTTCCAG CGCGGTATGT TCGTCGTCCG |
| | GCAAATGGAG |
| 1651 | TTGGGGCTCT TCGATATGAT GATTTACAGT GAAAGCGACG |
| | AATGGCGTCT |
| 1701 | GAAAAACTGG CAGCAGGTTT TAGACAGCGT GCGCAAAGAA |
| | GTcGCCGTCA |
| 1751 | TCCAACCGCC CGAATACAAC CGCTTCGCCA ACAGCTTCGG |
| | CCacatctTC |
| 1801 | GCcggcGGCT ATTCCGCAGG CTATTACAGC TACGCATGGG |
| | CCGAAGTCCt |
| 1851 | cAGCACCGAT GCCTACGCCG CCTTTGAAGA AAGcGACGac |
| | gtcGCCGCCA |
| 1901 | CAGGCAAACG CTTCTGGCAA GAAAtccttg ccgtcggcgg |
| | ctCCCGCAGC |
| 1951 | gcgGCGGAAT CCTTCAAAGC CTTCCGCGGA CGCGAACCGA |
| | GCATAGACGC |
| 2001 | AGTGCTGCGG CAaagcggtT TCGACAACGC gGCttgA |

This corresponds to the amino acid sequence <SEQ ID 53; ORF 128.ng>:

g128.pep

| | |
|---|---|
| 1 | MIDNALLHLG EEPRFNQIQT EDIKPAVQTA IAEARGQIAA |
| | VKAQTHTGWA |
| 51 | NTVERLTGIT ERVGRIWGVV SHLNSVVDTP ELRAVYNELM |
| | PEITVFFTEI |
| 101 | GQDIELYNRF KTIKNSPEFA TLSPAQKTKL DHDLRDFVLS |
| | GAELPPERQA |
| 151 | ELAKLQTEGA QLSAKFSQNV LDATDAFGIY FDDAAPLAGI |
| | PEDALAMFAA |
| 201 | AAQSEGKTGY KIGLQIPHYL AVIQYAGNRE LREQIYRAYV |
| | TRASELSNDG |
| 251 | KFDNTANIDR TLENALKTAK LLGFKNYAEL SLATKMADTP |
| | EQVLNFLHDL |
| 301 | ARRAKPYAEK DLAEVKAFAR EHLGLADPQP WDLSYAGEKL |
| | REAKYAFSET |
| 351 | EVKKYFPVGK VLAGLFAQIK KLYGIGFAEK TVPVWHKDVR |
| | YFELQQNGKT |
| 401 | IGGVYMDLYA REGKRGGAWM NDYKGRRRFA DGTLQLPTAY |
| | LVCNFAPPVG |
| 451 | GKEARLSHDE ILTLFHETGH GLHHLLTQVD ELGVSGINGV |
| | EWDAVELPSQ |
| 501 | FMENFVWEYN VLAQMSAHEE TGEPLPKELF DKMLAAKNFQ |
| | RGMFLVRQME |
| 551 | FALFDMMIYS ESDECRLKNW QQVLDSVRKE VAVIQPPEYN |
| | RFANSFGHIF |
| 601 | AGGYSAGYYS YAWAEVLSTD AYAAFEESDD VAATGKRFWQ |
| | EILAVGGSRS |
| 651 | AAESFKAFRG REPSIDALLR QSGFDNAA* |

ORF 128 shows 91.7% identity over a 475 aa overlap with a predicted ORF (ORF 128.ng) from N. gonorrhoeae:

| m128/g128 | |
|---|---|
| | 10 20 30 40 50 60 |
| g128.pep | MIDNALLHLGEEPRFNQIQTEDIKPAVQTAIAEARGQIAAVKAQTHTGWANTVERLTGIT |

| |||||||||||||:||:|||||||:|||||||| ||||:||||||||||||| |||||

| | |
|---|---|
| m128 | MTDNALLHLGEEPRFDQIKTEDIKPALQTAIAEAREQIAAIKAQTHTGWANTVEPLTGIT |
| | 10 20 30 40 50 60 |
| | 70 80 90 100 110 120 |
| g128.pep | ERVGRIWGVVSHLNSVVDTPELRAVYNELMPEITVFTTEIGQDIELYNRFKTIKNSPEFA |

|||||||||||||| |:||||||||||||||||||||||||||||||||||||||||||

| | |
|---|---|
| m128 | ERVGRIWGVVSHLNCVADTPELRAVYNELMPEITVFTTEIGQDIELYNRFKTIKNSPEFD |
| | 70 80 90 100 110 120 |
| | 130 140 150 160 170 180 |
| g128.pep | TLSPAQKTKLDHDLRDFVLSGAELPPERQAELAKLQTEGAQLSAKFSQNVLDATDAFGIY |

||||||||||:|

| | |
|---|---|
| m128 | TLSPAQKTKLNH |
| | 130 |
| | // |
| | 340 350 360 |
| g128.pep | YAGEKLREAKYAFSETEVKKYFPVGKVLAG |

||:||||||||||||| |||||||| || |

| | |
|---|---|
| m128 | TTYASEKLREAKYAFSETXVKKYFPVGXVLNG |
| | 10 20 30 |
| | 370 380 390 400 410 420 |
| g128.pep | LFAQIKKLYGIGFAEKTVPVWHKDVRYFELQQNGKTIGGVYMDLYAREGKRGGAWMNDYK |

|||| ||||||||:||||||||||||| ||||||::||||||||||||||||||||||||

| | |
|---|---|
| m128 | LFAQXKKLYGIGTTEKTVPVWHKDVRYXELQQNGEXIGGVYMDLYAREGKRGGAWMNDYK |
| | 40 50 60 70 80 90 |
| | 430 440 450 460 470 480 |
| g128.pep | GRRRFADGTLQLPTAYLVCNFAPPVGGKEARLSHDEILTLFHETGHGLHHLLTQVDELGV |

|||||:||||||||||||||||||||||:|||||||||| |||||||||||||||||||||

| | |
|---|---|
| m128 | GRRRFSDGTLQLPTAYLVCNFAPPVGGRiEARLSHDEILILFHETGHGLHHLLTQVDELGV |
| | 100 110 120 130 140 150 |
| | 490 500 510 520 530 540 |
| g128.pep | SGINGVEWDAVELPSQFMENFVWEYNVLAQMSAHEETGEPLPKELFDKMLAAKNFQRGMF |

|||||| ||||||||||||||||||||||| ||||||| |||||| || ||||||| |||

| | |
|---|---|
| m128 | SGINGVXWDAVELPSQFMENFVWEYNVLAQXSAHEETGVPLPKELXDKXLAAKNFQXGMF |
| | 160 170 180 190 200 210 |
| | 550 560 570 580 590 600 |
| g128.pep | LVRQMEFALFDMMIYSESDECRLKNWQQVLDSVRICEVAVIQPPEYNRFANSFGHIFAGGY |

||| |||||||||||||:|| ||||||||||||||:||||||||||||| ||||||||||

| | |
|---|---|
| m128 | XVRQXEFALFDMMIYSEDDEGRLKNWQQVLDSVRKKVAVIQPPEYNRFALSFGHIFAGGY |
| | 220 230 240 250 260 270 |
| | 610 620 630 640 650 660 |
| g128.pep | SAGYYSYAWAEVLSTDAYAAFEESDDVAATGKRFWQEILAVGGSRSAAESFKAFRGREPS |

||: ||||||||||:||||||||||||||||||||||||||| |||:|||||||||||||

| | |
|---|---|
| m128 | SAAXYSYAWAEVLSADAYAAFEESDDVAATGKRFWQEILAVGXSRSGAESFKAFRGREPS |
| | 280 290 300 310 320 330 |
| | 670 679 |
| g128.pep | IDALLRQSGFDNAAX |

||||||:||||||:

| | |
|---|---|
| m128 | IDALLRHSGFDNAVX |
| | 340 |

The following partial DNA sequence was identified in N. meningitidis <SEQ ID 54>:

| a128.seq | |
|---|---|
| 1 | ATGACTGACA ACGCACTGCT CCATTTGGGC GAAGAACCCC GTTTTGATCA |
| 51 | AATCAAAACC GAAGACATCA AACCCGCCCT GCAAACCGCC ATTGCCGAAG |
| 101 | CGCGCGAACA AATCGCCGCC ATCAAAGCCC AAACGCACAC CGGCTGGGCA |
| 151 | AACACTGTCG AACCCCTGAC CGGCATCACC GAACGCGTCG GCAGGATTTG |
| 201 | GGGCGTGGTG TCGCACCTCA ACTCCGTCAC CGACACGCCC GAACTGCGCG |
| 251 | CCGCCTACAA TGAATTAATG CCCGAAATTA CCGTCTTCTT CACCGAAATC |
| 301 | GGACAAGACA TCGAGCTGTA CAACCGCTTC AAAACCATCA AAAACTCCCC |
| 351 | CGAGTTCGAC ACCCTCTCCC ACGCGCAAAA AACCAAACTC AACCACGATC |
| 401 | TGCGCGATTT CGTCCTCAGC GGCGCGGAAC TGCCGCCCGA ACAGCAGGCA |
| 451 | GAATTGGCAA AACTGCAAAC CGAAGGCGCG CAACTTTCCG CCAAATTCTC |
| 501 | CCAAAACGTC CTAGACGCGA CCGACGCGTT CGGCATTTAC TTTGACGATG |
| 551 | CCGCACCGCT TGCCGGCATT CCCGAAGACG CGCTCGCCAT GTTTGCCGCT |
| 601 | GCCGCGCAAA GCGAAGGCAA AACAGGCTAC AAAATCGGTT TGCAGATTCC |
| 651 | GCACTACCTC GCCGTCATCC AATACGCCGA CAACCGCAAA CTGCGCGAAC |
| 701 | AAATCTACCG CGCCTACGTT ACCCGCGCCA GCGAGCTTTC AGACGACGGC |
| 751 | AAATTCGACA ACACCGCCAA CATCGACCGC ACGCTCGAAA ACGCCCTGCA |
| 801 | AACCGCCAAA CTGCTCGGCT TCAAAAACTA CGCCGAATTG TCGCTGGGAA |
| 851 | CCAAAATGGC GGACACCCCC GAACAAGTTT TAAACTTCCT GCACGACCTC |
| 901 | GCCCGCCGCG CCAAACCCTA CGCCGAAAAA GACCTCGCCG AAGTCAAAGC |
| 951 | CTTCGCCCGC GAAAGCCTCG GCCTCGCCGA TTTGCAACCG TGGGACTTGG |
| 1001 | GCTACGCCGG CGAAAAACTG CGCGAAGCCA AATACGCATT CAGCGAAACC |
| 1051 | GAAGTCAAAA AATACTTCCC CGTCGGCAAA GTATTAAACG GACTGTTCGC |
| 1101 | CCAAATCAAA AAACTCTACG GCATCGGATT TACCGAAAAA ACCGTCCCCG |
| 1151 | TCTGGCACAA AGACGTGCGC TATTTTGAAT TGCAACAAAA CGGCGAAACC |
| 1201 | ATAGGCGGCG TTTATATGGA TTTGTACGCA CGCGAAGGCA AACGCGGCGG |
| 1251 | CGCGTGGATG AACGACTACA AAGGCCGCCG CCGTTTTTCA GACGGCACGC |
| 1301 | TGCAACTGCC CAGCCCCTAC CTCGTCTGCA ACTTCACCCC GCCCGTCGGC |
| 1351 | GGCAAAGAAG CCCGCTTGAG CCATGACGAA ATCCTCACCC TCTTCCACGA |
| 1401 | AACCGGACAC GGCCTGCACC AGCTGCTTAC CCAAGTCGAC GAACTGGGCG |
| 1451 | TATCCGGCAT CAACGGCGTA GAATGGGACG CAGTCGAACT GCCCAGTCAG |
| 1501 | TTTATGGAAA ATTTCGTTTG GGAATACAAT GTCTTGGCGC AAATGTCCGC |
| 1551 | CCACGAAGAA ACCGGCGTTC CCCTGCCGAA AGAACTCTTC GACAAAATGC |
| 1601 | TCGCCGCCAA AAACTTCCAA GGCGGAATGT TCCTCGTCCG CCAAATGGAG |
| 1651 | TTCGCCCTCT TTGATATGAT GATTTACAGC GAAGACGACG AAGGCCGTCT |
| 1701 | GAAAAACTGG CAACAGGTTT TAGACAGCGT GCGCAAAGAA GTCGCCGTCG |
| 1751 | TCCGACCGCC CGAATACAAC CGCTTCGCCA AGAGCTTCGG CCACATCTTC |
| 1801 | GCAGGCGGCT ATTCCGCAGG CTATTACAGC TACGCGTGGG CGGAAGTATT |
| 1851 | GAGCGCGGAC GCATACGCCG CCTTTGAAGA AAGCGACGAT GTCGCCGCCA |
| 1901 | CAGGCAAACG CTTTTGGCAG GAAATCCTCG CCGTCGGCGG ATCGCGCAGC |
| 1951 | GCGGCAGAAT CCTTCAAAGC CTTCCGCGGA CGCGAACCGA GCATAGACGC |
| 2001 | ACTCTTGCGC CACAGCGGCT TCGACAACGC GGCTTGA |

This corresponds to the amino acid sequence <SEQ ID 55; ORF 128.a>:

| a128.pep | |
|---|---|
| 1 | MTDNALLHLG EEPRFDQIKT EDIKPALQTA IAEAREQIAA IKAQTHTGWA |
| 51 | NTVEPLTGIT ERVGRIWGVV SHLNSVTDTP ELRAAYNELM PEITVFFTEI |
| 101 | GQDIELYNRF KTIKNSPEFD THSHAQKTKL NHDLRDFVLS GAELPPEQQA |
| 151 | ELAKLQTEGA QLSAKFSQNV LDATDAFGIY FDDAAPLAGI PEDALAMFAA |
| 201 | AAQSEGKTGY KIGLQIPHYL AVIQYADNRK LREQIYRAYV TRASELSDDG |
| 251 | KFDNTANIDR TLENALQTAK LLGFKNYAEL SLATKMADTP EQVLNFLHDL |
| 301 | ARRAKPYAEK DLAEVKAFAR ESLGLADLQP WDLGYAGEKL REAKYAFSET |
| 351 | EVKKYFPVGK VLNGLFAQIK KLYGIGTTEK TVPVWHKDVR YFELQQNGET |
| 401 | IGGVYMDLYA REGKRGGAWM NDYKGRRRFS DGTLQLPTAY LVCNTTPPVG |
| 451 | GKEARLSHDE ILTLFHETGH GLHHLLTQVD ELGVSGINGV EWDAVELPSQ |
| 501 | FMENFVWEYN VLAQMSAHEE TGVPLPKELF DKMLAAKNFQ RGMFLVRQME |
| 551 | FALFDMMIYS EDDEGRLKNW QQVLDSVRKE VAVVRPPEYN RFANSFGHIF |
| 601 | AGGYSAGYYS YAWAEVLSAD AYAAFEESDD VAATGKRFWQ EILAVGGSRS |
| 651 | AAESFKAFRG REPSIDALLR HSGFDNAA* |

| m128/a128 ORFs 128 and 128.a showed a 66.0% identity in 677 | |
|---|---|
| aa overlap | |
| | 10 20 30 40 50 60 |
| m128.pep | MTDNALLHLGEEPRFDQIKTEDIKPALQTAIAEAREQIAAIKAQThTGWANTVEPLTGIT |

|||||||||||||||||||||||||||||||||||||||||||||||||||||||||||

| | |
|---|---|
| a128 | MTDNALLHLGEEPRFDQIKTEDIKPALQTAIAEAREQIAAIKAQTHTGWANTVEPLTGIT |
| | 10 20 30 40 50 60 |
| | 70 80 90 100 110 120 |
| m128.pep | ERVGRIWGVVSHLNCVADTPELRAVYNELMPEITVFTTEIGQDIELYNRFKTIKNSPEFD |

|||||||||||||| |:|||||||||||||||||||||||||||||||||||

| | |
|---|---|
| a128 | ERVGRIWGVVSHLNSVTDTPELRAAYNELMPEITVFTTEIGQDIELYNRFKTIKNSPEFD |
| | 70 80 90100 110 120 |
| | 130 |
| m128.pep | TLSPAQKTKLNH---------------------------------------------- |

||| ||||||||

| | |
|---|---|
| a128 | TLSHAQKTKLNHDLRDFVLSGAELPPEQQAELAKLQTEGAQLSAKFSQNVLDATDAFGIY |
| | 130 140 150 160 170 180 |
| m128.pep | ------------------------------------------------------------ |
| a128 | FDDAAPLAGIPEDALAMFAAAAQSEGKTGYKIGLQIPHYLAVIQYADNRKLREQIYRAYV |
| | 190 200 210 220 230 240 |
| m128.pep | ------------------------------------------------------------ |
| a128 | TRASELSDDGKFDNTANIDRTLENALQTAKLLGFKNYAELSLATKMADTPEQVLNFLHDL |
| | 250 260 270 280 290 300 |
| | 140 150 |
| m128.pep | ----------------------------------YASEKLREAKYAFSETXVKKYFPVGX |

||:||||||||||||| ||||||||

| | |
|---|---|
| a128 | ARRAKPYAEKDLAEVKAFARESLGLADLQPWDLGYAGEKLREAKYAFSETEVKKYFPVGK |
| | 310 320 330 340 350 360 |
| | 160 170 180 190 200 210 |
| 128.pep | VLNGLFAQXKKLYGIGTTEKTVPVWHKDVRYXELQQNGEXIGGVYMDLYAREGKRGGAWM |

|||||||| |||||||||||||||||||||| |||||||:||||||||||||||||||||

| | |
|---|---|
| a128 | VLNGLFAQIKKLYGIGTTEKTVPVWHKDVRYFELQQNGETIGGVYMDLYAREGKRGGAWM |
| | 370 380 390 400 410 420 |
| | 220 230 240 250 260 270 |
| m128.pep | NDYKGRRRFSDGTLQLPTAYLVCNFAPPVGGREARLSHDEILILFHETGHGLHHLLTQVD |

|||||||||||||||||||||||||:|||||:|||||||||| |||||||||||||||||

| | |
|---|---|
| a128 | NDYKGRRRFSDGTLQLPTAYLVCNTTPPVGGKEARLSHDEILTLFHETGHGLHHLLTQVD |
| | 430 440 450 460 470 480 |
| | 280 290 300 310 320 330 |
| m128.pep | ELGVSGINGVXWDAVELPSQFMENFVWEYNVLAQXSAHEETGVPLPKELXDKLXLAAKNFQ |

|||||||||| ||||||||||||||||||||||| |||||||||||||| || |||||||

| | |
|---|---|
| a128 | ELGVSGINGVEWDAVELPSQFMENFVWEYNVLAQMSAHEETGVPLPKELFDKMLAAKNFQ |
| | 490 500 510 520 530 540 |
| | 340 350 360 370 380 390 |
| m128.pep | XGMFXVRQXEFALFDMMIYSEDDEGRLKNWQQVLDSVRKKVAVIQPPEYNRFALSFGHIF |

||| ||| ||||||||||||||||||||||||||||||:|||::|||||||| ||||||

| | |
|---|---|
| a128 | RGMFLVRQMEFALFDMMIYSEDDEGRLKNWQQVLDSVRKEVAVVRPPEYNRFANSFGHIF |
| | 550 560 570 580 590 600 |
| | 400 410 420 430 440 450 |
| m128.pep | AGGYSAAXYSYAWAEVLSADAYAAFEESDDVAATGKRFWQEILAVGXSRSGAESFKAFRG |

||||||: |||||||||||||||||||||||||||||||||||||| |||:|||||||||

| | |
|---|---|
| a128 | AGGYSAGYYSYAWAEVLSADAYAAFEESDDVAATGKRFWQEILAVGGSRSAAESFKAFRG |
| | 610 620 630 640 650 660 |
| | 460 470 |
| m128.pep | REPSIDALLRHSGFDNAVX |

|||||||||||||||||:

| | |
|---|---|
| a128 | REPSIDALLRHSGFDNAALX |
| | 670 |

Further work revealed the DNA sequence identified in N. meningitidis <SEQ ID 56>:

| m128-1.seq | |
|---|---|
| 1 | ATGACTGACA ACGCACTGCT CCATTTGGGC GAAGAACCCC GTTTTGATCA |
| 51 | AATCAAAACC GAAGACATCA AACCCGCCCT GCAAACCGCC ATCGCCGAAG |
| 101 | CGCGCGAACA AATCGCCGCC ATCAAAGCCC AAACGCACAC CGGCTGGGCA |
| 151 | AACACTGTCG AACCCCTGAC CGGCATCACC GAACGCGTCG GCAGGATTTG |
| 201 | GGGCGTGGTG TCGCACCTCA ACTCCGTCGC CGACACGCCC GAACTGCGCG |
| 251 | CCGTCTATAA CGAACTGATG CCCGAAATCA CCGTCTTCTT CACCGAAATC |
| 301 | GGACAAGACA TCGAGCTGTA CAACCGCTTC AAAACCATCA AAAATTCCCC |
| 351 | CGAATTCGAC ACCCTCTCCC CCGCACAAAA AACCAAACTC AACCACGATC |
| 401 | TGCGCGATTT CGTCCTCAGC GGCGCGGAAC TGCCGCCCGA ACAGCAGGCA |
| 451 | GAACTGGCAA AACTGCAAAC CGAAGGCGCG CAACTTTCCG CCAAATTCTC |
| 501 | CCAAAACGTC CTAGACGCGA CCGACGCGTT CGGCATTTAC TTTGACGATG |
| 551 | CCGCACCGCT TGCCGGCATT CCCGAAGACG CGCTCGCCAT GTTTGCCGCC |
| 601 | GCCGCGCAAA GCGAAAGCAA AACAGGCTAC AAAATCGGCT TGCAGATTCC |
| 651 | ACACTACCTC GCCGTCATCC AATACGCCGA CAACCGCGAA CTGCGCGAAC |
| 701 | AAATCTACCG CGCCTACGTT ACCCGCGCCA GCGAACTTTC AGACGACGGC |
| 751 | AAATTCGACA ACACCGCCAA CATCGACCGC ACGCTCGCAA ACGCCCTGCA |
| 801 | AACCGCCAAA CTGCTCGGCT TCAAAAACTA CGCCGAATTG TCGCTGGCAA |
| 851 | CCAAAATGGC GGACACGCCC GAACAAGTTT TAAAGTTCCT GCACGACCTC |
| 901 | GCCCGCCGCG CCAAACCCTA CGCCGAAAAA GACCTCGCCG AAGTCAAAGC |
| 951 | CTTCGCCCGC GAAAGCCTGA ACCTCGCCGA TTTGCAACCG TGGGACTTGG |
| 1001 | GCTACGCCAG CGAAAAACTG CGCGAAGCCA AATACGCGTT CAGCGAAACC |
| 1051 | GAAGTCAAAA AATACTTCCC CGTCGGCAAA GTATTAAACG GACTGTTCGC |
| 1101 | CCAAATCAAA AAACTCTACG GCATCGGATT TACCGAAAAA ACCGTCCCCG |
| 1151 | TCTGGCACAA AGACGTGCGC TATTTTGAAT TGCAAGAAAA CGGCGAAACC |
| 1201 | ATAGGCGGCG TTTATATGGA TTTGTACGCA CGCGAAGGCA AACGCGGCGG |
| 1251 | CGCGTGGATG AACGACTACA AAGGCCGCCG CCGTTTTTCA GACGGCACGC |
| 1301 | TGCAACTGCC CACCGCCTAC CTCGTCTGCA ACTTCGCCCC ACCCGTCGGC |
| 1351 | GGCAGGGAAG CCCGCCTGAG CCACGACGAA ATCCTCATCC TCTTCCACGA |
| 1401 | AACCGGACAC GGGCTGCACC ACCTGCTTAC CCAAGTGGAC GAACTGGGCG |
| 1451 | TATCCGGCAT CAACGGCGTA GAATGGGACG CGGTCGAACT GCCCAGCCAG |
| 1501 | TTTATGGAAA ATTTCGTTTG GGAATACAAT GTCTTGGCAC AAATGTCAGC |
| 1551 | CCACGAAGAA ACCGGCGTTC CCCTGCCGAA AGAACTCTTC GACAAAATGC |
| 1601 | TCGCCGCCAA AAACTTCCAA CGCGGCATGT TCCTCGTCCG GCAAATGGAG |
| 1651 | TTCGCCCTCT TTGATATGAT GATTTACAGC GAAGACGACG AAGGCCGTCT |
| 1701 | GAAAAACTGG CAACAGGTTT TAGACAGCGT GCGCAAAAAA GTCGCCGTCA |
| 1751 | TCCAGCCGCC CGAATACAAC CGCTTCGCCT TGAGCTTCGG CCACATCTTC |
| 1801 | GCAGGCGGCT ATTCCGCAGG CTATTACAGC TACGCGTGGG CGGAAGTATT |
| 1851 | GAGCGCGGAC GCATACGCCG CCTTTGAAGA AAGCGACGAT GTCGCCGCCA |
| 1901 | CAGGCAAACG CTTTTGGCAG GAAATCCTCG CCGTCGGCGG ATCGCGCAGC |
| 1951 | GCGGCAGAAT CCTTCAAAGC CTTCCGCGGC CGCGAACCGA GCATAGACGC |
| 2001 | ACTCTTGCGC CACAGCGGTT TCGACAACGC GGTCTGA |

This corresponds to the amino acid sequence <SEQ ID 57; ORF 128-1>:

| m128-1.pep. | |
|---|---|
| 1 | MTDNALLHLG EEPRFDQIKT EDIKPALQTA IAEAREQIAA IKAQTHTGWA |
| 51 | NTVEPLTGIT ERVGRIWGVV SHLNSVADTP ELRAVYNELM PEITVFTTEI |
| 101 | GQDIELYNRF KTIKNSPEFD TLSPAQKTKL NHDLRDFVLS GAELPPEQQA |
| 151 | ELAKLQTEGA QLSAKFSQNV LDATDAFGIY FDDAAPLAGI PEDALAMFAA |
| 201 | AAQSESKTGY KIGLQIPHYL AVIQYADNRE LREQIYRAYV TRASELSDDG |
| 251 | KFDNTANIDR TLANALQTAK LLGFKNYAEL SLATKMADTP EQVLNFLHDL |
| 301 | ARRAKPYAEK DLAEVKAFAR ESLNLADLQP WDLGYASEKL REAKYAFSET |
| 351 | EVKKYFPVGK VLNGLFAQIK KLYGIGTTEK TVPVWHKDVR YFELQQNGET |
| 401 | IGGVYMDLYA REGKRGGAWM NDYKGRRRFS DGTLQLPTAY LVCNFAPPVG |
| 451 | GREARLSHDE ILILFHETGH GLHHLLTQVD ELGVSGINGV EWDAVELPSQ |
| 501 | FMENFVWEYN VLAQMSAHEE TGVPLPKELF DKMLAAKNFQ RGMFLVRQME |
| 551 | FALFDMMIYS EDDEGRLKNW QQVLDSVRKK VAVIQPPEYN RFALSFGHIF |
| 601 | AGGYSAGYYS YAWAEVLSAD AYAAFEESDD VAATGKRFWQ EILAVGGSRS |
| 651 | AAESFKAFRG REPSIDALLR HSGFDNAV* |

The following partial DNA sequence was identified in N. gonorrhoeae <SEQ ID 58>:

| g128-1.seq (partial) | |
|---|---|
| 1 | ATGATTGACA ACGCACTGCT CGACTTGGGC GAAGAACCCC GTTTTAATCA |
| 51 | AATCAAAACC GAAGACATCA AACCCGCCGT CCAAACCGCC ATCGCCGAAG |
| 101 | CGCGCGGACA AATCGCCGCC GTCAAAGCGC AAACGCACAG CGGCTGGGCG |
| 151 | AACACCGTCG AGCGTCTGAC CGGCATCACC GAACGCGTCG GCAGGATTTG |
| 201 | GGGCGTCGTG TCCCATCTCA ACTCCGTCGT CGACACGCCC GAACTGCGCG |
| 251 | CCGTCTATAA CGAACTGATG CCTGAAATCA CCGTCTTCTT CACCGAAATC |
| 301 | GGACAAGACA TCGAACTGTA CAACCGCTTC AAAACCATCA AAAATTCCCC |
| 351 | CGAATTTGCA ACGCTTTCCC CCGCACAAAA AACCAAGCTC GATCACGACC |
| 401 | TGCGCGATTT CGTATTGAGC GGCGCGGAAC TGCCGCCCGA ACGGCAGGCA |
| 451 | GAACTGGCAA AACTGCAAAC CGAAGGCGCG CAACTTTCCG CCAAATTCTC |
| 501 | CCAAAACGTC CTAGACGCGA CCGACGCGTT CGGCATTTAC TTTGACGATG |
| 551 | CCGCACCGCT TGCCGGCATT CCCGAAGACG CGCTCGCCAT GTTTGCCGCC |
| 601 | GCCGCGCAAA GCGAAGGCAA AACAGGTTAC AAAATCGGCT TGCAGATTCC |
| 651 | GCACTACCTT GCCGTTATCC AATACGCCGG CAACCGCGAA CTGCGCGAAC |
| 701 | AAATCTACCG CGCCTACGTT ACCCGTGCCA GCGAACTTTC AAACGACGGC |
| 751 | AAATTCGACA ACACCGCCAA CATCGACCGC ACGCTCGAAA ACGCATTGAA |
| 801 | AACCGCCAAA CTGCTCGGCT TTAAAAATTA CGCCGAATTG TCGCTGGCAA |
| 851 | CCAAAATGGC GGACACGCCC GAACAGGTTT TAAACTTCCT GCACGACCTC |
| 901 | GCCCGCCGCG CCAAACCCTA CGCCGAAAAA GACCTCGCCG AAGTCAAAGC |
| 951 | CTTCGCCCGC GAACACCTCG GTCTCGCCGA CCCGCAGCCG TGGGACTTGA |
| 1001 | GCTACGCCGG CGAAAAACTG CGCGAAGCCA AATACGCATT CAGCGAAACC |
| 1051 | GAAGTCAAAA AATACTTCCC CGTCGGCAAA GTTCTGGCAG GCCTGTTCGC |
| 1101 | CCAAATCAAA AAACTCTACG GCATCGGATT CGCCGAAAAA ACCGTTCCCG |
| 1151 | TCTGGCACAA AGACGTGCGC TATTTTGAAT TGCAACAAAA CGGCAAAACC |
| 1201 | ATCGGCGGCG TTTATATGGA TTTGTACGCA CGCGAAGGCA AACGCGGCGG |
| 1251 | CGCGTGGATG AACGACTACA AAGGCCGCCG CCGCTTTGCC GACGGCACGC |
| 1301 | TGCAACTGCC CACCGCCTAC CTCGTCTGCA ACTTCGCCCC GCCCGTCGGC |
| 1351 | GGCAAAGAAG CGCGTTTAAG CCACGACGAA ATCCTCACCC TCTTCCACGA |
| 1401 | AACCGGCCAC GGACTGCACC ACCTGCTTAC CCAAGTGGAC GAACTGGGCG |
| 1451 | TGTCCGGCAT CAACGGCGTA AAA |

This corresponds to the amino acid sequence <SEQ ID 59; ORF 128-1.ng>:

| g128-1.pep (partial) | |
|---|---|
| 1 | MIDNALLHLG EEPRFNQIKT EDIKPAVQTA IAEARGQIAA VKAQTHTGWA |
| 51 | NTVERLTGIT ERVGRIWGVV SHLNSVVDTP ELRAVYNELM PEITVFFTEI |
| 101 | GQDIELYNRF KTIKNSPEFA TLSPAQKTKL DHDLRDFVLS GAELPPERQA |
| 151 | ELAKLQTEGA QLSAKFSQNV LDATDAFGIY FDDAAPLAGI PEDALAMFAA |
| 201 | AAQSEGKTGY KIGLQIPHYL AVIQYAGNRE LREQIYRAYV TRASELSNDG |
| 251 | KFDNTANIDR TLENALKTAK LLGFKNYAEL SLATKMADTP EQVLNFLHDL |
| 301 | ARRAKPYAEK DLAEVKAFAR EHLGLADPQP WDLSYAGEKL REAKYAFSET |
| 351 | EVKKYFPVGK VLAGLFAQIK KLYGIGFAEK TVPVWHKDVR YFELQQNGKT |
| 401 | IGGVYMDLYA REGKRGGAWM NDYKGRRRFA DGTLQLPTAY LVCNFAPPVG |
| 451 | GKEARLSHDE ILTLFHETGH GLHHLLTQVD ELGVSGINGV K |

| m128-1/g128-1 ORFs 128-1 and 128-1.ng showed a 94.5% | |
|---|---|
| identity in 491 aa overlap | |
| | 10 20 30 40 50 60 |
| g128-1.pep | MIDNALLHLGEEPRFNQIKTEDIKPAVQTAIAEARGQIAAVKAQTHTGWANTVERITGIT |

| |||||||||||||:||||||||||:|||||||| ||||:||||||||||||| |||||

| | |
|---|---|
| m128-1 | MTDNALLHLGEEPRFDQIKTEDIKPALQTAIAEAREQIAAIKAQTHTGWANTVEPLTGIT |
| | 10 20 30 40 50 60 |
| | 70 80 90 100 110 120 |
| g128-1.pep | ERVGRIWGVVSHLNSVVDTPELRAVYNELMPEITVFFTEIGQDIELYNRIFKTIKNSPEFA |

||||||||||||||||:||||||||||||||||||||||||||||||||||||||||||

| | |
|---|---|
| m128-1 | ERVGRIWGVVSHLNSVADTPELRAVYNELMPEITVFFTEIGQDIELYNRFKTIKNSPEFD |
| | 70 80 90100 110 120 |
| | 130 140 150 160 170 180 |
| g128-1.pep | TLSPAQKTKLDHDLRDFVLSGAELPPERQAELAKLQTEGAQLSAKFSQNVLDATDAFGIY |

||||||||||:||||||||||||||||:||||||||||||||||||||||||||||||||

| | |
|---|---|
| m128-1 | TLSPAQKTKLNHDLRDFVLSGAELPPEQQAELAKLQTEGAQLSAKFSQNVLDATDAFGIY |
| | 130 140 150 160 170 180 |
| | 190 200 210 220 230 240 |
| g128-1.pep | FDDAAPLAGIPEDALAMFAAAAQSEGKTGYKIGLQIPHYLAVIQYAGNRELREQIYRAYV |

||||||||||||||||||||||||||||||:|||||||||||||||||||| |||||||||||||

| | |
|---|---|
| m128-1 | FDDAAPLAGIPEDALAMFAAAAQSESKTGYKIGLQIPHYLAVIQYADNRELREQIYRAYV |
| | 190 200 210 220 230 240 |
| | 250 260 270 280 290 300 |
| g128-1.pep | TRASELSNDGKFDNTANIDRTLENALKTAKLLGFKNYAELSLATKMADTPEQVLNFLHDL |

|||||||:|||||||||||||| |||:||||||||||||||||||||||||||||||||

| | |
|---|---|
| m128-1 | TRASELSDDGKFDNTANIDRTLANALQTAKLLGFKNYAELSLATKMADTPEQVLNFLHDL |
| | 250 260 270 280 290 300 |
| | 310 320 330 340 350 360 |
| g128-1.pep | ARRAKPYAEKDLAEVKAFAREHLGLADPQPWDLSYAGEKLREAKYAFSETEVKKYFPVGK |

||||||||||||||||||||| |:||| |||||:||:||||||||||||||||||||||||

| | |
|---|---|
| m128-1 | ARRAKPYAEKDLAEVKAFARESLNLADLQPWDLGYASEKLREAKYAFSETEVKKYFPVGK |
| | 310 320 330 340 350 360 |
| | 370 380 390 400 410 420 |
| g128-1.pep | VLAGLFAQIKKLYGIGFAEKTVPVWHKDVRYFELQQNGKTIGGVYMDLYAREGKRGGAWM |

|| ||||||||||||||:||||||||||||||||||||:|||||||||||||||||||||

| | |
|---|---|
| m128-1 | VLNGLFAQIKKLYGIGTTEKTVPVWHKDVRYFELQQNGETIGGVYMDLYAREGKRGGAWM |
| | 370 380 390 400 410 420 |
| | 430 440 450 460 470 480 |
| g128-1.pep | NDYKGRRRFADGTLQLPTAYLVCNFAPPVGGKEARLSHDEILTLFHETGHGLHHLLTQVD |

|||||||||:|||||||||||||||||||||:|||||||||| |||||||||||||||||

| | |
|---|---|
| m128-1 | NDYKGRRRFSDGTLQLPTAYLVCNFAPPVGGREARLSHDEILILFHETGHGLHHLLTQVD |
| | 430 440 450 460 470 480 |
| | 490 |
| g128-1.pep | ELGVSGINGVK |

||||||||||:

| | |
|---|---|
| m128-1 | ELGVSGINGVEWDAVELPSQFMENFVWEYNVLAQMSAHEETGVPLPKELFDKMLAAKNFQ |
| | 490 500 510 520 530 540 |

The following DNA sequence was identified in N. meningitidis <SEQ ID 60>:

| a128-1.seq | |
|---|---|
| 1 | ATGACTGACA ACGCACTGCT CCATTTGGGC GAAGAACCCC GTTTTGATCA |
| 51 | AATCAAAACC GAAGACATCA AACCCGCCCT GCAAACCGCC ATTGCCGAAG |
| 101 | CGCGCGAACA AATCGCCGCC ATCAAAGCCC AAACGCACAC CGGCTGGGCA |
| 151 | AACACTGTCG AACCCCTGAC CGGCATCACC GAACGCGTCG GCAGGATTTG |
| 201 | GGGCGTGGTG TCGCACCTCA ACTCCGTCAC CGACACGCCC GAACTGCGCG |
| 251 | CCGCCTACAA TGAATTAATG CCCGAAATTA CCGTCTTCTT CACCGAAATC |
| 301 | GGACAAGACA TCGAGCTGTA CAACCGCTTC AAAACCATCA AAAACTCCCC |
| 351 | CGAGTTCGAC ACCCTCTCCC ACGCGCAAAA AACCAAACTC AACCACGATC |
| 401 | TGCGCGATTT CGTCCTCAGC GGCGCGGAAC TGCCGCCCGA ACAGCAGGCA |
| 451 | GAATTGGCAA AACTGCAAAC CGAAGGCGCG CAACTTTCCG CCAAATTCTC |
| 501 | CCAAAACGTC CTAGACGCGA CCGACGCGTT CGGCATTTAC TTTGACGATG |
| 551 | CCGCACCGCT TGCCGGCATT CCCGAAGACG CGCTCGCCAT GTTTGCCGCT |
| 601 | GCCGCGCAAA GCGAAGGCAA AACAGGCTAC AAAATCGGTT TGCAGATTCC |
| 651 | GCACTACCTC GCCGTCATCC AATACGCCGA CAACCGCAAA CTGCGCGAAC |
| 701 | AAATCTACCG CGCCTACGTT ACCCGCGCCA GCGAGCTTTC AGACGACGGC |
| 751 | AAATTCGACA ACACCGCCAA CATCGACCGC ACGCTCGAAA ACGCCCTGCA |
| 801 | AACCGCCAAA CTGCTCGGCT TCAAAAACTA CGCCGAATTG TCGCTGGCAA |
| 851 | CCAAAATGGC GGACACCCCC GAACAAGTTT TAAACTTCCT GCACGACCTC |
| 901 | GCGCGCCGCG CCAAACCCTA CGCCGAAAAA GACCTCGCCG AAGTCAAAGC |
| 951 | CTTCGCCCGC GAAAGCCTCG GCCTGGCCGA TTTGCAACCG TGGGACTTGG |
| 1001 | GCTACGCCGG CGAAAAACTG CGCGAAGCCA AATACGCATT CAGCGAAACC |
| 1051 | GAAGTCAAAA AATACTTCCC CGTCGGCAAA GTATTAAACG GACTGTTCGC |
| 1101 | CCAAATCAAA AAACTCTACG GCATCGGATT TACCGAAAAA ACCGTCCCCG |
| 1151 | TCTGGCACAA AGACGTGCGC TATTTTGAAT TGCAACAAAA CGGCGAAACC |
| 1201 | ATAGGCGGCG TTTATATGGA TTTGTACGCA CGCGAAGGCA AACGCGGCGG |
| 1251 | CGCGTGGATG AACGACTACA AAGGCCGCCG CCGTTTTTCA GACGGCACGC |
| 1301 | TGCAACTGCC CACCGCCTAC CTCGTCTGCA ACTTCACCCC GCCCGTCGGC |
| 1351 | GGCAAAGAAG CCCGCTTGAG CCATGACGAA ATCCTCACCC TCTTCCACGA |
| 1401 | AACCGGACAC GGCCTGCACC ACCTGCTTAC CCAAGTCGAC GAACTGGGCG |
| 1451 | TATCCGGCAT CAACGGCGTA GAATGGGACG CAGTCGAACT GCCCAGTCAG |
| 1501 | TTTATGGAAA ATTTCGTTTG GGAATACAAT GTCTTGGCGC AAATGTCCGC |
| 1551 | CCACGAAGAA ACCGGCGTTC CCCTGCCGAA AGAACTCTTC GACAAAATGC |
| 1601 | TCGCCGCCAA AAACTTCCAA CGCGGAATGT TCCTCGTCCG CCAAATGGAG |
| 1651 | TTCGCCCTCT TTGATATGAT GATTTACAGC GAAGACGACG AAGGCCGTCT |
| 1701 | GAAAAACTGG CAACAGGTTT TAGACAGGGT GCGCAAAGAA GTCGCCGTCG |
| 1751 | TCCGACCGCC CGAATACAAC CGCTTCGCCA ACAGCTTCGG CCACATCTTC |
| 1801 | GCAGGCGGCT ATTCCGCAGG CTATTACAGC TACGCGTGGG CGGAAGTATT |
| 1851 | GAGCGCGGAC GCATACGCCG CCTTTGAAGA AAGCGACGAT GTCGCCGCCA |
| 1901 | CAGGCAAACG CTTTTGGCAG GAAATCGTCG CCGTCGGCGG ATCGCGCAGC |
| 1951 | GCGGCAGAAT CCTTCAAAGC CTTCCGCGGA CGCGAACCGA GCATAGACGC |
| 2001 | ACTCTTGCGC CACAGCGGCT TCGACAACGC GGCTTGA |

This corresponds to the amino acid sequence <SEQ ID 61; ORF 128-1.a>:

| a128-1.pep | |
|---|---|
| 1 | MTDNALLHLG EEPRFDQIKT EDIKPALQTA IAEAREQIAA IKAQTHTGWA |
| 51 | NTVEPLTGIT ERVGRIWGVV SHLNSVTDTP ELRAAYNELM PEITVFTTEI |
| 101 | GQDIELYNRF KTTKNSPEFD THSHAQKTKL NHDLRDFVLS GAELPPEQQA |
| 151 | ELAKLQTEGA QLSAKFSQNV LDATDAFGIY FDDAAPLAGI PEDALAMFAA |
| 201 | AAQSEGKTGY KIGLQIPHYL AVIQYADNRK LREQIYRAYV TRASELSDDG |
| 251 | KFDNTANIDR TLENALQTAK LLGFKNYAEL SLATKMADTP EQVLNFLHDL |
| 301 | ARRAKPYAEK DLAEVKAFAR ESLGLADLQP WDLGYAGEKL REAKYAFSET |
| 351 | EVKKYFPVGK VLNGLFAQIK KLYGIGTTEK TVPVWHKDVR YFELQQNGET |
| 401 | IGGVYMDLYA REGKRGGAWM NDYKGRRRFS DGTLQLPTAY LVCNTTPPVG |
| 451 | GKEARLSHDE ILTLFHETGH GLHHLLTQVD ELGVSGINGV EWDAVELPSQ |
| 501 | FMENFVWEYN VLAQMSAHEE TGVPLPKELF DKMLAAKNFQ RGMFLVRQME |
| 551 | FALFDMMIYS EDDEGRLKNW QQVLDSVRKE VAVVRPPEYN RFANSFGHIF |
| 601 | AGGYSAGYYS YAWAEVLSAD AYAAFEESDD VAATGKRFWQ EILAVGGSRS |
| 651 | AAESFKAFRG REPSIDALLR HSGFDNAA* |

| m128-1/a128-1 ORFs 128-1 and 128-1.a showed a 97.8% identity | |
|---|---|
| in 677 aa overlap | |
| | 10 20 30 40 50 60 |
| a128-1.pep | MTDNALLHLGEEPRFDQIKTEDIKPALQTAIAEAREQIAAIKAQTHTGWANTVEPLTGIT |

||||||||||||||||||||||||||||||||||||||||||||||||||||||||||||

| | |
|---|---|
| m128-1 | MTDNALLHLGEEPRFDQIKTEDIKPALQTAIAEAREQIAAIKAQTHTGWANTVEPLTGIT |
| | 10 20 30 40 50 60 |
| | 70 80 90 100 110 120 |
| a128-1.pep | ERVGRIWGVVSHLNSVTDTPELRAAYNELMPEITVFTTEIGQDIELYNRFKTTKNSPEFD |

||||||||||||||||:|||||||:|||||||||||||||||||||||||||||||||||

| | |
|---|---|
| m128-1 | ERVGRIWGVVSHLNSVADTPELRAVYNELMPEITVFTTEIGQDIELYNRFKTTKNSPEFD |
| | 70 80 90 100 110 120 |
| | 130 140150 160 170 180 |
| a128-1.pep | TLSHAQKTKLNHDLRDFVLSGAELPPEQQAELAKLQTEGAQLSAKFSQNVLDATDAFGIY |

||| ||||||||||||||||||||||||||||||||||||||||||||

| | |
|---|---|
| m128-1 | TLSPAQKTKLNHDLRDFVLSGAELPPEQQAELAKLQTEGAQLSAKFSQNVLDATDAFGIY |
| | 130 140 150 160 170 180 |
| | 190 200 210 220 230 240 |
| a128-1.pep | FDDAAPLAGIPEDALAMFAAAAQSEGKTGYKIGLQIPHYLAVIQYADNRKLREQIYRAYV |

|||||||||||||||||||||||||:|||||||||||||||||||||||:|||||||||||

| | |
|---|---|
| m128-1 | FDDAAPLAGIPEDALAMFAAAAQSESKTGYKIGLQIPHYLAVIQYADNRELREQIYRAYV |
| | 190 200 210 220 230 240 |
| | 250 260 270 280 290 300 |
| a128-1.pep | TRASELSDDGKFDNTANIDRTLENALQTAKLLGFKNYAELSLATKMADTPEQVLNFLHDL |

|||||||||||||||||||| |||||||||||||||||||||||||||||||||||||

| | |
|---|---|
| m128-1 | TRASELSDDGKFDNTANIDRTLANALQTAKLLGFKNYAELSLATKMADTPEQVLNFLHDL |
| | 250 260 270 280 290 300 |
| | 310 320 330 340 350 360 |
| a128-1.pep | ARRAKPYAEKDLAEVKAFARESLGLADLQPWDLGYAGEKLREAKYAFSETEVKKYFPVGK |

|||||||||||||||||||||||:||||||||||||:|||||||||||||||||||||||

| | |
|---|---|
| m128-1 | ARRAKPYAEKDLAEVKAFARESLNLADLQPWDLGYASEKLREAKYAFSETEVKKYFPVGK |
| | 310 320 330 340 350 360 |
| | 370 380 390 400 410 420 |
| a128-1.pep | VLNGLFAQIKKLYGIGTTEKTVPVWHKDVRYFELQQNGETTGGVYMDLYAREGKRGGAWM |

||||||||||||||||||||||||||||||||||||||||||||||||||||||||||||

| | |
|---|---|
| m128-1 | VLNGLFAQIKKLYGIGTTEKTVPVWHKDVRYFELQQNGETTGGVYMDLYAREGKRGGAWM |
| | 370 380 390 400 410 420 |
| | 430 440 450 460 470 480 |
| a128-1.pep | NDYKGRRRFSDGTLQLPTAYLVCNTTPPVGGKEARLSHDEILTLFHETGHGLHHLLTQVD |

|||||||||||||||||||||||:|||||:|||||||||| |||||||||||||||||

| | |
|---|---|
| m128-1 | NDYKGRRRFSDGTLQLPTAYLVCNFAPPVGGREARLSHDEILILFHETGHGLHHLLTQVD |
| | 430 440 450 460 470 480 |
| | 490 500 510 520 530 540 |
| a128-1.pep | ELGVSGINGVEWDAVELPSQFMENFVWEYNVLAQMSAHEETGVPLPKELFDKMLAAKNFQ |

||||||||||||||||||||||||||||||||||||||||||||||||||||||||||||

| | |
|---|---|
| m128-1 | ELGVSG1NGVEWDAVELPSQFMENFVWEYNVLAQMSAHEETGVPLPKELFDKMLAAKNFQ |
| | 490 500 510 520 530 540 |
| | 550 560 570 580 590 600 |
| a128-1.pep | RGMFLVRQMEFALFDMMIYSEDDEGRLKNWQQVLDSVRKEVAVVRPPEYNRFANSFGHIF |

|||||||||||||||||||||||||||||||||||||||:|||::|||||||| ||||||

| | |
|---|---|
| m128-1 | RGMFLVRQMEFALFDMMIYSEDDEGRLKNWQQVLDSVRXKVAVIQPPEYNRFALSFGHIF |
| | 550 560 570 580 590 600 |
| | 610 620 630 640 650 660 |
| a128-1.pep | AGGYSAGYYSYAWAEVLSADAYAAFEESDDVAATGKRFWQEILAVGGSRSAAESFKAPRG |

||||||||||||||||||||||||||||||||||||||||||||||||||||||||||||

| | |
|---|---|
| m128-1 | AGGYSAGYYSYAWAEVLSADAYAAFEESDDVAATGKRFWQEILAVGGSRSAAESFKAPRG |
| | 610 620 630 640 650 660 |
| | 670 679 |
| a128-1.pep | REPSIDALLRHSGFDNAAX |

|||||||||||||||||:

| | |
|---|---|
| m128-1 | REPSIDALLRHSGFDNAVX |
| | 670 |

The following partial DNA sequence was identified in N. meningitidis <SEQ ID 62>:

| m206.seq | |
|---|---|
| 1 | ATGTTTCCCC CCGACAAAAC CCTTTTCCTC TGTCTCAGCG CACTGCTCCT |
| 51 | CGCCTCATGC GGCACGACCT CCGGCAAACA CCGCCAACCG AAACCCAAAC |
| 101 | AGACAGTCCG GCAAATCCAA GCCGTCCGCA TCAGCCACAT CGACCGCACA |
| 151 | CAAGGCTCGC AGGAACTCAT GCTCCACAGC CTCGGACTCA TCGGCACGCG |
| 201 | CTACAAATGG GGCGGCAGCA GCACCGCAAC CGGCTTCGAT TGCAGCGGCA |
| 251 | TGATTCAATT CGTTTACAAr AACGCCCTCA ACGTCAAGCT GCCGCGCACC |
| 301 | GCCCGCGACA TGGCGGCGGC AAGCCGsAAA ATCCCCGAcA GCCGCyTCAA |
| 351 | GGCCGGCGAC CTCGTATTCT TCAACACCGG CGGCGCACAC CGCTACTCAC |
| 401 | ACGTCGGACT CTACATCGGC AACGGCGAAT TCATCCATGC CCCCAGCAGC |
| 451 | GGCAAAACCA TCAAAACCGA AAAACTCTCC ACACCGTTTT ACGCCAAAAA |
| 501 | CTACCTCGGC GCACATACTT TTTTTACAGA ATGA |

This corresponds to the amino acid sequence <SEQ ID 63; ORF 206>:

m206.pep

| | |
|---|---|
| 1 | MFPPDKTLFL CLSALLLASC GTTSGKHRQP KPKQTVRQIQ |
| | AVRISHIDRT |
| 51 | QGSQELMLHS LGLIGTPYKW GGSSTATGFD CSGMIQFVYK |
| | NALNVKLPRT |
| 101 | ARDMAAASRK IPDSRXKAGD LVFFNTGGAH RYSHVGLYIG |
| | NGEFIHAPSS |
| 151 | GKTIKTEKLS TPFYAKNYLG AHTFFTE* |

The following partial DNA sequence was identified in N. gonorrhoeae <SEQ ID 64>:

g206.seq

| | |
|---|---|
| 1 | atgttttccc ccgacaaaac ccttttcctc tgtctcggcg |
| | cactgctcct |
| 51 | cgcctcatgc ggcacgacct ccggcaaaca ccgccaaccg |
| | aaacccaaac |
| 101 | agacagtccg gcaaatccaa gccgtccgca tcagccacat |
| | cggccgcaca |
| 151 | caaggctcgc aggaactcat gctccacagc ctcggactca |
| | tcggcacgcc |
| 201 | ctacaaatgg ggcggcagca gcaccgcaac cggcttcgac |
| | tgcagcggca |
| 251 | tgattcaatt ggtttacaaa aacgccctca acgtcaagct |
| | gccgcgcacc |
| 301 | gcccgcgaca tggcggcggc aagccgcaaa atccccgaca |
| | gccgcctcaa |
| 351 | ggccggcgac atcgtattct tcaacaccgg cggcgcacac |
| | cgctactcac |
| 401 | acgtcggact ctacatcggc aacggcgaat tcatccatgc |
| | ccccggcagc |
| 451 | ggcaaaacca tcaaaaccga aaaactctcc acaccgtttt |
| | acgccaaaaa |
| 501 | ctaccttgga gcgcatacgt tttttacaga atga |

This corresponds to the amino acid sequence <SEQ ID 65; ORF 206.ng>:

g206.pep

| | |
|---|---|
| 1 | MFSPDKTLFL CLGALLLASC GTTSGKHRQP KPKQTVRQIQ |
| | AVRISHIGRT |
| 51 | QGSQELMLHS LGLIGTPYKW GGSSTATGFD CSGMIQLVYK |
| | NALNVKLPRT |
| 101 | ARDMAAASRK IPDSRLKAGD IVFFNTGGAH RYSHVGLYIG |
| | NGEFIHAPGS |
| 151 | GKTIKTEKLS TPFYAKNYLG AHTFFTE* |

ORF 206 shows 96.0% identity over a 177 aa overlap with a predicted ORF (ORF 206.ng) from N. gonorrhoeae:

| m206/g206 | |
|---|---|
| | 10 20 30 40 50 60 |
| m206.pep | MFPPDKTLFLCLSALLLASCGTTSGKHRQPKPKQTVRQIQAVRISHIDRTQGSQELMLHS |
| | \|\| \|\|\|\|\|\|\|\|\|:\|\|\|\|\|\|\|\|\|\|\|\|\|\|\|\|\|\|\|\|\|\|\|\|\|\|\|\|\|\|\|\|\|\| \|\|\|\|\|\|\|\|\|\|\|\| |
| g206 | MFSPDKTLFLCLGALLLASCGTTSGKHRQPKPKQTVRQIQAVRISHIGRTQGSQELMLHS |
| | 10 20 30 40 50 60 |
| | 70 80 90 100 110 120 |
| m206.pep | LGLIGTPYKWGGSSTATGFDCSGMIQFVYKNALNVKLPRTARDMAAASRKIPDSRXKAGD |
| | \|\|\|\|\|\|\|\|\|\|\|\|\|\|\|\|\|\|\|\|\|\|\|\|\|\|:\|\|\|\|\|\|\|\|\|\|\|\|\|\|\|\|\|\|\|\|\|\|\|\|\|\|\|\| \|\|\|\| |
| g206 | LGLIGTPYKWGGSSTATGFDCSGMIQLVYKNALNVKLPRTARDMAAASRKIPDSRLKAGD |
| | 70 80 90 100 110 120 |
| | 130 140 150 160 170 |
| m206.pep | LVFFNTGGAHRYSHVGLYIGNGEFIHAPSSGKTIKTEKLSTPFYAKNYLGAHTFFTEX |
| | :\|\|\|\|\|\|\|\|\|\|\|\|\|\|\|\|\|\|\|\|\|\|\|\|\|\|\|:\|\|\|\|\|\|\|\|\|\|\|\|\|\|\|\|\|\|\|\|\|\|\|\|\|\|\|\|\|\|\| |
| g206 | IVFFNTGGAHRYSHVGLYIGNGEFIHAPGSGKTIKTEKLSTPFYAKNYLGAHTFFTE |
| | 130 140 150 160 170 |

The following partial DNA sequence was identified in N. meningitidis <SEQ ID 66>:

a206.seq

| | |
|---|---|
| 1 | ATGTTTCCCC CCGACAAAAC CCTTTTCCTC TGTCTCAGCG |
| | CAGTGGTCCT |
| 51 | CGCCTCATGC GGCACGACCT CCGGCAAACA CCGCCAACCG |
| | AAACCCAAAC |
| 101 | AGACAGTCCG GCAAATCCAA GGCGTCCGCA TCAGGCACAT |
| | CGACCGCACA |
| 151 | CAAGGCTCGC AGGAACTCAT GGTCCACAGC CTCGGACTCA |
| | TCGGCACGCC |
| 201 | CTACAAATGG GGCGGCAGCA GCACCGCAAC CGGCTTCGAT |
| | TGCAGCGGCA |
| 251 | TGATTCAATT CGTTTACAAA AACGCCCTCA ACGTCAAGCT |
| | GCCGCGCACC |
| 301 | GGCCGCGACA TGGCGGCGGC AAGCCGCAAA ATCCGCGAGA |
| | GCCGCCTTAA |
| 351 | GGCCGGCGAC CTCGTATTCT TCAACACCGG CGGCGGACAC |
| | CGCTACTCAC |
| 401 | ACGTCGGACT CTATATCGGC AACGGCGAAT TGATCCATGC |
| | GCCCAGCAGC |
| 451 | GGCAAAACCA TCAAAACCGA AAAAGTCTCC ACACCGTTTT |
| | ACGCCAAAAA |
| 501 | CTACCTCGGC GCACATACTT TCTTTACAGA ATGA |

This corresponds to the amino acid sequence <SEQ ID 67; ORF 206.a>:

| a206.pep | | |
|---|---|---|
| | 1 | MFPPDKTLFL CLSALLLASC GTTSGKHRQP KPKQTVRQIQ |
| | | AVRISHIDRT |
| | 51 | QGSQELMLHS LGLIGTPYKW GGSSTATGFD CSGMIQFVYK |
| | | NALNVKLPRT |
| | 101 | ARDMAAASRK IPDSRLKAGD LVFFNTGGAH RYSHVGLYIG |
| | | NGEFIHAPSS |
| | 151 | GKTIKTEKLS TPFYAKNYLG AHTFFTE* |

| | |
|---|---|
| m206/a206 | ORFs 206 and 206.a showed a 99.4% identity in 177 aa overlap |
| | 10 20 30 40 50 60 |
| m206.pep | MFPPDKTLFLCLSALLLASCGTTSGKHRQPKPKQTVRQIQAVRISHIDRTQGSQELMLHS |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| a206 | MFPPDKTLFLCLSALLLASCGTTSGKHRQPKPKQTVRQIQAVRISHIDRTQGSQELMLHS |
| | 10 20 30 40 50 60 |
| | 70 80 90 100 110 120 |
| m206.pep | LGLIGTPYKWGGSSTATGFDCSGMIQFVYKNALNVKLPRTARDMAAASRKIPDSRXKAGD |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| \|\|\|\| |
| a206 | LGLIGTPYKWGGSSTATGFDCSGMIQFVYKNALNVKLPRTARDMAAASRKIPDSRLKAGD |
| | 70 80 90 100 110 120 |
| | 130 140 150 160 170 |
| m206.pep | LVFFNTGGAHRYSHVGLYIGNGEFIHAPSSGKTIKTEKLSTPFYAKNYLGAHTFFTEX |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| a206 | LVFFNTGGAHRYSHVGLYIGNGEFIHAPSSGKTIKTEKLSTPFYAKNYLGAHTFFTEX |
| | 130 140 150 160 170 |
| 287 | |

The following partial DNA sequence was identified in N. meningitidis <SEQ ID 68>:

m287.seq

| | |
|---|---|
| 1 | ATGTTTAAAC GCAGCGTAAT CGCAATGGCT TGTATTTTTG |
| | CCCTTTCAGC |
| 51 | CTGCGGGGGC GGCGGTGGCG GATCGCCCGA TGTCAAGTCG |
| | GCGGACACGC |
| 101 | TGTCAAAACC TGCCGCCCCT GTTGTTTCTG AAAAAGAGAC |
| | AGAGGCAAAG |
| 151 | GAAGATGCGC CACAGGCAGG TTCTCAAGGA CAGGGCGCGC |
| | CATCCGCACA |
| 201 | AGGCAGTCAA GATATGGCGG CGGTTTCGGA AGAAAATACA |
| | GGCAATGGCG |
| 251 | GTGCGGTAAC AGCGGATAAT CCCAAAAATG AAGACGAGGT |
| | GGCACAAAAT |
| 301 | GATATGCCGC AAAATGCCGC CGGTACAGAT AGTTCGACAC |
| | CGAATCACAC |
| 351 | CCCGGATCCG AATATGCTTG CCGGAAATAT GGAAAATCAA |
| | GCAACGGATG |
| 401 | CCGGGGAATC GTCTCAGCCG GCAAACCAAC CGGATATGGC |
| | AAATGCGGCG |
| 451 | GACGGAATGC AGGGGGACGA TCCGTCGGCA GGCGGGCAAA |
| | ATGCCGGCAA |
| 501 | TACGGCTGCC CAAGGTGCAA ATCAAGCCGG AAACAATCAA |
| | GCCGCCGGTT |
| 551 | CTTCAGATCC CATGCCCGCG TCAAACCCTG CACCTGCGAA |
| | TGGCGGTAGC |
| 601 | AATTTTGGAA GGGTTGATTT GGCTAATGGC GTTTTGATTG |
| | ACGGGCCGTC |
| 651 | GCAAAATATA ACGTTGACCC ACTGTAAAGG CGATTCTTGT |
| | AGTGGCAATA |
| 701 | ATTTCTTGGA TGAAGAAGTA CAGCTAAAAT CAGAATTTGA |
| | AAAATTAAGT |
| 751 | GATGCAGACA AAATAAGTAA TTACAAGAAA GATGGGAAGA |
| | ATGATAAATT |
| 801 | TGTCGGTTTG GTTGCCGATA GTGTGCAGAT GAAGGGAATC |
| | AATCAATATA |
| 851 | TTATCTTTTA TAAACCTAAA CCCACTTCAT TTGCGCGATT |
| | TAGGCGTTCT |
| 901 | GCACGGTCGA GGCGGTCGCT TCCGGCCGAG ATGCCGCTGA |
| | TTCCCGTCAA |
| 951 | TCAGGCGGAT ACGCTGATTG TCGATGGGGA AGCGGTCAGC |
| | CTGACGGGGC |
| 1001 | ATTCCGGGAA TATGTTCGCG CCCGAAGGGA ATTACCGGTA |
| | TCTGACTTAC |
| 1051 | GGGGCGGAAA AATTGCCCGG CGGATCGTAT GGCCTTCGTG |
| | TTCAAGGCGA |
| 1101 | ACCGGCAAAA GGCGAAATGC TTGCGGGCGC GGCCGTGTAC |
| | AACGGCGAAG |
| 1151 | TAGTGCATTT CCATACGGAA AAGGGCGGTG CGTACCCGAG |
| | GAGGGGCAGG |
| 1201 | TTTGCCGCAA AAGTCGATTT CGGCAGCAAA TCTGTGGACG |
| | GCATTATCGA |
| 1251 | CAGCGGCGAT GATTTGCATA TGGGTACGCA AAAATTCAAA |
| | GCCGCCATCG |
| 1301 | ATGGAAAGGG CTTTAAGGGG ACTTGGACGG AAAATGGCAG |
| | CGGGGATGTT |
| 1351 | TCCGGAAAGT TTTACGGCCC GGCCGGCGAG GAAGTGGCGG |
| | GAAAATACAG |
| 1401 | CTATCGCCCG ACAGATGCGG AAAAGGGCGG ATTGGGCGTG |
| | TTTGCCGGCA |
| 1451 | AAAAAGAGCA GGATTGA |

This corresponds to the amino acid sequence <SEQ ID 69; ORF 287>:

m287.pep

| | |
|---|---|
| 1 | MFKRSVIAMA CIFALSACGG GGGGSPDVKS ADTLSKPAAP |
| | VVSEKETEAK |
| 51 | EDAPQAGSQG QGAPSAQGSQ DMAAVSEENT GNGGAVTADN |
| | PKNEDEVAQN |
| 101 | DMPQNAAGTD SSTPNHTPDP NMLAGNMENQ ATDAGESSQP |
| | ANQPDMANAA |
| 151 | DGMQGDDPSA GGQNAGNTAA QGANQAGNNQ AAGSSDPIPA |
| | SNPAPANGGS |
| 201 | NFGRVDLANG VLIDGPSQNI TLTHCKGDSC SGNNFLDEEV |
| | QLKSEFEKLS |
| 251 | DADKISNYKK DGKNDKFVGL VADSVQMKGI NQYIIFYKPK |
| | PTSFARFRRS |
| 301 | ARSRRSLPAE MPLIPVNQAD TLIVDGEAVS LTGHSGNIFA |
| | PEGNYRYLTY |
| 351 | GAEKLPGGSY ALRVQGEPAK GEMLAGAAVY NGEVLHFHTE |
| | NGRPYPTRGR |
| 401 | FAAKVDFGSK SVDGIIDSGD DLHMGTQKFK AAIDGNGFKG |
| | TWTENGSGDV |
| 451 | SGKFYGPAGE EVAGKYSYRP TDAEKGGFGV FAGKKEQD* |

The following partial DNA sequence was identified in N. gonorrhoeae <SEQ ID 70>:

g287.seq

| | |
|---|---|
| 1 | atgtttaaac gcagtgtgat tgcaatggct tgtatttttc |
| | ccctttcagc |
| 51 | ctgtgggggc ggcggtggcg gatcgcccga tgtcaagtcg |
| | gcggacacgc |
| 101 | cgtcaaaacc ggccgccccc gttgttgctg aaaatgccgg |
| | ggaaggggtg |
| 151 | ctgccgaaag aaaagaaaga tgaggaggca gcgggcggtg |
| | cgccgcaagc |
| 201 | cgatacgcag gacgcaaccg ccggagaagg cagccaagat |
| | atggcggcag |
| 251 | tttcggcaga aaatacaggc aatggcggtg cggcaacaac |
| | ggacaacccc |
| 301 | aaaaatgaag acgcgggggc gcaaaatgat atgccgcaaa |
| | atgccgccga |
| 351 | atccgcaaat caaacaggga acaaccaacc cgccggttct |
| | tcagattccg |
| 401 | cccccgcgtc aaaccctgcc cctgcgaatg gcggtagcga |
| | ttttggaagg |
| 451 | acgaacgtgg gcaattctgt tgtgattgac ggaccgtcgc |
| | aaaatataac |
| 501 | gttgacccac tgtaaaggcg attcttgtaa tggtgataat |
| | ttattggatg |
| 551 | aagaagcacc gtcaaaatca gaatttgaaa aattaagtga |
| | tgaagaaaaa |
| 601 | attaagcgat ataaaaaaga cgagcaacgg gagaattttg |
| | tcggtttggt |
| 651 | tgctgacagg gtaaaaaagg atggaactaa caaatatatc |
| | atcttctata |
| 701 | cggacaaacc acctactcgt tctgcacggt cgaggaggtc |
| | gcttccggcc |
| 751 | gagattccgc tgattcccgt caatcaggcc gatacgctga |
| | ttgtggatgg |
| 801 | ggaagcggtc agcctgacgg ggcattccgg caatatcttc |
| | gcgcccgaag |
| 851 | ggaattaccg gtatctgact tacggggcgg aaaaattgcc |
| | cggcggatcg |
| 901 | tatgccctcc gtgtgcaagg cgaaccggca aaaggcgaaa |
| | tgcttgttgg |
| 951 | cacggccgtg tacaacggcg aagtgctgca tttccatatg |
| | gaaaacggcc |
| 1001 | gtccgtaccc gtccggaggc aggtttgccg caaaagtcga |
| | tttcggcagc |
| 1051 | aaatctgtgg acggcattat cgacagcggc gatgatttgc |
| | atatgggtac |
| 1101 | gcaaaaattc aaagccgcca tcgatggaaa cggctttaag |
| | gggacttgga |
| 1151 | cggaaaatgg cggcggggat gtttccggaa ggttttacgg |
| | cccggccggc |
| 1201 | gaggaagtgg cgggaaaata cagctatcgc ccgacagatg |
| | ctgaaaaggg |
| 1251 | cggattcggc gtgtttgccg gcaaaaaaga tcgggattga |

This corresponds to the amino acid sequence <SEQ ID 71; ORF 287.ng>:

| g287.pep | | |
|---|---|---|
| | 1 | MFKRSVIAMA CIFPLSACGG GGGGSPDVKS ADTPSKPAAP VVAENAGEGV |
| | 51 | LPKEKKDEEA AGGAPQADTQ DATAGEGSQD MAAVSAENTG NGGAATTDNP |
| | 101 | KNEDAGAQND MPQNAAESAN QTGNNQPAGS SDSAPASNPA PANGGSDFGR |
| | 151 | TNVGNSVVID GPSQNITLTH CKGDSCNGDN LLDEEAPSKS EFEKLSDEEK |
| | 201 | IKRYKKDEQR ENFVGLVADR VKKDGTNKYI IFYTDKPPTR SARSRRSLPA |
| | 251 | EIPLIPVNQA DTLIVDGEAV SLTGHSGNIF APEGNYRYLT YGAEKLPGGS |
| | 301 | YALRVQGEPA KGEMLVGTAV YNGEVLHFHM ENGRPYPSGG RFAAKVDFGS |
| | 351 | KSVDGIIDSG DDLHMGTQKF KAAIDGNGFK GTWTENGGGD VSGRFYGPAG |
| | 401 | EEVAGKYSYR PTDAEKGGFG VFAGKKDRD* |

| | |
|---|---|
| m287/g287 | ORFs 287 and 287.ng showed a 70.1% identity in 499 aa overlap |
| | 10 20 30 40 49 |
| m287.pep | MFKRSVIAMACIFALSACGGGGGGSPDVKSADTLSKPAAPVVSE-----------KETEA |
| | \|\|\|\|\|\|\|\|\|\|\|\|\| \|\|\|\|\|\|\|\|\|\|\|\|\|\|\|\|\|\|\| \|\|\|\|\|\|\|\|:\| \|:\|\| |
| g287 | MFKRSVIAMACIFPLSACGGGGGGSPDVKSADTPSKPAAPVVAENAGEGVLPKEKKDEEA |
| | 10 20 30 40 50 60 |
| | 50 60 70 80 90 100 109 |
| m287.pep | KEDAPQAGSQGQGAPSAQGSQDMAAVSEENTGNGGAVTADNPKNEDEVAQNDMPQNAAGT |
| | \|\|\|\| :\| \| :::\|\|\|\|\|\|\|\|\| \|\|\|\|\|\|\|\|:\|:\|\|\|\|\|\|\| \|\|\|\|\|\|\|\|\|\| |
| g287 | AGGAPQADTQD--ATAGEGSQDMAAVSAENTGNGGAATTDNPKNEDAGAQNDMPQNAA-- |
| | 70 80 90 100 110 |
| | 110 120 130 140 150 160 169 |
| m287.pep | DSSTPNHTPDPNMLAGNMENQATDAGESSQPANQPDMANAADGMQGDDPSAGGQNAGNTA |
| g287 | ------------------------------------------------------------ |
| | 170 180 190 200 210 220 229 |
| m287.pep | AQGANQAGNNQAAGSSDPIPASNPAPANGGSNFGRVDLANGVLIDGPSQNITLTHCKGDS |
| | ::\|\|\|:\|\|\|\| \|\|\|\|\| \|\|\|\|\|\|\|\|\|\|\|\|:\|\|\|::::\|:\|:\|\|\|\|\|\|\|\|\|\|\|\|\|\|\|\|\| |
| g287 | -ESANQTGNNQPAGSSDSAPASNPAPANGGSDFGRTNVGNSVVIDGPSQNITLTHCKGDS |
| | 120 130 140 150 160 170 |
| | 230 240 250 260 270 280 289 |
| m287.pep | CSGNNFLDEEVQLKSEFEKLSDADKISNYKKDGKNDKFVGLVADSVQMKGINQYIIFYKP |
| | \|:\|:\|:\|\|\|\|: \|\|\|\|\|\|\|\|\| :\|\|: \|\|\|\| : ::\|\|\|\|\|\|\| \|: \| \|:\|\|\|\| |
| g287 | CNGDNLLDEEAPSKSEFEKLSDEEKIKRYKKDEQRENFVGLVADRVKKDGTNKYIIFYTD |
| | 180 190 200 210 220 230 |
| | 290 300 310 320 330 340 349 |
| m287.pep | KPTSFARFRRSARSRRSLPAEMPLIPVNQADTLIVDGEAVSLTGHSGNIFAPEGNYRYLT |
| | \|\|: \|\|\|\|\|\|\|\|\|\|\|\|:\|\|\|\|\|\|\|\|\|\|\|\|\|\|\|\|\|\|\|\|\|\|\|\|\|\|\|\|\|\|\|\|\|\|\|\|\|\| |
| g287 | KPPT-----RSARSRRSLPAEIPLIPVNQADTLIVDGEAVSLTGHSGNIFAPEGNYRYLT |
| | 240 250 260 270 280 290 |
| | 350 360 370 380 390 400 409 |
| m287.pep | YGAEKLPGGSYALRVQGEPAKGEMLAGAAVYNGEVLHFHTENGRPYPTRGRFAAKVDFGS |
| | \|\|\|\|\|\|\|\|\|\|\|\|\|\|\|\|\|\|\|\|\|\|\|\|\|:\|:\|\|\|\|\|\|\|\|\|\|\| \|\|\|\|\|\|\|: \|\|\|\|\|\|\|\|\|\|\| |
| g287 | YGAEKLPGGSYALRVQGEPAKGEMLVGTAVYNGEVLHFHMENGRPYPSGGRFAAKVDFGS |
| | 300 310 320 330 340 350 |
| | 410 420 430 440 450 460 469 |
| m287.pep | KSVDGIIDSGDDLHMGTQKFKAAIDGNGFKGTWTENGSGDVSGKFYGPAGEEVAGKYSYR |
| | \|\|\|\|\|\|\|\|\|\|\|\|\|\|\|\|\|\|\|\|\|\|\|\|\|\|\|\|\|\|\|\|\|\|\|\|\|:\|\|\|\|\|:\|\|\|\|\|\|\|\|\|\|\|\|\|\|\|\| |
| g287 | KSVDGIIDSGDDLHMGTQKFKAAIDGNGFKGTWTENGGGDVSGRFYGPAGEEVAGKYSYR |
| | 360 370 380 390 400 410 |
| | 470 480 489 |
| m287.pep | PTDAEKGGFGVFAGKKEQDX |
| | \|\|\|\|\|\|\|\|\|\|\|\|\|\|\|\|::\|\| |
| g287 | PTDAEKGGFGVFAGKKDRDX |
| | 420 430 |

The following partial DNA sequence was identified in N. meningitidis <SEQ ID 72>:

a287.seq

| | |
|---|---|
| 1 | ATGTTTAAAC GCAGTGTGAT TGCAATGGCT TGTATTGTTG |
| | CCCTTTCAGC |
| 51 | CTGTGGGGGC GGCGGTGGCG GATCGCCCGA TGTTAAGTCG |
| | GCGGACACGC |
| 101 | TGTCAAAACC TGCCGCCCCT GTTGTTACTG AAGATGTCGG |
| | GGAAGAGGTG |
| 151 | CTGCCGAAAG AAAAGAAAGA TGAGGAGGGG GTGAGTGGTG |
| | CGCCGCAAGC |
| 201 | CGATACGCAG GACGCAACCG CCGGAAAAGG CGGTCAAGAT |
| | ATGGCGGCAG |
| 251 | TTTCGGCAGA AAATACAGGC AATGGCGGTG CGGCAACAAC |
| | GGATAATCCC |
| 301 | GAAAATAAAG ACGAGGGACC GCAAAATGAT ATGCCGCAAA |
| | ATGCCGCCGA |
| 351 | TACAGATAGT TCGAGACCGA ATCACAGCCC TGCACCGAAT |
| | ATGCCAACCA |
| 401 | GAGATATGGG AAACCAAGCA CCGGATGCCG GGGAATCGGC |
| | ACAACCGGCA |
| 451 | AACCAACCGG ATATGGCAAA TGCGGCGGAC GGAATGCAGG |
| | GGGACGATCC |
| 501 | GTCGGCAGGG GAAAATGCCG GCAATACGGC AGATCAAGCT |
| | GCAAATCAAG |
| 551 | CTGAAAACAA TCAAGTCGGC GGCTGTCAAA ATCCTGGCTG |
| | TTGAACCAAT |
| 601 | CCTAACGCCA CGAATGGCGG CAGCGATTTT GGAAGGATAA |
| | ATGTAGCTAA |
| 651 | TGGCATCAAG CTTGACAGCG GTTCGGAAAA TGTAACGTTG |
| | ACACATTGTA |
| 701 | AAGACAAAGT ATGCGATAGA GATTTCTTAG ATGAAGAAGC |
| | ACCACCAAAA |
| 751 | TGAGAATTTG AAAAATTAAG TGATGAAGAA AAAATTAATA |
| | AATATAAAAA |
| 801 | AGACGAGCAA CGAGAGAATT TTGTCGGTTT GGTTGCTGAC |
| | AGGGTAGAAA |
| 851 | AGAATGGAAC TAACAAATAT GTCATCATTT ATAAAGACAA |
| | GTCCGCTTCA |
| 901 | TCTTCATCTG CGCGATTCAG GCGTTCTGCA CGGTCGAGGC |
| | GGTCGCTTCC |
| 951 | GGCCGAGATG CCGCTGATTC CCGTCAATCA GGCGGATACG |
| | CTGATTGTCG |
| 1001 | ATGGGGAAGC GGTCAGCCTG ACGGGGGATT CCGGCAATAT |
| | CTTCGCGCCC |
| 1051 | GAAGGGAATT ACCGGTATCT GACTTACGGG GCGGAAAAAT |
| | TGTCCGGCGG |
| 1101 | ATCGTATGCC CTCAGTGTGC AAGGCGAACC GGCAAAAGGC |
| | GAAATGCTTG |
| 1151 | CGGGCACGGC GGTGTACAAC GGCGAAGTGC TGCATTTCCA |
| | TATGGAAAAC |
| 1201 | GGGCGTCCGT CCCCGTCCGG AGGCAGGTTT GCCGCAAAAG |
| | TCGATTTCGG |
| 1251 | CAGCAAATCT GTGGACGGCA TTATCGACAG CGGCGATGAT |
| | TTGCATATGG |
| 1301 | GTACGCAAAA ATTCAAAGCC GTTATCGATG GAAACGGCTT |
| | TAAGGGGACT |
| 1351 | TGGACGGAAA ATGGCGGCGG GGATGTTTCC GGAAGGTTTT |
| | ACGGCCCGGC |
| 1401 | CGGCGAAGAA GTGGCGGGAA AATACAGCTA TCGCCCGACA |
| | GATGCGGAAA |
| 1451 | AGGGCGGATT CGGCGTGTTT GCCGGCAAAA AAGAGCAGGA |
| | TTGA |

This corresponds to the amino acid sequence <SEQ ID 73; ORF 287.a>:

| a287.pep | | |
|---|---|---|
| | 1 | MFKRSVIAMA CIVALSACGG GGGGSPDVKS ADTLSKPAAP VVTEDVGEEV |
| | 51 | LPKEKKDEEA VSGAPQADTQ DATAGKGGQD MAAVSAENTG NGGAATTDNP |
| | 101 | ENKDEGPQND MPQNAADTDS STPNHTPAPN MPTRDMGNQA PDAGESAQPA |
| | 151 | NQPDMANAAD GMQGDDPSAG ENAGNTADQA ANQAENNQVG GSQNPASSTN |
| | 201 | PNATNGGSDF GRINVANGIK LDSGSENVTL THCKDKVCDR DFLDEEAPPK |
| | 251 | SEFEKLSDEE KINKYKKDEQ RENFVGLVAD RVEKNGTNKY VIIYKDKSAS |
| | 301 | SSSARFRRSA RSRRSLPAEM PLIPVNQADT LIVDGEAVSL TGHSGNIFAP |
| | 351 | EGNYRYLTYG AEKLSGGSYA LSVQGEPAKG EMLAGTAVYN GEVLHFHMEN |
| | 401 | GRPSPSGGRF AAKVDFGSKS VDGIIDSGDD LHMGTQKFKA VIDGNGFKGT |
| | 451 | WTENGGGDVS GRFYGPAGEE VAGKYSYRPT DAEKGGFGVF AGKKEQD* |

| | |
|---|---|
| m287/a287 | ORFs 287 and 287.a showed a 77.2% identity in 501 aa overlap |
| | 10 20 30 40 49 |
| m287.pep | MFKRSVIAMACIFALSACGGGGGGSPDVKSADTLSKPAAPVVSE-----------KETEA |
| | \|\|\|\|\|\|\|\|\|\|\|\| \|\|\|\|\|\|\|\|\|\|\|\|\|\|\|\|\|\|\|\|\|\|\|\|\|\|\|\|\|:\| \|: \|\| |
| a287 | MFKRSVIAMACIVALSACGGGGGGSPDVKSADTLSKPAAPVVTEDVGEEVLPKEKKDEEA |
| | 10 20 30 40 50 60 |
| | 50 60 70 80 90 100 109 |
| m287.pep | KEDAPQAGSQGQGAPSAQGSQDMAAVSEENTGNGGAVTADNPKNEDEVAQNDMPQNAAGT |
| | \|\|\|\| :\| \|:::\|:\|\|\|\|\|\|\| \|\|\|\|\|\|\|\|:\|:\|\|\|:\|:\|\| \|\|\|\|\|\|\|\|\| \| |
| a287 | VSGAPQADTQ--DATAGKGGQDMAAVSAENTGNGGAATTDNPENKDEGPQNDMPQNAADT |
| | 70 80 90 100 110 |
| | 110 120 130 140 150 160 169 |
| m287.pep | DSSTPNHTPDPNMLAGNMENQATDAGESSQPANQPDMANAADGMQGDDPSAGGQNAGNTA |
| | \|\|\|\|\|\|\|\|\| \|\|\| : : \|\|\| \|\|\|\|\|:\|\|\|\|\|\|\|\|\|\|\|\|\|\|\|\|\|\|\|\|\|\|\| :\|\|\|\|\|\| |
| a287 | DSSTPNHTPAPNMPTRDMGNQAPDAGESAQPANQPDMANAADGMQGDDPSAG-ENAGNTA |
| | 120 130 140 150 160 170 |
| | 170 180 190 200 210 220 229 |
| m287.pep | AQGANQAGNNQAAGSSDPIPASNIPAPANGGSNFGRVDLANGVLIDGPSQNITLTHCKGDS |
| | \|:\|\|\|\| \|\|\|::\|\|::\| ::\|\| :\|\|\|\|:\|\|\|:::\|\|\|: :\|: \|:\|:\|\|\|\|\|\| |
| a287 | DQAANQAENNQVGGSQNPASSTNPNATNGGSDFGRINVANGIKLDSGSENVTLTHCKDKV |
| | 180 190 200 210 220 230 |
| | 230 240 250 260 270 280 289 |
| m287.pep | CSGNNFLDEEVQLKSEFEKLSDADKISNYKKDGKNDKFVGLVADSVQMKGINQYIIFYKP |
| | \|: :\|\|\|\|\|: \|\|\|\|\|\|\|\|\| :\|\|::\|\|\|\| : ::\|\|\|\|\|\|\| \|: :\| \|:\|:\|:\|\| |
| a287 | CD-RDFLDEEAPPKSEFEKLSDEEKINKYKKDEQRENFVGLVADRVEKNGTNKYVIIYKD |
| | 240 250 260 270 280 290 |
| | 290 300 310 320 330 340 |
| m287.pep | KLP--TSFARFRRSARSRRSLPAEMPLIPVNQADTLIVDGEAVSLTGHSGNIFAPEGNYRY |
| | \| :\| \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| a287 | KSASSSSARFRRSARSRRSLPAEMPLIPVNQADTLIVDGEAVSLTGHSGNIFAPEGNYRY |
| | 300 310 320 330 340 350 |
| | 350 360 370 380 390 400 |
| m287.pep | LTYGAEKLPGGSYALRVQGEPAKGEMLAGAAVYNGEVLHFHTENGRPYPTRGRFAAKVDF |
| | \|\|\|\|\|\|\|\| \|\|\|\|\|\| \|\|\|\|\|\|\|\|\|\|\|\|:\|\|\|\|\|\|\|\|\|\|\| \|\|\|\|\| \|: \|\|\|\|\|\|\|\|\|\| |
| a287 | LTYGAEKLSGGSYALSVQGEPAKGEMLAGTAVYNGEVLHFHMENGRPSPSGGRFAAKVDF |
| | 360 370 380 390 400 410 |
| | 410 420 430 440 450 460 |
| m287.pep | GSKSVDGIIDSGDDLHMGTQKFKAAIDGNGFKGTWTENGSGDVSGKFYGPAGEEVAGKYS |
| | \|\|\|\|\|\|\|\|\|\|\|\|\|\|\|\|\|\|\|\|\|\|\|:\|\|\|\|\|\|\|\|\|\|\|\|\|\|:\|\|\|\|\|:\|\|\|\|\|\|\|\|\|\|\|\|\|\|\| |
| a287 | GSKSVDGIIDSGDDLHMGTQKFKAVIDGNGFKGTWTENGGGDVSGRFYGPAGEEVAGKYS |
| | 420 430 440 450 460 470 |
| | 470 480 489 |
| m287.pep | YRPTDAEKGGFGVFAGKKEQDX |
| | \|\|\|\|\|\|\|\|\|\|\|\|\|\|\|\|\|\|\|\|\|\| |
| a287 | YRPTDAEKGGFGVFAGKKEQDX |
| | 480 490 |
| 406 | |

The following partial DNA sequence was identified in N. meningitidis <SEQ ID 74>:

m406.seq

| | |
|---|---|
| 1 | ATGCAAGCAC GGCTGCTGAT ACCTATTCTT TTTTCAGTTT |
| | TTATTTTATC |
| 51 | CGCCTGCGGG ACACTGACAG GTATTCCATC GCATGGCGGA |
| | GGTAAACGCT |
| 101 | TTGCGGTCGA ACAAGAACTT GTGGCCGCTT CTGCCAGAGC |
| | TGCCGTTAAA |
| 151 | GACATGGATT TACAGGCATT ACACGGACGA AAAGTTGCAT |
| | TGTACATTGC |
| 201 | CACTATGGGC GACCAAGGTT CAGGCAGTTT GACAGGGGGT |
| | CGCTACTCCA |
| 251 | TTGATGCACT GATTCGTGGC GAATACATAA ACAGCCCTGC |
| | CGTCCGTACC |
| 301 | GATTACACCT ATCCACGTTA CGAAACCACC GCTGAAACAA |
| | CATCAGGCGG |
| 351 | TTTGACAGGT TTAACCACTT CTTTATCTAC ACTTAATGCC |
| | CCTGCACTCT |
| 401 | CTCGCACCCA ATCAGACGGT AGCGGAAGTA AAAGCAGTCT |
| | GGGCTTAAAT |
| 451 | ATTGGCGGGA TGGGGGATTA TCGAAATGAA ACCTTGACGA |
| | CTAACCCGCG |
| 501 | CGACACTGCC TTTCTTTCCC ACTTGGTACA GACCGTATTT |
| | TTCCTGCGCG |
| 551 | GCATAGACGT TGTTTCTCCT GCCAATGCCG ATACAGATGT |
| | GTTTATTAAC |
| 601 | ATCGACGTAT TCGGAACGAT ACGCAACAGA ACCGAAATGC |
| | ACCTATACAA |
| 651 | TGCCGAAACA CTGAAAGCCC AAACAAAACT GGAATATTTC |
| | GCAGTAGACA |
| 701 | GAACCAATAA AAAATTGCTC ATCAAACCAA AAACCAATGC |
| | GTTTGAAGCT |
| 751 | GCCTATAAAG AAAATTACGC ATTGTGGATG GGGCCGTATA |
| | AAGTAAGCAA |
| 801 | AGGAATTAAA CCGACGGAAG GATTAATGGT CGATTTCTCC |
| | GATATCCGAC |
| 851 | CATACGGCAA TCATACGGGT AACTCCGCCC CATCCGTAGA |
| | GGCTGATAAC |
| 901 | AGTCATGAGG GGTATGGATA CAGCGATGAA GTAGTGCGAC |
| | AACATAGACA |
| 951 | AGGACAACCT TGA |

This corresponds to the amino acid sequence <SEQ ID 75; ORF 406>:

m406.pep

| | |
|---|---|
| 1 | MQARLLIPIL FSVFILSACG TLTGIPSHGG GKRFAVEQEL |
| | VAASARAAVK |
| 51 | DMDLQALHGR KVALYIATMG DQGSGSLTGG RYSIDALIRG |
| | EYINSPAVRT |
| 101 | DYTYPRYETT AETTSGGLTG LTTSLSTLNA PALSRTQSDG |
| | SGSKSSLGLN |
| 151 | IGGMGDYRNE TLTTNPRDTA FLSHLVQTVF FLRGIDVVSP |
| | ANADTDVFIN |
| 201 | IDVFGTIRNR TEMHLYNAET LKAQTKLEYF AVDRTNKKLL |
| | IKPKTNAFEA |
| 251 | AYKENYALWM GPYKVSKGIK PTEGLMVDFS DIRPYGNHTG |
| | NSAPSVEADN |
| 301 | SHEGYGYSDE VVRQHRQGQP * |

The following partial DNA sequence was identified in N. gonorrhoeae <SEQ ID 76>:

g406.seq

| | |
|---|---|
| 1 | ATGCGGGCAC GGCTGCTGAT ACCTATTCTT TTTTCAGTTT |
| | TTATTTTATC |
| 51 | CGCCTGCGGG ACACTGACAG GTATTCCATC GCATGGCGGA |
| | GGCAAACGCT |
| 101 | TCGCGGTCGA ACAAGAACTT GTGGCCGCTT CTGCCAGAGC |
| | TGCCGTTAAA |
| 151 | GACATGGATT TACAGGCATT ACACGGACGA AAAGTTGCAT |
| | TGTACATTGC |
| 201 | AACTATGGGC GACCAAGGTT CAGGCAGTTT GACAGGGGGT |
| | CGCTACTCCA |
| 251 | TTGATGCACT GATTCGCGGC GAATACATAA ACAGCCCTGC |
| | CGTCCGCACC |
| 301 | GATTACACCT ATCCGCGTTA CGAAACCACC GCTGAAACAA |
| | CATCAGGCGG |
| 351 | TTTGACGGGT TTAACCACTT CTTTATCTAC ACTTAATGCC |
| | CCTGCACTCT |
| 401 | CGCGCACCCA ATCAGACGGT AGCGGAAGTA GGAGCAGTCT |
| | GGGCTTAAAT |
| 451 | ATTGGCGGGA TGGGGGATTA TCGAAATGAA ACCTTGACGA |
| | CCAACCCGCG |
| 501 | CGACACTGCC TTTCTTTCCC ACTTGGTGCA GACCGTATTT |
| | TTCCTGCGCG |
| 551 | GCATAGACGT TGTTTCTCCT GCCAATGCCG ATACAGATGT |
| | GTTTATTAAC |
| 601 | ATCGACGTAT TCGGAACGAT ACGCAACAGA ACCGAAATGC |
| | ACCTATACAA |
| 651 | TGCCGAAACA CTGAAAGCCC AAACAAAACT GGAATATTTC |
| | GCAGTAGACA |
| 701 | GAACCAATAA AAAATTGCTC ATCAAACCCA AAACCAATGC |
| | GTTTGAAGCT |
| 751 | GCCTATAAAG AAAATTACGC ATTGTGGATG GGGCCGTATA |
| | AAGTAAGCAA |
| 801 | AGGAATCAAA CCGACGGAAG GATTGATGGT CGATTTCTCC |
| | GATATCCAAC |
| 851 | CATACGGCAA TCATACGGGT AACTCCGCCC CATCCGTAGA |
| | GGCTGATAAC |
| 901 | AGTCATGAGG GGTATGGATA CAGCGATGAA GCAGTGCGAC |
| | AACATAGACA |
| 951 | AGGGCAACCT TGA |

This corresponds to the amino acid sequence <SEQ ID 77; ORF 406.ng>:

g406.pep

| | |
|---|---|
| 1 | MRARLLIPIL FSVFILSACG TLTGIPSHGG GKRFAVEQEL |
| | VAASARAAVK |
| 51 | DMDLQALHGR KVALYIATMG DQGSGSLTGG RYSIDALIRG |
| | EYINSPAVRT |
| 101 | DYTYPRYETT AETTSGGLTG LTTSLSTLNA PALSRTQSDG |
| | SGSRSSLGLN |
| 151 | IGGMGDYRNE TLTTNPRDTA FLSHLVQTVF FLRGIDVVSP |
| | ANADTDVFIN |
| 201 | IDVFGTIRNR TEMHLYNAET LKAQTKLEYF AVDRTNKKLL |
| | IKPKTNAFEA |
| 251 | AYKENYALWM GPYDVSKGIK PTEGLMVDFS DIQPYGNHTG |
| | NSAPSVEADN |
| 301 | SHEGYGYSDE AVRQHRQGQP * |

ORF 406.ng shows 98.8% identity over a 320 aa overlap with a predicted ORF (ORF406.a) from N. gonorrhoeae:

| g406/m406 | |
|---|---|
| | 10 20 30 40 50 60 |
| g406.pep | MRARLLIPILFSVFILSACGTLTGIPSHGGGKRFAVEQELVAASARAAVKDMDLQALHGR |
| | \|:\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m406 | MQARLLIPILFSVFILSACGTLTGIPSHGGGKRFAVEQELVAASARAAVKDMDLQALHGR |
| | 10 20 30 40 50 60 |
| | 70 80 90 100 110 120 |
| g406.pep | KVALYIATMGDQGSGSLTGGRYSIDALIRGEYINSPAVRTDYTYPRYETTAETTSGGLTG |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m406 | KVALYIATMGDQGSGSLTGGRYSIDALIRGEYINSPAVRTDYTYPRYETTAETTSGGLTG |
| | 70 80 90 100 110 120 |
| | 130 140 150 160 170 180 |
| g406.pep | LTTSLSTLNAPALSRTQSDGSGSRSSLGLNIGGMGDYRNETLTTNPRDTAFLSHLVQTVF |
| | \|\|\|\|\|\|\|\|\|\|\|\|\|\|\|\|\|\|\|\|\|\|\|:\|\|\|\|\|\|\|\|\|\|\|\|\|\|\|\|\|\|\|\|\|\|\|\|\|\|\|\|\|\|\|\|\|\|\|\| |
| m406 | LTTSLSTLNAPALSRTQSDGSGSKSSLGLNIGGMGDYRNETLTTNPRDTAFLSHLVQTVF |
| | 130 140 150 160 170 180 |
| | 190 200 210 220 230 240 |
| g406.pep | FLRGIDVVSPANADTDVFINIDVFGTIRNRTEMHLYNAETLKAQTKLEYFAVDRTNKKLL |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| m406 | FLRGIDVVSPANADTDVFINTDVFGTIRNRTEMHLYNAETLKAQTKLEYFAVDRTNKKLL |
| | 190 200 210 220 230 240 |
| | 250 260 270 280 290 300 |
| g406.pep | IKPKTNAFEAAYKENYALWMGPYKVSKGIKPTEGLMVDFSDIQPYGNHTGNSAPSVEADN |
| | \|\|\|\|\|\|\|\|\|\|\|\|\|\|\|\|\|\|\|\|\|\|\|\|\|\|\|\|\|\|\|\|\|\|\|\|\|\|\|\|\|\|:\|\|\|\|\|\|\|\|\|\|\|\|\|\|\|\|\| |
| m406 | IKPKTNAFEAAYKENYALWMGPYKVSKGIKPTEGLMVDFSDIRPYGNHTGNSAPSVEADN |
| | 250 260 270 280 290 300 |
| | 310 320 |
| g406.pep | SHEGYGYSDEAVRQHRQGQPX |
| | \|\|\|\|\|\|\|\|\|\|:\|\|\|\|\|\|\|\|\|\| |
| m406 | SHEGYGYSDEVVRQHRQGQPX |
| | 310 320 |

The following partial DNA sequence was identified in N. meningitidis <SEQ ID 78>:

a406.seq

| | |
|---|---|
| 1 | ATGCAAGCAC GGCTGCTGAT ACCTATTCTT TTTTCAGTTT |
| | TTATTTTATC |
| 51 | CGCCTGCGGG ACACTGACAG GTATTCCATC GCATGGCGGA |
| | GGTAAACGCT |
| 101 | TCGCGGTCGA ACAAGAACTT GTGGCCGCTT CTGCCAGAGC |
| | TGCCGTTAAA |
| 151 | GACATGGATT TACAGGCATT ACACGGACGA AAAGTTGCAT |
| | TGTACATTGC |
| 201 | AACTATGGGC GACCAAGGTT CAGGCAGTTT GACAGGGGGT |
| | CGCTACTCCA |
| 251 | TTGATGCACT GATTCGTGGC GAATACATAA ACAGCCCTGC |
| | CGTCCGTACC |
| 301 | GATTACACCT ATCCACGTTA CGAAACCACC GCTGAAACAA |
| | CATCAGGCGG |
| 351 | TTTGACAGGT TTAACCACTT CTTTATCTAC ACTTAATGCC |
| | CCTGCACTCT |
| 401 | CGCGCACCCA ATCAGACGGT AGCGGAAGTA AAAGCAGTCT |
| | GGGCTTAAAT |
| 451 | ATTGGCGGGA TGGGGGATTA TCGAAATGAA ACCTTGACGA |
| | CTAACCCGCG |
| 501 | CGACACTGCC TTTCTTTCCC ACTTGGTACA GACCGTATTT |
| | TTCCTGCGCG |
| 551 | GCATAGACGT TGTTTCTCCT GCCAATGCCG ATACGGATGT |
| | GTTTATTAAC |
| 601 | ATCGACGTAT TCGGAACGAT ACGCAACAGA ACCGAAATGC |
| | ACGTATACAA |
| 651 | TGCCGAAACA CTGAAAGCCC AAACAAAACT GGAATATTTC |
| | GCAGTAGACA |
| 701 | GAACCAATAA AAAATTGCTC ATCAAACCAA AAACCAATGC |
| | GTTTGAAGCT |
| 751 | GCCTATAAAG AAAATTACGC ATTGTGGATG GGACCGTATA |
| | AAGTAAGCAA |
| 801 | AGGAATTAAA GCGACAGAAG GATTAATGGT CGATTTCTCC |
| | GATATCCAAC |
| 851 | CATACGGGAA TCATATGGGT AACTCTGCCC GATCCGTAGA |
| | GGCTGATAAC |
| 901 | AGTCATGAGG GGTATGGATA CAGCGATGAA GCAGTGCGAC |
| | GACATAGACA |
| 951 | AGGGCAACCT TGA |

This corresponds to the amino acid sequence <SEQ ID 79; ORF 406.a>:

| a406.pep | | |
|---|---|---|
| | 1 | MQARLLIPIL FSVFILSACG TLTGIPSHGG GKRFAVEQEL VAASARAAVK |
| | 51 | DMDLQALHGR KVALYIATMG DQGSGSLTGG RYSIDALIRG EYINSPAVRT |
| | 101 | DYTYPRYETT AETTSGGLTG LTTSLSTLNA PALSRTQSDG SGSKSSLGLN |
| | 151 | IGGMGDYRNE TLTTNPRDTA FLSHLVQTVF FLRGIDVVSP ANADTDVFIN |
| | 201 | IDVFGTIRNR TEMHLYNAET LKAQTKLEYF AVDRTNKKLL IKPKTNAFEA |
| | 251 | AYKENYALWM GPYKVSKGIK PTEGLMVDFS DIQPYGNHMG NSAPSVEADN |
| | 301 | SHEGYGYSDE AVRRHRQGQP * |

| | |
|---|---|
| m406/a406 | ORFs 406 and 406.a showed a 98.8% identity in 320 aa overlap |
| | 10 20 30 40 50 60 |
| m406.pep | MQARLLIPILFSVFILSACGTLTGIPSHGGGKRFAVEQELVAASARAAVKDMDLQALHGR |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| a406 | MQARLLIPILFSVFILSAGGTLTGIPSHGGGKRFAVEQELVAASARAAVKDMDLQALHGR |
| | 10 20 30 40 50 60 |
| | 70 80 90 100 110 120 |
| m406.pep | KVALYIATMGDQGSGSLTGGRYSIDALIRGEYINSPAVRTDYTYPRYETTAETTSGGLTG |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| a406 | KVALYIATMGDQGSGSLTGGRYSIDALIRGEYINSPAVRTDYTYPRYETTAETTSGGLTG |
| | 70 80 90 100 110 120 |
| | 130 140 150 160 170 180 |
| m406.pep | LTTSLSTLNAPALSRTQSDGSGSKSSLGLNIGGMGDYRNETLTTNPRDTAFLSHLVQTVF |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| a406 | LTTSLSTLNAPALSRTQSDGSGSKSSLGLNIGGMGDYRNETLTTNPRDTAFLSHLVQTVF |
| | 130 140 150 160 170 180 |
| | 190 200 210 220 230 240 |
| m406.pep | FLRGIDVVSPANADTDVFINIDVFGTIRNRTEMHLYNAETLKAQTKLEYFAVDRTNKKLL |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| |
| a406 | FLRGIDVVSPANADTDVFINIDVFGTIRNRTEMHLYNAETLKAQTKLEYFAVDRTNKKLL |
| | 190 200 210 220 230 240 |
| | 250 260 270 280 290 300 |
| m406.pep | IKPKTNAFEAAYKENYALWMGPYKVSKGIKPTEGLMVDFSDIRPYGNHTGNSAPSVEADN |
| | \|\|\|\|\|\|\|\|\|\|\|\|\|\|\|\|\|\|\|\|\|\|\|\|\|\|\|\|\|\|\|\|\|\|\|\|\|\|\|\|\|\|:\|\|\|\|\| \|\|\|\|\|\|\|\|\|\|\| |
| a406 | IKLPKTNAFEAAYKENYALWMGPYKVSKGIKPTEGLMVDFSDIQPYGNHMGNSAPSVEAN |
| | 250 260 270 280 290 300 |
| | 310 320 |
| m406.pep | SHEGYGYSDEVVRQHRQGQPX |
| | \|\|\|\|\|\|\|\|\|\|:\|\|:\|\|\|\|\|\|\| |
| a406 | SHEGYGYSDEAVRRHRQGQPX |
| | 310 320 |

The following partial DNA sequence was identified in N. meningitidis <SEQ ID 80>:

m726.seq

| | |
|---|---|
| 1 | ATGACCATCT ATTTCAAAAA CGGCTTTTAC GAGGACACAT |
| | TGGGCGGCAT |
| 51 | CCCCGAAGGC GCGGTTGCCG TCCGCGCCGA AGAATACGCC |
| | GCCCTTTTGG |
| 101 | CAGGACAGGC GCAGGGCGGG CAGATTGCCG CAGATTCCGA |
| | CGGCCGCCCC |
| 151 | GTTTTAACCC CGCCGCGCCC GTCCGATTAC CACGAATGGG |
| | ACGGCAAAAA |
| 201 | ATGGAAAATC AGCAAAGCCG CCGCCGCCGC CCGTTTCGCC |
| | AAACAAAAAA |
| 251 | CCGCCTTGGC ATTCCGCCTC GCGGAAAAGG CGGACGAACT |
| | CAAAAACAGC |
| 301 | CTCTTGGCGG GCTATCCCCA AGTGGAAATC GACAGCTTTT |
| | ACAGGCAGGA |
| 351 | AAAAGAAGCC CTCGCGCGGC AGGCGGACAA CAACGCCCCG |
| | ACCCCGATGC |
| 401 | TGGCGCAAAT CGCCGCCGCA AGGGGCGTGG AATTGGACGT |
| | TTTGATTGAA |
| 451 | AAAGTTATCG AAAAATCCGC CCGCCTGGCT GTTGCCGCCG |
| | GCGCGATTAT |
| 501 | CGGAAAGCGT CAGCAGCTCG AAGACAAATT GAACACCATC |
| | GAAACCGCGC |
| 551 | CCGGATTGGA CGCGCTGGAA AAGGAAATCG AAGAATGGAC |
| | GCTAAACATC |
| 601 | GGCTGA |

This corresponds to the amino acid sequence <SEQ ID 81; ORF 726>:

m726.pep

| | |
|---|---|
| 1 | MTIYFKNGFY DDTLGGIPEG AVAVRAEEYA ALLAGQAQGG |
| | QIAADSDGRP |
| 51 | VLTPPRPSDY HEWDGKKWKI SKAAAAARFA KQKTALAFRL |
| | AEKADELKNS |
| 101 | LLAGYPQVEI DSFYRQEKEA LARQADNNAP TPMLAQIAAA |
| | RGVELDVLIE |
| 151 | KVIEKSARLA VAAGAIIGKR QQLEDKLNTI ETAPGLDALE |
| | KEIEEWTLNI |
| 201 | G* |

The following partial DNA sequence was identified in N. meningitidis <SEQ ID 82>:

m907-2.seq

| | |
|---|---|
| 1 | ATGAGAAAAC CGACCGATAC CCTACCCGTT AATCTGCAAC |
| | GCCGCCGCCT |
| 51 | GTTGTGTGCC GCCGGTGCGT TGTTGCTCAG TCCTCTGGCG |
| | CACGCCGGCG |
| 101 | CGCAACGTGA GGAAACGCTT GCCGACGATG TGGCTTCCGT |
| | GATGAGGAGT |
| 151 | TCTGTCGGCA GCGTCAATCC GCCGAGGCTG GTGTTTGACA |
| | ATCCGAAAGA |
| 201 | GGGCGAGCGT TGGTTGTCTG CCATGTCGGC ACGTTTGGCA |
| | AGGTTCGTCC |
| 251 | CCGAGGAGGA GGAGCGGCGC AGGCTGCTGG TCAATATCCA |
| | GTACGAAAGC |
| 301 | AGCCGGGCCG GTTTGGATAC GCAGATTGTG TTGGGGCTGA |
| | TTGAGGTGGA |
| 351 | AAGCGCGTTC CGCCAGTATG CAATCAGCGG TGTCGGCGCG |
| | CGCGGCCTGA |
| 401 | TGCAGGTTAT GCCGTTTTGG AAAAACTACA TCGGCAAACC |
| | GGCGCACAAC |
| 451 | CTGTTCGACA TCCGCACCAA CCTGCGTTAC GGCTGTACCA |
| | TCCTGCGCCA |
| 501 | TTACCGGAAT CTTGAAAAAG GCAACATCGT CCGCGCGCTT |
| | GCCCGCTTTA |
| 551 | ACGGCAGCTT GGGCAGCAAT AAATATCCGA ACGCCGTTTT |
| | GGGCGCGTGG |
| 601 | CGCAACCGCT GGCAGTGGCG TTGA |

This corresponds to the amino acid sequence <SEQ ID 83; ORF 907-2>:

m907-2.pep

| | |
|---|---|
| 1 | MRKPTDTLPV NLQRRRLLCA AGALLLSPLA HAGAQREETL |
| | ADDVASVMRS |
| 51 | SVGSVNPPRL VFDNPKEGER WLSAMSARLA RFVPEEEERR |
| | RLLVNIQYES |
| 101 | SRAGLDTQIV LGLIEVESAF RQYAISGVGA RGLMQVMPFW |
| | KNYIGKPAHN |
| 151 | LFDIRTNLRY GCTILRHYRN LEKGNIVRAL ARFNGSLGSN |
| | KYPNAVLGAW |
| 201 | RNRWQWR* |

The following partial DNA sequence was identified in N. meningitidis <SEQ ID 84>:

m953.seq

| | |
|---|---|
| 1 | ATGAAAAAAA TCATCTTCGC CGCACTCGCA GCCGCCGCCA |
| | TCAGTACTGC |
| 51 | CTCCGCCGCC ACCTACAAAG TGGACGAATA TCACGCCAAC |
| | GCCCGTTTCG |
| 101 | CCATCGACCA TTTCAACACC AGCACCAACG TCGGCGGTTT |
| | TTACGGTCTG |
| 151 | ACCGGTTCCG TCGAGTTCGA CCAAGCAAAA CGCGACGGTA |
| | AAATCGACAT |
| 201 | CACCATCCCC ATTGCCAACC TGCAAAGCGG TTCGCAACAC |
| | TTTACCGACC |
| 251 | ACCTGAAATC AGCCGACATC TTCGATGCCG CCCAATATCC |
| | GGACATCCGC |
| 301 | TTTGTTTCCA CCAAATTCAA CTTCAACGGC AAAAAACTGG |
| | TTTCCGTTGA |
| 351 | CGGCAACCTG ACCATGCACG GCAAAACCGC CCCCGTCAAA |
| | CTCAAAGCCG |
| 401 | AAAAATTCAA CTGCTACCAA AGCCCGATGG AGAAAACCGA |
| | AGTTTGTGGC |
| 451 | GGCGACTTCA GCACCACCAT CGACCGCACC AAATGGGGCA |
| | TGGACTACCT |
| 501 | CGTTAACGTT GGTATGACCA AAAGCGTCCG CATCGACATC |
| | CAAATCGAGG |
| 551 | CAGCCAAACA ATAA |

This corresponds to the amino acid sequence <SEQ ID 85; ORF 953>:

m953.pep

| | |
|---|---|
| 1 | MKKIIFAALA AAAISTASAA TYKVDEYHAN ARFAIDHFNT |
| | STNVGGFYGL |
| 51 | TGSVEFDQAK RDGKIDITIP IANLQSGSQH FTDHLKSADI |
| | FDAAQYPDIR |
| 101 | FVSTKFNFNG KKLVSVDGNL TMHGKTAPVK LKAEKFNCYQ |
| | SPMEKTEVCG |
| 151 | GDFSTTIDRT KWGMDYLVNV GMTKSVRIDI QIEAAKQ* |

The following partial DNA sequence was identified in N. meningitidis <SEQ ID 86>:

orf1-1.seq

| | |
|---|---|
| 1 | ATGAAAACAA CCGACAAACG GACAACCGAA ACACACCGCA |
| | AAGCCCCGAA |
| 51 | AACCGGCCGC ATCCGCTTCT CGCCTGCTTA CTTAGCCATA |
| | TGCCTGTCGT |
| 101 | TCGGCATTCT TCCCCAAGCC TGGGCGGGAC ACACTTATTT |
| | CGGCATCAAC |
| 151 | TACCAATACT ATCGCGACTT TGCCGAAAAT AAAGGCAAGT |
| | TTGCAGTCGG |
| 201 | GGCGAAAGAT ATTGAGGTTT ACAACAAAAA AGGGGAGTTG |
| | GTCGGCAAAT |
| 251 | CAATGACAAA AGCCCCGATG ATTGATTTTT CTGTGGTGTC |
| | GCGTAACGGC |
| 301 | GTGGCGGCAT TGGTGGGCGA TCAATATATT GTGAGCGTGG |
| | CACATAACGG |
| 351 | CGGCTATAAC AACGTTGATT TTGGTGCGGA AGGAAGAAAT |
| | CCCGATCAAC |
| 401 | ATCGTTTTAC TTATAAAATT GTGAAACGGA ATAATTATAA |
| | AGCAGGGACT |
| 451 | AAAGGCCATC CTTATGGCGG CGATTATCAT ATGCCGCGTT |
| | TGCATAAATT |
| 501 | TGTCACAGAT GCAGAACCTG TTGAAATGAC CAGTTATATG |
| | GATGGGCGGA |
| 551 | AATATATCGA TCAAAATAAT TACCCTGACC GTGTTCGTAT |
| | TGGGGCAGGC |
| 601 | AGGCAATATT GGCGATCTGA TGAAGATGAG CCCAATAACC |
| | GCGAAAGTTC |
| 651 | ATATCATATT GCAAGTGCGT ATTCTTGGCT CGTTGGTGGC |
| | AATACCTTTG |
| 701 | CACAAAATGG ATCAGGTGGT GGCACAGTCA ACTTAGGTAG |
| | TGAAAAAATT |
| 751 | AAACATAGCC CATATGGTTT TTTACCAACA GGAGGCTCAT |
| | TTGGCGACAG |
| 801 | TGGCTCACCA ATGTTTATCT ATGATGCCCA AAAGCAAAAG |
| | TGGTTAATTA |
| 851 | ATGGGGTATT GCAAACGGGC AACCCCTATA TAGGAAAAAG |
| | CAATGGCTTC |
| 901 | CAGCTGGTTC GTAAAGATTG GTTCTATGAT GAAATCTTTG |
| | CTGGAGATAC |
| 951 | CCATTCAGTA TTCTACGAAC CACGTCAAAA TGGGAAATAC |
| | TCTTTTAACG |
| 1001 | ACGATAATAA TGGCACAGGA AAAATCAATG CCAAACATGA |
| | ACACAATTCT |
| 1051 | CTGCCTAATA GATTAAAAAC ACGAACCGTT CAATTGTTTA |
| | ATGTTTCTTT |
| 1101 | ATCCGAGACA GCAAGAGAAC CTGTTTATCA TGCTGCAGGT |
| | GGTGTCAACA |
| 1151 | GTTATCGACC CAGACTGAAT AATGGAGAAA ATATTTCCTT |
| | TATTGACGAA |
| 1201 | GGAAAAGGCG AATTGATACT TACCAGCAAC ATCAATCAAG |
| | GTGCTGGAGG |
| 1251 | ATTATATTTC CAAGGAGATT TTACGGTCTC GCCTGAAAAT |
| | AACGAAACTT |
| 1301 | GGCAAGGCGC GGGCGTTCAT ATCAGTGAAG ACAGTACCGT |
| | TACTTGGAAA |
| 1351 | GTAAACGGCG TGGCAAACGA CCGCCTGTCC AAAATCGGCA |
| | AAGGCACGCT |
| 1401 | GCACGTTCAA GCCAAAGGGG AAAACCAAGG CTCGATCAGC |
| | GTGGGCGACG |
| 1451 | GTACAGTCAT TTTGGATCAG CAGGCAGACG ATAAAGGCAA |
| | AAAACAAGCC |
| 1501 | TTTAGTGAAA TCGGCTTGGT CAGCGGCAGG GGTACGGTGC |
| | AACTGAATGC |
| 1551 | CGATAATCAG TTCAACCCCG ACAAACTCTA TTTCGGCTTT |
| | CGCGGCGGAC |
| 1601 | GTTTGGATTT AAACGGGCAT TCGCTTTCGT TCCACCGTAT |
| | TCAAAATACC |
| 1651 | GATGAAGGGG CGATGATTGT CAACCACAAT CAAGACAAAG |
| | AATCCACCGT |
| 1701 | TACCATTACA GGCAATAAAG ATATTGCTAC AACCGGCAAT |
| | AACAACAGCT |
| 1751 | TGGATAGCAA AAAAGAAATT GCCTACAACG GTTGGTTTGG |
| | CGAGAAAGAT |
| 1801 | ACGACCAAAA CGAACGGGCG GCTCAACCTT GTTTACCAGC |
| | CCGCCGCAGA |
| 1851 | AGACCGCACC CTGCTGCTTT CCGGCGGAAC AAATTTAAAC |
| | GGCAACATCA |
| 1901 | CGCAAACAAA CGGCAAACTG TTTTTCAGCG GCAGACCAAC |
| | ACCGCACGCC |
| 1951 | TACAATCATT TAAACGACCA TTGGTCGCAA AAAGAGGGCA |
| | TTCCTCGCGG |
| 2001 | GGAAATCGTG TGGGACAACG ACTGGATCAA CCGCACATTT |
| | AAAGCGGAAA |
| 2051 | ACTTCCAAAT TAAAGGCGGA CAGGCGGTGG TTTCCCGCAA |
| | TGTTGCCAAA |
| 2101 | GTGAAAGGCG ATTGGCATTT GAGCAATCAC GCCCAAGCAG |
| | TTTTTGGTGT |
| 2151 | CGCACCGCAT CAAAGCCACA CAATCTGTAC ACGTTCGGAC |
| | TGGACGGGTC |
| 2201 | TGACAAATTG TGTCGAAAAA ACCATTACCG ACGATAAAGT |
| | GATTGCTTCA |
| 2251 | TTGACTAAGA CCGACATCAG CGGCAATGTC GATCTTGCCG |
| | ATCACGCTCA |
| 2301 | TTTAAATCTC ACAGGGCTTG CCACACTCAA CGGCAATCTT |
| | AGTGCAAATG |
| 2351 | GCGATACACG TTATACAGTC AGCCACAACG CCACCCAAAA |
| | CGGCAACCTT |
| 2401 | AGCCTCGTGG GCAATGCCCA AGCAACATTT AATCAAGCCA |
| | CATTAAACGG |
| 2451 | CAACACATCG GCTTCGGGCA ATGCTTCATT TAATCTAAGC |
| | GACCACGCCG |
| 2501 | TACAAAACGG CAGTCTGACG CTTTCCGGCA ACGCTAAGGC |
| | AAACGTAAGC |
| 2551 | CATTCCGCAC TCAACGGTAA TGTCTCCCTA GCCGATAAGG |
| | CAGTATTCCA |
| 2601 | TTTTGAAAGC AGCCGCTTTA CCGGACAAAT CAGCGGCGGC |
| | AAGGATACGG |
| 2651 | CATTACACTT AAAAGACAGC GAATGGACGC TGCCGTCAGG |
| | CACGGAATTA |
| 2701 | GGCAATTTAA ACCTTGACAA CGCCACCATT ACACTCAATT |
| | CCGCCTATCG |
| 2751 | CCACGATGCG GCAGGGGCGC AAACCGGCAG TGCGACAGAT |
| | GCGCCGCGCC |
| 2801 | GCCGTTCGCG CCGTTCGCGC CGTTCCCTAT TATCCGTTAC |
| | ACCGCCAACT |
| 2851 | TCGGTAGAAT CCCGTTTCAA CACGCTGACG GTAAACGGCA |
| | AATTGAACGG |
| 2901 | TCAGGGAACA TTCCGCTTTA TGTCGGAACT CTTCGGCTAC |
| | CGCAGCGACA |
| 2951 | AATTGAAGCT GGCGGAAAGT TCCGAAGGCA CTTACACCTT |
| | GGCGGTCAAC |
| 3001 | AATACCGGCA ACGAACCTGC AAGCCTCGAA CAATTGACGG |
| | TAGTGGAAGG |
| 3051 | AAAAGACAAC AAACCGCTGT CCGAAAACCT TAATTTCACC |
| | CTGCAAAACG |
| 3101 | AACACGTCGA TGCCGGCGCG TGGCGTTACC AACTCATCCG |
| | CAAAGACGGC |
| 3151 | GAGTTCCGCC TGCATAATCC GGTCAAAGAA CAAGAGCTTT |
| | CCGACAAACT |
| 3201 | CGGCAAGGCA GAAGCCAAAA AACAGGCGGA AAAAGACAAC |
| | GCGCAAAGCC |
| 3251 | TTGACGCGCT GATTGCGGCC GGGCGCGATG CCGTCGAAAA |
| | GACAGAAAGC |
| 3301 | GTTGCCGAAC CGGCCCGGCA GGCAGGCGGG GAAAATGTCG |
| | GCATTATGCA |
| 3351 | GGCGGAGGAA GAGAAAAAAC GGGTGCAGGC GGATAAAGAC |
| | ACCGCCTTGG |
| 3401 | CGAAACAGCG CGAAGCGGAA ACCCGGCCGG CTACCACCGC |
| | CTTCCCCCGC |
| 3451 | GCCCGCCGCG CCCGCCGGGA TTTGCCGCAA CTGCAACCCC |
| | AACCGCAGCC |
| 3501 | CCAACCGCAG CGCGACCTGA TCAGCCGTTA TGCCAATAGC |
| | GGTTTGAGTG |
| 3551 | AATTTTCCGC CACGCTCAAC AGCGTTTTCG CCGTACAGGA |
| | CGAATTAGAC |
| 3601 | CGCGTATTTG CCGAAGACCG CCGCAACGCC GTTTGGACAA |
| | GCGGCATCCG |
| 3651 | GGACACCAAA CACTACCGTT CGCAAGATTT CCGCGCCTAC |
| | CGCCAACAAA |
| 3701 | CCGACCTGCG CCAAATCGGT ATGCAGAAAA ACCTCGGCAG |
| | CGGGCGCGTC |
| 3751 | GGCATCCTGT TTTCGCACAA CCGGACCGAA AACACCTTCG |
| | ACGACGGCAT |
| 3801 | CGGCAACTCG GCACGGCTTG CCCACGGCGC CGTTTTCGGG |
| | CAATACGGCA |
| 3851 | TCGACAGGTT CTACATCGGC ATCAGCGCGG GCGCGGGTTT |
| | TAGCAGCGGC |
| 3901 | AGCCTTTCAG ACGGCATCGG AGGCAAAATC CGCCGCCGCG |
| | TGCTGCATTA |
| 3951 | CGGCATTCAG GCACGATACC GCGCCGGTTT CGGCGGATTC |
| | GGCATCGAAC |
| 4001 | CGCACATCGG CGCAACGCGC TATTTCGTCC AAAAAGCGGA |
| | TTACCGCTAC |
| 4051 | GAAAACGTCA ATATCGCCAC CCCCGGCCTT GCATTCAACC |
| | GCTACCGCGC |
| 4101 | GGGCATTAAG GCAGATTATT CATTCAAACC GGCGCAACAC |
| | ATTTCCATCA |
| 4151 | CGCCTTATTT GAGCCTGTCC TATACCGATG CCGCTTCGGG |
| | CAAAGTCCGA |
| 4201 | ACACGCGTCA ATACCGCCGT ATTGGCTCAG GATTTCGGCA |
| | AAACCCGCAG |
| 4251 | TGCGGAATGG GGCGTAAACG CCGAAATCAA AGGTTTCAGG |
| | CTGTCCCTCC |
| 4301 | ACGCTGCCGC CGCCAAAGGC CCGCAACTGG AAGCGCAACA |
| | CAGCGCGGGC |
| 4351 | ATCAAATTAG GCTACCGCTG GTAA |

This corresponds to the amino acid sequence <SEQ ID 87; ORF orf1-1>:

orf1-1.pep

| | |
|---|---|
| 1 | MKTTDKRTTE THRKAPKTGR IRFSPAYLAI CLSFGILPQA |
| | WAGHTYFGIN |
| 51 | YQYYRDFAEN KGKFAVGAKD IEVYNKKGEL VGKSMTKAPM |
| | IDFSVVSRNG |
| 101 | VAALVGDQYI VSVAHNGGYN NVDFGAEGRN PDQHRFTYKI |
| | VKRNNYKAGT |
| 151 | KGHPYGGDYH MPRLHKFVTD AEPVEMTSYM DGRKYIDQNN |
| | YPDRVRIGAG |
| 201 | RQYWRSDEDE PNNRESSYHI ASAYSWLVGG NTFAQNGSGG |
| | GTVNLGSEKI |
| 251 | KHSPYGFLPT GGSFGDSGSP MFIYDAQKQK WLINGVLQTG |
| | NPYIGKSNGF |
| 301 | QLVRKDWFYD EIFAGDTHSV FYEPRQNGKY SFNDDNNGTG |
| | KINAKHEHNS |
| 351 | LPNRLKTRTV QLFNVSLSET AREPVYHAAG GVNSYRPRLN |
| | NGENISFIDE |
| 401 | GKGELILTSN INQGAGGLYF QGDFTVSPEN NETWQGAGVH |
| | ISEDSTVTWK |
| 451 | VNGVANDRLS KIGKGTLHVQ AKGENQGSIS VGDGTVILDQ |
| | QADDKGKKQA |
| 501 | FSEIGLVSGR GTVQLNADNQ FNPDKLYFGF RGGRLDLNGH |
| | SLSFHRIQNT |
| 551 | DEGAMIVNHN QDKESTVTIT GNKDIATTGN NNSLDSKKEI |
| | AYNGWFGEKD |
| 601 | TTKTNGRLNL VYQPAAEDRT LLLSGGTNLN GNITQTNGKL |
| | FFSGRPTPHA |
| 651 | YNHLNDHWSQ KEGIPRGEIV WDNDWINRTF KAENFQIKGG |
| | QAVVSRNVAK |
| 701 | VKGDWHLSNH AQAVFGVAPH QSHTICTRSD WTGLTNCVEK |
| | TITDDKVIAS |
| 751 | LTKTDISGNV DLADHAHLNL TGLATLNGNL SANGDTRYTV |
| | SHNATQNGNL |
| 801 | SLVGNAQATF NQATLNGNTS ASGNASFNLS DHAVQNGSLT |
| | LSGNAKANVS |
| 851 | HSALNGNVSL ADKAVFHFES SRFTGQISGG KDTALHLKDS |
| | EWTLPSGTEL |
| 901 | GNLNLDNATI TLNSAYRHDA AGAQTGSATD APRRRSRRSR |
| | RSLLSVTPPT |
| 951 | SVESRFNTLT VNGKLNGQGT FRFMSELFGY RSDKLKLAES |
| | SEGTYTLAVN |
| 1001 | NTGNEPASLE QLTVVEGKDN KPLSENLNFT LQNEHVDAGA |
| | WRYQLIRKDG |
| 1051 | EFRLHNPVKE QELSDKLGKA EAKKQAEKDN AQSLDALIAA |
| | GRDAVEKTES |
| 1101 | VAEPARQAGG ENVGIMQAEE EKKRVQADKD TALAKQREAE |
| | TRPATTAFPR |
| 1151 | ARRARRDLPQ LQPQPQPQPQ RDLISRYANS GLSEFSATLN |
| | SVFAVQDELD |
| 1201 | RVFAEDRRNA VWTSGIRDTK HYRSQDFRAY RQQTDLRQIG |
| | MQKNLGSGRV |
| 1251 | GILFSHNRTE NTFDDGIGNS ARLAHGAVFG QYGIDRFYIG |
| | ISAGAGFSSG |
| 1301 | SLSDGIGGKI RRRVLHYGIQ ARYRAGFGGF GIEPHIGATR |
| | YFVQKADYRY |
| 1351 | ENVNIATPGL AFNRYRAGIK ADYSFKPAQH ISITPYLSLS |
| | YTDAASGKVR |
| 1401 | TRVNTAVLAQ DFGKTRSAEW GVNAEIKGFT LSLHAAAAKG |
| | PQLEAQHSAG |
| 1451 | IKLGYRW* |

The following partial DNA sequence was identified in N. meningitidis <SEQ ID 88>:

orf46-2.seq

| | |
|---|---|
| 1 | TTGGGCATTT CCCGCAAAAT ATCCCTTATT CTGTCCATAC |
| | TGGCAGTGTG |
| 51 | CCTGCCGATG CATGCACACG CCTCAGATTT GGCAAACGAT |
| | TCTTTTATCC |
| 101 | GGCAGGTTCT CGACCGTCAG CATTTCGAAC CCGACGGGAA |
| | ATACCACCTA |
| 151 | TTCGGCAGCA GGGGGGAACT TGCCGAGCGC AGCGGCCATA |
| | TCGGATTGGG |
| 201 | AAAAATACAA AGCCATCAGT TGGGCAACCT GATGATTCAA |
| | CAGGCGGCCA |
| 251 | TTAAAGGAAA TATCGGCTAC ATTGTCCGCT TTTCCGATCA |
| | CGGGCACGAA |
| 301 | GTCCATTCCC CCTTCGACAA CCATGCCTCA CATTCCGATT |
| | CTGATGAAGC |
| 351 | CGGTAGTCCC GTTGACGGAT TTAGCCTTTA CCGCATCCAT |
| | TGGGACGGAT |
| 401 | ACGAACACCA TCCCGCCGAC GGCTATGACG GGCCACAGGG |
| | CGGCGGCTAT |
| 451 | CCCGCTCCCA AAGGCGCGAG GGATATATAC AGCTACGACA |
| | TAAAAGGCGT |
| 501 | TGCCCAAAAT ATCCGCCTCA ACCTGACCGA CAACCGCAGC |
| | ACCGGACAAC |
| 551 | GGCTTGCCGA CCGTTTCCAC AATGCCGGTA GTATGCTGAC |
| | GCAAGGAGTA |
| 601 | GGCGACGGAT TCAAACGCGC CACCCGATAC AGCCCCGAGC |
| | TGGACAGATC |
| 651 | GGGCAATGCC GCCGAAGCCT TCAACGGCAC TGCAGATATC |
| | GTTAAAAACA |
| 701 | TCATCGGCGC GGCAGGAGAA ATTGTCGGCG CAGGCGATGC |
| | CGTGCAGGGC |
| 751 | ATAAGCGAAG GCTCAAACAT TGCTGTCATG CACGGCTTGG |
| | GTCTGCTTTC |
| 801 | CACCGAAAAC AAGATGGCGC GCATCAACGA TTTGGCAGAT |
| | ATGGCGCAAC |
| 851 | TCAAAGACTA TGCCGCAGCA GCCATCCGCG ATTGGGCAGT |
| | CCAAAACCCC |
| 901 | AATGCCGCAC AAGGCATAGA AGCCGTCAGC AATATCTTTA |
| | TGGCAGCCAT |
| 951 | CCCCATCAAA GGGATTGGAG CTGTTCGGGG AAAATACGGC |
| | TTGGGCGGCA |
| 1001 | TCACGGCACA TCCTATCAAG CGGTCGCAGA TGGGCGCGAT |
| | CGCATTGCCG |
| 1051 | AAAGGGAAAT CCGCCGTCAG CGACAATTTT GCCGATGCGG |
| | CATACGCCAA |
| 1101 | ATACCCGTCC CCTTACCATT CCCGAAATAT CCGTTCAAAC |
| | TTGGAGCAGC |
| 1151 | GTTACGGCAA AGAAAACATC ACCTCCTCAA CCGTGCCGCC |
| | GTCAAACGGC |
| 1201 | AAAAATGTCA AACTGGCAGA CCAACGCCAC CCGAAGACAG |
| | GCGTACCGTT |
| 1251 | TGACGGTAAA GGGTTTCCGA ATTTTGAGAA GCACGTGAAA |
| | TATGATACGA |
| 1301 | AGCTCGATAT TCAAGAATTA TCGGGGGGCG GTATACCTAA |
| | GGCTAAGCCT |
| 1351 | GTGTTTGATG CGAAACCGAG ATGGGAGGTT GATAGGAAGC |
| | TTAATAAATT |
| 1401 | GACAACTCGT GAGCAGGTGG AGAAAAATGT TCAGGAAATA |
| | AGGAACGGTA |
| 1451 | ATATAAACAG TAACTTTAGC CAACATGCTC AACTAGAGAG |
| | GGAAATTAAT |
| 1501 | AAACTAAAAT CTGCCGATGA AATTAATTTT GCAGATGGAA |
| | TGGGAAAATT |
| 1551 | TACCGATAGC ATGAATGACA AGGCTTTTAG TAGGCTTGTG |
| | AAATCAGTTA |
| 1601 | AAGAGAATGG CTTCACAAAT CCAGTTGTGG AGTACGTTGA |
| | AATAAATGGA |
| 1651 | AAAGCATATA TCGTAAGAGG AAATAATRGG GTTTTTGCTG |
| | CAGAATACCT |
| 1701 | TGGCAGGATA CATGAATTAA AATTTAAAAA AGTTGACTTT |
| | CCTGTTCCTA |
| 1751 | ATACTAGTTG GAAAAATCCT ACTGATGTCT TGAATGAATC |
| | AGGTAATGTT |
| 1801 | AAGAGACCTC GTTATAGGAG TAAATAA |

This corresponds to the amino acid sequence <SEQ ID 89; ORF orf46-2>:

orf46-2.pep

| | |
|---|---|
| 1 | LGISRKISLI LSILAVCLPM HAHASDLAND SFIRQVLDRQ |
| | HFEPDGKYHL |
| 51 | FGSRGELAER SGHIGLGKIQ SHQLGNLMIQ QAAIKGNIGY |
| | IVRFSDHGHE |
| 101 | VHSPFDNHAS HSDSDEAGSP VDGFSLYRIH WDGYEHHPAD |
| | GYDGPQGGGY |
| 151 | PAPKGARDIY SYDIKGVAQN IRLNLTDNRS TGQRLADRFH |
| | NAGSMLTQGV |
| 201 | GDGFKRATRY SPELDRSGNA AEAFNGTADI VKNIIGAAGE |
| | IVGAGDAVQG |
| 251 | ISEGSNIAVM HGLGLLSTEN KMARINDLAD MAQLKDYAAA |
| | AIRDWAVQNP |
| 301 | NAAQGIEAVS NIFMAAIPIK GIGAVRGKYG LGGITAHPIK |
| | RSQMGAIALP |
| 351 | KGKSAVSDNF ADAAYAKYPS PYHSRNIRSN LEQRYGKENI |
| | TSSTVPPSNG |
| 401 | KNVKLADQRH PKTGVPFDGK GFPNFEKHVK YDTKLDIQEL |
| | SGGGIPKAKP |
| 451 | VFDAKPRWEV DRKLNKLTTR EQVEKNVQEI RNGNINSNFS |
| | QHAQLEREIN |
| 501 | KLKSADEINF ADGMGKFTDS MNDKAFSRLV KSVKENGFTN |
| | PVVEYVEING |
| 551 | KAYIVRGNNR VFAAEYLGRI HELKFKKVDF PVPNTSWKNP |
| | TDVLNESGNV |
| 601 | KRPRYRSK* |

Using the above-described procedures, the following oligonucleotide primers were employed in the polymerase chain reaction (PCR) assay in order to clone the ORFs as indicated:

TABLE 1

Restriction

| | | | |
|---|---|---|---|
| ORF | Primer | Sequence | sites |

| | | | |
|---|---|---|---|
| 279 | Forward | CGCGGATCCCATATG-TTGCCTGCAAT | BamHI-NdeI |
| | | CACGATT | |
| | | <SEQ ID 90> | |
| | Reverse | CCCGCTCGAG-TTTAGAAGCGGGCGGC | XhoI |
| | | AA | |
| | | <SEQ ID 91> | |
| 519 | Forward | CGCGGATCCCATATG-TTCAAATCCTT | BamHI-NdeI |
| | | TGTCGTCA | |
| | | <SEQ ID 92> | |
| | Reverse | CCCGCTCGAG-TTTGGCGGTTTTGCTG | XhoI |
| | | C | |
| | | <SEQ ID 93> | |
| 576 | Forward | CGCGGATCCCATATG-GCCGCCCCCGC | BamHI-NdeI |
| | | ATCT | |
| | | <SEQ ID 94> | |
| | Reverse | CCCGCTCGAG-ATTTACTTTTTTGATG | XhoI |
| | | TCGAC | |
| | | <SEQ ID 95> | |
| 919 | Forward | CGCGGATCCCATATG-TGCCAAAGCAA | BamHI-NdeI |
| | | GAGCATC | |
| | | <SEQ ID 96> | |
| | Reverse | CCCGCTCGAG-CGGGCGGTATTCGGG | XhoI |
| | | <SEQ ID 97> | |
| 121 | Forward | CGCGGATCCCATATG-GAAACACAGCT | BamHI-NdeI |
| | | TTACAT | |
| | | <SEQ ID 98> | |
| | Reverse | CCCGCTCGAG-ATAATAATATCCCGCG | XhoI |
| | | CCC | |
| | | <SEQ ID 99> | |
| 128 | Forward | CGCGGATCCCATATG-ACTGACAACGC | BamHI-NdeI |
| | | ACT | |
| | | <SEQ ID 100> | |
| | Reverse | CCCGCTCGAG-GACCGCGTTGTCGAAA | XhoI |
| | | <SEQ ID 101> | |
| 206 | Forward | CGCGGATCCCATATG-AAACACCGCCA | BamHI-NdeI |
| | | ACCGA | |
| | | <SEQ ID 102> | |
| | Reverse | CCCGCTCGAG-TTCTGTAAAAAAAGTA | XhoI |
| | | TGTGC | |
| | | <SEQ ID 103> | |
| 287 | Forward | CCGGAATTCTAGCTAGC-CTTTCAGCC | EcoRI-NheI |
| | | TGCGGG | |
| | | <SEQ ID 104> | |
| | Reverse | CCCGCTCGAG-ATCCTGCTCTTTTTTG | XhoI |
| | | CC | |
| | | <SEQ ID 105> | |
| 406 | Forward | CGCGGATCCCATATG-TGCGGGACACT | BamHI-NdeI |
| | | GACAG | |
| | | <SEQ ID 106> | |
| | Reverse | CCCGCTCGAG-AGGTTGTCCTTGTCTA | XhoI |
| | | TG | |
| | | <SEQ ID 107> | |

### EXAMPLE 2

### Expression of ORF 919

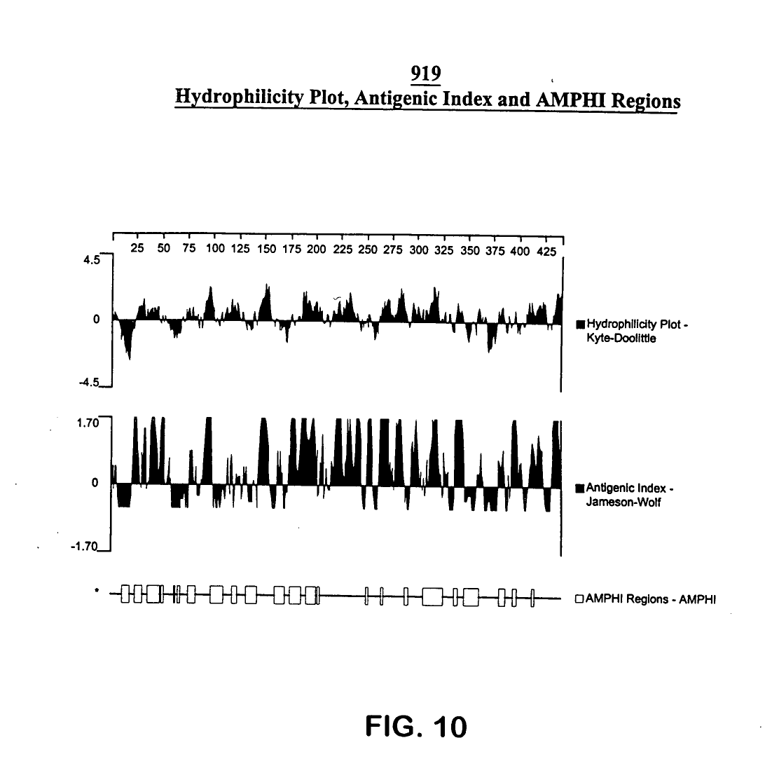

The primer described in Table 1 for ORF 919 was used to locate and clone ORF 919. The predicted gene 919 was cloned in pET vector and expressed in E. coli. The product of protein expression and purification was analyzed by SDS-PAGE. In panel A) is shown the analysis of 919-His fusion protein purification. Mice were immunized with the purified 919-His and sera were used for Western blot (panel B), FACS analysis (panel C), bactericidal assay (panel D), and ELISA assay (panel E). Symbols: M1, molecular weight marker; PP, purified protein, TP, N. meningitidis total protein extract; OMv, N. meningitidis outer membrane vesicle preparation. Arrows indicate the position of the main recombinant protein product (A) and the N. meningitidis immunoreactive band (B). These experiments confirm that 919 is a surface-exposed protein and that it is a useful immunogen. The hydrophilicity plots, antigenic index, and amphipatic regions of ORF 919 are provided in FIG. 10. The AMPHI program is used to predict putative T-cell epitopes (Gao et al 1989, J. Immunol 143:3007; Roberts et al. 1996, AIDS Res Human Retroviruses 12:593; Quakyi et al. 1992, Scand J Immunol Suppl 11:9). The nucleic acid sequence of ORF 919 and the amino acid sequence encoded thereby is provided in Example 1.

### EXAMPLE 3

### Expression of ORF 279

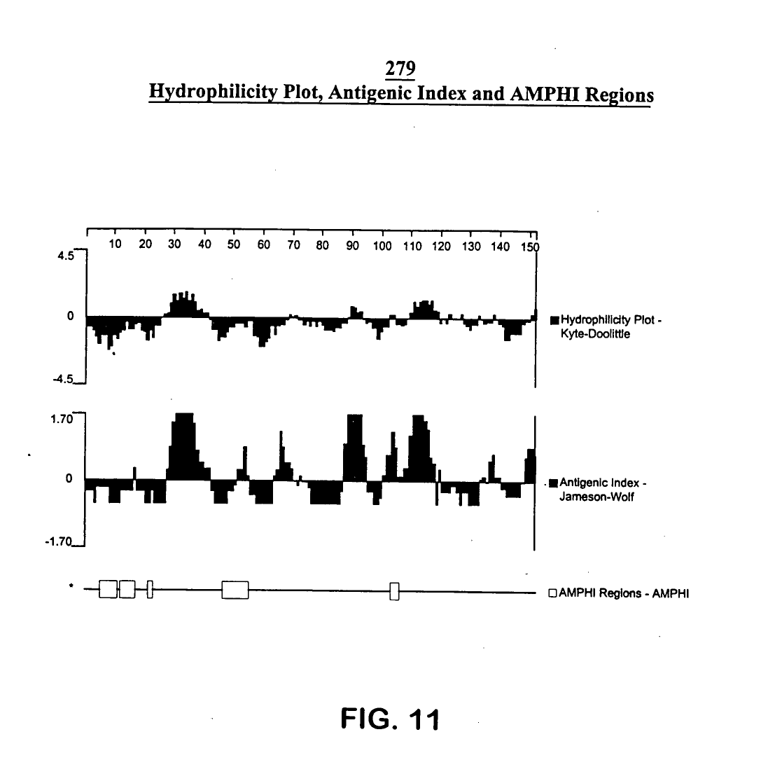

The primer described in Table 1 for ORF 279 was used to locate and clone ORF 279. The predicted gene 279 was cloned in pGex vector and expressed in E. coli. The product of protein expression and purification was analyzed by SDS-PAGE. In panel A) is shown the analysis of 279-GST purification. Mice were immunized with the purified 279-GST and sera were used for Western blot analysis (panel B), FACS analysis (panel C), bactericidal assay (panel D), and ELISA assay (panel E). Symbols: M1, molecular weight marker; TP, N. meningitidis total protein extract; OMV, N. meningitidis outer membrane vescicle preparation. Arrows indicate the position of the main recombinant protein product (A) and the N. meningitidis immunoreactive band (B). These experiments confirm that 279 is a surface-exposed protein and that it is a useful immunogen. The hydrophilicity plots, antigenic index, and amphipatic regions of ORF 279 are provided in FIG. 11. The AMPHI program is used to predict putative T-cell epitopes (Gao et al 1989, J. Immunol 143:3007; Roberts et al. 1996, AIDS Res Human Retroviruses 12:593; Quakyi et al. 1992, Scand J Immunol Suppl 11:9). The nucleic acid sequence of ORF 279 and the amino acid sequence encoded thereby is provided in Example 1.

### EXAMPLE 4

### Expression of ORF 576

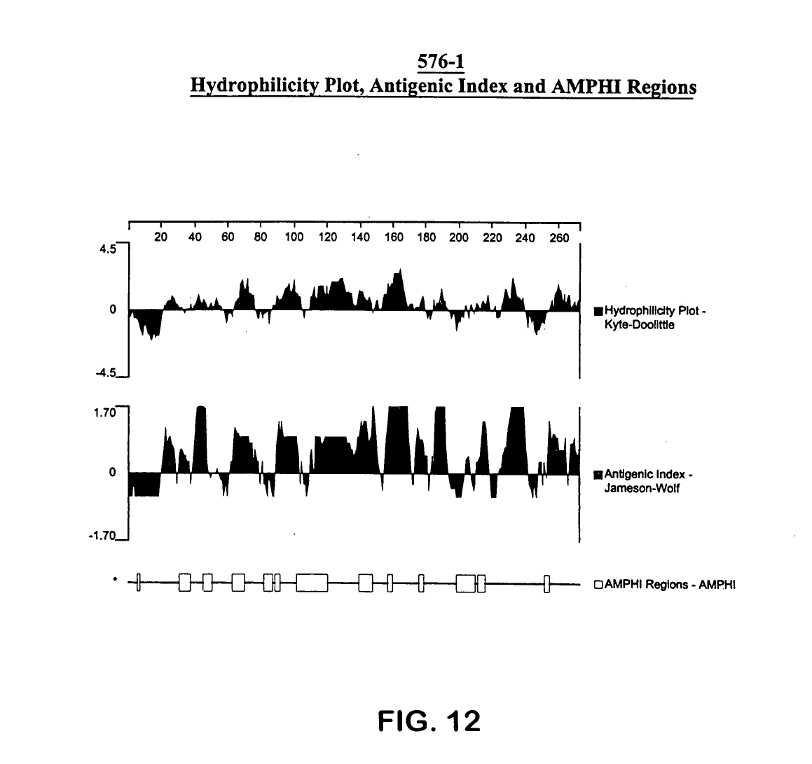

The primer described in Table 1 for ORF 576 was used to locate and clone ORF 576. The predicted gene 576 was cloned in pGex vector and expressed in E. coli. The product of protein purification was analyzed by SDS-PAGE. In panel A) is shown the analysis of 576-GST fusion protein purification. Mice were immunized with the purified 576-GST and sera were used for Western blot (panel B), FACS analysis (panel C), bactericidal assay (panel D), and ELISA assay (panel E). Symbols: M1, molecular weight marker; TP, N. meningitidis total protein extract; OMV, N. meningitidis outer membrane vescicle preparation. Arrows indicate the position of the main recombinant protein product (A) and the N. meningitidis immunoreactive band (B). These experiments confirm that ORF 576 is a surface-exposed protein and that it is a useful immunogen. The hydrophilicity plots, antigenic index, and amphipatic regions of ORF 576 are provided in FIG. 12. The AMPHI program is used to predict putative T-cell epitopes (Gao et al 1989, J. Immunol 143:3007; Roberts et al. 1996, AIDS Res Human Retroviruses 12:593; Quakyi et al. 1992, Scand J Immunol Suppl 11:9). The nucleic acid sequence of ORF 576 and the amino acid sequence encoded thereby is provided in Example 1.

### EXAMPLE 5

### Expression of ORF 519

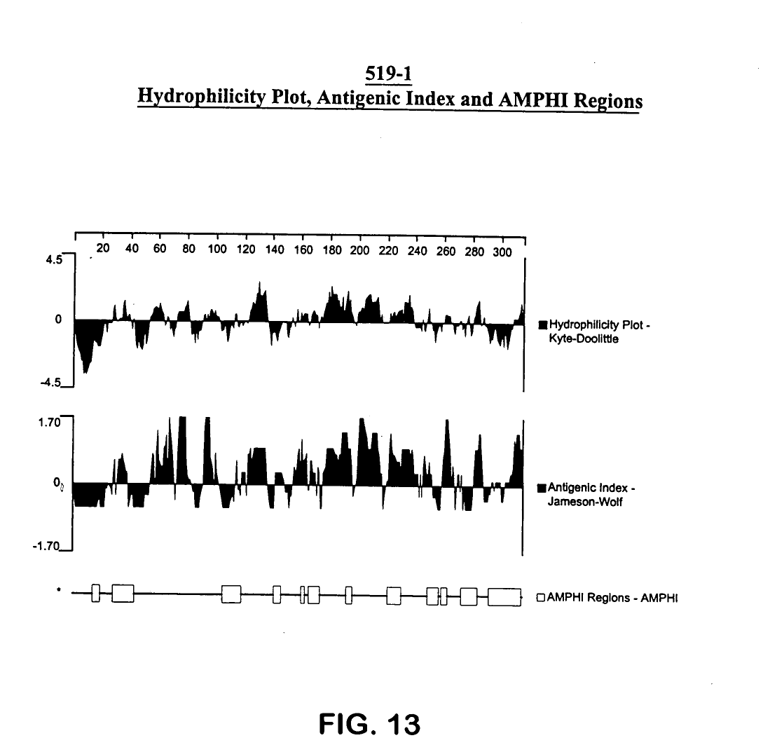

The primer described in Table 1 for ORF 519 was used to locate and clone ORF 519. The predicted gene 519 was cloned in pET vector and expressed in E. coli. The product of protein purification was analyzed by SDS-PAGE. In panel A) is shown the analysis of 519-His fusion protein purification. Mice were immunized with the purified 519-His and sera were used for Western blot (panel B), FACS analysis (panel C), bactericidal assay (panel D), and ELISA assay (panel E). Symbols: M1, molecular weight marker; TP, N. meningitidis total protein extract; OMV, N. meningitidis outer membrane vesicle preparation. Arrows indicate the position of the main recombinant protein product (A) and the N. meningitidis immunoreactive band (B). These experiments confirm that 519 is a surface-exposed protein and that it is a useful immunogen. The hydrophilicity plots, antigenic index, and amphipatic regions of ORF 519 are provided in FIG. 13. The AMPHI program is used to predict putative T-cell epitopes (Gao et al 1989, J. Immunol 143:3007; Roberts et al. 1996, AIDS Res Human Retroviruses 12:593; Quakyi et al. 1992, Scand J Immunol Suppl 11:9). The nucleic acid sequence of ORF 519 and the amino acid sequence encoded thereby is provided in Example 1.

### EXAMPLE 6

### Expression of ORF 121

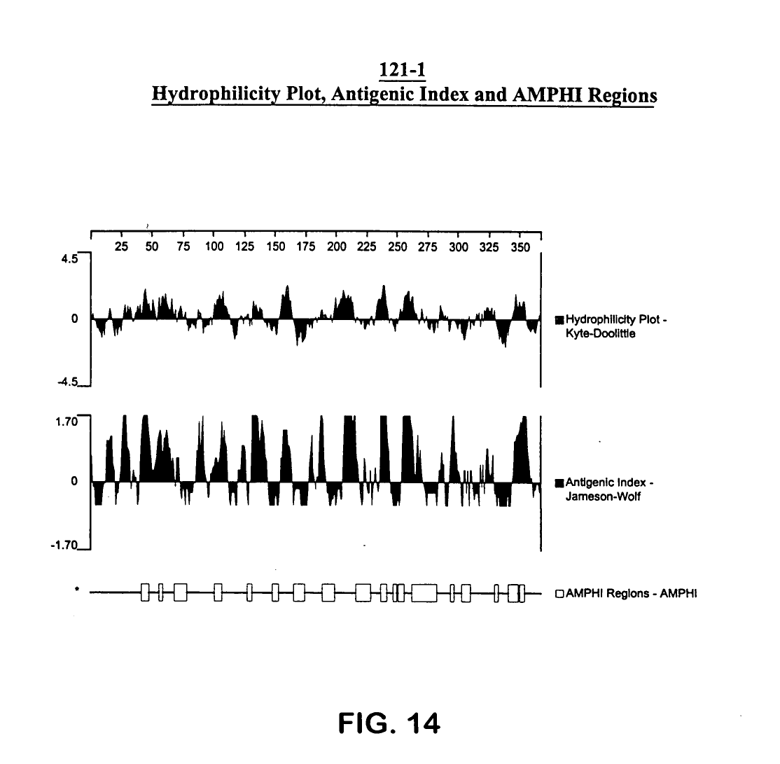

The primer described in Table 1 for ORF 121 was used to locate and clone ORF 121. The predicted gene 121 was cloned in pET vector and expressed in E. coli. The product of protein purification was analyzed by SDS-PAGE. In panel A) is shown the analysis of 121-His fusion protein purification. Mice were immunized with the purified 121-His and sera were used for Western blot analysis (panel B), FACS analysis (panel C), bactericidal assay (panel D), and ELISA assay (panel E). Results show that 121 is a surface-exposed protein. Symbols: M1, molecular weight marker; TP, N. meningitidis total protein extract; OMV, N. meningitidis outer membrane vescicle preparation. Arrows indicate the position of the main recombinant protein product (A) and the N. meningitidis immunoreactive band (B). These experiments confirm that 121 is a surface-exposed protein and that it is a useful immunogen. The hydrophilicity plots, antigenic index, and amphipatic regions of ORF 121 are provided in FIG. 14. The AMPHI program is used to predict putative T-cell epitopes (Gao et al 1989, J. Immunol 143:3007; Roberts et al. 1996, AIDS Res Human Retroviruses 12:593; Quakyi et al. 1992, Scand J Immunol Suppl 11:9). The nucleic acid sequence of ORF 121 and the amino acid sequence encoded thereby is provided in Example 1.

### EXAMPLE 7

### Expression of ORF 128

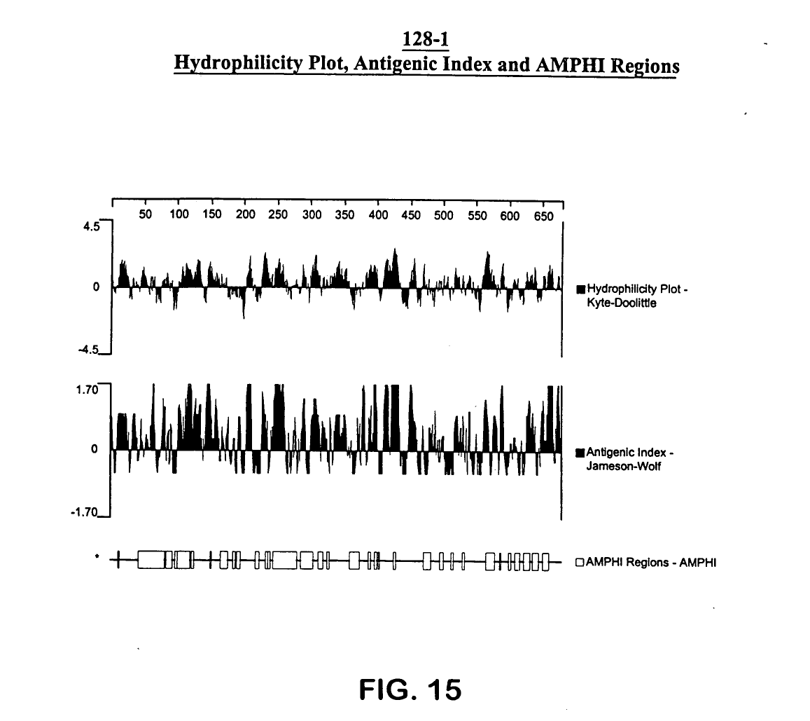

The primer described in Table 1 for ORF 128 was used to locate and clone ORF 128. The predicted gene 128 was cloned in pET vector and expressed in E. coli. The product of protein purification was analyzed by SDS-PAGE. In panel A) is shown the analysis of 128-His purification. Mice were immunized with the purified 128-His and sera were used for Western blot analysis (panel B), FACS analysis (panel C), bactericidal assay (panel D) and ELISA assay (panel E). Results show that 128 is a surface-exposed protein. Symbols: M1, molecular weight marker; TP, N. meningitidis total protein extract; OMV, N. meningitidis outer membrane vesicle preparation. Arrows indicate the position of the main recombinant protein product (A) and the N. meningitidis immunoreactive band (B). These experiments confirm that 128 is a surface-exposed protein and that it is a useful immunogen. The hydrophilicity plots, antigenic index, and amphipatic regions of ORF 128 are provided in FIG. 15. The AMPHI program is used to predict putative T-cell epitopes (Gao et al 1989, J. Immunol 143:3007; Roberts et al. 1996, AIDS Res Human Retroviruses 12:593; Quakyi et al. 1992, Scand J Immunol Suppl 11:9). The nucleic acid sequence of ORF 128 and the amino acid sequence encoded thereby is provided in Example 1.

### EXAMPLE 8

### Expression of ORF 206

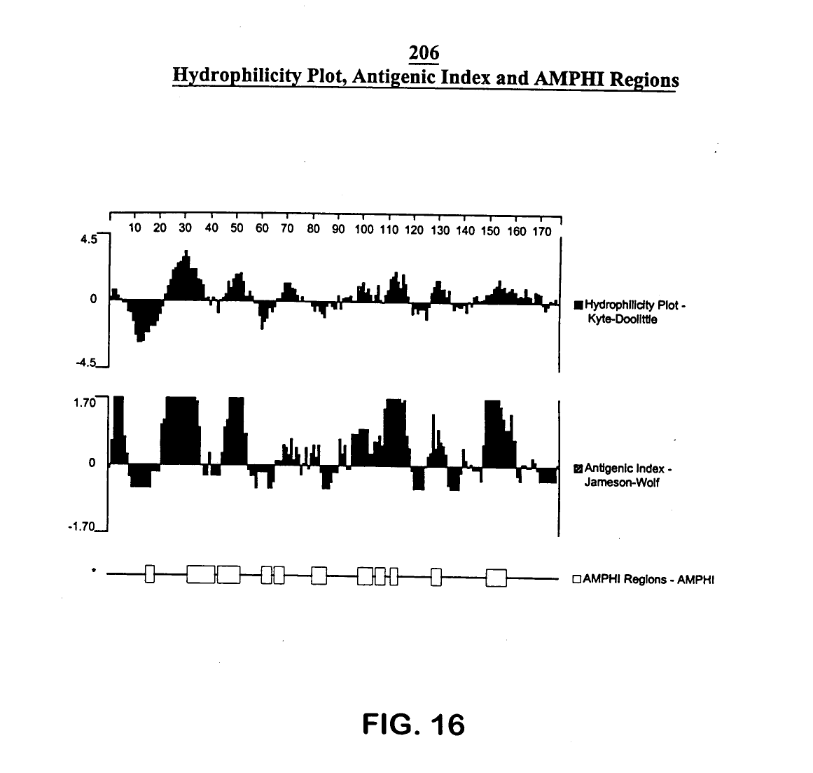

The primer described in Table 1 for ORF 206 was used to locate and clone ORF 206. The predicted gene 206 was cloned in pET vector and expressed in E. coli. The product of protein purification was analyzed by SDS-PAGE. In panel A) is shown the analysis of 206-His purification. Mice were immunized with the purified 206-His and sera were used for Western blot analysis (panel B). It is worth noting that the immunoreactive band in protein extracts from meningococcus is 38 kDa instead of 17 kDa (panel A). To gain information on the nature of this antibody staining we expressed ORF 206 in E. coli without the His-tag and including the predicted leader peptide. Western blot analysis on total protein extracts from E. coli expressing this native form of the 206 protein showed a reactive band at a position of 38 kDa, as observed in meningococcus. We conclude that the 38 kDa band in panel B) is specific and that anti-206 antibodies, likely recognize a multimeric protein complex. In panel C is shown the FACS analysis, in panel D the bactericidal assay, and in panel E) the ELISA assay. Results show that 206 is a surface-exposed protein. Symbols: M1, molecular weight marker; TP, N. meningitidis total protein extract; OMV, N. meningitidis outer membrane vesicle preparation. Arrows indicate the position of the main recombinant protein product (A) and the N. meningitidis immunoreactive band (B). These experiments confirm that 206 is a surface-exposed protein and that it is a useful immunogen. The hydrophilicity plots, antigenic index, and amphipatic regions of ORF 519 are provided in FIG. 16. The AMPHI program is used to predict putative T-cell epitopes (Gao et al 1989, J. Immunol 143:3007; Roberts et al. 1996, AIDS Res Human Retroviruses 12:593; Quakyi et al. 1992, Scand J Immunol Suppl 11:9). The nucleic acid sequence of ORF 206 and the amino acid sequence encoded thereby is provided in Example 1.

### EXAMPLE 9

### Expression of ORF 287

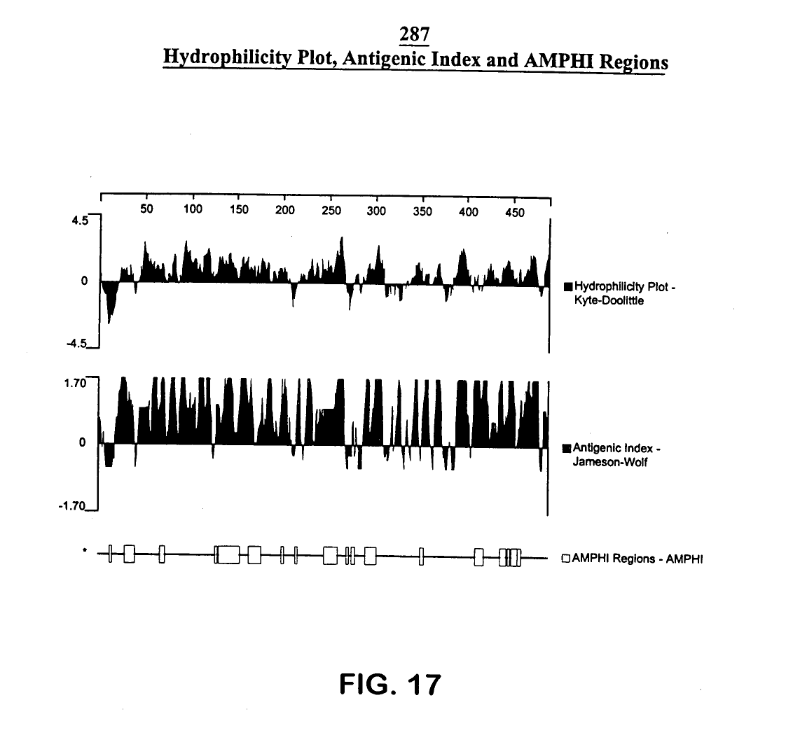

The primer described in Table 1 for ORF 287 was used to locate and clone ORF 287. The predicted gene 287 was cloned in pGex vector and expressed in E. coli. The product of protein purification was analyzed by SDS-PAGE. In panel A) is shown the analysis of 287-GST fusion protein purification. Mice were immunized with the purified 287-GST and sera were used for FACS analysis (panel B), bactericidal assay (panel C), and ELISA assay (panel D). Results show that 287 is a surface-exposed protein. Symbols: M1, molecular weight marker. Arrow indicates the position of the main recombinant protein product (A). These experiments confirm that 287 is a surface-exposed protein and that it is a useful immunogen. The hydrophilicity plots, antigenic index, and amphipatic regions of ORF 287 are provided in FIG. 17. The AMPHI program is used to predict putative T-cell epitopes (Gao et al 1989, J. Immunol 143:3007; Roberts et al. 1996, AIDS Res Human Retroviruses 12:593; Quakyi et al. 1992, Scand J Immunol Suppl 11:9). The nucleic acid sequence of ORF 287 and the amino acid sequence encoded thereby is provided in Example 1.

### EXAMPLE 10

### Expression of ORF 406

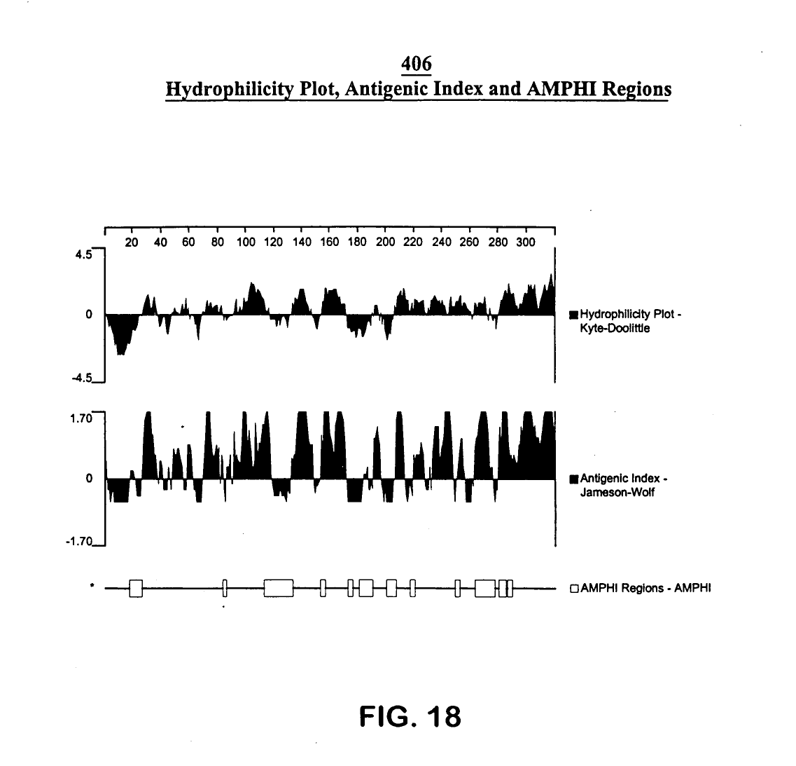

The primer described in Table 1 for ORF 406 was used to locate and clone ORF 406. The predicted gene 406 was cloned in pET vector and expressed in E. coli. The product of protein purification was analyzed by SDS-PAGE. In panel A) is shown the analysis of 406-His fusion protein purification. Mice were immunized with the purified 406-His and sera were used for Western blot analysis (panel B), FACS analysis (panel C), bactericidal assay (panel D), and ELISA assay (panel E). Results show that 406 is a surface-exposed protein. Symbols: M1, molecular weight marker; TP, N. meningitidis total protein extract; OMV, N. meningitidis outer membrane vescicle preparation. Arrows indicate the position of the main recombinant protein product (A) and the N. meningitidis immunoreactive band (B). These experiments confirm that 406 is a surface-exposed protein and that it is a useful immunogen. The hydrophilicity plots, antigenic index, and amphipatic regions of ORF 406 are provided in FIG. 18. The AMPHI program is used to predict putative T-cell epitopes (Gao et al 1989, J. Immunol 143:3007; Roberts et al. 1996, AIDS Res Human Retroviruses 12:593; Quakyi et al. 1992, Scand J Immunol Suppl 11:9). The nucleic acid sequence of ORF 406 and the amino acid sequence encoded thereby is provided in Example 1.

The foregoing examples are intended to illustrate but not to limit the invention.

1. A protein comprising the amino acid sequence encoded by nucleotides 564043 to 565882 of SEQ ID NO 1.

2. A protein comprising an amino acid sequence which sequence has 50% or greater identity to the amino acid sequence encoded by nucleotides 564043 to 565882 of SEQ ID NO 1.

3. A protein comprising a fragment of 7 or more amino acids of the amino acid sequence encoded by nucleotides 564043 to 565882 of SEQ ID NO 1.

4. The protein of claim 3, wherein the fragment comprises an epitope from the amino acid sequence encoded by nucleotides 564043 to 565882 of SEQ ID NO 1.

5. An antibody which binds to a protein according to any one of claims 1 to 4.

6. The antibody of claim 5, wherein the antibody is a monoclonal antibody.

7. A nucleic acid which encodes a protein according to any one of claims 1 to 4.

8. The nucleic acid of claim 7, comprising nucleotides 564043 to 565882 of SEQ ID NO 1.

9. A nucleic acid comprising a fragment of 10 or more nucleotides from within nucleotides 564043 to 565882 of SEQ ID NO 1.

10. A nucleic acid comprising a nucleotide sequence which sequence has 50% or greater identity to nucleotides 564043 to 565882 of SEQ ID NO 1.

11. A nucleic acid comprising a nucleotide sequence complementary to a nucleic acid sequence as defined in claim 7.

12. A nucleic acid comprising a nucleotide sequence complementary to a nucleic acid sequence as defined in claim 9.

13. A nucleic acid comprising a nucleotide sequence complementary to a nucleic acid sequence as defined in claim 10.

14. A nucleic acid which can hybridise to the nucleic acid of claim 7 under high stringency conditions.

15. A nucleic acid which can hybridise to the nucleic acid of claim 9 under high stringency conditions.

16. A nucleic acid which can hybridise to the nucleic acid of claim 10 under high stringency conditions.

17. A composition comprising a protein, a nucleic acid, or an antibody according to claim 1.

18. A composition comprising a protein, a nucleic acid, or an antibody according to claim 2.

19. A composition comprising a protein, a nucleic acid, or an antibody according to claim 3.

20. A protein comprising the amino acid sequence encoded by nucleotides 1812090 to 1812753 of SEQ ID NO 1.

21. A protein comprising an amino acid sequence which sequence has 50% or greater identity to the amino acid sequence encoded by nucleotides 1812090 to 1812753 of SEQ ID NO 1.

22. A protein comprising a fragment of 7 or more amino acids of the amino acid sequence encoded by nucleotides 1812090 to 1812753 of SEQ ID NO 1.

23. The protein of claim 22, wherein the fragment comprises an epitope from the amino acid sequence encoded by nucleotides 1812090 to 1812753 of SEQ ID NO 1.

24. An antibody which binds to a protein according to any one of claims 20 to 23.

25. The antibody of claim 24, wherein the antibody is a monoclonal antibody.

26. A nucleic acid which encodes a protein according to any one of claims 20 to 23.

27. The nucleic acid of claim 26, comprising nucleotides 1812090 to 1812753 of SEQ ID NO 1.

28. A nucleic acid comprising a fragment of 10 or more nucleotides from within nucleotides 1812090 to 1812753 of SEQ ID NO 1.

29. A nucleic acid comprising a nucleotide sequence which sequence has 50% or greater identity to nucleotides 1812090 to 1812753 of SEQ ID NO 1.

30. A nucleic acid comprising a nucleotide sequence complementary to a nucleic acid sequence as defined in any one of claims 20 to 23.

31. A nucleic acid which can hybridise to the nucleic acid of any one of claims 20 to 23 under high stringency conditions.

32. A composition comprising a protein, a nucleic acid, or an antibody according to any one of claims 20 to 23.

33. A composition according to claim 32 being a vaccine composition or a diagnostic composition.
